(19) United States
(12) Patent Application Publication
(10) Pub. No.: US 2024/0246902 A1
(43) Pub. Date: Jul. 25, 2024

(54) PRODRUGS OF 6-DIAZO-5-OXO-L-NORLEUCINE

(71) Applicants: THE JOHNS HOPKINS UNIVERSITY, Baltimore, MD (US); ÚSTAV ORGANICKÉ CHEMIE A BIOCHEMIE AV CR, V.V.I., Prague (CZ)

(72) Inventors: Pavel MAJER, Sykesville, MD (US); Lukas TENORA, Praha (CZ); Katerina NOVOTNA, Dolni Libchavy (CZ); Ivan SNAJDR, Praha (CZ); Barbara SLUSHER, Kingsville, MD (US); Rana RAIS, West Friendship, MD (US); Jesse ALT, Nottingham, MD (US)

(21) Appl. No.: 18/557,760

(22) PCT Filed: Apr. 29, 2022
(86) PCT No.: PCT/US2022/027015

§ 371 Date: Oct. 27, 2023

Related U.S. Application Data
(60) Provisional application No. 63/181,852, filed on Apr. 29, 2021.

(51) Int. Cl. C07C 245/18; A61K 31/655; C07D 209/20; C07D 213/56; C07D 233/06; C07D 401/12; C07D 403/12; C07D 471/18

(52) U.S. Cl. CPC C07C 245/18; A61K 31/655; C07D 209/20; C07D 213/56; C07D 233/06; C07D 401/12; C07D 403/12; C07D 471/18

(57) ABSTRACT

The present disclosure provides prodrugs of 6-diazo-5-oxo-L-norleucine (DON) for use in treating or preventing a disease, disorder, or condition in which the inhibition of glutamine-utilizing enzymes provides a benefit.

### STATEMENT OF GOVERNMENTAL INTEREST

This invention was made with government support under grant numbers NS103927 and CA229451 awarded by the National Institutes of Health. The government has certain rights in the invention.

### BACKGROUND OF THE INVENTION

### Field of Invention

The present disclosure provides prodrugs of 6-diazo-5-oxo-L-norleucine (DON) for use in treating or preventing a disease, disorder, or condition in which the inhibition of glutamine-utilizing enzymes provides a benefit.

### Background

DON is a glutamine antagonist that exhibits promising activity in preclinical models to treat a variety of diseases such as cancer. See, e.g., Ahluwalia et al., Pharmac The. 46:243-371 (1990). But the clinical development of DON has been hampered by its dose-limiting toxicity in humans, especially in the intestinal epithelium. See, e.g., Rosenfeld and Roberts, Cancer Research 41:1324-1328 (1981) and Lynch et al., Am J Clin Oncol (CCT) 5:541-543 (1982). Administering DON as a prodrug may help mitigate this toxicity. See, e.g., Lemberg et al., Mol Cancer Ther 17(9): 1824-1832 (2018).

WO 2017/023774 and WO 2019/071110 disclose prodrugs of DON for the treatment of cancer and other diseases. There exists a need for prodrugs of DON with improved properties for administration to a subject.

### BRIEF SUMMARY OF THE INVENTION

In one aspect, the present disclosure provides compounds represented by any one of Formulae I-III, and the pharmaceutically acceptable salts and solvates, e.g., hydrates, thereof, collectively referred to as “Compounds of the Disclosure.” Compounds of the Disclosure are prodrugs that release 6-diazo-5-oxo-L-norleucine (DON) and thus can be used to treat diseases, disorders, and conditions responsive to the inhibition of glutamine-utilizing enzymes.

In another aspect, the present disclosure provides methods of treating or preventing a disease, disorder, or condition e.g., cancer, in a subject in need thereof comprising administering a therapeutically effective amount of a Compound of the Disclosure to the subject, e.g., a human patient. The disease, disorder, or condition is, for example, cancer, an immune disorder, or a neurological disease.

In another aspect, the present disclosure provides a method of inhibiting glutamine-utilizing enzymes, comprising administering to a subject in need thereof a therapeutically effective amount of a Compound of the Disclosure. Diseases, disorders, or conditions wherein excess and/or aberrant glutamine activity is implicated include, but are not limited to, infection, cancer, autoimmune diseases, neurodegenerative or neurological diseases, and other central nervous system disorders.

In another aspect, the present disclosure provides a Compound of the Disclosure for use in the treatment or prevention of a disease, disorder, or condition in a subject.

In another aspect, the present disclosure provides a use of a Compound of the Disclosure for the manufacture of a medicament for treating or preventing a disease, disorder, or condition in a subject.

In another aspect, the present disclosure provides a pharmaceutical composition comprising a Compound of the Disclosure and a pharmaceutically acceptable carrier. These pharmaceutical compositions are referred to herein as “Compositions of the Disclosure.”

In another aspect, the present disclosure provides a kit comprising a Compound of the Disclosure and instructions, e.g., a package insert, for using the Compound of the Disclosure of treating or preventing a disease, disorder, or condition.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims.

It is to be understood that both the foregoing summary and the following detailed description are exemplary and explanatory only, and are not restrictive of the invention as claimed.

### BRIEF DESCRIPTION OF THE DRAWINGS

FIG. 1A and FIG. 1B are line graphs showing the pharmacokinetics (PK) of Compound 12b in CES1 KO mice. FIG. 1A shows the PK of DON release. FIG. 1B shows the PK of Compound 12b (intact prodrug). Data points represent compound accumulation in tumor, plasma, or jejunum. Errors bars represent standard error of the mean (SEM).

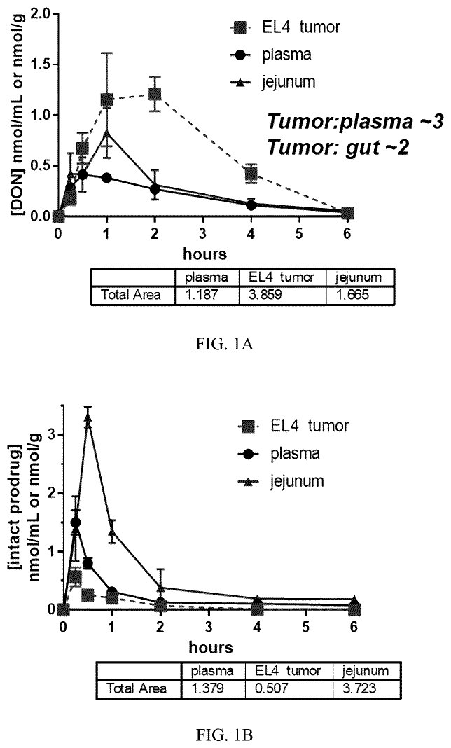

FIG. 2A and FIG. 2B are line graphs showing the PK of Compound 15a in CES1 KO mice. FIG. 2A shows the PK of DON release. FIG. 2B shows the PK of Compound 15a (intact prodrug). Data points represent compound accumulation in tumor, plasma, or jejunum. Errors bars represent standard error of the mean (SEM).

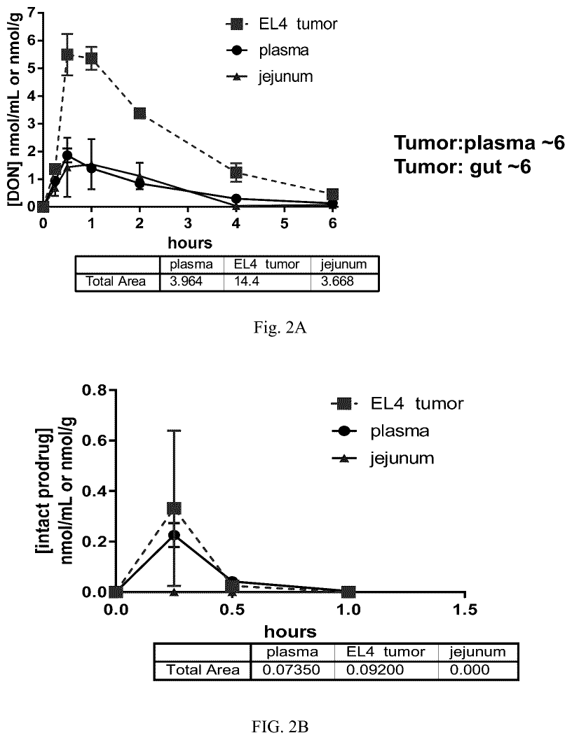

FIG. 3A and FIG. 3B are line graphs showing the pharmacokinetics of Compound 14b in CES1 KO mice. FIG. 3A shows the PK of DON release. FIG. 3B shows the PK of Compound 14b (intact prodrug). Data points represent compound accumulation in tumor, plasma, or jejunum. Errors bars represent standard error of the mean (SEM).

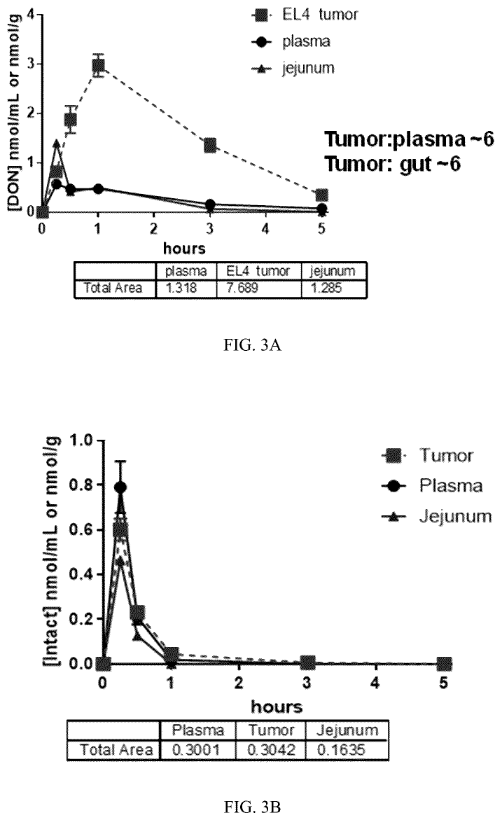

FIG. 4A and FIG. 4B are bar graphs showing amount of DON released from Compound 23a (FIG. 4A) and the amount of intact Compound 23a (FIG. 4B) in at the times indicated in plasma, EL4 tumor, jejunum, and brain.

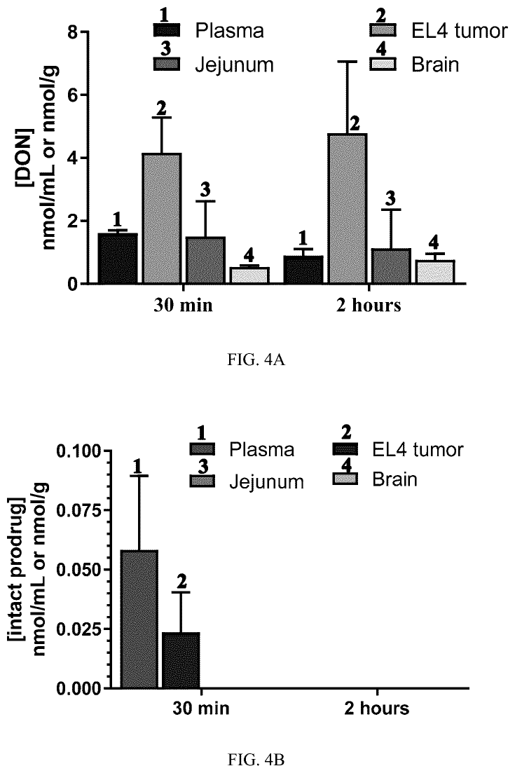

FIG. 5A and FIG. 5B are bar graphs showing amount of DON released from Compound 12d (FIG. 5A) and the amount of intact Compound 12d (FIG. 5B) in at the times indicated in plasma, EL4 tumor, jejunum, and brain.

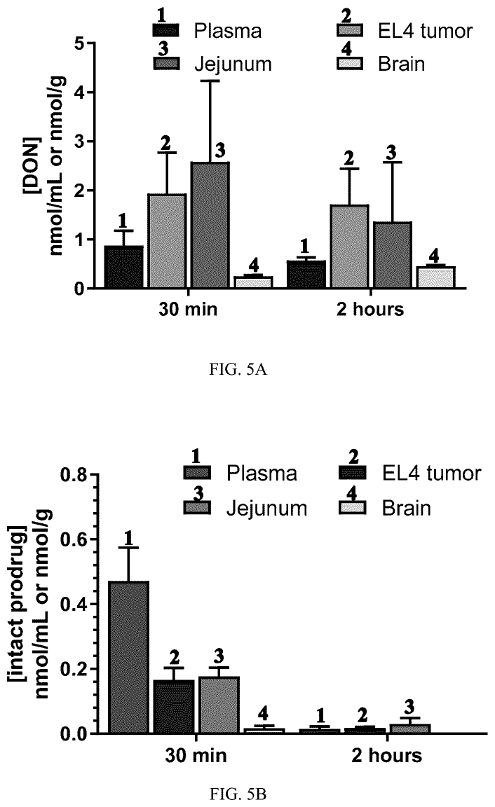

FIG. 6A and FIG. 6B are bar graphs showing amount of DON released from Compound 20a (FIG. 6A) and the amount of intact Compound 12d (FIG. 6B) in at the times indicated in plasma, EL4 tumor, jejunum, and brain.

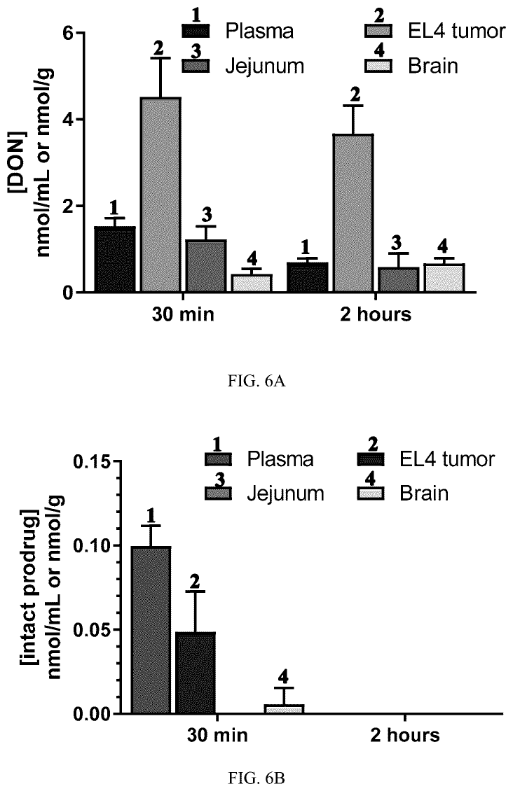

### DETAILED DESCRIPTION OF THE INVENTION

### I. Compounds of the Disclosure

In one embodiment, a Compound of the Disclosure is a compound of Formula I.

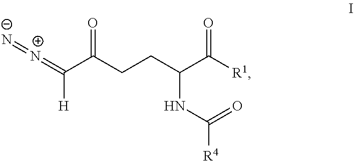

or a pharmaceutically acceptable salt thereof, wherein:
R¹ is selected from the group consisting of —OR² and —NR^{3a}R^{3b};
R² is selected from the group consisting of hydrogen, C₁-C₆ alkyl, C₁-C₆ haloalkyl C₁-C₆ alkenyl, C₃-C₂₀ heteroalkyl, and C₃-C₆ cycloalkyl;
R^{3a} and R^{3b} are independently selected from the group consisting of hydrogen and C₁-C₄ alkyl; or
R^{3a} and R^{3b} taken together with the nitrogen atom to which they are attached from a 4- to 8-membered heterocyclo;
R⁴ is selected from the group consisting of C₁-C₆ alkyl, C₁-C₆ haloalkyl, (amino)C₁-C₆ alkyl, (amino)(aryl)C₁-C₆ alkyl, optionally substituted heteroaryl, —CH(R^{5a})N(R^{6a})C(═O)R^{7a}, —(CH₂)ₘ—N(R^{6c})C(═O)R^{7c}, and —OR⁸;
R^{5a} is selected from the group consisting of hydrogen, optionally substituted C₁-C₆ alkyl, optionally substituted aryl, (heterocyclo)C₁-C₄ alkyl, (aryl)C₁-C₄ alkyl, and (heteroaryl)C₁-C₄ alkyl,
R^{6a} is selected from the group consisting of hydrogen and C₁-C₄ alkyl;
R^{7a} is selected from the group consisting of C₁-C₆ alkyl, C₁-C₆ haloalkyl, optionally substituted C₃-C₅ cycloalkyl, optionally substituted 4- to 10-membered heterocyclo, optionally substituted heteroaryl, (amino)C₁-C₄ alkyl, (heterocyclo)C₁-C₄ alkyl, and —CH(R^{5b})N(R^{6b})C(═O)R^{7b},
R⁸ is optionally substituted 4- to 10-membered heterocyclo;
R^{5b} is selected from the group consisting of hydrogen, optionally substituted C₁-C₆ alkyl, (aryl)C₁-C₄ alkyl, and (heteroaryl)C₁-C₄ alkyl,
R^{6b} is selected from the group consisting of hydrogen and C₁-C₄ alkyl;
R^{7b} is selected from the group consisting of C₁-C₆ alkyl, C₁-C₆ haloalkyl, optionally substituted C₃-C₈ cycloalkyl, optionally substituted 4- to 10-membered heterocyclo, optionally substituted heteroaryl, (amino)C₁-C₄ alkyl, and (heterocyclo)C₁-C₄ alkyl;
R^{6c} is selected from the group consisting of hydrogen and C₁-C₄ alkyl;
R^{7c} is selected from the group consisting of C₁-C₆ alkyl, C₁-C₆ haloalkyl, optionally substituted C₃-C₈ cycloalkyl, optionally substituted 4- to 10-membered heterocyclo, optionally substituted heteroaryl, (amino)C₁-C₄ alkyl, and (heterocyclo)C₁-C₄ alkyl; and
m is 2, 3, 4, or 5.

In another embodiment, the compound of Formula I is not a compound of Table 1.

TABLE 1

| Compound | Structure |
|---|---|
| 1′ | 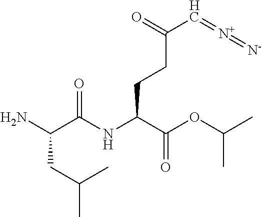 [SMILES: CC(C)C[C@H](N)C(=O)N[C@@H](CCC(=O)C=[N+]=[N-])C(=O)OC(C)C] |
| 2′ | 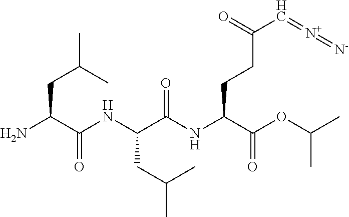 [SMILES: CC(C)C[C@H](NC(=O)[C@@H](N)CC(C)C)C(=O)N[C@@H](CCC(=O)C=[N+]=[N-])C(=O)OC(C)C] |
| 3′ | 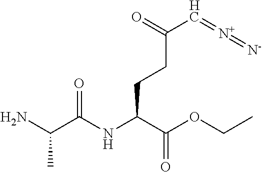 [SMILES: CCOC(=O)[C@H](CCC(=O)C=[N+]=[N-])NC(=O)[C@H](C)N] |
| 4′ | 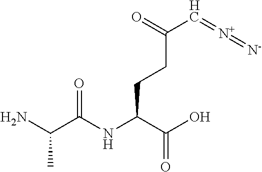 [SMILES: C[C@H](N)C(=O)N[C@@H](CCC(=O)C=[N+]=[N-])C(=O)O] |
| 5′ | 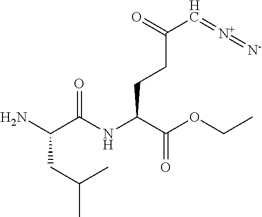 [SMILES: CCOC(=O)[C@H](CCC(=O)C=[N+]=[N-])NC(=O)[C@@H](N)CC(C)C] |
| 6′ | 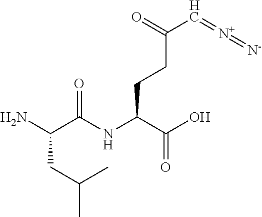 [SMILES: CC(C)C[C@H](N)C(=O)N[C@@H](CCC(=O)C=[N+]=[N-])C(=O)O] |
| 7′ | 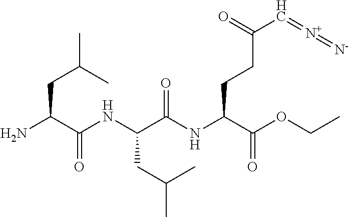 [SMILES: CCOC(=O)[C@H](CCC(=O)C=[N+]=[N-])NC(=O)[C@H](CC(C)C)NC(=O)[C@@H](N)CC(C)C] |
| 8′ | 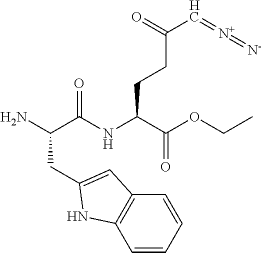 [SMILES: CCOC(=O)[C@H](CCC(=O)C=[N+]=[N-])NC(=O)[C@@H](N)Cc1cc2ccccc2[nH]1] |
| 9′ | 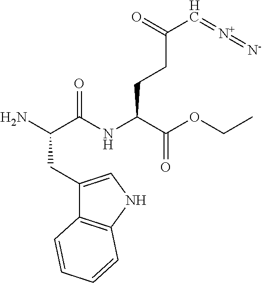 [SMILES: CCOC(=O)[C@H](CCC(=O)C=[N+]=[N-])NC(=O)[C@@H](N)Cc1c[nH]c2ccccc12] |
| 10′ | 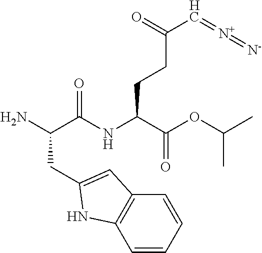 [SMILES: CC(C)OC(=O)[C@H](CCC(=O)C=[N+]=[N-])NC(=O)[C@@H](N)Cc1cc2ccccc2[nH]1] |
| 11′ | 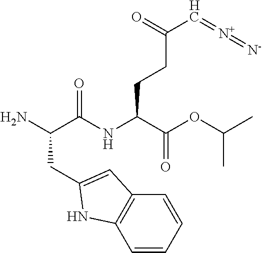 [SMILES: CC(C)OC(=O)[C@H](CCC(=O)C=[N+]=[N-])NC(=O)[C@@H](N)Cc1cc2ccccc2[nH]1] |
| 12′ | 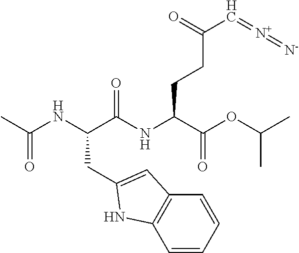 [SMILES: CC(=O)N[C@@H](Cc1cc2ccccc2[nH]1)C(=O)N[C@@H](CCC(=O)C=[N+]=[N-])C(=O)OC(C)C] |
| 13′ | 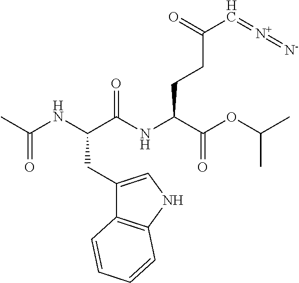 [SMILES: CC(=O)N[C@@H](Cc1c[nH]c2ccccc12)C(=O)N[C@@H](CCC(=O)C=[N+]=[N-])C(=O)OC(C)C] |
| 14′ | 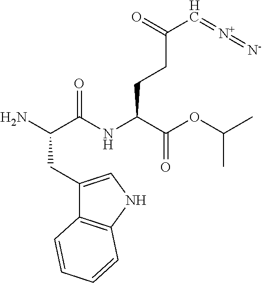 [SMILES: CC(C)OC(=O)[C@H](CCC(=O)C=[N+]=[N-])NC(=O)[C@@H](N)Cc1c[nH]c2ccccc12] |
| 15′ | 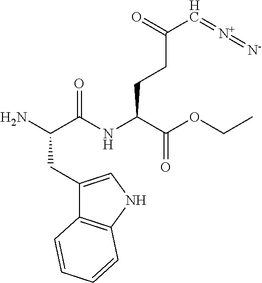 [SMILES: CCOC(=O)[C@H](CCC(=O)C=[N+]=[N-])NC(=O)[C@@H](N)Cc1c[nH]c2ccccc12] |
| 16′ | 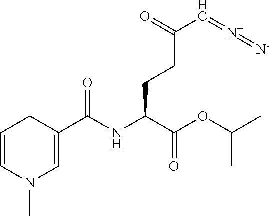 [SMILES: CC(C)OC(=O)[C@H](CCC(=O)C=[N+]=[N-])NC(=O)C1=CN(C)C=CC1] |
| 17′ | 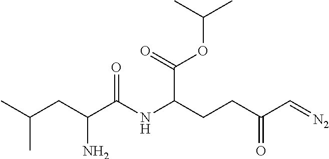 |
| 18′ | 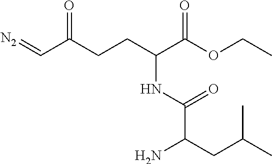 |
| 19′ | 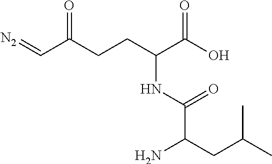 |
| 20′ | 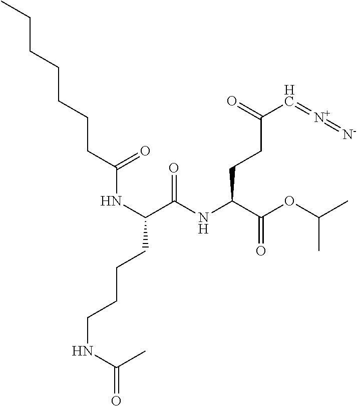 [SMILES: CCCCCCCC(=O)N[C@@H](CCCCNC(C)=O)C(=O)N[C@@H](CCC(=O)C=[N+]=[N-])C(=O)OC(C)C] |
| 21′ | 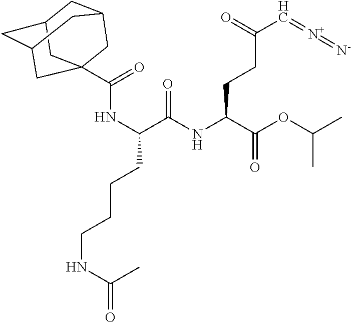 [SMILES: CC(=O)NCCCC[C@H](NC(=O)C12CC3CC(CC(C3)C1)C2)C(=O)N[C@@H](CCC(=O)C=[N+]=[N-])C(=O)OC(C)C] |
| 22′ | 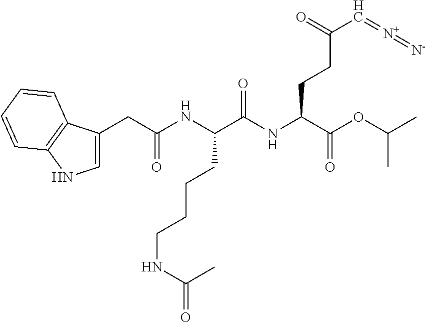 [SMILES: CC(=O)NCCCC[C@H](NC(=O)Cc1c[nH]c2ccccc12)C(=O)N[C@@H](CCC(=O)C=[N+]=[N-])C(=O)OC(C)C] |
| 23′ | 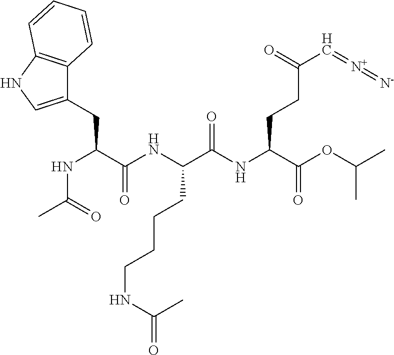 [SMILES: CC(=O)NCCCC[C@H](NC(=O)[C@H](Cc1c[nH]c2ccccc12)NC(C)=O)C(=O)N[C@@H](CCC(=O)C=[N+]=[N-])C(=O)OC(C)C] |
| 24′ | 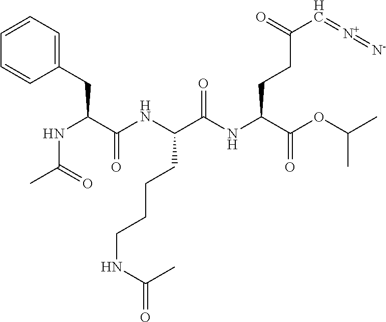 [SMILES: CC(=O)NCCCC[C@H](NC(=O)[C@H](Cc1ccccc1)NC(C)=O)C(=O)N[C@@H](CCC(=O)C=[N+]=[N-])C(=O)OC(C)C] |
| 25′ | 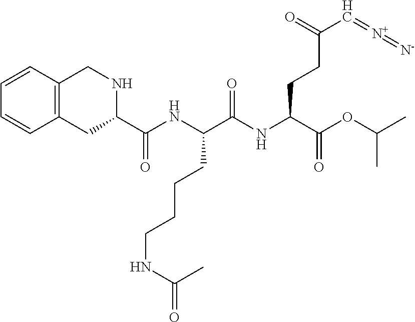 [SMILES: CC(=O)NCCCC[C@H](NC(=O)[C@@H]1Cc2ccccc2CN1)C(=O)N[C@@H](CCC(=O)C=[N+]=[N-])C(=O)OC(C)C] |
| 26′ | 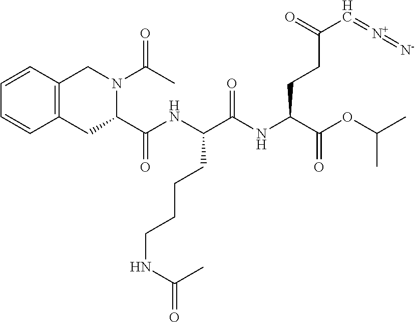 [SMILES: CC(=O)NCCCC[C@H](NC(=O)[C@@H]1Cc2ccccc2CN1C(C)=O)C(=O)N[C@@H](CCC(=O)C=[N+]=[N-])C(=O)OC(C)C] |
| 27′ | 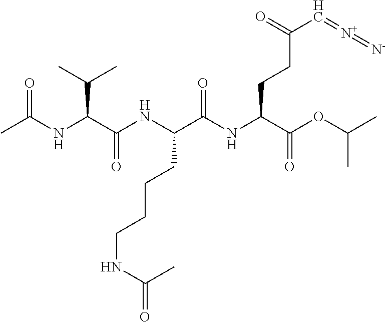 [SMILES: CC(=O)NCCCC[C@H](NC(=O)[C@@H](NC(C)=O)C(C)C)C(=O)N[C@@H](CCC(=O)C=[N+]=[N-])C(=O)OC(C)C] |
| 28′ | 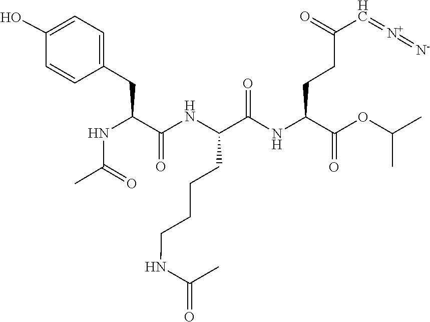 [SMILES: CC(=O)NCCCC[C@H](NC(=O)[C@H](Cc1ccc(O)cc1)NC(C)=O)C(=O)N[C@@H](CCC(=O)C=[N+]=[N-])C(=O)OC(C)C] |
| 29′ | 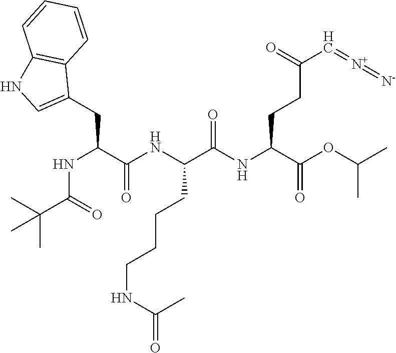 [SMILES: CC(=O)NCCCC[C@H](NC(=O)[C@H](Cc1c[nH]c2ccccc12)NC(=O)C(C)(C)C)C(=O)N[C@@H](CCC(=O)C=[N+]=[N-])C(=O)OC(C)C] |

In another embodiment, a Compound of the Disclosure is a compound of Formula II:

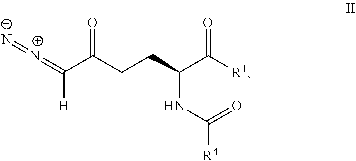

or a pharmaceutically acceptable salt thereof, wherein R¹ and R⁴ are as defined in connection with Formula I.

In another embodiment, a Compound of the Disclosure is a compound of Formula III:

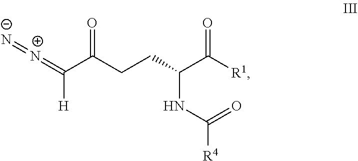

or a pharmaceutically acceptable salt thereof, wherein R¹ and R⁴ are as defined in connection with Formula I.

In another embodiment, a Compound of the Disclosure is a compound of any one of Formulae I-III, or a pharmaceutically acceptable salt thereof, wherein R¹ is —OR². In another embodiment, R² is selected from the group consisting of hydrogen, —CH₃, -CD₃, —CH₂CH₃, —CH(CH₃)₂, —C(CH₃)₃, —CH(CH₂F)₂, —CH₂CH═CH₂, and —OCH₂CH₂OCH₂CH₂OCH₃.

In another embodiment, a Compound of the Disclosure is a compound of any one of Formulae I-III, or a pharmaceutically acceptable salt thereof, wherein R¹ is —NR^{3a}R^{3b}. In another embodiment, R^{3a} and R^{3b} are independently selected from the group consisting of hydrogen and methyl. In another embodiment, R^{3a} and R^{3b} are methyl.

In another embodiment, a Compound of the Disclosure is a compound of any one of Formulae I-III, or a pharmaceutically acceptable salt thereof, wherein R⁴ is C₁-C₆ alkyl. In another embodiment, R⁴ is selected from the group consisting of —CH₃, —CH₂CH₃, and —(CH₂)₄CH₃.

In another embodiment, a Compound of the Disclosure is a compound of any one of Formulae I-III, or a pharmaceutically acceptable salt thereof, wherein R⁴ is C₁-C₆ haloalkyl. In another embodiment, R⁴ is —CHCl₂.

In another embodiment, a Compound of the Disclosure is a compound of any one of Formulae I-III, or a pharmaceutically acceptable salt thereof, wherein R⁴ is (amino)C₁-C₆ alkyl. In another embodiment, R⁴ is —CH₂N(CH₃)₂.

In another embodiment, a Compound of the Disclosure is a compound of any one of Formulae I-III, or a pharmaceutically acceptable salt thereof, wherein R⁴ is optionally substituted heteroaryl. In another embodiment, R⁴ is 2-pyridyl, 3-pyridyl, or 4-pyridyl.

In another embodiment, a Compound of the Disclosure is a compound of any one of Formulae I-III, or a pharmaceutically acceptable salt thereof, wherein R⁴ is —OR⁸. In another embodiment, R⁸ is:

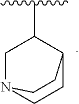 [SMILES: *C1CN2CCC1CC2 ; "."]

In another embodiment, a Compound of the Disclosure is a compound of any one of Formulae I-III, or a pharmaceutically acceptable salt thereof, wherein R⁴ is —(CH₂)ₘ—N(R^{6c})C(═O)R^{7c}. In another embodiment, m is 3. In another embodiment, R^{6c} is hydrogen. In another embodiment, R^{7c} is 4- to 10-membered heterocyclo. In another embodiment, R^{7c} is:

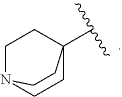 [SMILES: *C12CCN(CC1)CC2 ; "."]

In another embodiment, a Compound of the Disclosure is a compound of any one of Formulae I-III, or a pharmaceutically acceptable salt thereof, wherein R⁴ is —CH(R^{5a})N(R^{6a})C(═O)R^{7a}. In another embodiment, R⁴ is:

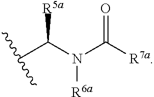

In another embodiment, R⁴ is:

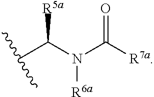

In another embodiment, R^{6a} is hydrogen. In another embodiment, R^{5a} is hydrogen. In another embodiment, R^{5a} is optionally substituted C₁-C₆ alkyl. In another embodiment, R^{5a} is selected from the group consisting of —CH₃, —CH₂CH(CH₃)₂, and —CH₂C(CH₃)₃. In another embodiment, R^{5a} is optionally substituted aryl. In another embodiment, R^{5a} is optionally substituted phenyl. In another embodiment, R^{5a} is (heterocyclo)C₁-C₄ alkyl. In another embodiment, R^{5a} is (aryl)C₁-C₄ alkyl. In another embodiment, R^{5a} is:

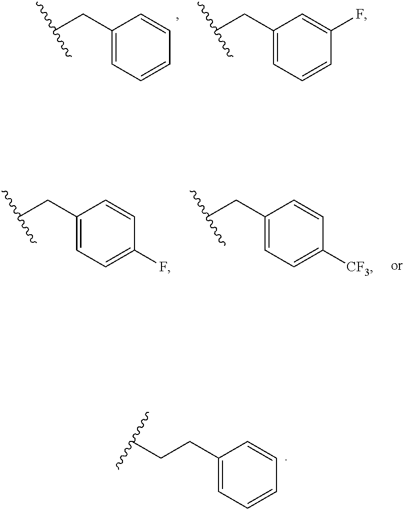

In another embodiment, R^{5a} is (heteroaryl)C₁-C₄ alkyl. In another embodiment, R^{5a} is selected from the group consisting of:

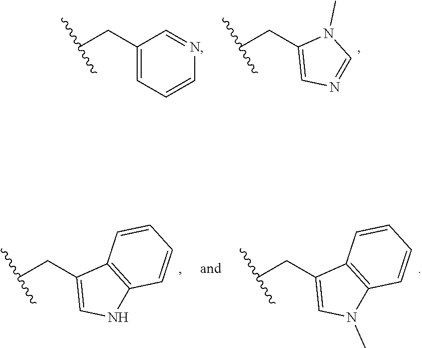

In another embodiment, R^{7a} is C₁-C₄ alkyl. In another embodiment, R^{7a} is selected from the group consisting of —CH₃, —CH(CH₃)₂, —C(CH₃)₃, —CH₂CH₂CH₃, and —CH₂CH(CH₃)₂. In another embodiment, R^{7a} is C₁-C₄ haloalkyl. In another embodiment, R^{7a} is —CHCl₂. In another embodiment, R^{7a} is C₃-C₈ cycloalkyl. In another embodiment, R^{7a} is cyclopropyl. In another embodiment, R^{7a} is optionally substituted 4- to 10-membered heterocyclo. In another embodiment, R^{7a} is selected from the group consisting of:

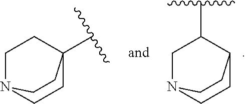 [SMILES: *C12CCN(CC1)CC2 ; "and" ; *C1CN2CCC1CC2 ; "."]

In another embodiment, R^{7a} is (amino)C₁-C₄ alkyl. In another embodiment, R^{7a} is selected from the group consisting of —CH₂N(CH₃)₂ and —CH₂N(CH₂CH₃)₂.

In another embodiment, R^{7a} is (heterocyclo)C₁-C₄ alkyl. In another embodiment, R^{7a} is

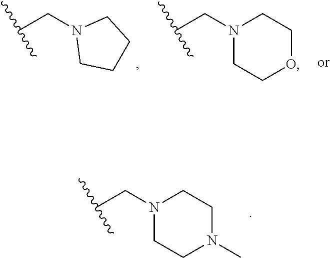

In another embodiment, R^{7a} is —CH(R^{5b})N(R^{6b})C(═O)R^{7b}.

In another embodiment, R^{7a} is —CH₂N(H)C(═O)CH₂N(CH₃)₂.

In another embodiment, a Compound of the Disclosure is a compound of Table 2, or a pharmaceutically acceptable salt thereof.

TABLE 2

| Compound | Structure | Name |
|---|---|---|
| 5a | 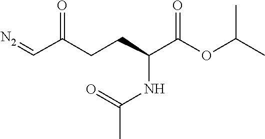 | isopropyl (S)-2-acetamido- 6-diazo-5-oxohexanoate |
| 5b | 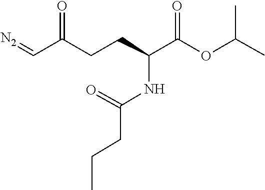 | isopropyl (S)-2- butyramido-6-diazo-5- oxohexanoate |
| 5c | 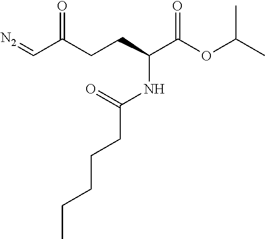 | isopropyl (S)-6-diazo-2- hexanamido-5- oxohexanoate |
| 5d | 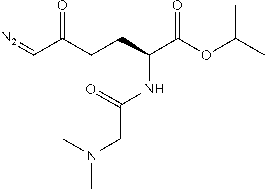 | isopropyl (S)-6-diazo-2-(2- (dimethylamino)acetamido)- 5-oxohexanoate |
| 5e | 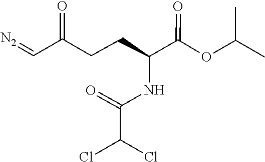 | isopropyl (S)-6-diazo-2- (2,2-dichloroacetamido)-5- oxohexanoate |
| 5f | 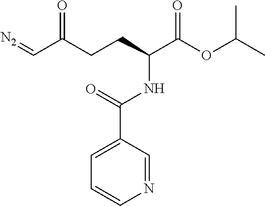 | isopropyl (S)-6-diazo-2- (nicotinamido)-5- oxohexanoate |
| 5g | 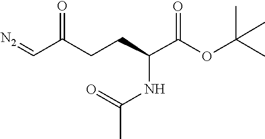 | tert-butyl (S)-2-acetamido- 6-diazo-5-oxohexanoate |
| 5h | 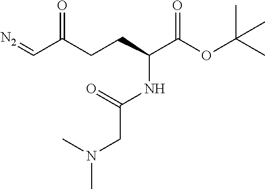 | tert-butyl (S)-6-diazo-2-(2- (dimethylamino)acetamido)- 5-oxohexanoate |
| 5i | 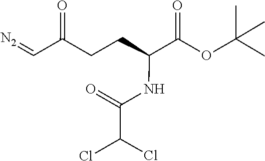 | tert-butyl (S)-6-diazo-2- (2,2-dichloroacetamido)-5- oxohexanoate |
| 5j | 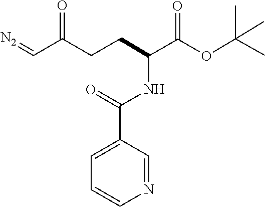 | tert-butyl (S)-6-diazo-2- (nicotinamido)-5- oxohexanoate |
| 5k | 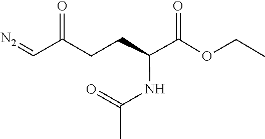 | ethyl (S)-2-acetamido-6- diazo-5-oxohexanoate |
| 5l | 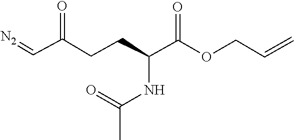 | allyl (S)-2-acetamido-6- diazo-5-oxohexanoate |
| 5m | 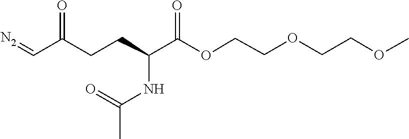 | 2-(2-methoxyethoxy)ethyl (S)-2-acetamido-6-diazo-5- oxohexanoate |
| 8a | 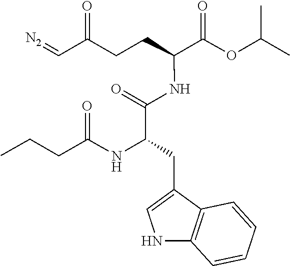 | isopropyl (S)-2-((S)-2- butyramido-3-(1H-indol-3- yl)propanamido)-6-diazo- 5-oxohexanoate |
| 8b | 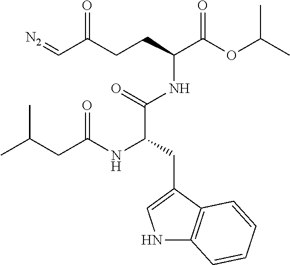 | isopropyl (S)-2-((S)-3-(1H- indol-3-y1)-2-(3- methylbutanamido) propanamido)-6-diazo-5- oxohexanoate |
| 8c | 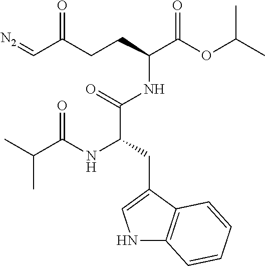 | isopropyl (2S)-2-(3-(1H- indol-3-yl)-2- isobutyramidopropanamido)- 6-diazo-5-oxohexanoate |
| 8d | 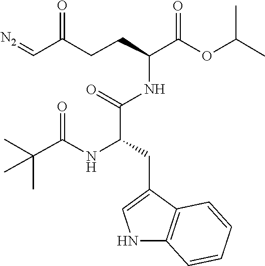 | isopropyl (2S)-2-(3-(1H- indol-3-yl)-2- pivalamidopropanamido)- 6-diazo-5-oxohexanoate |
| 8e | 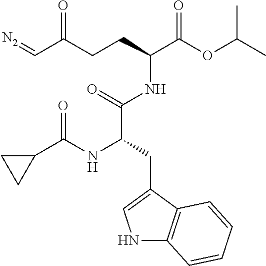 | isopropyl (S)-2-((S)-2- (cyclopropanecarboxamido)- 3-(1H-indol-3- yl)propanamido)-6-diazo- 5-oxohexanoate |
| 8f | 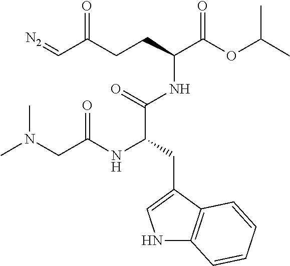 | isopropyl (S)-6-diazo-2- ((S)-2-(2- (dimethylamino)acetamido)- 3-(1H-indol-3- yl)propanamido)-5- oxohexanoate |
| 8g | 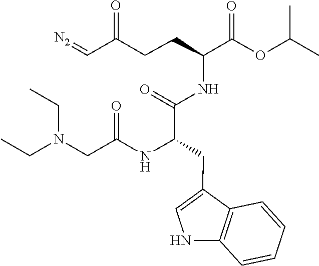 | isopropyl (S)-6-diazo-2- ((S)-2-(2- (diethylamino)acetamido)- 3-(1H-indol-3- yl)propanamido)-5- oxohexanoate |
| 8h | 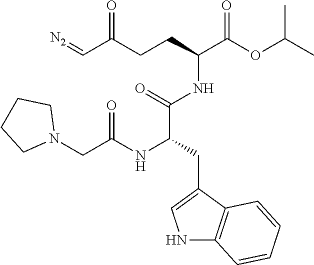 | isopropyl (S)-2-((S)-3-(1H- indol-3-yl)-2-(2- (pyrrolidin-1- yl)acetamido)propanamido)- 6-diazo-5-oxohexanoate |
| 8i | 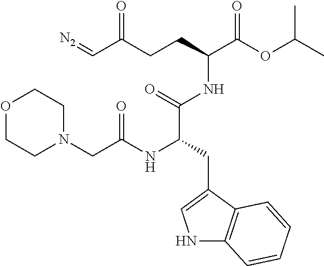 | isopropyl (S)-2-((S)-3-(1H- indol-3-yl)-2-(2- morpholinoacetamido) propanamido)-6-diazo-5- oxohexanoate |
| 8j | 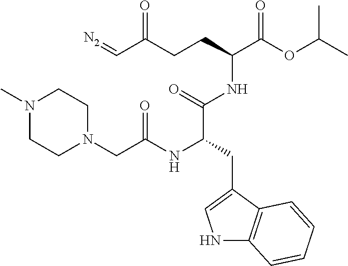 | isopropyl (S)-2-((S)-3-(1H- indol-3-yl)-2-(2-(4- methylpiperazin-1- yl)acetamido)propanamido)- 6-diazo-5-oxohexanoate |
| 8k | 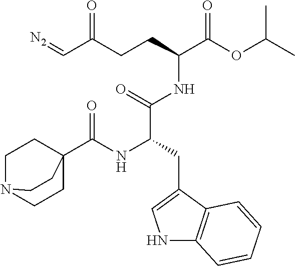 | isopropyl (S)-2-((S)-3-(1H- indol-3-yl)-2-(quinuclidine- 4- carboxamido)propanamido)- 6-diazo-5-oxohexanoate |
| 8l | 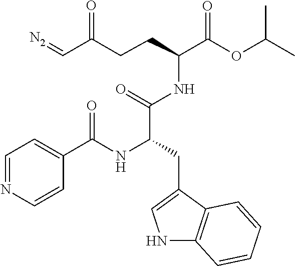 | isopropyl (S)-2-((S)-3-(1H- indol-3-yl)-2- (isonicotinamido) propanamido)-6-diazo-5- oxohexanoate |
| 8m | 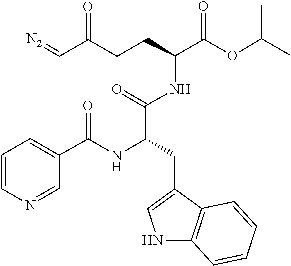 | isopropyl (S)-2-((S)-3-(1H- indol-3-yl)-2- (nicotinamido)propanamido)- 6-diazo-5-oxohexanoate |
| 8n | 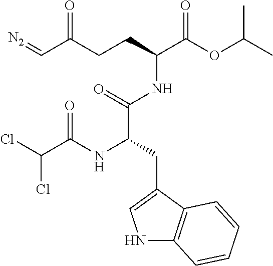 | isopropyl (S)-6-diazo-2- ((S)-2-(2,2- dichloroacetamido)-3-(1H- indol-3-yl)propanamido)-5- oxohexanoate |
| 8o | 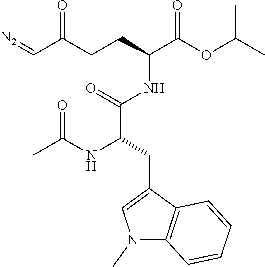 | isopropyl (S)-2-((S)-2- acetamido-3-(1-methyl-1H- indol-3-yl)propanamido)-6- diazo-5-oxohexanoate |
| 8p | 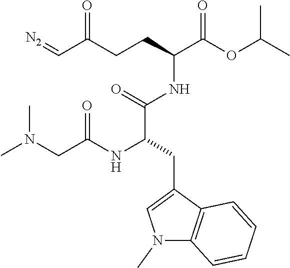 | isopropyl (S)-6-diazo-2- ((S)-2-(2- (dimethylamino)acetamido)- 3-(1-methyl-1H-indol-3- yl)propanamido)-5- oxohexanoate |
| 11a | 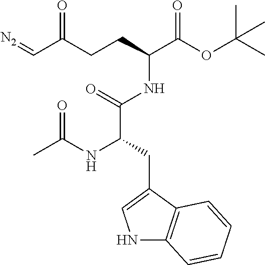 | tert-butyl (S)-2-((S)-2- acetamido-3-(1H-indol-3- yl)propanamido)-6-diazo- 5-oxohexanoate |
| 11b | 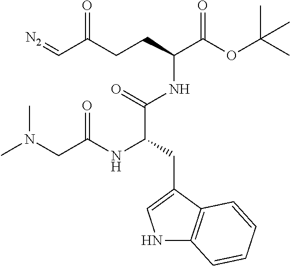 | tert-butyl (S)-6-diazo-2- ((S)-2-(2- (dimethylamino)acetamido)- 3-(1H-indol-3- yl)propanamido)-5- oxohexanoate |
| 11c | 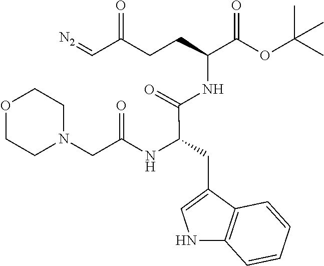 | tert-butyl (S)-2-((S)-3-(1H- indol-3-yl)-2-(2- morpholinoacetamido) propanamido)- 6-diazohexanoate |
| 11d | 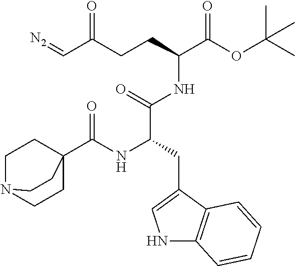 | tert-butyl (S)-2-((S)-3-(1H- indol-3-yl)-2-(quinuclidine- 4- carboxamido)propanamido)- 6-diazo-5-oxohexanoate |
| 12a | 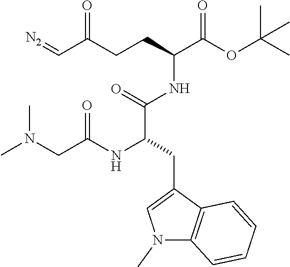 | tert-butyl (S)-6-diazo-2- ((S)-2-(2- (dimethylamino)acetamido)- 3-(1-methyl-1H-indol-3- yl)propanamido)-5- oxohexanoate |
| 12b | 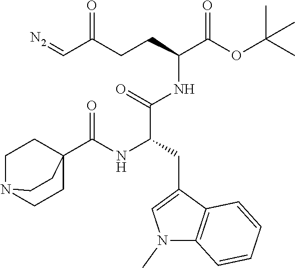 | tert-butyl (S)-6-diazo-2- ((S)-3-(1-methyl-1H-indol- 3-yl)-2-(quinuclidine-4- carboxamido)propanamido)- 5-oxohexanoate |
| 12c | 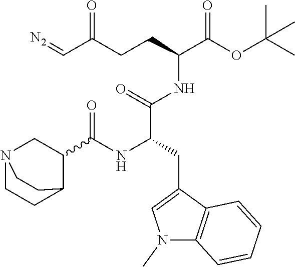 | tert-butyl (2S)-6-diazo-2- ((2S)-3-(1-methyl-1H- indol-3-yl)-2-(quinuclidine- 3- carboxamido)propanamido)- 5-oxohexanoate |
| 12d | 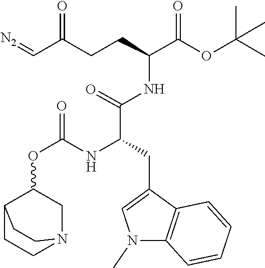 | tert-butyl (2S)-6-diazo-2- ((2S)-3-(1-methyl-1H- indol-3-yl)-2- (((quinuclidin-3- yloxy)carbonyl)amino) propanamido)- 5-oxohexanoate |
| 12g | 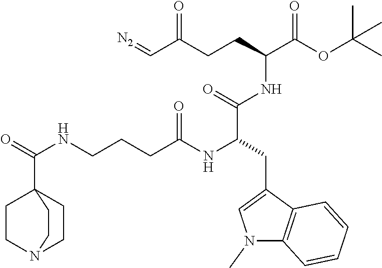 | tert-butyl (S)-6-diazo-2- ((S)-3-(1-methyl-1H-indol- 3-yl)-2-(4-(quinuclidine-4- carboxamido)butanamido) propanamido)-5- oxohexanoate |
| 12h | 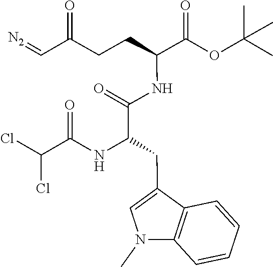 | tert-butyl (S)-6-diazo-2- ((S)-2-(2,2- dichloroacetamido)-3-(1- methyl-1H-indol-3- yl)propanamido)-5- oxohexanoate |
| 12k | 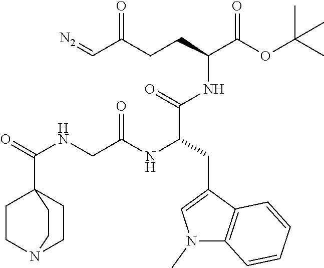 | tert-butyl (S)-6-diazo-2- ((S)-3-(1-methyl-1H-indol- 3-yl)-2-(2-(quinuclidine-4- carboxamido)acetamido) propanamido)-5- oxohexanoate |
| 13a | 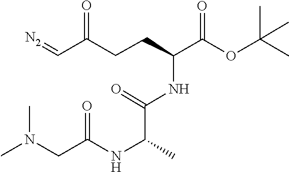 | tert-butyl (S)-6-diazo-2- ((S)-2-(2- (dimethylamino)acetamido) propanamido)-5- oxohexanoate |
| 14a | 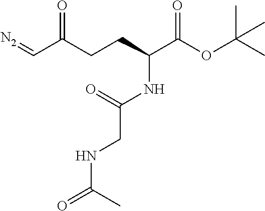 | tert-butyl (S)-2-(2- acetamidoacetamido)-6- diazo-5-oxohexanoate |
| 14b | 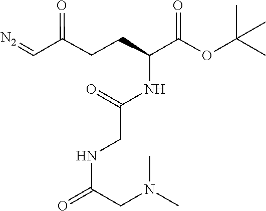 | tert-butyl (S)-6-diazo-2-(2- (2- (dimethylamino)acetamido) acetamido)-5-oxohexanoate |
| 14c | 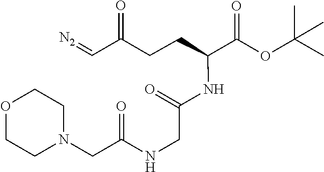 | tert-butyl (S)-6-diazo-2-(2- (2- morpholinoacetamido) acetamido)-5-oxohexanoate |
| 14d | 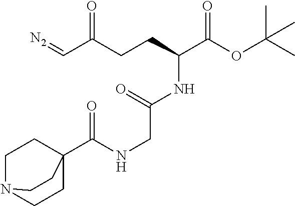 | tert-butyl (S)-6-diazo-5- oxo-2-(2-(quinuclidine-4- carboxamido)acetamido) hexanoate |
| 15a | 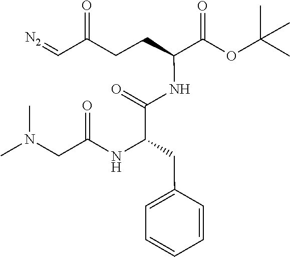 | tert-butyl (S)-6-diazo-2- ((S)-2-(2- (dimethylamino)acetamido)- 3-phenylpropanamido)-5- oxohexanoate |
| 15b | 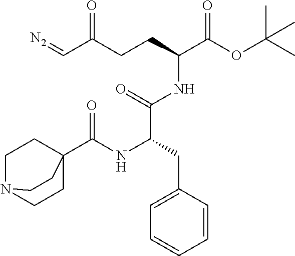 | tert-butyl (S)-6-diazo-5- oxo-2-((S)-3-phenyl-2- (quinuclidine-4- carboxamido)propanamido) hexanoate |
| 15c | 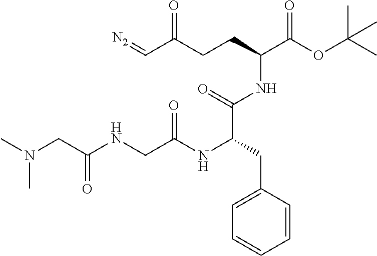 | tert-butyl (9S,12S)-9- benzyl-12-(4-diazo-3- oxobutyl)-2-methyl-4,7,10- trioxo-2,5,8,11- tetraazatridecan-13-oate |
| 16a | 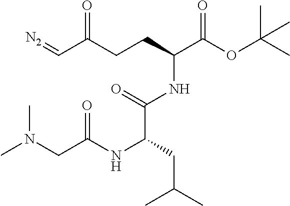 | tert-butyl (S)-6-diazo-2- ((S)-2-(2- (dimethylamino)acetamido)- 4-methylpentanamido)-5- oxohexanoate |
| 17a | 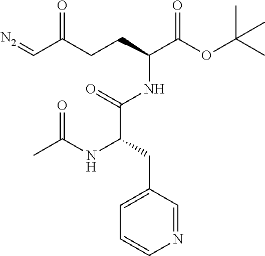 | tert-butyl (S)-2-((S)-2- acetamido-3-(pyridin-3- yl)propanamido)-6-diazo- 5-oxohexanoate |
| 17b | 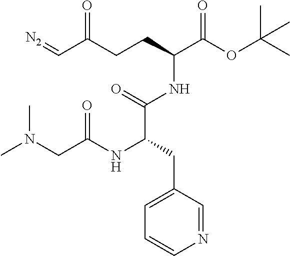 | tert-butyl (S)-6-diazo-2- ((S)-2-(2- (dimethylamino)acetamido)- 3-(pyridin-3- yl)propanamido)-5- oxohexanoate |
| 18a | 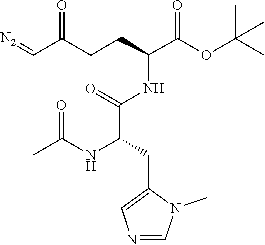 | tert-butyl (S)-2-((S)-2- acetamido-3-(1-methyl-1H- imidazol-5- yl)propanamido)-6-diazo- 5-oxohexanoate |
| 18b | 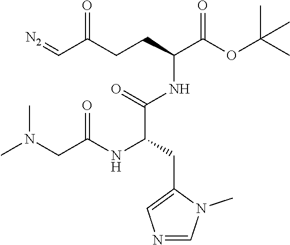 | tert-butyl (S)-6-diazo-2- ((S)-2-(2- (dimethylamino)acetamido)- 3-(1-methyl-1H-imidazol- 5-yl)propanamido)-5- oxohexanoate |
| 19a | 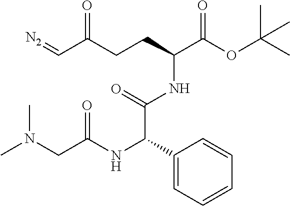 | tert-butyl (S)-6-diazo-2- ((S)-2-(2- (dimethylamino)acetamido)- 2-phenylacetamido)-5- oxohexanoate |
| 20a | 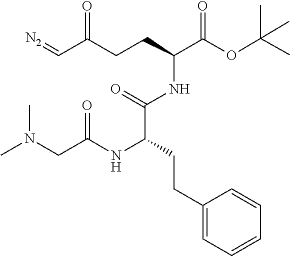 | tert-butyl (S)-6-diazo-2- ((S)-2-(2- (dimethylamino)acetamido)- 4-phenylbutanamido)-5- oxohexanoate |
| 21a | 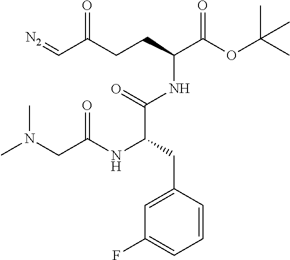 | tert-butyl (S)-6-diazo-2- ((S)-2-(2- (dimethylamino)acetamido)- 3-(3- fluorophenyl)propanamido)- 5-oxohexanoate |
| 22a | 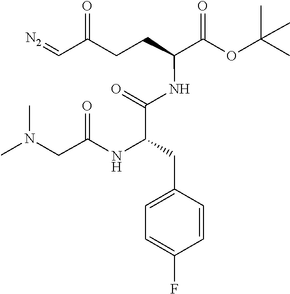 | tert-butyl (S)-6-diazo-2- ((S)-2-(2- (dimethylamino)acetamido)- 3-(4- fluorophenyl)propanamido)- 5-oxohexanoate |
| 23a | 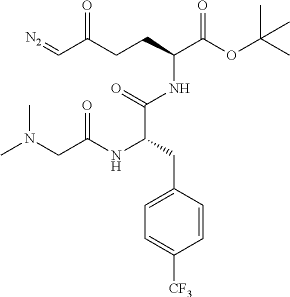 | tert-butyl (S)-6-diazo-2- ((S)-2-(2- (dimethylamino)acetamido)- 3-(4- (trifluoromethyl)phenyl) propanamido)- 5-oxohexanoate |
| 24a | 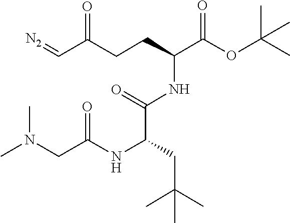 | tert-butyl (S)-6-diazo-2- ((S)-2-(2- (dimethylamino)acetamido)- 4,4- dimethylpentanamido)-5- oxohexanoate |
| 25a | 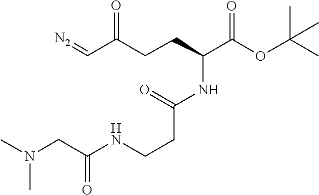 | tert-butyl (S)-6-diazo-2-(3- (2- (dimethylamino)acetamido) propanamido)-5- oxohexanoate |
| 26a | 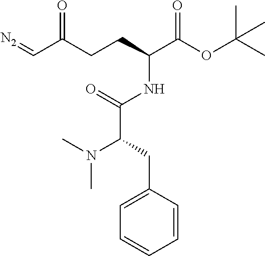 | tert-butyl (S)-6-diazo-2- ((S)-2-(dimethylamino)-3- phenylpropanamido)-5- oxohexanoate |
| 28a | 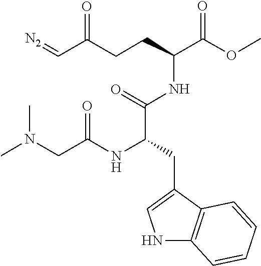 | methyl (S)-6-diazo-2-((S)- 2-(2- (dimethylamino)acetamido)- 3-(1H-indol-3- yl)propanamido)-5- oxohexanoate |
| 28b | 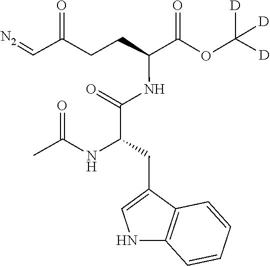 | methyl-d3 (S)-2-((S)-2- acetamido-3-(1H-indol-3- yl)propanamido)-6-diazo- 5-oxohexanoate |
| 28c | 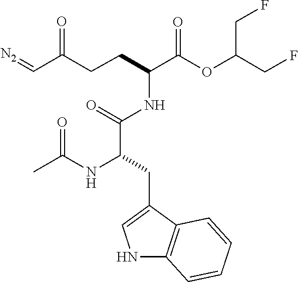 | 1,3-difluoropropan-2-yl (S)-2-((S)-2-acetamido-3- (1H-indol-3- yl)propanamido)-6-diazo- 5-oxohexanoate |
| 30a | 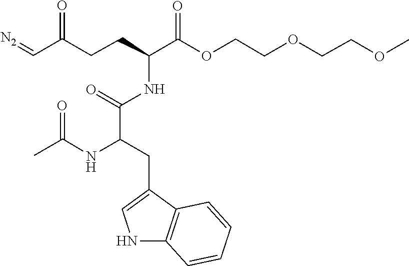 | 2-(2-methoxyethoxy)ethyl (2S)-2-(2-acetamido-3- (1H-indol-3- yl)propanamido)-6-diazo- 5-oxohexanoate |
| 30b | 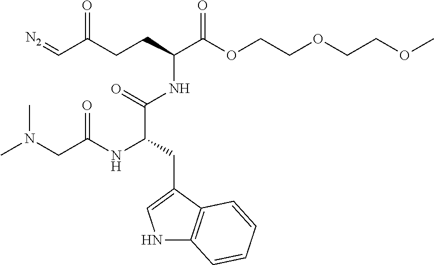 | 2-(2-methoxyethoxy)ethyl (2S)-6-diazo-2-(2-(2- (dimethylamino)acetamido)- 3-(1H-indol-3- yl)propanamido)-5- oxohexanoate |
| 31a | 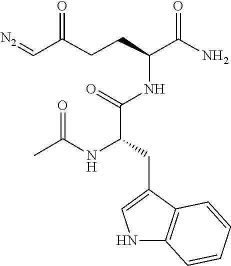 | (S)-2-((S)-2-acetamido-3- (1H-indol-3- yl)propanamido)-6-diazo- 5-oxohexanamide |
| 31b | 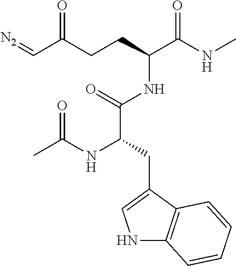 | (S)-2-((S)-2-acetamido-3- (1H-indol-3- yl)propanamido)-6-diazo- N-methyl-5- oxohexanamide |
| 31c | 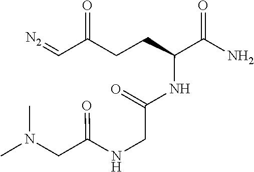 | (S)-6-diazo-2-(2-(2- (dimethylamino)acetamido) acetamido)-5- oxohexanamide |
| 31d | 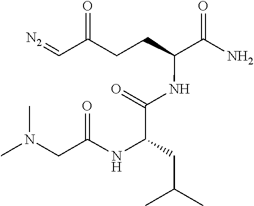 | (S)-6-diazo-2-((S)-2-(2- (dimethylamino)acetamido)- 4-methylpentanamido)-5- oxohexanamide |
| 31e | 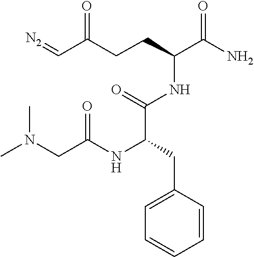 | (S)-6-diazo-2-((S)-2-(2- (dimethylamino)acetamido)- 3-phenylpropanamido)-5- oxohexanamide |
| 31f | 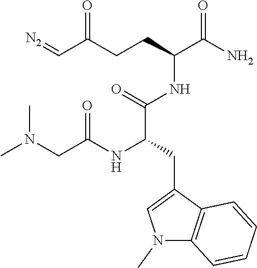 | (S)-6-diazo-2-((S)-2-(2- (dimethylamino)acetamido)- 3-(1-methyl-1H-indol-3- yl)propanamido)-5- oxohexanamide |

A Compound of the Disclosure contains at least one chiral center and thus, in one embodiment, is enantiomerically enriched, e.g., the enantiomeric excess or “ee” of is about 500 or more as measured by chiral HPLC. In another embodiment, the ee is about 1000. In another embodiment, the ee is about 200%. In another embodiment, the ee is about 3000. In another embodiment, the ee is about 40%. In another embodiment, the ee is about 50%. In another embodiment, the ee is about 60%. In another embodiment, the ee is about 70%. In another embodiment, the ee is about 80%. In another embodiment, the ee is about 85%. In another embodiment, the ee is about 90%. In another embodiment, the ee is about 91%. In another embodiment, the ee is about 92%. In another embodiment, the ee is about 93%. In another embodiment, the ee is about 94%. In another embodiment, the ee is about 95%. In another embodiment, the ee is about 96%. In another embodiment, the ee is about 97%. In another embodiment, the ee is about 98%. In another embodiment, the ee is about 99%.

The present disclosure encompasses the preparation and use of pharmaceutically acceptable salts of Compounds of the Disclosure. The term “pharmaceutically acceptable salt” as used herein, refers to any salt, e.g., obtained by reaction with an acid or a base, of a Compound of the Disclosure that is physiologically tolerated in the subject, e.g., a human, or zwitterionic forms of a Compound of the Disclosure. Pharmaceutically acceptable salts of Compounds of the Disclosure can be prepared during the final isolation and purification of the compounds or separately by reacting the compound with a suitable acid. The pharmaceutically acceptable salts of Compounds of the Disclosure can, for example, be acid addition salts formed with pharmaceutically acceptable acids. Examples of acids which can be employed to form pharmaceutically acceptable salts include, but are not limited to, inorganic acids such as nitric, boric, hydrochloric, hydrobromic, sulfuric, and phosphoric, and organic acids such as oxalic, maleic, succinic, and citric. Non-limiting examples of salts of compounds of the disclosure include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulfonate, and p-toluenesulfonate salts. Examples of bases which can be employed to form pharmaceutically acceptable salts include, but are not limited to, alkali metal, e.g., sodium, hydroxides, alkaline earth metal, e.g., magnesium hydroxides, ammonia, and compounds of formula NW₄⁺, wherein W is C₁₋₄ alkyl, and the like.

In addition, available amino groups present in the Compounds of the Disclosure can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference to Compounds of the Disclosure appearing herein is intended to include compounds of Compounds of the Disclosure as well as pharmaceutically acceptable salts, hydrates, or solvates thereof.

The present disclosure also encompasses the preparation and use of solvates of Compounds of the Disclosure. Solvates typically do not significantly alter the physiological activity or toxicity of the compounds, and as such may function as pharmacological equivalents. The term “solvate” as used herein is a combination, physical association and/or solvation of a Compound of the Disclosure with a solvent molecule such as, e.g. a disolvate, monosolvate or hemisolvate, where the ratio of solvent molecule to compound of the present disclosure is about 2:1, about 1:1, or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compounds of the Disclosure can be present as solvated forms with a pharmaceutically acceptable solvent, such as water, methanol, and ethanol, and it is intended that the disclosure includes both solvated and unsolvated forms of Compounds of the Disclosure. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by E. C. van Tonder et al., AAPS Pharm. Sci. Tech., 5 (1): Article 12 (2004), and A. L. Bingham et al., Chem. Commun. 603-604 (2001). Atypical, non-limiting, process of preparing a solvate would involve dissolving a Compound of the Disclosure in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvate in a crystal of the solvate.

### II. Pharmaceutical Compositions

In addition to administering a Compound of the Disclosure as a raw chemical, it may be administered as part of a pharmaceutical composition.

In one embodiment, the disclosure provides a pharmaceutical composition comprising a Compound of the Disclosure and a pharmaceutically acceptable carrier. These pharmaceutical compositions are referred to herein as “Compositions of the Disclosure.” The pharmaceutically acceptable carrier can be selected from one or more pharmaceutically acceptable excipients, vehicles, and/or auxiliaries. The phrases “pharmaceutically acceptable carrier,” “pharmaceutically acceptable vehicle,” and “pharmaceutically acceptable excipient” are used interchangeably and encompass any of the standard pharmaceutical carriers, solvents, surfactants, or vehicles. Suitable pharmaceutically acceptable vehicles include aqueous vehicles, e.g., normal saline, 5% dextrose, lactated Ringer's solution, or any other sterile fluid designed to be compatible with administration, e.g., by intravenous infusion, to a subject, and nonaqueous vehicles. Standard pharmaceutical carriers and their formulations are described in Remington's Pharmaceutical Sciences, Mack Publishing Co., Easton, PA, 19th ed. 1995.

A Composition of the Disclosure can contain from about 0.01 to 99 percent by weight, e.g., from about 0.25 to 75 percent by weight, of a Compound of the Disclosure, e.g., about 1%, about 5%, about 10%, about 15%, about 20%, about 25%, about 30%, about 35%, about 40%, about 45%, about 50%, about 55%, about 60%, about 65%, about 70%, or about 75% by weight of a Compound of the Disclosure.

The pharmaceutical compositions provided herein are manufactured by means of conventional mixing, granulating, Dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries can be suitable flow-regulating agents and lubricants. Suitable auxiliaries include, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions or suspensions of Compounds of the Disclosure. Suitable lipophilic solvents or vehicles include fatty oils, for example, sesame oil, or synthetic fatty acid esters, for example, ethyl oleate or triglycerides or polyethylene glycol-400. Aqueous suspensions may contain substances which increase the viscosity of the suspension including, for example, sodium carboxymethyl cellulose, sorbitol, and/or dextran. Optionally, the suspension may also contain stabilizers.

### III. Therapeutic Methods

In another embodiment, the disclosure provides methods for treating a disease, disorder, or condition in a subject in need thereof, comprising administering a therapeutically effective amount of a Compound of the Disclosure or Composition of the Disclosure to the subject.

In another embodiment, the disclosure provides methods for treating a disease, disorder, or condition in a subject in need thereof, comprising administering a therapeutically effective amount of a Compound of the Disclosure or Composition of the Disclosure to the subject in combination with one or more optional therapeutic agents.

In another embodiment, the disclosure provides a Compound of the Disclosure or Composition of the Disclosure for use in treating a disease, disorder, or condition in a subject.

In another embodiment, the disclosure provides a Compound of the Disclosure or Composition of the Disclosure for use in treating a disease, disorder, or condition in a subject in combination with one or more optional therapeutic agents.

In another embodiment, the disclosure provides the use Compound of the Disclosure or Composition of the Disclosure for the manufacture of a medicament for treating a disease, disorder, or condition in a subject.

In another embodiment, the disclosure provides the use Compound of the Disclosure or Composition of the Disclosure for the manufacture of a medicament for treating a disease, disorder, or condition in a subject in combination with one or more optional therapeutic agents.

In another embodiment, the Compound of the Disclosure or Composition of the Disclosure is administered parenterally to the subject. In another embodiment, the Compound of the Disclosure or Composition of the Disclosure is administered intravenously to the subject. In another embodiment, the Compound of the Disclosure or Composition of the Disclosure is administered subcutaneously to the subject.

In another embodiment, the Compound of the Disclosure or Composition of the Disclosure is administered to the subject according to an intermittent dosing schedule. For example, the Pharmaceutical Formulation of the Disclosure may be administered to a subject three days a week on non-consecutive days, e.g., Monday-Wednesday-Friday, or five days a week, e.g., Monday through Friday.

In another embodiment, the disease, disorder, or condition is a neurodegenerative or neurological disorder. A “neurodegenerative disorder” is a disease, disorder, or condition that is characterized by the progressive loss of the structure or function of neurons (e.g., degeneration or dysfunction of neurons or other neural cells). Glutaminase-catalyzed hydrolysis of glutamine to glutamate is a predominant source of brain glutamate. Normal central nervous system (CNS) synaptic transmission uses glutamate as the major excitatory amino acid neurotransmitter. Excessive glutamatergic signaling, known as excitotoxicity, is believed to cause CNS damage in various neurodegenerative diseases, such as stroke, amyotrophic lateral sclerosis (ALS), Huntington's disease, Alzheimer's disease, and HIV-associated dementia. In another embodiment, the neurodegenerative disorder is multiple sclerosis (MS).

In another embodiment, the disease, disorder, or condition is a cognitive deficit. e.g., a cognitive deficit due to, or associated with, neurodegenerative disorders, such as multiple sclerosis, Parkinson's disease, schizophrenia, Alzheimer's disease (AD), autism, or cognitive deficits due to neuroinflammation such as cerebral malaria or encephalitis. As used herein, a “cognitive deficit” refers to a disease, disorder, or condition that is characterized by impairment of the mental processes of perception, learning, memory, judgment, and/or reasoning. In some embodiments, the cognitive deficit is selected from the group consisting of dementia, and mild to moderate cognitive decline (the latter resulting in gradual incapacitation of daily activities).

As used herein, the term “dementia” refers to a terminal disease or disorder that involves inability to think, learn, and remember such that a person's daily functioning is affected among other disabilities such as seizures and motor detects. As used herein, the term “cognitive decline” refers to a gradual decrease in a person's mental processes of perception, learning, memory, judgment, and reasoning. A “mild cognitive decline” refers to a decrease in a person's mental processes of perception, memory, judgment, and reasoning that is less than a 40% decrease, less than a 30% decrease, less than a 20% decrease, or less than a 10% decrease as compared to the person's cognitive ability before the cognitive decline occurred.

Some embodiments of the disclosure relate to correcting cognitive defects associated other neurodegenerative diseases, disorders, or conditions of the nervous systems, such as or associated with alcoholism, Alexander's disease, Alper's disease, ataxia telangiectasia, Batten disease (also known as Spielmeyer-Vogt-Sjogren-Batten disease), Canavan disease, Cockayne syndrome, diabetic neuropathy, frontotemporal lobar degeneration, HIV-associated dementia, Kennedy's disease, Krabbe's disease, neuroborreliosis, Machado-Joseph disease (Spinocerebellar ataxia type 3), wet or dry macular degeneration, Niemann Pick disease, Pelizaeus-Merzbacher Disease, photoreceptor degenerative diseases, such as retinitis pigmentosa and associated diseases, Refsum's disease, Sandhoffs disease, Schilder's disease, subacute combined degeneration of spinal cord secondary to pernicious anemia, Spielmeyer-Vogt-Sjogren-Batten disease (also known as Batten disease), spinocerebellar ataxia (multiple types with varying characteristics), Steele-Richardson-Olszewski disease, and tabes dorsalis.

Some embodiments of the disclosure relate to correcting cognitive defects associated with a wide range of genetic brain diseases. For example, genetic brain diseases may include but are not limited to Adrenoleukodystrophy, Agenesis of the Corpus Callosum, Aicardi Syndrome, Alpers' Disease. Alzheimer's Disease, Barth Syndrome, Batten Disease, CADASIL, Cerebellar Degeneration, Fabry's Disease, Gerstmann-Straussler-Scheinker Disease, Huntington's Disease and other Triplet Repeat Disorders, Leigh's Disease, Lesch-Nyhan Syndrome, Menkes Disease, Mitochondrial Myopathies and NINDS Colpocephaly.

Some embodiments of the disclosure relate to correcting cognitive defects associated with one or more conditions that are secondary to a disease, disorder, condition, or therapy having a primary effect outside of the nervous system selected from the group consisting of: peripheral neuropathy or neuralgia caused by diabetes, cancer, hepatitis, hepatic encephalopathy, kidney dysfunction, Colorado tick fever, diphtheria, leprosy, Lyme disease, polyarteritis nodosa, rheumatoid arthritis, sarcoidosis, Sjogren syndrome, syphilis, systemic lupus erythematosus, viral encephalitis, and amyloidosis. In some embodiments, the cognitive deficit is associated with hepatic encephalopathy. In some embodiments, the cognitive deficit is associated with viral encephalitis.

Some embodiments of the disclosure relate to correcting cognitive defects associated with a neurodegenerative disease, disorder, or condition associated with pain selected from the group consisting of chronic pain, fibromyalgia, spinal pain, carpel tunnel syndrome, pain from cancer, arthritis, sciatica, headaches, pain from surgery, muscle spasms, back pain, visceral pain, pain from injury, dental pain, neuralgia, such as neurogenic or neuropathic pain, nerve inflammation or damage, shingles, herniated disc, a torn ligament, and diabetes.

Some embodiments of the disclosure relate to correcting cognitive defects associated with a neurodegenerative disease, disorder, or condition that is associated with one or more injuries to the nervous system. In particular embodiments, the one or more injuries to the nervous system is related to nerve damage caused by exposure to one or more agents selected from the group consisting of toxic compounds, heavy metals, industrial solvents, drugs, chemotherapeutic agents, dapsone, cholesterol lowering drugs, heart or blood pressure medications, and metronidazole.

Some embodiments of the disclosure relate to correcting cognitive defects associated with a psychiatric disorder. In particular embodiments, the psychiatric disorder is selected from the group consisting of schizophrenia, delusional disorder, schizoaffective disorder, schizophreniform, shared psychotic disorder, psychosis, paranoid personality disorder, schizoid personality disorder, borderline personality disorder, anti-social personality disorder, narcissistic personality disorder, obsessive-compulsive disorder, delirium, dementia, mood disorders, bipolar disorder, depression, stress disorder, panic disorder, agoraphobia, social phobia, post-traumatic stress disorder, anxiety disorder, and impulse control disorders.

In another embodiment, the disease, disorder, or condition is an immune disorder. As used herein, the term “immune disorder” includes diseases involving the immune system that can include but not be limited to allergies, autoimmune diseases, immune complex diseases, immunodeficiency diseases and cancers of the immune system. Autoimmunity is the failure of an organism to recognize its own constituent parts (down to the sub-molecular levels) as “self”, which results in an immune response against its own cells and tissues. Any disease that results from such an aberrant immune response is termed an autoimmune disease. An unwanted immune response may be, for example, immune responses associated with an autoimmune disorder, transplants, allergies, or inflammatory disorders.

Exemplary autoimmune diseases include inflammatory responses, such as inflammatory skin diseases, including psoriasis and dermatitis (e.g. atopic dermatitis); dermatomyositis; systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions, such as eczema and asthma, and other conditions involving infiltration of T cells and chronic inflammatory responses; atherosclerosis; leukocyte adhesion deficiency; rheumatoid arthritis; systemic lupus erythematosus (SLE); diabetes mellitus (e.g. Type I diabetes mellitus or insulin dependent diabetes mellitus); multiple sclerosis; Reynaud's syndrome; autoimmune thyroiditis; allergic encephalomyelitis; Sjogren's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Bechet disease; giant cell arteritis; immune complex nephritis; IgA nephropathy; IgM polyneuropathies; immune thrombocytopenic purpura (ITP) or autoimmune thrombocytopenia and autoimmune hemolytic diseases, Hashimoto's thyroiditis, Wegener's granulomatosis, cold agglutinin disease associated with indolent lymphoma, acquired factor VIII inhibitors disease, etc.

In another embodiment, the disease, disorder, or condition is a pathology due to or associated with CNS inflammation due to an infection. In another embodiment, the disease, disorder, or condition is cereberal malaria. In another embodiment, the disease, disorder, or condition is a pathology due to or associated with CNS inflammation not involving an infection. In another embodiment, the disease, disorder, or condition is amyotrophic lateral sclerosis (ALS). In another embodiment, the disease, disorder, or condition is Alzheimer's Disease. In another embodiment, the disease, disorder, or condition is Parkinson's Disease. In another embodiment, the disease, disorder, or condition is neuromyelitis optica. In another embodiment, the disease, disorder, or condition is ARDS. In another embodiment, the disease, disorder, or condition is arthritis. In another embodiment, the disease, disorder, or condition is asthma. In another embodiment, the disease, disorder, or condition allograft rejection during cell, tissue, or organ transplantation. In another embodiment, the disease, disorder, or condition is cerebral malaria. In another embodiment, the disease, disorder, or condition is lupus. In another embodiment, the disease, disorder, or condition is pneumonitis. In another embodiment, the disease, disorder, or condition is pulmonary fibrosis.

In another embodiment, the disease, disorder, or condition is cancer.

In one embodiment, the cancer is a solid tumor.

In another embodiment, the cancer is a hematological cancer. In another embodiment, the hematological cancer is acute lymphocytic leukemia, chronic lymphocytic leukemia (including B-cell chronic lymphocytic leukemia), or acute myeloid leukemia.

In another embodiment, the cancer is any one or more of the cancers of Table 3.

TABLE 3

| | | | |
|---|---|---|---|
| adrenal cancer | acinic cell carcinoma | acoustic neuroma | acral lentigious |
| | | | melanoma |
| acrospiroma | acute eosinophilic | acute erythroid | acute lymphoblastic |
| | leukemia | leukemia | leukemia |
| acute | acute monocytic | acute promyelocytic | adenocarcinoma |
| megakaryoblastic | leukemia | leukemia | |
| leukemia | | | |
| adenoid cystic | adenoma | adenomatoid | adenosquamous |
| carcinoma | | odontogenic tumor | carcinoma |
| adipose tissue | adrenocortical | adult T-cell | aggressive NK-cell |
| neoplasm | carcinoma | leukemia/lymphoma | leukemia |
| AIDS-related | alveolar | alveolar soft part | ameloblastic fibroma |
| lymphoma | rhabdomyosarcoma | sarcoma | |
| anaplastic large | anaplastic thyroid | angioimmunoblastic | angiomyolipoma |
| cell lymphoma | cancer | T-cell lymphoma | |
| angiosarcoma | astrocytoma | atypical teratoid | B-cell chronic |
| | | rhabdoid tumor | lymphocytic leukemia |
| B-cell | B-cell lymphoma | basal cell carcinoma | biliary tract cancer |
| prolymphocytic | | | |
| leukemia | | | |
| bladder cancer | blastoma | bone cancer | Brenner tumor |
| Brown tumor | Burkitt's lymphoma | breast cancer | brain cancer |
| carcinoma | carcinoma in situ | carcinosarcoma | cartilage tumor |
| cementoma | myeloid sarcoma | chondroma | chordoma |
| choriocarcinoma | choroid plexus | clear-cell sarcoma of | craniopharyngioma |
| | papilloma | the kidney | |
| cutaneous T-cell | cervical cancer | colorectal cancer | Degos disease |
| lymphoma | | | |
| desmoplastic small | diffuse large B-cell | dysembryoplastic | dysgerminoma |
| round cell tumor | lymphoma | neuroepithelial tumor | |
| embryonal | endocrine gland | endodermal sinus | enteropathy- |
| carcinoma | neoplasm | tumor | associated T-cell |
| | | | lymphoma |
| esophageal cancer | fetus in fetu | fibroma | fibrosarcoma |
| follicular | follicular thyroid | ganglioneuroma | gastrointestinal cancer |
| lymphoma | cancer | | |
| germ cell tumor | gestational | giant cell | giant cell tumor of |
| | choriocarcinoma | fibroblastoma | the bone |
| glial tumor | glioblastoma | glioma | gliomatosis cerebri |
| | multiforme | | |
| glucagonoma | gonadoblastoma | granulosa cell tumor | gynandroblastoma |
| gallbladder cancer | gastric cancer | hairy cell leukemia | hemangioblastoma |
| head and neck | hemangiopericytoma | hematological cancer | hepatoblastoma |
| cancer | | | |
| hepatosplenic T- | Hodgkin's lymphoma | non-Hodgkin's | invasive lobular |
| cell lymphoma | | lymphoma | carcinoma |
| intestinal cancer | kidney cancer | laryngeal cancer | lentigo maligna |
| lethal midline | leukemia | leydig cell tumor | liposarcoma |
| carcinoma | | | |
| lung cancer | lymphangioma | lymphangiosarcoma | lymphoepithelioma |
| lymphoma | acute lymphocytic | acute myelogeous | chronic lymphocytic |
| | leukemia | leukemia | leukemia |
| liver cancer | small cell lung cancer | non-small cell lung | MALT lymphoma |
| | | cancer | |
| malignant fibrous | malignant peripheral | malignant triton tumor | mantle cell lymphoma |
| histiocytoma | nerve sheath tumor | | |
| marginal zone B- | mast cell leukemia | mediastinal germ cell | medullary carcinoma |
| cell lymphoma | | tumor | of the breast |
| medullary thyroid | medulloblastoma | melanoma | meningioma |
| cancer | | | |
| merkel cell cancer | mesothelioma | metastatic urothelial | mixed Mullerian |
| | | carcinoma | tumor |
| mucinous tumor | multiple myeloma | muscle tissue | mycosis fungoides |
| | | neoplasm | |
| myxoid | myxoma | myxosarcoma | nasopharyngeal |
| liposarcoma | | | carcinoma |
| neurinoma | neuroblastoma | neurofibroma | neuroma |
| nodular melanoma | ocular cancer | oligoastrocytoma | oligodendroglioma |
| oncocytoma | optic nerve sheath | optic nerve tumor | oral cancer |
| | meningioma | | |
| osteosarcoma | ovarian cancer | Pancoast tumor | papillary thyroid |
| | | | cancer |
| paraganglioma | pinealoblastoma | pineocytoma | pituicytoma |
| pituitary adenoma | pituitary tumor | plasmacytoma | polyembryoma |
| precursor T- | primary central | primary effusion | preimary peritoneal |
| lymphoblastic | nervous system | lymphoma | cancer |
| lymphoma | lymphoma | | |
| prostate cancer | pancreatic cancer | pharyngeal cancer | pseudomyxoma |
| | | | periotonei |
| renal cell | renal medullary | retinoblastoma | rhabdomyoma |
| carcinoma | carcinoma | | |
| rhabdomyosarcoma | Richter's | rectal cancer | sarcoma |
| | transformation | | |
| Schwannomatosis | seminoma | Sertoli cell tumor | sex cord-gonadal |
| | | | stromal tumor |
| signet ring cell | skin cancer | small blue round cell | small cell carcinoma |
| carcinoma | | tumors | |
| soft tissue sarcoma | somatostatinoma | soot wart | spinal tumor |
| splenic marginal | squamous cell | synovial sarcoma | Sezary's disease |
| zone lymphoma | carcinoma | | |
| small intestine | squamous carcinoma | stomach cancer | T-cell lymphoma |
| cancer | | | |
| testicular cancer | thecoma | thyroid cancer | transitional cell |
| | | | carcinoma |
| throat cancer | urachal cancer | urogenital cancer | urothelial carcinoma |
| uveal melanoma | uterine cancer | verrucous carcinoma | visual pathway glioma |
| vulvar cancer | vaginal cancer | Waldenstrom's | Warthin's tumor |
| | | macroglobulinemia | |
| Wilms' tumor | | | |

In another embodiment, the cancer is any one or more of the cancers of Table 4.

TABLE 4

| | |
|---|---|
| acute lymphocytic leukemia (ALL) | acute eosinophilic leukemia |
| acute myeloid leukemia (AML) | acute erythroid leukemia |
| chronic lymphocytic leukemia (CLL) | acute lymphoblastic leukemia |
| small lymphocytic lymphoma (SLL) | acute megakaryoblastic leukemia |
| multiple myeloma (MM) | acute monocytic leukemia |
| Hodgkins lymphoma (HL) | acute promyelocytic leukemia |
| non-Hodgkin's lymphoma (NHL) | acute myelogeous leukemia |
| mantle cell lymphoma (MCL) | B-cell prolymphocytic leukemia |
| marginal zone B-cell lymphoma | B-cell lymphoma |
| splenic marginal zone lymphoma | MALT lymphoma |
| follicular lymphoma (FL) | precursor T-lymphoblastic lymphoma |
| Waldenstrom's macroglobulinemia (WM) | T-cell lymphoma |
| diffuse large B-cell lymphoma (DLBCL) | mast cell leukemia |
| marginal zone lymphoma (MZL) | adult T cell leukemia/lymphoma |
| hairy cell leukemia (HCL) | aggressive NK-cell leukemia |
| Burkitt's lymphoma (BL) | angioimmunoblastic T-cell lymphoma |
| Richter's transformation | |

In another embodiment, the cancer is selected from the group consisting of squamous cell carcinoma of the head and neck, adenocarcinoma squamous cell carcinoma of the esophagus, adenocarcinoma of the stomach, adenocarcinoma of the colon, hepatocellular carcinoma, cholangiocarcinoma of the biliary system, adenocarcinoma of gall bladder, adenocarcinoma of the pancreas, ductal carcinoma in situ of the breast, adenocarcinoma of the breast, adenocarcinoma of the lungs, squamous cell carcinoma of the lungs, transitional cell carcinoma of the bladder, squamous cell carcinoma of the bladder, squamous cell carcinoma of the cervix, adenocarcinoma of the cervix, endometrial carcinoma, penile squamous cell carcinoma, and squamous cell carcinoma of the skin.

In another embodiment, a precancerous tumor is selected from the group consisting of leukoplakia of the head and neck, Barrett's esophagus, metaplasia of the stomach, adenoma of the colon, chronic hepatitis, bile duct hyperplasia, pancreatic intraepithelial neoplasia, atypical adenomatous hyperplasia of the lungs, dysplasia of the bladder, cervical intraepithelial neoplasia, penile intraepithelial neoplasia, and actinic keratosis of the skin.

In another embodiment, the cancer is selected from the group consisting of hepatocellular carcinoma, glioblastoma, lung cancer, breast cancer, head and neck cancer, prostate cancer, melanoma, and colorectal cancer.

In another embodiment, the cancer is selected from the group consisting of colorectal cancer, breast cancer, lymphoma, melanoma, kidney cancer, and lung cancer.

In another embodiment, the cancer has become resistant to conventional cancer treatments. The term “conventional cancer treatments” as used herein refers to any cancer drugs, biologics, or radiotherapy, or combination of cancer drugs and/or biologics and/or radiotherapy that have been tested and/or approved for therapeutic use in humans by the U.S. Food and Drug Administration, European Medicines Agency, or similar regulatory agency.

The therapeutic methods provided herein comprise administering a Compound of the Disclosure or Composition of the Disclosure in an amount which is effective to achieve its intended purpose. While individual needs vary, determination of optimal ranges of effective amounts of each component is within the skill of the art. Typically, a Compound of the Disclosure is administered in an amount from about 0.05 mg/kg to about 500 mg/kg, about 0.05 mg/kg to about 100 mg/kg, about 0.05 mg/kg to about 50 mg/kg, or about 0.05 mg/kg to about 10 mg/kg. The dosage of a composition can be at any dosage including, but not limited to, about 0.05 mg/week to about 25 mg/week. Particular doses include 0.05, 1, 2, 5, 10, 20, 500, or 100 mg/kg per week. These dosages are exemplary, but there can be individual instances in which higher or lower dosages are merited, and such are within the scope of this disclosure. In practice, the physician determines the actual dosing regimen that is most suitable for an individual patient, which can vary with the age, weight, and response of the particular patient.

The unit dose for, e.g., oral, subcutaneous, or intravenous administration, may comprise from about 0.01 to about 1000 mg, e.g., about 0.01 to about 100 mg of a Compound of the Disclosure. In one embodiment, the unit dose is 0.05 mg, 1 mg, 3 mg, 5 mg, 7 mg, 9 mg, 10 mg 12 mg, 14 mg, 15 mg, 17 mg, 20 mg, 22 mg, 25 mg, 27 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg. The unit dose may be administered one or more times daily, e.g., as one or more tablets or capsules. The unit does may also be administered by IV or subcutaneously to the subject. In practice, the physician determines the actual dosing regimen that is most suitable for an individual patient, which can vary with the age, weight, and response of the particular patient.

### IV. Optional Therapeutic Agents

In some therapeutic methods and uses of the disclosure, a Compound of the Disclosure or Composition of the Disclosure is administered to a subject having a disease, disorder, or condition, e.g., cancer, as a single agent. In other therapeutic methods and uses of the disclosure, a Pharmaceutical Formulation of the Disclosure is administered to a subject having cancer in combination with one or more optional therapeutic agents. In one embodiment, a Compound of the Disclosure or Composition of the Disclosure is administered in combination with one optional therapeutic agent. In another embodiment, a Compound of the Disclosure or Composition of the Disclosure is administered in combination with two optional therapeutic agents. In another embodiment, a Compound of the Disclosure or Composition of the Disclosure is administered in combination with three optional therapeutic agents. Optional therapeutic agents useful in treating disease, disorder, or condition, e.g., cancer, in a subject include those known in the art as well as those developed in the future.

Optional therapeutic agents are administered in an amount to provide their desired therapeutic effect. The effective dosage range for each optional therapeutic agent is known in the art, and the optional therapeutic agent is administered to an individual in need thereof within such established ranges.

A Compound of the Disclosure or Composition of the Disclosure, and the optional therapeutic agent(s) can be administered separately as multi-unit doses in any order, e.g., wherein a Compound of the Disclosure is administered before the optional therapeutic agent(s), or vice versa. One or more doses of a Compound of the Disclosure or Composition of the Disclosure, and the optional therapeutic agent(s) can be administered to the subject.

In one embodiment, the optional therapeutic agent is an immune checkpoint inhibitor. Examples of immune checkpoint inhibitors include PD-1 inhibitors, PD-L1 inhibitors, CTLA-4 inhibitors, LAG3 inhibitors, TIM3 inhibitors, cd47 inhibitors, and B7-H1 inhibitors. Thus, in one embodiment, the immune checkpoint inhibitor is selected from the group consisting of a PD-1 inhibitor, a PD-L1 inhibitor, a CTLA-4 inhibitor, a LAG3 inhibitor, a TIM3 inhibitor, and a cd47 inhibitor.

In another embodiment, the immune checkpoint inhibitor is a programmed cell death (PD-1) inhibitor. PD-1 is a T-cell coinhibitory receptor that plays a pivotal role in the ability of tumor cells to evade the host's immune system. Blockage of interactions between PD-1 and PD-L1, a ligand of PD-1, enhances immune function and mediates antitumor activity. Examples of PD-1 inhibitors include antibodies that specifically bind to PD-1. Particular anti-PD-1 antibodies include, but are not limited to nivolumab, pembrolizumab, STI-A1014, pidilzumab, and cemiplimab-rwlc. For a general discussion of the availability, methods of production, mechanism of action, and clinical studies of anti-PD-1 antibodies, see U.S. 2013/0309250, U.S. Pat. Nos. 6,808,710, 7,595,048, 8,008,449, 8,728,474, 8,779,105, 8,952,136, 8,900,587, 9,073,994, 9,084,776, and Naido et al., British Journal of Cancer 111:2214-19 (2014).

In another embodiment, the immune checkpoint inhibitor is a PD-L1 (also known as B7-H1 or CD274) inhibitor. Examples of PD-L1 inhibitors include antibodies that specifically bind to PD-L1. Particular anti-PD-L1 antibodies include, but are not limited to, avelumab, atezolizumab, durvalumab, and BMS-936559. For a general discussion of the availability, methods of production, mechanism of action, and clinical studies, see U.S. Pat. No. 8,217,149, U.S. 2014/0341917, U.S. 2013/0071403, WO 2015036499, and Naido et al., British Journal of Cancer 111:2214-19 (2014).

In another embodiment, the immune checkpoint inhibitor is a CTLA-4 inhibitor. CTLA-4, also known as cytotoxic T-lymphocyte antigen 4, is a protein receptor that downregulates the immune system. CTLA-4 is characterized as a “brake” that binds costimulatory molecules on antigen-presenting cells, which prevents interaction with CD₂₈ on T cells and also generates an overtly inhibitory signal that constrains T cell activation. Examples of CTLA-4 inhibitors include antibodies that specifically bind to CTLA-4. Particular anti-CTLA-4 antibodies include, but are not limited to, ipilimumab and tremelimumab. For a general discussion of the availability, methods of production, mechanism of action, and clinical studies, see U.S. Pat. Nos. 6,984,720, 6,207,156, and Naido et al., British Journal of Cancer 111:2214-19 (2014).

In another embodiment, the immune checkpoint inhibitor is a LAG3 inhibitor. LAG3, Lymphocyte Activation Gene 3, is a negative co-simulatory receptor that modulates T cell homeostatis, proliferation, and activation. In addition, LAG3 has been reported to participate in regulatory T cells (Tregs) suppressive function. A large proportion of LAG3 molecules are retained in the cell close to the microtubule-organizing center, and only induced following antigen specific T cell activation. U.S. 2014/0286935. Examples of LAG3 inhibitors include antibodies that specifically bind to LAG3. Particular anti-LAG3 antibodies include, but are not limited to, GSK2831781. For a general discussion of the availability, methods of production, mechanism of action, and studies, see, U.S. 2011/0150892, U.S. 2014/0093511, U.S. 20150259420, and Huang et al., Immunity 21:503-13 (2004).

In another embodiment, the immune checkpoint inhibitor is a TIM3 inhibitor. TIM3, T-cell immunoglobulin and mucin domain 3, is an immune checkpoint receptor that functions to limit the duration and magnitude of T_{H}1 and T_{C}1 T-cell responses. The TIM3 pathway is considered a target for anticancer immunotherapy due to its expression on dysfunctional CD8⁺T cells and Tregs, which are two reported immune cell populations that constitute immunosuppression in tumor tissue. Anderson, Cancer Immunology Research 2:393-98 (2014). Examples of TIM3 inhibitors include antibodies that specifically bind to TIM3. For a general discussion of the availability, methods of production, mechanism of action, and studies of TIM3 inhibitors, see U.S. 20150225457, U.S. 20130022623, U.S. Pat. No. 8,522,156, Ngiow et al., Cancer Res 71: 6567-71 (2011), Ngiow, et al., Cancer Res 71:3540-51 (2011), and Anderson, Cancer Immunology Res 2:393-98 (2014).

In another embodiment, the immune checkpoint inhibitor is a cd47 inhibitor. See Unanue, E. R., PNAS 110:10886-87 (2013).

The term “antibody” is meant to include intact monoclonal antibodies, polyclonal antibodies, multispecific antibodies formed from at least two intact antibodies, and antibody fragments, so long as they exhibit the desired biological activity. In another embodiment, “antibody” is meant to include soluble receptors that do not possess the Fc portion of the antibody. In one embodiment, the antibodies are humanized monoclonal antibodies and fragments thereof made by means of recombinant genetic engineering.

Another class of immune checkpoint inhibitors include polypeptides that bind to and block PD-1 receptors on T-cells without triggering inhibitor signal transduction. Such peptides include B7-DC polypeptides, B7-H1 polypeptides, B7-1 polypeptides and B7-2 polypeptides, and soluble fragments thereof, as disclosed in U.S. Pat. No. 8,114,845.

Another class of immune checkpoint inhibitors include compounds with peptide moieties that inhibit PD-1 signaling. Examples of such compounds are disclosed in U.S. Pat. No. 8,907,053.

Another class of immune checkpoint inhibitors include inhibitors of certain metabolic enzymes, such as indoleamine 2,3 dioxygenase (IDO), which is expressed by infiltrating myeloid cells and tumor cells, and isocitrate dehydrogenase (IDH), which is mutated in leukemia cells. Mutants of the IDH enzyme lead to increased levels of 2-hydroxyglutarate (2-HG), which prevent myeloid differentiation. Stein et al., Blood 130:722-31 (2017); Wouters, Blood 130:693-94 (2017). Particular mutant IDH blocking agents include, but are not limited to, ivosidenib and enasidenib mesylate. Dalle and DiNardo, Ther Adv Hematol 9(7):163-73 (2018); Nassereddine et al., Onco Targets Ther 12:303-08 (2018). The IDO enzyme inhibits immune responses by depleting amino acids that are necessary for anabolic functions in T cells or through the synthesis of particular natural ligands for cytosolic receptors that are able to alter lymphocyte functions. Pardoll, Nature Reviews. Cancer 12:252-64 (2012); Löb, Cancer Immunol Immunother 58:153-57 (2009). Particular IDO blocking agents include, but are not limited to, levo-1-methyl typtophan (L-1MT) and 1-methyl-tryptophan (1MT). Qian et al., Cancer Res 69:5498-504 (2009); and Löb et al., Cancer Immunol Immunother 58:153-7 (2009).

In one embodiment, the immune checkpoint inhibitor is nivolumab, pembrolizumab, pidilizumab, STI-A1110, avelumab, atezolizumab, durvalumab, STI-A1014, ipilimumab, tremelimumab, GSK2831781, BMS-936559 or MED14736.

In another embodiment, the optional therapeutic agent is an epigenetic drug. As used herein, the term “epigenetic drug” refers to a therapeutic agent that targets an epigenetic regulator. Examples of epigenetic regulators include the histone lysine methyltransferases, histone arginine methyl transferases, histone demethylases, histone deacetylases, histone acetylases, and DNA methyltransferases. Histone deacetylase inhibitors include, but are not limited to, vorinostat and panobinostat lactate.

Additional examples of conventional therapies and anticancer agents that can be used in combination with a Pharmaceutical Formulation of the Disclosure include surgery, radiotherapy, e.g., gamma-radiation, neutron beam radiotherapy, electron beam radiotherapy, proton therapy, brachytherapy, and systemic radioactive isotopes, endocrine therapy, a biologic response modifier, e.g., an interferon, an interleukin, tumor necrosis factor (TNF), hyperthermia and cryotherapy, an agent to attenuate any adverse effect (e.g., an antiemetic), and any other approved biologic therapy or chemotherapy, e.g., a treatment regimen that uses drugs to stop the growth of cancer cells, either by killing the cells or by stopping them from dividing. Chemotherapy may be given by mouth, injection, or infusion, or on the skin, depending on the type and stage of the cancer being treated.

Nonlimiting exemplary antiproliferative compounds include an aromatase inhibitor; an anti-estrogen; an anti-androgen; a gonadorelin agonist; a topoisomerase I inhibitor; a topoisomerase II inhibitor; a microtubule active agent; an alkylating agent, e.g., temozolomide; a retinoid, a carontenoid, or a tocopherol; a cyclooxygenase inhibitor; an MMP inhibitor; an mTOR inhibitor; an antimetabolite; a platin compound; a methionine aminopeptidase inhibitor; a bisphosphonate; an antiproliferative antibody; a heparanase inhibitor; an inhibitor of Ras oncogenic isoforms; a telomerase inhibitor; a proteasome inhibitor; a compound used in the treatment of hematologic malignancies; a Flt-3 inhibitor; an Hsp90 inhibitor; a kinesin spindle protein inhibitor; a MEK inhibitor; an antitumor antibiotic; a nitrosourea; a compound targeting/decreasing protein or lipid kinase activity, a compound targeting/decreasing protein or lipid phosphatase activity, or any further anti-angiogenic compound.

Nonlimiting exemplary aromatase inhibitors include steroids, such as atamestane, exemestane, and formestane, and non-steroids, such as aminoglutethimide, roglethimide, pyridoglutethimide, trilostane, testolactone, ketokonazole, vorozole, fadrozole, anastrozole, and letrozole.

Nonlimiting anti-estrogens include tamoxifen, fulvestrant, raloxifene, and raloxifene hydrochloride. Anti-androgens include, but are not limited to, bicalutamide and apalutamide. Gonadorelin agonists include, but are not limited to, abarelix, goserelin, and goserelin acetate.

Nonlimiting exemplary topoisomerase I inhibitors include topotecan, gimatecan, irinotecan, camptothecin and its analogues, 9-nitrocamptothecin, and the macromolecular camptothecin conjugate PNU-166148. Topoisomerase II inhibitors include, but are not limited to, anthracyclines, such as doxorubicin, daunorubicin, epirubicin, idarubicin, and nemorubicin; anthraquinones, such as mitoxantrone and losoxantrone; and podophillotoxines, such as etoposide and teniposide.

Microtubule active agents include microtubule stabilizing, microtubule destabilizing compounds, and microtubulin polymerization inhibitors including, but not limited to, taxanes, such as paclitaxel and docetaxel; discodermolides; cochicine and epothilones and derivatives thereof.

Nonlimiting exemplary alkylating agents include cyclophosphamide, ifosfamide, melphalan, trabectedin, and nitrosoureas, such as carmustine and lomustine.

Nonlimiting exemplary matrix metalloproteinase inhibitors (“MMP inhibitors”) include collagen peptidomimetic and nonpeptidomimetic inhibitors, tetracycline derivatives, batimastat, marimastat, prinomastat, metastat, BMS-279251, BAY 12-9566, TAA211, MMI270B, and AAJ996.

Nonlimiting exemplary mTOR inhibitors include compounds that inhibit the mammalian target of rapamycin (mTOR) and possess antiproliferative activity such as sirolimus, everolimus, CCI-779, and ABT578.

Nonlimiting exemplary antimetabolites include 5-fluorouracil (5-FU), capecitabine, gemcitabine, DNA demethylating compounds, such as 5-azacytidine and decitabine, methotrexate and edatrexate, and folic acid antagonists, such as pemetrexed.

Nonlimiting exemplary platin compounds include carboplatin, cis-platin, cisplatinum, and oxaliplatin.

Nonlimiting exemplary methionine aminopeptidase inhibitors include bengamide or a derivative thereof and PPI-2458.

Nonlimiting exemplary bisphosphonates include etridonic acid, clodronic acid, tiludronic acid, pamidronic acid, alendronic acid, ibandronic acid, risedronic acid, and zoledronic acid.

Nonlimiting exemplary heparanase inhibitors include compounds that target, decrease, or inhibit heparin sulfate degradation, such as PI-88 and OGT2115.

Nonlimiting exemplary compounds which target, decrease, or inhibit the oncogenic activity of Ras include farnesyl transferase inhibitors, such as L-744832, DK8G557, tipifarnib, and lonafarnib.

Nonlimiting exemplary telomerase inhibitors include compounds that target, decrease, or inhibit the activity of telomerase, such as compounds that inhibit the telomerase receptor, such as telomestatin.

Nonlimiting exemplary proteasome inhibitors include compounds that target, decrease, or inhibit the activity of the proteasome including, but not limited to, bortezomib. In some embodiments, the proteasome inhibitor is carfilzomib or ixazomib.

Nonlimiting exemplary FMS-like tyrosine kinase inhibitors, which are compounds targeting, decreasing or inhibiting the activity of FMS-like tyrosine kinase receptors (Flt-3R), include gilteritinib, interferon, I-β-D-arabinofuransylcytosine (ara-c), and bisulfan; and ALK inhibitors, which are compounds that target, decrease, or inhibit anaplastic lymphoma kinase, include alectinib, brigatinib, and lorlatinib.

Nonlimiting exemplary Flt-3 inhibitors include PKC412, midostaurin, a staurosporine derivative, SU11248, MLN518, and gilteritinib.

Nonlimiting exemplary HSP90 inhibitors include compounds targeting, decreasing, or inhibiting the intrinsic ATPase activity of HSP90; or degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins, or antibodies that inhibit the ATPase activity of HSP90, such as 17-allylamino,17-demethoxygeldanamycin (17AAG), a geldanamycin derivative; other geldanamycin related compounds; radicicol and HDAC inhibitors.

Nonlimiting exemplary protein tyrosine kinase and/or serine and/or threonine kinase inhibitors or lipid kinase inhibitors, include a) a compound targeting, decreasing, or inhibiting the activity of the platelet-derived growth factor-receptors (PDGFR), such as a compound that targets, decreases, or inhibits the activity of PDGFR, including olaratumab and N-phenyl-2-pyrimidine-amine derivatives, such as imatinib, SUlOl, SU6668, and GFB-111; b) a compound targeting, decreasing, or inhibiting the activity of the fibroblast growth factor-receptors (FGFR), such as erdafitinib and lenvatinib; c) a compound targeting, decreasing, or inhibiting the activity of the insulin-like growth factor receptor I (IGF-IR), such as brigatinib; d) a compound targeting, decreasing, or inhibiting the activity of the vascular endothelial growth factor-receptors (VEGFR), such as lenvatinib; e) a compound targeting, decreasing, or inhibiting the activity of the Trk receptor tyrosine kinase family, or ephrin B4 inhibitors, such as larotrectinib; f) a compound targeting, decreasing, or inhibiting the activity of the Axl receptor tyrosine kinase family; g) a compound targeting, decreasing, or inhibiting the activity of the Ret receptor tyrosine kinase, such as alectinib; h) a compound targeting, decreasing, or inhibiting the activity of the Kit/SCFR receptor tyrosine kinase, such as imatinib; i) a compound targeting, decreasing, or inhibiting the activity of the c-Kit receptor tyrosine kinases, such as imatinib; j) a compound targeting, decreasing, or inhibiting the activity of members of the c-Abl family, their gene-fusion products (e.g. Bcr-Abl kinase) and mutants, such as an N-phenyl-2-pyrimidine-amine derivative, such as imatinib or nilotinib; PD180970; AG957; NSC 680410; PD173955; or dasatinib; k) a compound targeting, decreasing, or inhibiting the activity of members of the protein kinase C (PKC) and Raf family of serine/threonine kinases, members of the MEK, SRC, JAK, FAK, PDK1, PKB/Akt, and Ras/MAPK family members, and/or members of the cyclin-dependent kinase family (CDK), such as a staurosporine derivative disclosed in U.S. Pat. No. 5,093,330, such as midostaurin; examples of further compounds include UCN-01, safingol, BAY 43-9006, bryostatin 1, perifosine; ilmofosine; RO 318220 and RO 320432; GO 6976; Isis 3521; LY333531/LY379196; a isochinoline compound; a farnesyl transferase inhibitor; PD184352 or QAN697, or AT7519; abemaciclib; binimetinib; cobimetinib; encorafenib; neratinib; palbociclib; ribociclib; 1) a compound targeting, decreasing or inhibiting the activity of a protein-tyrosine kinase, such as acalabrutinib, imatinib mesylate or a tyrphostin, such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); m) a compound targeting, decreasing, or inhibiting the activity of the epidermal growth factor family of receptor tyrosine kinases (EGFR, ErbB2, ErbB3, ErbB4 as homo- or heterodimers) and their mutants, such as brigatinib, CP 358774, ZD 1839, ZM 105180; trastuzumab, cetuximab, gefitinib, erlotinib, osimertinib, dacomitinib, necitumumab, neratinib, OSI-774, Cl-1033, EKB-569, GW-2016, antibodies ELI, E2.4, E2.5, E6.2, E6.4, E2.11, E6.3 and E7.6.3, and 7H-pyrrolo-[2,3-d]pyrimidine derivatives; n) a compound targeting, decreasing or inhibiting the activity of a phosphatidylinositol 3-kinase (PI3K), such as alpelisib, copanlisib, and duvelisib; and o) a compound targeting, decreasing, or inhibiting the activity of the c-Met receptor.

Nonlimiting exemplary compounds that target, decrease, or inhibit the activity of a protein or lipid phosphatase include inhibitors of phosphatase 1, phosphatase 2A, or CDC25, such as okadaic acid or a derivative thereof.

Further anti-angiogenic compounds include compounds having another mechanism for their activity unrelated to protein or lipid kinase inhibition, e.g., thalidomide and TNP-470.

Additional, nonlimiting, exemplary chemotherapeutic compounds, one or more of which may be used in combination with a Formulation of the Disclosure include: avastin, daunorubicin, adriamycin, Ara-C, VP-16, teniposide, mitoxantrone, idarubicin, carboplatinum, PKC412, 6-mercaptopurine (6-MP), fludarabine phosphate, octreotide, SOM230, FTY720, 6-thioguanine, cladribine, 6-mercaptopurine, pentostatin, hydroxyurea, 2-hydroxy-1H-isoindole-1,3-dione derivatives, 1-(4-chloroanilino)-4-(4-pyridylmethyl)phthalazine or a pharmaceutically acceptable salt thereof, 1-(4-chloroanilino)-4-(4-pyridylmethyl)phthalazine succinate, angiostatin, endostatin, anthranilic acid amides, ZD4190, ZD6474, SU5416, SU6668, bevacizumab, rhuMAb, rhuFab, macugon; FLT-4 inhibitors, FLT-3 inhibitors, VEGFR-2 IgGI antibody, RPI 4610, porfimer sodium, anecortave, triamcinolone, hydrocortisone, 11-a-epihydrocotisol, cortex olone, 17a-hydroxyprogesterone, corticosterone, desoxycorticosterone, testosterone, estrone, dexamethasone, fluocinolone, a plant alkaloid, a hormonal compound and/or antagonist, a biological response modifier, such as a lymphokine or interferon, an antisense oligonucleotide or oligonucleotide derivative, shRNA, and siRNA.

A number of suitable optional therapeutic, e.g., anticancer, agents, are contemplated for use in the therapeutic methods provided herein. Indeed, the methods provided herein can include, but are not limited to, administration of numerous optional therapeutic agents such as: agents that induce apoptosis; polynucleotides (e.g., anti-sense, ribozymes, siRNA); polypeptides (e.g., enzymes and antibodies); biological mimetics (e.g., gossypol or BH3 mimetics); agents that bind (e.g., oligomerize or complex) with a Bcl-2 family protein such as Bax; alkaloids; alkylating agents; antitumor antibiotics; antimetabolites; hormones; platinum compounds; monoclonal or polyclonal antibodies (e.g., antibodies conjugated with anticancer drugs, toxins, defensins), toxins; radionuclides; biological response modifiers (e.g., interferons (e.g., IFN-α) and interleukins (e.g., IL-2)); adoptive immunotherapy agents; hematopoietic growth factors; agents that induce tumor cell differentiation (e.g., all-trans-retinoic acid); gene therapy reagents (e.g., antisense therapy reagents and nucleotides); tumor vaccines; angiogenesis inhibitors; proteosome inhibitors: NF-KB modulators; anti-CDK compounds; HDAC inhibitors; and the like. Numerous other examples of optional therapeutic agents such as chemotherapeutic compounds and anticancer therapies suitable for co-administration with the disclosed compounds are known to those skilled in the art.

In certain embodiments, optional therapeutic agents comprise agents that induce or stimulate apoptosis. Agents that induce or stimulate apoptosis include, for example, agents that interact with or modify DNA, such as by intercalating, cross-linking, alkylating, or otherwise damaging or chemically modifying DNA. Agents that induce apoptosis include, but are not limited to, radiation (e.g., X-rays, gamma rays, UV); tumor necrosis factor (TNF)-related factors (e.g., TNF family receptor proteins, TNF family ligands, TRAIL, antibodies to TRAIL-R1 or TRAIL-R2); kinase inhibitors (e.g., epidermal growth factor receptor (EGFR) kinase inhibitor). Additional anticancer agents include: vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC)); antisense molecules; antibodies (e.g., HERCEPTIN, RITUXAN, ZEVALIN, and AVASTIN); anti-estrogens (e.g., raloxifene and tamoxifen); anti-androgens (e.g., flutamide, apalutamide, bicalutamide, finasteride, aminoglutethamide, ketoconazole, and corticosteroids); BCL-2 inhibitors (e.g., venetoclax); cyclooxygenase 2 (COX-2) inhibitors (e.g., celecoxib, meloxicam, NS-398, and non-steroidal anti-inflammatory drugs (NSAIDs)); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine (DTIC), dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; staurosporine, and the like.

In still other embodiments, the therapeutic methods provided herein include administering to a subject having cancer (a cancer patient) therapeutically effective amounts of a Formulation of the Disclosure, an immune checkpoint inhibitor, and at least one additional optional therapeutic agent, e.g., an anti-hyperproliferative or antineoplastic agent selected from alkylating agents, antimetabolites, and natural products (e.g., herbs and other plant and/or animal derived compounds).

Alkylating agents suitable for use in the present methods include, but are not limited to: 1) nitrogen mustards (e.g., mechlorethamine, cyclophosphamide, ifosfamide, melphalan (L-sarcolysin); and chlorambucil); 2) ethylenimines and methylmelamines (e.g., hexamethylmelamine and thiotepa); 3) alkyl sulfonates (e.g., busulfan); 4) nitrosoureas (e.g., carmustine (BCNU); lomustine (CCNU); semustine (methyl-CCNU); and streptozocin (streptozotocin)); and 5) triazenes (e.g., dacarbazine (DTIC; dimethyltriazenoimid-azolecarboxamide).

In some embodiments, antimetabolites suitable for use in the present methods include, but are not limited to: 1) folic acid analogs (e.g., methotrexate (amethopterin)); 2) pyrimidine analogs (e.g., fluorouracil (5-fluorouracil; 5-FU), floxuridine (fluorode-oxyuridine; FudR), and cytarabine (cytosine arabinoside)); and 3) purine analogs (e.g., mercaptopurine (6-mercaptopurine; 6-MP), thioguanine (6-thioguanine; TG), and pentostatin (2′-deoxycoformycin)).

In still further embodiments, chemotherapeutic agents suitable for use in the methods of the present disclosure include, but are not limited to: 1) vinca alkaloids (e.g., vinblastine (VLB), vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin (cis-DDP) and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide).

Any oncolytic agent that is routinely used in a cancer therapy context finds use in the therapeutic methods of the present disclosure. For example, the U.S. Food and Drug Administration (FDA) maintains a formulary of oncolytic agents approved for use in the United States. International counterpart agencies to the FDA maintain similar formularies. Those skilled in the art will appreciate that the “product labels” required on all U.S. approved chemotherapeutics describe approved indications, dosing information, toxicity data, and the like, for the exemplary agents.

Anticancer agents further include compounds which have been identified to have anticancer activity. Examples include, but are not limited to, 3-AP, 12-O-tetradecanoylphorbol-13-acetate, 17AAG, 852A, ABI-007, ABR-217620, ABT-751, ADI-PEG 20, AE-941, AG-013736, AGRO100, alanosine, AMG 706, antibody G250, antineoplastons, AP23573, apaziquone, APC8015, atiprimod, ATN-161, atrasenten, azacitidine, BB-10901, BCX-1777, bevacizumab, BG00001, bicalutamide, BMS 247550, bortezomib, bryostatin-1, buserelin, calaspargase pegol-mknl, calcitriol, CCI-779, CDB-2914, cefixime, cetuximab, CG0070, cilengitide, clofarabine, combretastatin A4 phosphate, CP-675,206, CP-724,714, CpG 7909, curcumin, daratumumab, decitabine, DENSPM, dinutuximab, doxercalciferol, E7070, E7389, ecteinascidin 743, efaproxiral, eflornithine, EKB-569, elotuzumab, enzastaurin, erlotinib, exisulind, fenretinide, flavopiridol, fludarabine, flutamide, fotemustine, FR901228, G17DT, galiximab, gefitinib, genistein, glasdegib, glufosfamide, GTI-2040, histrelin, HKI-272, homoharringtonine, HSPPC-96, hu14.18-interleukin-2 fusion protein, HuMax-CD₄, iloprost, imiquimod, infliximab, inotuzumab ozogamicin, interleukin-12, IPI-504, irofulven, ixabepilone, lapatinib, lenalidomide, lestaurtinib, leuprolide, LMB-9 immunotoxin, lonafarnib, luniliximab, lutetium Lu 177 dotatate, mafosfamide, MB07133, MDX-010, MLN2704, mogamulizumab-kpkc, monoclonal antibody 3F8, monoclonal antibody J591, motexafin, moxetumomab pasudotox-tdfk, MS-275, MVA-MUC1-IL2, nilutamide, niraparib, nitrocamptothecin, nolatrexed dihydrochloride, nolvadex, NS-9, O6-benzylguanine, oblimersen sodium, ONYX-015, oregovomab, OSI-774, panitumumab, paraplatin, PD-0325901, pemetrexed, PHY906, pioglitazone, pirfenidone, pixantrone, polatuzumab vedotin-piiq, PS-341, PSC 833, PXD101, pyrazoloacridine, R115777, RAD001, ranpirnase, rebeccamycin analogue, rhuAngiostatin protein, rhuMab 2C4, rosiglitazone, rubitecan, rucaparib, S-1, S-8184, satraplatin, SB-, 15992, SGN-0010, SGN-40, sonidegib, sorafenib, SR31747A, ST1571, SU011248, suberoylanilide hydroxamic acid, suramin, tagraxofusp-erzs, talabostat, talampanel, talazoparib, tariquidar, temsirolimus, TGFa-PE38 immunotoxin, thalidomide, thymalfasin, tipifarnib, tirapazamine, TLK286, trabectedin, trifluridine and tipiracil hydrochloride, trimetrexate glucuronate, TroVax, UCN-1, valproic acid, vinflunine, VNP40101M, volociximab, vorinostat, VX-680, ZD1839, ZD6474, zileuton, and zosuquidar trihydrochloride.

In one embodiment, the optional therapeutic agent comprises one of the anti-cancer drugs or anti-cancer drug combinations listed in Table 5.

TABLE 5

| | | | |
|---|---|---|---|
| Abemaciclib | Abiraterone | Abraxane (Paclitaxel | ABVD |
| | Acetate | Albumin-stabilized | |
| | | Nanoparticle Formulation) | |
| ABVE | ABVE-PC | AC | Acalabrutinib |
| AC-T | Actemra | Adcetris (Brentuximab | ADE |
| | (Tocilizumab) | Vedotin) | |
| Ado-Trastuzumab | Adriamycin | Afatinib Dimaleate | Afinitor |
| Emtansine | (Doxorubicin | | (Everolimus) |
| | Hydrochloride) | | |
| Akynzeo | Aldara | Aldesleukin | Alecensa |
| (Netupitant and | (Imiquimod) | | (Alectinib) |
| Palonosetron | | | |
| Hydrochloride) | | | |
| Alectinib | Alemtuzumab | Alimta (Pemetrexed | Aliqopa |
| | | Disodium) | (Copanlisib |
| | | | Hydrochloride) |
| Alkeran for | Alkeran Tablets | Aloxi (Palonosetron | Alunbrig |
| Injection | (Melphalan) | Hydrochloride) | (Brigatinib) |
| (Melphalan | | | |
| Hydrochloride) | | | |
| Ameluz | Amifostine | Aminolevulinic Acid | Anastrozole |
| (Aminolevulinic | | | |
| Acid) | | | |
| Apalutamide | Aprepitant | Aranesp (Darbepoetin | Aredia |
| | | Alfa) | (Pamidronate |
| | | | Disodium) |
| Arimidex | Aromasin | Arranon (Nelarabine) | Arsenic Trioxide |
| (Anastrozole) | (Exemestane) | | |
| Arzerra | Asparaginase | Atezolizumab | Avastin |
| (Ofatumumab) | Erwinia | | (Bevacizumab) |
| | chrysanthemi | | |
| Avelumab | Axicabtagene | Axitinib | Azacitidine |
| | Ciloleucel | | |
| Azedra | Bavencio | BEACOPP | Beleodaq |
| (Iobenguane I 131) | (Avelumab) | | (Belinostat) |
| Belinostat | Bendamustine | Bendeka (Bendamustine | BEP |
| | Hydrochloride | Hydrochloride) | |
| Besponsa | Bevacizumab | Bexarotene | Bicalutamide |
| (Inotuzumab | | | |
| Ozogamicin) | | | |
| BiCNU | Binimetinib | Bleomycin | Blinatumomab |
| (Carmustine) | | | |
| Blincyto | Bortezomib | Bosulif (Bosutinib) | Bosutinib |
| (Blinatumomab) | | | |
| Braftovi | Brentuximab | Brigatinib | BuMel |
| (Encorafenib) | Vedotin | | |
| Busulfan | Busulfex | Cabazitaxel | Cabometyx |
| | (Busulfan) | | (Cabozantinib-S- |
| | | | Malate) |
| Cabozantinib-S- | CAF | Calquence | Campath |
| Malate | | (Acalabrutinib) | (Alemtuzumab) |
| Camptosar | Capecitabine | CAPOX | Carac |
| (Irinotecan | | | (Fluorouracil-- |
| Hydrochloride) | | | Topical) |
| Carboplatin | CARBOPLATIN- | Carfilzomib | Carmustine |
| | TAXOL | | |
| Carmustine | Casodex | CEM | Cemiplimab-rwlc |
| Implant | (Bicalutamide) | | |
| Ceritinib | Cerubidine | Cervarix (Recombinant | Cetuximab |
| | (Daunorubicin | HPV Bivalent Vaccine) | |
| | Hydrochloride) | | |
| CEV | Chlorambucil | CHLORAMBUCIL- | CHOP |
| | | PREDNISONE | |
| Cisplatin | Cladribine | Clofarabine | Clolar |
| | | | (Clofarabine) |
| CMF | Cobimetinib | Cometriq (Cabozantinib- | Copanlisib |
| | | S-Malate) | Hydrochloride |
| COPDAC | Copiktra | COPP | COPP-ABV |
| | (Duvelisib) | | |
| Cosmegen | Cotellic | Crizotinib | CVP |
| (Dactinomycin) | (Cobimetinib) | | |
| Cyclophosphamide | Cyramza | Cytarabine | Cytarabine |
| | (Ramucirumab) | | Liposome |
| Cytosar-U | Dabrafenib | Dacarbazine | Dacogen |
| (Cytarabine) | | | (Decitabine) |
| Dacomitinib | Dactinomycin | Daratumumab | Darbepoetin Alfa |
| Darzalex | Dasatinib | Daunorubicin | Daunorubicin |
| (Daratumumab) | | Hydrochloride | Hydrochloride |
| | | | and Cytarabine |
| | | | Liposome |
| Decitabine | Defibrotide | Defitelio (Defibrotide | Degarelix |
| | Sodium | Sodium) | |
| Denileukin | Denosumab | DepoCyt (Cytarabine | Dexamethasone |
| Diftitox | | Liposome) | |
| Dexrazoxane | Dinutuximab | Docetaxel | Doxil |
| Hydrochloride | | | (Doxorubicin |
| | | | Hydrochloride |
| | | | Liposome) |
| Doxorubicin | Doxorubicin | Dox-SL (Doxorubicin | Durvalumab |
| Hydrochloride | Hydrochloride | Hydrochloride Liposome) | |
| | Liposome | | |
| Duvelisib | Efudex | Eligard (Leuprolide | Elitek |
| | (Fluorouracil-- | Acetate) | (Rasburicase) |
| | Topical) | | |
| Ellence | Elotuzumab | Eloxatin (Oxaliplatin) | Eltrombopag |
| (Epirubicin | | | Olamine |
| Hydrochloride) | | | |
| Emend | Empliciti | Enasidenib Mesylate | Encorafenib |
| (Aprepitant) | (Elotuzumab) | | |
| Enzalutamide | Epirubicin | EPOCH | Epoetin Alfa |
| | Hydrochloride | | |
| Epogen | Erbitux | Eribulin Mesylate | Erivedge |
| (Epoetin Alfa) | (Cetuximab) | | (Vismodegib) |
| Erleada | Erlotinib | Erwinaze (Asparaginase | Ethyol |
| (Apalutamide) | Hydrochloride | Erwinia chrysanthemi) | (Amifostine) |
| Etopophos | Etoposide | Etoposide Phosphate | Evacet |
| (Etoposide | | | (Doxorubicin |
| Phosphate) | | | Hydrochloride |
| | | | Liposome) |
| Everolimus | Evista (Raloxifene | Evomela (Melphalan | Exemestane |
| | Hydrochloride) | Hydrochloride) | |
| 5-FU (Fluorouracil | 5-FU | Fareston (Toremifene) | Farydak |
| Injection) | (Fluorouracil-- | | (Panobinostat |
| | Topical) | | lactate) |
| Faslodex | FEC | Femara (Letrozole) | Filgrastim |
| (Fulvestrant) | | | |
| Firmagon | Fludarabine | Fluoroplex | Fluorouracil |
| (Degarelix) | Phosphate | (Fluorouracil--Topical) | Injection |
| Fluorouracil-- | Flutamide | FOLFIRI | FOLFIRI- |
| Topical | | | BEVACIZUMAB |
| FOLFIRI- | FOLFIRINOX | FOLFOX | Folotyn |
| CETUXIMAB | | | (Pralatrexate) |
| Fostamatinib | FU-LV | Fulvestrant | Fusilev |
| Disodium | | | (Leucovorin |
| | | | Calcium) |
| Gardasil | Gardasil 9 | Gazyva (Obinutuzumab) | Gefitinib |
| (Recombinant | (Recombinant | | |
| HPV Quadrivalent | HPV Nonavalent | | |
| Vaccine) | Vaccine) | | |
| Gemcitabine | GEMCITABINE- | GEMCITABINE- | Gemtuzumab |
| Hydrochloride | CISPLATIN | OXALIPLATIN | Ozogamicin |
| Gemzar | Gilotrif (Afatinib | Gleevec (Imatinib | Gliadel Wafer |
| (Gemcitabine | Dimaleate) | Mesylate) | (Carmustine |
| Hydrochloride) | | | Implant) |
| Glucarpidase | Goserelin Acetate | Granisetron | Granisetron |
| | | | Hydrochloride |
| Granix | Halaven (Eribulin | Hemangeol (Propranolol | Herceptin |
| (Filgrastim) | Mesylate) | Hydrochloride) | (Trastuzumab) |
| HPV Bivalent | HPV Nonavalent | HPV Quadrivalent | Hycamtin |
| Vaccine, | Vaccine, | Vaccine, Recombinant | (Topotecan |
| Recombinant | Recombinant | | Hydrochloride) |
| Hydrea | Hydroxyurea | Hyper-CVAD | Ibrance |
| (Hydroxyurea) | | | (Palbociclib) |
| Ibritumomab | Ibrutinib | ICE | Iclusig (Ponatinib |
| Tiuxetan | | | Hydrochloride) |
| Idarubicin | Idelalisib | Idhifa (Enasidenib | Ifex (Ifosfamide) |
| Hydrochloride | | Mesylate) | |
| Ifosfamide | IL-2 | Imatinib Mesylate | Imbruvica |
| | (Aldesleukin) | | (Ibrutinib) |
| Imfinzi | Imiquimod | Imlygic (Talimogene | Inlyta (Axitinib) |
| (Durvalumab) | | Laherparepvec) | |
| Inotuzumab | Interferon Alfa- | Interleukin-2 | Intron A |
| Ozogamicin | 2b, Recombinant | (Aldesleukin) | (Recombinant |
| | | | Interferon Alfa-2b) |
| Iobenguane I 131 | Ipilimumab | Iressa (Gefitinib) | Irinotecan |
| | | | Hydrochloride |
| Irinotecan | Istodax | Ivosidenib | Ixabepilone |
| Hydrochloride | (Romidepsin) | | |
| Liposome | | | |
| Ixazomib Citrate | Ixempra | Jakafi (Ruxolitinib | JEB |
| | (Ixabepilone) | Phosphate) | |
| Jevtana | Kadcyla (Ado- | Kepivance (Palifermin) | Keytruda |
| (Cabazitaxel) | Trastuzumab | | (Pembrolizumab) |
| | Emtansine) | | |
| Kisqali | Kymriah | Kyprolis (Carfilzomib) | Lanreotide |
| (Ribociclib) | (Tisagenlecleucel) | | Acetate |
| Lapatinib | Larotrectinib | Lartruvo (Olaratumab) | Lenalidomide |
| Ditosylate | Sulfate | | |
| Lenvatinib | Lenvima | Letrozole | Leucovorin |
| Mesylate | (Lenvatinib | | Calcium |
| | Mesylate) | | |
| Leukeran | Leuprolide | Levulan Kerastik | Libtayo |
| (Chlorambucil) | Acetate | (Aminolevulinic Acid) | (Cemiplimab-rwlc) |
| LipoDox | Lomustine | Lonsurf (Trifluridine and | Lorbrena |
| (Doxorubicin | | Tipiracil Hydrochloride) | (Lorlatinib) |
| Hydrochloride | | | |
| Liposome) | | | |
| Lorlatinib | Lumoxiti | Lupron (Leuprolide | Lupron Depot |
| | (Moxetumomab | Acetate) | (Leuprolide |
| | Pasudotox-tdfk) | | Acetate) |
| Lutathera | Lutetium (Lu | Lynparza (Olaparib) | Marqibo |
| (Lutetium Lu | 177-Dotatate) | | (Vincristine |
| 177-Dotatate) | | | Sulfate |
| | | | Liposome) |
| Matulane | Mechlorethamine | Megestrol Acetate | Mekinist |
| (Procarbazine | Hydrochloride | | (Trametinib) |
| Hydrochloride) | | | |
| Mektovi | Melphalan | Melphalan | Mercaptopurine |
| (Binimetinib) | | Hydrochloride | |
| Mesna | Mesnex (Mesna) | Methotrexate | Methylnaltrexone |
| | | | Bromide |
| Midostaurin | Mitomycin C | Mitoxantrone | Mogamulizumab-kpkc |
| | | Hydrochloride | |
| Moxetumomab | Mozobil | Mustargen | MVAC |
| Pasudotox-tdfk | (Plerixafor) | (Mechlorethamine | |
| | | Hydrochloride) | |
| Myleran | Mylotarg | Nanoparticle Paclitaxel | Navelbine |
| (Busulfan) | (Gemtuzumab | (Paclitaxel Albumin- | (Vinorelbine |
| | Ozogamicin) | stabilized Nanoparticle | Tartrate) |
| | | Formulation) | |
| Necitumumab | Nelarabine | Neratinib Maleate | Nerlynx |
| | | | (Neratinib Maleate) |
| Netupitant and | Neulasta | Neupogen (Filgrastim) | Nexavar |
| Palonosetron | (Pegfilgrastim) | | (Sorafenib Tosylate) |
| Hydrochloride | | | |
| Nilandron | Nilotinib | Nilutamide | Ninlaro |
| (Nilutamide) | | | (Ixazomib Citrate) |
| Niraparib Tosylate | Nivolumab | Nplate (Romiplostim) | Obinutuzumab |
| Monohydrate | | | |
| Odomzo (Sonidegib) | OEPA | Ofatumumab | OFF |
| Olaparib | Olaratumab | Omacetaxine | Oncaspar |
| | | Mepesuccinate | (Pegaspargase) |
| Ondansetron | Onivyde | Ontak (Denileukin | Opdivo |
| Hydrochloride | (Irinotecan | Diftitox) | (Nivolumab) |
| | Hydrochloride | | |
| | Liposome) | | |
| OPPA | Osimertinib | Oxaliplatin | Paclitaxel |
| Paclitaxel | PAD | Palbociclib | Palifermin |
| Albumin-stabilized | | | |
| Nanoparticle | | | |
| Formulation | | | |
| Palonosetron | Palonosetron | Pamidronate Disodium | Panitumumab |
| Hydrochloride | Hydrochloride | | |
| | and Netupitant | | |
| Panobinostat | Pazopanib | PCV | PEB |
| Lactate | Hydrochloride | | |
| Pegaspargase | Pegfilgrastim | Peginterferon Alfa-2b | PEG-Intron |
| | | | (Peginterferon |
| | | | Alfa-2b) |
| Pembrolizumab | Pemetrexed | Perjeta (Pertuzumab) | Pertuzumab |
| | Disodium | | |
| Plerixafor | Pomalidomide | Pomalyst | Ponatinib |
| | | (Pomalidomide) | Hydrochloride |
| Portrazza | Poteligeo | Pralatrexate | Prednisone |
| (Necitumumab) | (Mogamulizumab-kpkc) | | |
| Procarbazine | Procrit | Proleukin (Aldesleukin) | Prolia |
| Hydrochloride | (Epoetin Alfa) | | (Denosumab) |
| Promacta | Propranolol | Provenge (Sipuleucel-T) | Purinethol |
| (Eltrombopag | Hydrochloride | | (Mercaptopurine) |
| Olamine) | | | |
| Purixan | Radium 223 | Raloxifene | Ramucirumab |
| (Mercaptopurine) | Dichloride | Hydrochloride | |
| Rasburicase | R-CHOP | R-CVP | Recombinant |
| | | | Human |
| | | | Papillomavirus |
| | | | (HPV) Bivalent |
| | | | Vaccine |
| Recombinant Human | Recombinant Human | Recombinant Interferon | Regorafenib |
| Papillomavirus (HPV) | Papillomavirus (HPV) | Alfa-2b | |
| Nonavalent Vaccine | Quadrivalent Vaccine | | |
| Relistor | R-EPOCH | Retacrit (Epoetin Alfa) | Revlimid |
| (Methylnaltrexone | | | (Lenalidomide) |
| Bromide) | | | |
| Rheumatrex | Ribociclib | R-ICE | Rituxan |
| (Methotrexate) | | | (Rituximab) |
| Rituxan Hycela | Rituximab | Rituximab and | Rolapitant |
| (Rituximab and | | Hyaluronidase Human | Hydrochloride |
| Hyaluronidase | | | |
| Human) | | | |
| Romidepsin | Romiplostim | Rubidomycin | Rubraca |
| | | (Daunorubicin | (Rucaparib |
| | | Hydrochloride) | Camsylate) |
| Rucaparib | Ruxolitinib | Rydapt (Midostaurin) | Sancuso |
| Camsylate | Phosphate | | (Granisetron) |
| Sclerosol | Siltuximab | Sipuleucel-T | Somatuline Depot |
| Intrapleural | | | (Lanreotide Acetate) |
| Aerosol (Talc) | | | |
| Sonidegib | Sorafenib | Sprycel (Dasatinib) | STANFORD V |
| | Tosylate | | |
| Sterile Talc | Steritalc (Talc) | Stivarga (Regorafenib) | Sunitinib Malate |
| Powder (Talc) | | | |
| Sustol | Sutent (Sunitinib | Sylatron (Peginterferon | Sylvant |
| (Granisetron) | Malate) | Alfa-2b) | (Siltuximab) |
| Synribo | Tabloid | TAC | Tafinlar |
| (Omacetaxine | (Thioguanine) | | (Dabrafenib) |
| Mepesuccinate) | | | |
| Tagrisso | Talc | Talimogene | Tamoxifen |
| (Osimertinib) | | Laherparepvec | Citrate |
| Tarabine PFS | Tarceva (Erlotinib | Targretin (Bexarotene) | Tasigna |
| (Cytarabine) | Hydrochloride) | | (Nilotinib) |
| Tavalisse | Taxol (Paclitaxel) | Taxotere (Docetaxel) | Tecentriq |
| (Fostamatinib | | | (Atezolizumab) |
| Disodium) | | | |
| Temodar | Temozolomide | Temsirolimus | Thalidomide |
| (Temozolomide) | | | |
| Thalomid | Thioguanine | Thiotepa | Tibsovo |
| (Thalidomide) | | | (Ivosidenib) |
| Tisagenlecleucel | Tocilizumab | Tolak (Fluorouracil-- | Topotecan |
| | | Topical) | Hydrochloride |
| Toremifene | Torisel | Totect (Dexrazoxane | TPF |
| | (Temsirolimus) | Hydrochloride) | |
| Trabectedin | Trametinib | Trastuzumab | Treanda |
| | | | (Bendamustine |
| | | | Hydrochloride) |
| Trexall | Trifluridine and | Trisenox (Arsenic | Tykerb (Lapatinib |
| (Methotrexate) | Tipiracil | Trioxide) | Ditosylate) |
| | Hydrochloride | | |
| Unituxin | Uridine Triacetate | VAC | Valrubicin |
| (Dinutuximab) | | | |
| Valstar | Vandetanib | VAMP | Varubi |
| (Valrubicin) | | | (Rolapitant |
| | | | Hydrochloride) |
| Vectibix | VeIP | Velcade (Bortezomib) | Vemurafenib |
| (Panitumumab) | | | |
| Venclexta | Venetoclax | Verzenio (Abemaciclib) | Vidaza |
| (Venetoclax) | | | (Azacitidine) |
| Vinblastine | Vincristine | Vincristine Sulfate | Vinorelbine |
| Sulfate | Sulfate | Liposome | Tartrate |
| VIP | Vismodegib | Vistogard (Uridine | Vitrakvi |
| | | Triacetate) | (Larotrectinib |
| | | | Sulfate) |
| Vizimpro | Voraxaze | Vorinostat | Votrient |
| (Dacomitinib) | (Glucarpidase) | | (Pazopanib |
| | | | Hydrochloride) |
| Vyxeos | Xalkori | Xeloda (Capecitabine) | XELIRI |
| (Daunorubicin | (Crizotinib) | | |
| Hydrochloride | | | |
| and Cytarabine | | | |
| Liposome) | | | |
| XELOX | Xgeva | Xofigo (Radium 223 | Xtandi |
| | (Denosumab) | Dichloride) | (Enzalutamide) |
| Yervoy | Yescarta | Yondelis (Trabectedin) | Zaltrap (Ziv- |
| (Ipilimumab) | (Axicabtagene | | Aflibercept) |
| | Ciloleucel) | | |
| Zarxio | Zejula (Niraparib | Zelboraf (Vemurafenib) | Zevalin |
| (Filgrastim) | Tosylate | | (Ibritumomab |
| | Monohydrate) | | Tiuxetan) |
| Zinecard | Ziv-Aflibercept | Zofran (Ondansetron | Zoladex |
| (Dexrazoxane | | Hydrochloride) | (Goserelin |
| Hydrochloride) | | | Acetate) |
| Zoledronic Acid | Zolinza | Zometa (Zoledronic | Zydelig |
| | (Vorinostat) | Acid) | (Idelalisib) |
| Zykadia | Zytiga | | |
| (Ceritinib) | (Abiraterone | | |
| | Acetate) | | |

For a more detailed description of anticancer agents and other optional therapeutic agents, those skilled in the art are referred to any number of instructive manuals including, but not limited to, the Physician's Desk Reference and to Goodman and Gilman's “Pharmaceutical Basis of Therapeutics” tenth edition, Eds. Hardman et al., 2002.

In another embodiment, the methods of treating cancer provided herein comprise administering a Formulation of the Disclosure to a subject in combination with radiation therapy and, optionally, an immune checkpoint inhibitor. The methods provided herein are not limited by the types, amounts, or delivery and administration systems used to deliver the therapeutic dose of radiation to a patient. For example, the patient may receive photon radiotherapy, particle beam radiation therapy, other types of radiotherapies, and combinations thereof. In some embodiments, the radiation is delivered to the patient using a linear accelerator. In still other embodiments, the radiation is delivered using a gamma knife.

The source of radiation can be external or internal to the patient. External radiation therapy is most common and involves directing a beam of high-energy radiation to a tumor site through the skin using, for instance, a linear accelerator. While the beam of radiation is localized to the tumor site, it is nearly impossible to avoid exposure of normal, healthy tissue. However, external radiation is usually well tolerated by patients. Internal radiation therapy involves implanting a radiation-emitting source, such as beads, wires, pellets, capsules, particles, and the like, inside the body at or near the tumor site including the use of delivery systems that specifically target cancer cells (e.g., using particles attached to cancer cell binding ligands). Such implants can be removed following treatment, or left in the body inactive. Types of internal radiation therapy include, but are not limited to, brachytherapy, interstitial irradiation, intracavity irradiation, radioimmunotherapy, and the like.

The patient may optionally receive radiosensitizers (e.g., metronidazole, misonidazole, intra-arterial Budr, intravenous iododeoxyuridine (IudR), nitroimidazole, 5-substituted-4-nitroimidazoles, 2H-isoindolediones, [[(2-bromoethyl)-amino]methyl]-nitro-1H-imidazole-1-ethanol, nitroaniline derivatives, DNA-affinic hypoxia selective cytotoxins, halogenated DNA ligand, 1,2,4 benzotriazine oxides, 2-nitroimidazole derivatives, fluorine-containing nitroazole derivatives, benzamide, nicotinamide, acridine-intercalator, 5-thiotretrazole derivative, 3-nitro-1,2,4-triazole, 4,5-dinitroimidazole derivative, hydroxylated texaphrins, cisplatin, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide, paclitaxel, heat (hyperthermia), and the like), radioprotectors (e.g., cysteamine, aminoalkyl dihydrogen phosphorothioates, amifostine (WR 2721), IL-1, IL-6, and the like). Radiosensitizers enhance the killing of tumor cells. Radioprotectors protect healthy tissue from the harmful effects of radiation.

Any type of radiation can be administered to a patient, so long as the dose of radiation is tolerated by the patient without unacceptable negative side-effects. Suitable types of radiotherapy include, for example, ionizing (electromagnetic) radiotherapy (e.g., X-rays or gamma rays) or particle beam radiation therapy (e.g., high linear energy radiation). Ionizing radiation is defined as radiation comprising particles or photons that have sufficient energy to produce ionization, i.e., gain or loss of electrons (as described in, for example, U.S. Pat. No. 5,770,581 incorporated herein by reference in its entirety). The effects of radiation can be at least partially controlled by the clinician. In one embodiment, the dose of radiation is fractionated for maximal target cell exposure and reduced toxicity.

In one embodiment, the total dose of radiation administered to a patient is about 0.01 Gray (Gy) to about 100 Gy. In another embodiment, about 10 Gy to about 65 Gy (e.g., about 15 Gy, 20 Gy, 25 Gy, 30 Gy, 35 Gy, 40 Gy, 45 Gy, 50 Gy, 55 Gy, or 60 Gy) are administered over the course of treatment. While in some embodiments a complete dose of radiation can be administered over the course of one day, the total dose is ideally fractionated and administered over several days. Desirably, radiotherapy is administered over the course of at least about 3 days, e.g., at least 5, 7, 10, 14, 17, 21, 25, 28, 32, 35, 38, 42, 46, 52, or 56 days (about 1-8 weeks). Accordingly, a daily dose of radiation will comprise approximately 1-5 Gy (e.g., about 1 Gy, 1.5 Gy, 1.8 Gy, 2 Gy, 2.5 Gy, 2.8 Gy, 3 Gy, 3.2 Gy, 3.5 Gy, 3.8 Gy, 4 Gy, 4.2 Gy, or 4.5 Gy), or 1-2 Gy (e.g., 1.5-2 Gy). The daily dose of radiation should be sufficient to induce destruction of the targeted cells. If stretched over a period, in one embodiment, radiation is not administered every day, thereby allowing the animal to rest and the effects of the therapy to be realized. For example, radiation desirably is administered on 5 consecutive days, and not administered on 2 days, for each week of treatment, thereby allowing 2 days of rest per week. However, radiation can be administered 1 day/week, 2 days/week, 3 days/week, 4 days/week, 5 days/week, 6 days/week, or all 7 days/week, depending on the animal's responsiveness and any potential side effects. Radiation therapy can be initiated at any time in the therapeutic period. In one embodiment, radiation is initiated in week 1 or week 2, and is administered for the remaining duration of the therapeutic period. For example, radiation is administered in weeks 1-6 or in weeks 2-6 of a therapeutic period comprising 6 weeks for treating, for instance, a solid tumor. Alternatively, radiation is administered in weeks 1-5 or weeks 2-5 of a therapeutic period comprising 5 weeks. These exemplary radiotherapy administration schedules are not intended, however, to limit the methods provided herein.

### V. Kits

In another embodiment, the present disclosure provides kits comprising a Compound of the Disclosure or Composition of the Disclosure packaged in a manner that facilitates their use to practice methods of the present disclosure.

In one embodiment, the kit includes a Compound of the Disclosure or Composition of the Disclosure packaged in a container, such as a sealed bottle or vessel, with a label affixed to the container or included in the kit that describes use of the compound or composition to practice the method of the disclosure. In one embodiment, the compound or composition is packaged in a unit dosage form. The kit may include a single dose or multiple doses of a Compound of the Disclosure or Composition of the Disclosure.

### VI. Particular Embodiments

The disclosure provides the following particular embodiments.

Embodiment 1. A compound of Formula I:

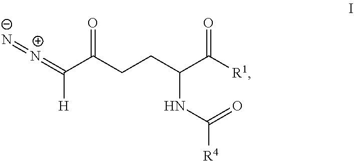

or a pharmaceutically acceptable salt thereof, wherein:
R¹ is selected from the group consisting of —OR² and —NR^{3a}R^{3b};
R² is selected from the group consisting of hydrogen, C₁-C₆ alkyl, C₁-C₆ haloalkyl C₁-C₆ alkenyl, C₃-C₂₀ heteroalkyl, and C₃-C₆ cycloalkyl;
R^{3a} and R^{3b} are independently selected from the group consisting of hydrogen and C₁-C₄ alkyl; or
R^{3a} and R^{3b} taken together with the nitrogen atom to which they are attached from a 4- to 8-membered heterocyclo;
R⁴ is selected from the group consisting of C₁-C₆ alkyl, C₁-C₆ haloalkyl, (amino)C₁-C₆ alkyl, (amino)(aryl)C₁-C₆ alkyl, optionally substituted heteroaryl, —CH(R^{5a})N(R^{6a})C(═O)R^{7a}, —(CH₂)ₘ—N(R^{6c})C(═O)R^{7c}, and —OR⁸;
R^{5a} is selected from the group consisting of hydrogen, optionally substituted C₁-C₆ alkyl, optionally substituted aryl, (heterocyclo)C₁-C₄ alkyl, (aryl)C₁-C₄ alkyl, and (heteroaryl)C₁-C₄ alkyl;
R^{6a} is selected from the group consisting of hydrogen and C₁-C₄ alkyl;
R^{7a} is selected from the group consisting of C₁-C₆ alkyl, C₁-C₆ haloalkyl, optionally substituted C₃-C₈ cycloalkyl, optionally substituted 4- to 10-membered heterocyclo, optionally substituted heteroaryl, (amino)C₁-C₄ alkyl, (heterocyclo)C₁-C₄ alkyl, and —CH(R^{5b})N(R^{6b})C(═O)R^{7b},
R⁸ is optionally substituted 4- to 10-membered heterocyclo;
R^{5b} is selected from the group consisting of hydrogen, optionally substituted C₁-C₆ alkyl, (aryl)C₁-C₄ alkyl, and (heteroaryl)C₁-C₄ alkyl,
R^{6b} is selected from the group consisting of hydrogen and C₁-C₄ alkyl;
R^{7b} is selected from the group consisting of C₁-C₆ alkyl, C₁-C₆ haloalkyl, optionally substituted C₃-C₈ cycloalkyl, optionally substituted 4- to 10-membered heterocyclo, optionally substituted heteroaryl, (amino)C₁-C₄ alkyl, and (heterocyclo)C₁-C₄ alkyl;
R^{6c} is selected from the group consisting of hydrogen and C₁-C₄ alkyl;
R^{7c} is selected from the group consisting of C₁-C₆ alkyl, C₁-C₆ haloalkyl, optionally substituted C₃-C₈ cycloalkyl, optionally substituted 4- to 10-membered heterocyclo, optionally substituted heteroaryl, (amino)C₁-C₄ alkyl, and (heterocyclo)C₁-C₄ alkyl; and
m is 2, 3, 4, or 5,
with the proviso that the compound of Formula I is not a compound of Table 1.

Embodiment 2. The compound of Embodiment 1 of Formula II.

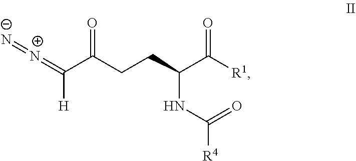

or a pharmaceutically acceptable salt thereof.

Embodiment 3. The compound of Embodiment 1 of Formula III:

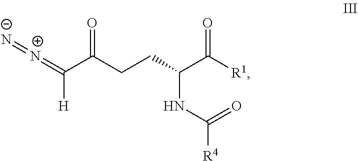

or a pharmaceutically acceptable salt thereof.

Embodiment 4. The compound of any one of Embodiments 1-3, or a pharmaceutically acceptable salt thereof, wherein R¹ is —OR².

Embodiment 5. The compound of Embodiment 4, or a pharmaceutically acceptable salt thereof, wherein R² is selected from the group consisting of hydrogen, —CH₃, —CD₃, —CH₂CH₃, —CH(CH₃)₂, —C(CH₃)₃, —CH(CH₂F)₂, —CH₂CH═CH₂, and —OCH₂CH₂OCH₂CH₂OCH₃.

Embodiment 6. The compound of any one of Embodiments 1-3, or a pharmaceutically acceptable salt thereof, wherein R¹ is —NR^{3a}R^{3b}.

Embodiment 7. The compound of Embodiment 6, or a pharmaceutically acceptable salt thereof, wherein R^{3a} and R^{3b} are independently selected from the group consisting of hydrogen and methyl.

Embodiment 8. The compound of any one of Embodiments 1-7, or a pharmaceutically acceptable salt thereof, wherein R⁴ is C₁-C₆ alkyl.

Embodiment 9. The compound of Embodiment 8, or a pharmaceutically acceptable salt thereof, wherein R⁴ is selected from the group consisting of —CH₃, —CH₂CH₃, and —(CH₂)₄CH₃.

Embodiment 10. The compound of any one of Embodiments 1-7, or a pharmaceutically acceptable salt thereof, wherein R⁴ is C₁-C₆ haloalkyl.

Embodiment 11. The compound of Embodiment 10, or a pharmaceutically acceptable salt thereof, wherein R⁴ is —CHCl₂.

Embodiment 12. The compound of any one of Embodiments 1-7, or a pharmaceutically acceptable salt thereof, wherein R⁴ is (amino)C₁-C₆ alkyl.

Embodiment 13. The compound of claim 12, or a pharmaceutically acceptable salt thereof, wherein R⁴ is —CH₂N(CH₃)₂.

Embodiment 14. The compound of any one of Embodiments 1-7, or a pharmaceutically acceptable salt thereof, wherein R⁴ is optionally substituted heteroaryl.

Embodiment 15. The compound of Embodiment 14, or a pharmaceutically acceptable salt thereof, wherein R⁴ is 2-pyridyl, 3-pyridyl, or 4-pyridyl.

Embodiment 16. The compound of any one of Embodiments 1-7, or a pharmaceutically acceptable salt thereof, wherein R⁴ is —OR⁸.

Embodiment 17. The compound of Embodiment 16, or a pharmaceutically acceptable salt thereof, wherein R⁸ is:

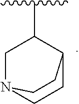 [SMILES: *C1CN2CCC1CC2 ; "."]

Embodiment 18. The compound of any one of Embodiments 1-7, or a pharmaceutically acceptable salt thereof, wherein R⁴ is —(CH₂)ₘ—N(R^{6c})C(═O)R^{7c}.

Embodiment 19. The compound of Embodiment 18, or a pharmaceutically acceptable salt thereof, wherein m is 3.

Embodiment 20. The compound of Embodiments 18 or 19, or a pharmaceutically acceptable salt thereof, wherein R^{6c} is hydrogen.

Embodiment 21. The compound of any one of Embodiments 18-20, or a pharmaceutically acceptable salt thereof, wherein R^{7c} is 4- to 10-membered heterocyclo.

Embodiment 22. The compound of Embodiment 21, or a pharmaceutically acceptable salt thereof, wherein R^{7c} is:

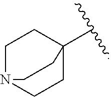

Embodiment 23. The compound of any one of Embodiments 1-7, or a pharmaceutically acceptable salt thereof, wherein R⁴ is —CH(R^{5a})N(R^{6a})C(═O)R^{7a}.

Embodiment 24. The compound of Embodiment 23, or a pharmaceutically acceptable salt thereof, wherein R⁴ is:

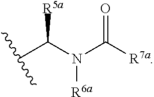

Embodiment 25. The compound of Embodiment 23, or a pharmaceutically acceptable salt thereof, wherein R⁴ is:

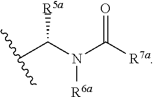

Embodiment 26. The compound of any one of Embodiments 23-25, or a pharmaceutically acceptable salt thereof, wherein R^{6a} is hydrogen.

Embodiment 27. The compound of Embodiments 23 or 26, or a pharmaceutically acceptable salt thereof, wherein R^{5a} is hydrogen.

Embodiment 28. The compound of any one of Embodiments 23-26, or a pharmaceutically acceptable salt thereof, wherein R^{5a} is optionally substituted C₁-C₆ alkyl.

Embodiment 29. The compound of Embodiment 28, or a pharmaceutically acceptable salt thereof, wherein R^{5a} is selected from the group consisting of —CH₃, —CH₂CH(CH₃)₂, and —CH₂C(CH₃)₃.

Embodiment 30. The compound of any one of Embodiments 23-26, or a pharmaceutically acceptable salt thereof, wherein R^{5a} is optionally substituted aryl.

Embodiment 31. The compound of Embodiment 30, or a pharmaceutically acceptable salt thereof, wherein R^{5a} is optionally substituted phenyl.

Embodiment 32. The compound of any one of Embodiments 23-26, or a pharmaceutically acceptable salt thereof, wherein R^{5a} is (heterocyclo)C₁-C₄ alkyl.

Embodiment 33. The compound of any one of Embodiments 23-26, or a pharmaceutically acceptable salt thereof, wherein R^{5a} is (aryl)C₁-C₄ alkyl.

Embodiment 34. The compound of Embodiment 33, wherein R^{5a} is selected from the group consisting of:

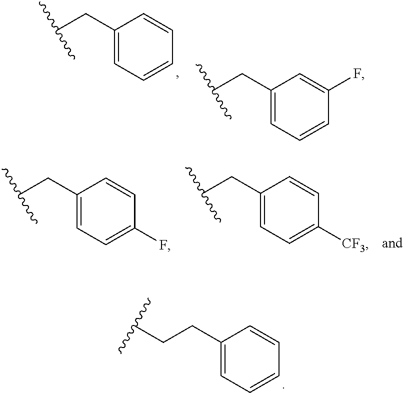

Embodiment 35. The compound of any one of Embodiments 23-26, or a pharmaceutically acceptable salt thereof, wherein R^{5a} is (heteroaryl)C₁-C₄ alkyl.

Embodiment 36. The compound of Embodiment 35, wherein R^{5a} is selected from the group consisting of:

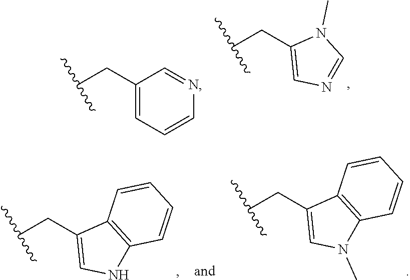

Embodiment 37. The compound of any one of Embodiments 23-36, or a pharmaceutically acceptable salt thereof, wherein R^{7a} is C₁-C₄ alkyl.

Embodiment 38. The compound of Embodiment 37, or a pharmaceutically acceptable salt thereof, wherein R^{7a} is selected from the group consisting of —CH₃, —CH(CH₃)₂, —C(CH₃)₃, —CH₂CH₂CH₃, and —CH₂CH(CH₃)₂.

Embodiment 39. The compound of any one of Embodiments 23-36, or a pharmaceutically acceptable salt thereof, wherein R^{7a} is C₁-C₄ haloalkyl.

Embodiment 40. The compound of Embodiment 39, or a pharmaceutically acceptable salt thereof, wherein R^{7a} is —CHCl₂.

Embodiment 41. The compound of any one of Embodiments 23-36, or a pharmaceutically acceptable salt thereof, wherein R^{7a} is C₃-C₈ cycloalkyl.

Embodiment 42. The compound of Embodiment 41, or a pharmaceutically acceptable salt thereof, wherein R^{7a} is cyclopropyl.

Embodiment 43. The compound of any one of Embodiments 23-36, or a pharmaceutically acceptable salt thereof, wherein R^{7a} is optionally substituted 4- to 10-membered heterocyclo.

Embodiment 44. The compound of Embodiment 43, or a pharmaceutically acceptable salt thereof, wherein R^{7a} is selected from the group consisting of:

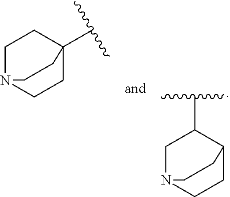

Embodiment 45. The compound of any one of Embodiments 23-36, or a pharmaceutically acceptable salt thereof, wherein R^{7a} is (amino)C₁-C₄ alkyl.

Embodiment 46. The compound of Embodiment 45, or a pharmaceutically acceptable salt thereof, wherein R^{7a} is selected from the group consisting of —CH₂N(CH₃)₂ and —CH₂N(CH₂CH₃)₂.

Embodiment 47. The compound of any one of Embodiments 23-36, or a pharmaceutically acceptable salt thereof, wherein R^{7a} is (heterocyclo)C₁-C₄ alkyl.

Embodiment 48. The compound of Embodiment 47, or a pharmaceutically acceptable salt thereof, wherein R^{7a} is selected from the group consisting of.

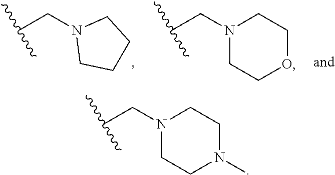

Embodiment 49. The compound of any one of Embodiments 23-36, or a pharmaceutically acceptable salt thereof, wherein R^{7a} is —CH(R^{5b})N(R^{6b})C(═O)R^{7b}.

Embodiment 50. The compound of Embodiment 49, or a pharmaceutically acceptable salt thereof, wherein R^{7a} is —CH₂N(H)C(═O)CH₂N(CH₃)₂.

Embodiment 51. The compound of Embodiment 1, or pharmaceutically acceptable salt thereof, selected from the group consisting of the compounds of Table 2.

Embodiment 52. A pharmaceutical composition comprising the compound of any one of Embodiments 1-51, or a pharmaceutically acceptable salt thereof, and a pharmaceutically acceptable carrier.

Embodiment 53. The pharmaceutical composition of Embodiment 52, wherein the pharmaceutically acceptable carrier comprises water.

Embodiment 54. A method of treating cancer in a subject in need thereof, the method comprising administering a therapeutically effective amount of the compound of any one of Embodiments 1-51, or a pharmaceutically acceptable salt thereof, to the subject.

Embodiment 55. The method of claim 54 further comprising administering an optional therapeutic agent to the subject.

Embodiment 56. The method of claim 55, wherein the optional therapeutic agent is an immune checkpoint inhibitor.

Embodiment 57. The method of claim 56, wherein the immune checkpoint inhibitor is selected from the group consisting of a PD-1 inhibitor, a PD-L1 inhibitor, a CTLA-4 inhibitor, a LAG3 inhibitor, a TIM3 inhibitor, and a cd47 inhibitor.

Embodiment 58. The method of any one of Embodiments 54-57, wherein the cancer is any one or more of the cancers of Table 3.

Embodiment 59. The method of any one of Embodiments 54-58, wherein the cancer is a solid tumor.

Embodiment 60. The method of any one of Embodiments 54-59, wherein the cancer is a hematological cancer.

Embodiment 61. The method of Embodiment 60, wherein the hematological cancer is acute lymphocytic leukemia, chronic lymphocytic leukemia (including B-cell chronic lymphocytic leukemia), or acute myeloid leukemia.

Embodiment 62. The method of any one of Embodiments 54-58, wherein the cancer is squamous cell carcinoma of the head and neck, adenocarcinoma squamous cell carcinoma of the esophagus, adenocarcinoma of the stomach, adenocarcinoma of the colon, hepatocellular carcinoma, cholangiocarcinoma of the biliary system, adenocarcinoma of gall bladder, adenocarcinoma of the pancreas, ductal carcinoma in situ of the breast, adenocarcinoma of the breast, adenocarcinoma of the lungs, squamous cell carcinoma of the lungs, transitional cell carcinoma of the bladder, squamous cell carcinoma of the bladder, squamous cell carcinoma of the cervix, adenocarcinoma of the cervix, endometrial carcinoma, penile squamous cell carcinoma, or squamous cell carcinoma of the skin.

Embodiment 63. The method of any one of Embodiments 54-58, wherein the cancer is hepatocellular carcinoma, glioblastoma, lung cancer, breast cancer, head and neck cancer, prostate cancer, melanoma, or colorectal cancer.

Embodiment 64. The method of any one of Embodiments 54-58, wherein the cancer is colorectal cancer, breast cancer, lymphoma, melanoma, kidney cancer, or lung cancer.

### VII. Definitions

The terms “a”, “an”, “the”, and similar referents in the context of describing the disclosure (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language, e.g., “such as,” provided herein, is intended to better illustrate the disclosure and is not a limitation on the scope of the disclosure unless otherwise claimed. No language in the specification should be construed as indicating any non-claimed element as essential to the practice of the disclosure.

The term “about,” as used herein, includes the recited number±10%. Thus, “about 10” means 9 to 11.

The term “DON prodrug” and the like as used herein refers to a pharmacologically inactive derivative of DON, that requires biotransformation within the target physiological system, e.g., a cancer cell, to release, or to convert the prodrug into DON. DON prodrugs are designed, e.g., to overcome problems associated with the stability, water solubility, toxicity, lack of specificity, limited bioavailability, etc. of DON.

As used herein, the terms “treat,” “treating,” “treatment,” “therapeutic methods,” and the like refer to eliminating, reducing, or ameliorating a disease, disorder, or condition, and/or symptoms associated therewith. Although not precluded, treating a disease, disorder or condition does not require that the disease, condition, or symptoms associated therewith be completely eliminated. The term “treat” and synonyms contemplate administering a therapeutically effective amount of a Compound of the Disclosure or Composition of the Disclosure to a subject in need of such treatment. The treatment can be orientated symptomatically, for example, to suppress symptoms. It can be effected over a short period, be oriented over a medium term, or can be a long-term treatment, for example within the context of a maintenance therapy.

As used herein, the terms “prevent,” “preventing,” and “prevention” refer to a method of preventing the onset of a disease, disorder, or condition and/or its attendant symptoms or barring a subject from acquiring a disease. As used herein, “prevent,” “preventing,” and “prevention” also include delaying the onset of a disease, disorder, or condition and/or its attendant symptoms and reducing a subject's risk of acquiring a disease. The terms “prevent,” “preventing” and “prevention” may include “prophylactic treatment,” which refers to reducing the probability of redeveloping a disease, disorder, or condition, or of a recurrence of a previously-controlled disease, disorder, or condition, in a subject who does not have, but is at risk of or is susceptible to, redeveloping a disease or condition or a recurrence of the disease, disorder, or condition.

The term “therapeutically effective amount,” as used herein, refers to that amount of the therapeutic agent sufficient to result in amelioration of one or more symptoms of a disease, disorder, or condition, or prevent advancement of a disease, disorder, or condition, or cause regression of the disease, disorder, or condition. For example, with respect to the treatment of cancer, in one embodiment, a therapeutically effective amount will refer to the amount of a therapeutic agent that causes a therapeutic response, e.g., normalization of blood counts, decrease in the rate of tumor growth, decrease in tumor mass, decrease in the number of metastases, increase in time to tumor progression, and/or increase subject survival time by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, or more.

The term “pharmaceutically acceptable carrier” or “pharmaceutically acceptable vehicle” encompasses any of the standard pharmaceutical carriers, solvents, surfactants, or vehicles. Suitable pharmaceutically acceptable vehicles include aqueous vehicles and nonaqueous vehicles. Standard pharmaceutical carriers and their formulations are described in Remington's Pharmaceutical Sciences, Mack Publishing Co., Easton, PA, 19th ed. 1995.

The term “disease or condition wherein the inhibition of glutamine-utilizing enzymes provides a benefit” and the like pertains to a disease, disorder, or condition in which glutamine is important or necessary, e.g., for the onset, progress, expression of that disease, disorder, or condition, or a disease, disorder, or a condition which is known to be treated by an glutamine antagonist. Examples of such conditions include, but are not limited to, cancer, an immune disorder, or a neurological disease or deficit. One of ordinary skill in the art is readily able to determine whether a compound treats a disease, disorder, or condition mediated by glutamine, for example, by in vitro and/or in vivo assays which conveniently can be used to assess the activity of particular compounds.

The terms “patient” and “subject” as used herein are synonymous terms referring to any human or animal that is in need of or might benefit from administration of a Compound of the Disclosure or Composition of the Disclosure. Foremost among such subjects are mammals, e.g., humans, although the methods and compositions provided herein are not intended to be so limited. Other subjects include veterinary animals, e.g., cows, sheep, pigs, horses, dogs, cats and the like. In one embodiment, the subject is a human. In one embodiment, the subject is an animal.

The term “halo” as used herein by itself or as part of another group refers to —Cl, —F, —Br, or —I.

The term “nitro” as used herein by itself or as part of another group refers to —NO2.

The term “cyano” as used herein by itself or as part of another group refers to —CN.

The term “hydroxy” as herein used by itself or as part of another group refers to —OH.

The term “alkyl” as used herein by itself or as part of another group refers to a straight- or branched-chain aliphatic hydrocarbon containing one to twelve carbon atoms, i.e., a C₁-C₁₂ alkyl, or the number of carbon atoms designated, e.g., a C₁ alkyl such as methyl, a C₂ alkyl such as ethyl, etc. In one embodiment, the alkyl is a C₁-C₁₀ alkyl. In another embodiment, the alkyl is a C₁-C₆ alkyl. In another embodiment, the alkyl is a C₁-C₄ alkyl. In another embodiment, the alkyl is a C₁-C₃ alkyl, i.e., methyl, ethyl, propyl, or isopropyl. Non-limiting exemplary C₁-C₁₂ alkyl groups include methyl, ethyl, propyl, isopropyl, butyl, sec-butyl, tert-butyl, iso-butyl, 3-pentyl, hexyl, heptyl, octyl, nonyl, and decyl.

The term “optionally substituted alkyl” as used herein by itself or as part of another group refers to an alkyl group that is either unsubstituted or substituted with one, two, or three substituents, wherein each substituent is independently nitro, haloalkoxy, aryloxy, aralkyloxy, alkylthio, sulfonamido, alkylcarbonyl, arylcarbonyl, alkylsulfonyl, arylsulfonyl, ureido, guanidino, carbamate, carboxy, alkoxycarbonyl, carboxyalkyl, —N(R^{56a})C(═O)R^{56b}, —N(R^{56c})S(═O)₂R^{56d}, —C(═O)R⁵⁷, —S(═O)R^{56e}, or —S(═O)₂R⁵⁸; wherein:
R^{56a} is hydrogen or alkyl;
R^{56b} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;
R^{56c} is hydrogen or alkyl;
R^{56d} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;
R^{56e} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;
R⁵⁷ is haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, or optionally substituted heteroaryl; and
R⁵⁸ is haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, or optionally substituted heteroaryl. Non-limiting exemplary optionally substituted alkyl groups include —CH(CO₂Me)CH₂CO₂Me and —CH(CH₃)CH₂N(H)C(═O)O(CH₃)₃.

The term “alkenyl” as used herein by itself or as part of another group refers to an alkyl group containing one, two, or three carbon-to-carbon double bonds. In one embodiment, the alkenyl group is a C₂-C₆ alkenyl group. In another embodiment, the alkenyl group is a C₂-C₄ alkenyl group. In another embodiment, the alkenyl group has one carbon-to-carbon double bond. Non-limiting exemplary alkenyl groups include ethenyl, propenyl, isopropenyl, butenyl, sec-butenyl, pentenyl, and hexenyl.

The term “optionally substituted alkenyl” as used herein by itself or as part of another refers to an alkenyl group that is either unsubstituted or substituted with one, two or three substituents, wherein each substituent is independently halo, nitro, cyano, hydroxy, amino (e.g., alkylamino, dialkylamino), haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted cycloalkyl, alkenyl, alkynyl, optionally substituted aryl, optionally substituted heteroaryl, or optionally substituted heterocyclo. Non-limiting exemplary optionally substituted alkenyl groups include —CH═CHPh.

The term “alkynyl” as used herein by itself or as part of another group refers to an alkyl group containing one, two, or three carbon-to-carbon triple bonds. In one embodiment, the alkynyl is a C₂-C₆ alkynyl. In another embodiment, the alkynyl is a C₂-C₄ alkynyl. In another embodiment, the alkynyl has one carbon-to-carbon triple bond. Non-limiting exemplary alkynyl groups include ethynyl, propynyl, butynyl, 2-butynyl, pentynyl, and hexynyl groups.

The term “optionally substituted alkynyl” as used herein by itself or as part of another group refers to an alkynyl group that is either unsubstituted or substituted with one, two or three substituents, wherein each substituent is independently halo, nitro, cyano, hydroxy, amino, e.g., alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted cycloalkyl, alkenyl, alkynyl, optionally substituted aryl, optionally substituted heteroaryl, or optionally substituted heterocyclo. Non-limiting exemplary optionally substituted alkynyl groups include —C≡CPh and —CH(Ph)C≡CH.

The term “haloalkyl” as used herein by itself or as part of another group refers to an alkyl group substituted by one or more fluorine, chlorine, bromine, and/or iodine atoms. In one embodiment, the alkyl is substituted by one, two, or three fluorine and/or chlorine atoms. In another embodiment, the alkyl is substituted by one, two, or three fluorine atoms. In another embodiment, the alkyl is a C₁-C₆ alkyl. In another embodiment, the alkyl is a C₁-C₄ alkyl. In another embodiment, the alkyl group is a C₁ or C₂ alkyl. Non-limiting exemplary haloalkyl groups include fluoromethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

The terms “hydroxyalkyl” or “(hydroxy)alkyl” as used herein by themselves or as part of another group refer to an alkyl group substituted with one, two, or three hydroxy groups. In one embodiment, the alkyl is a C₁-C₆ alkyl. In another embodiment, the alkyl is a C₁-C₄ alkyl. In another embodiment, the alkyl is a C₁ or C₂ alkyl. In another embodiment, the hydroxyalkyl is a monohydroxyalkyl group, i.e., substituted with one hydroxy group. In another embodiment, the hydroxyalkyl group is a dihydroxyalkyl group, i.e., substituted with two hydroxy groups. Non-limiting exemplary (hydroxyl)alkyl groups include hydroxymethyl, hydroxyethyl, hydroxypropyl and hydroxybutyl groups, such as 1-hydroxyethyl, 2-hydroxyethyl, 1,2-dihydroxyethyl, 2-hydroxypropyl, 3-hydroxypropyl, 3-hydroxybutyl, 4-hydroxybutyl, 2-hydroxy-1-methylpropyl, and 1,3-dihydroxyprop-2-yl.

The term “alkoxy” as used herein by itself or as part of another group refers to an alkyl group attached to a terminal oxygen atom. In one embodiment, the alkyl is a C₁-C₆ alkyl and resulting alkoxy is thus referred to as a “C₁-C₆ alkoxy.” In another embodiment, the alkyl is a C₁-C₄ alkyl group. Non-limiting exemplary alkoxy groups include methoxy, ethoxy, and tert-butoxy.

The term “haloalkoxy” as used herein by itself or as part of another group refers to a haloalkyl group attached to a terminal oxygen atom. In one embodiment, the haloalkyl group is a C₁-C₆ haloalkyl. In another embodiment, the haloalkyl group is a C₁-C₄haloalkyl group. Non-limiting exemplary haloalkoxy groups include fluoromethoxy, difluoromethoxy, trifluoromethoxy, and 2,2,2-trifluoroethoxy.

The term “alkylthio” as used herein by itself or as part of another group refers to an alkyl group attached to a terminal sulfur atom. In one embodiment, the alkyl group is a C₁-C₄ alkyl group. Non-limiting exemplary alkylthio groups include —SCH₃, and —SCH₂CH₃.

The terms “alkoxyalkyl” or “(alkoxy)alkyl” as used herein by themselves or as part of another group refers to an alkyl group substituted with one alkoxy group. In one embodiment, the alkoxy is a C₁-C₆ alkoxy. In another embodiment, the alkoxy is a C₁-C₄ alkoxy. In another embodiment, the alkyl is a C₁-C₆ alkyl. In another embodiment, the alkyl is a C₁-C₄ alkyl. Non-limiting exemplary alkoxyalkyl groups include methoxymethyl, methoxyethyl, methoxypropyl, methoxybutyl, ethoxymethyl, ethoxyethyl, ethoxypropyl, ethoxybutyl, propoxymethyl, iso-propoxymethyl, propoxyethyl, propoxypropyl, butoxymethyl, tert-butoxymethyl, isobutoxymethyl, see-butoxymethyl, and pentyloxymethyl.

The term “heteroalkyl” as used by itself or part of another group refers to unsubstituted straight- or branched-chain aliphatic hydrocarbons containing from three to twenty chain atoms, i.e., 3- to 20-membered heteroalkyl, or the number of chain atoms designated, wherein at least one —CH₂— is replaced with at least one of —O—, —N(H)—, —N(C₁-C₄ alkyl)-, or —S—. The —O—, —N(H)—, —N(C₁-C₄ alkyl)-, or —S— can independently be placed at any interior position of the aliphatic hydrocarbon chain so long as each —O—, —N(H)—, —N(C₁-C₄ alkyl)-, and —S— group is separated by at least two —CH₂— groups. In one embodiment, one —CH₂— group is replaced with one —O— group. In another embodiment, two —CH₂— groups are replaced with two —O— groups. In another embodiment, three —CH₂— groups are replaced with three —O— groups. In another embodiment, four —CH₂— groups are replaced with four —O— groups. Non-limiting exemplary heteroalkyl groups include —CH₂OCH₃, —CH₂OCH₂CH₂CH₃, —CH₂CH₂CH₂OCH₃, —CH₂CH₂OCH₂CH₂OCH₂CH₃, —CH₂CH₂OCH₂CH₂OCH₂CH₂OCH₂CH₃.

The term “cycloalkyl” as used herein by itself or as part of another group refers to saturated and partially unsaturated, e.g., containing one or two double bonds, monocyclic, bicyclic, or tricyclic aliphatic hydrocarbons containing three to twelve carbon atoms, i.e., a C₃₋₁₂ cycloalkyl, or the number of carbons designated, e.g., a C₃ cycloalkyl such a cyclopropyl, a C₄ cycloalkyl such as cyclobutyl, etc. In one embodiment, the cycloalkyl is bicyclic, i.e., it has two rings. In another embodiment, the cycloalkyl is monocyclic, i.e., it has one ring. In another embodiment, the cycloalkyl is a C₃₋₈ cycloalkyl. In another embodiment, the cycloalkyl is a C₃₋₆ cycloalkyl, i.e., cyclopropyl, cyclobutyl, cyclopentyl, or cyclohexyl. In another embodiment, the cycloalkyl is a C₅ cycloalkyl, i.e., cyclopentyl. In another embodiment, the cycloalkyl is a C₆ cycloalkyl, i.e., cyclohexyl. Non-limiting exemplary C₃₋₁₂ cycloalkyl groups include cyclopropyl, cyclobutyl, cyclopentyl, cyclohexyl, cycloheptyl, cyclooctyl, norbornyl, decalin, adamantyl, cyclohexenyl, and spiro[3.3]heptane.

The term “optionally substituted cycloalkyl” as used herein by itself or as part of another group refers to a cycloalkyl group that is either unsubstituted or substituted with one, two, or three substituents, wherein each substituent is independently halo, nitro, cyano, hydroxy, amino (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —C(═O)R⁵⁷, —N(R^{56c})S(═O)₂R^{56d}, —S(═O)R^{56e}, —S(═O)₂R⁵⁸, or —OR⁵⁹, wherein R^{56a}, R^{56b}, R^{56c}, R^{56d}, R^{56e}, R⁵⁷, and R⁵⁸ are as defined in connection with the term “optionally substituted alkyl” and R⁵⁹ is (hydroxy)alkyl or (amino)alkyl. The term optionally substituted cycloalkyl also includes cycloalkyl groups having fused optionally substituted aryl or optionally substituted heteroaryl groups such as

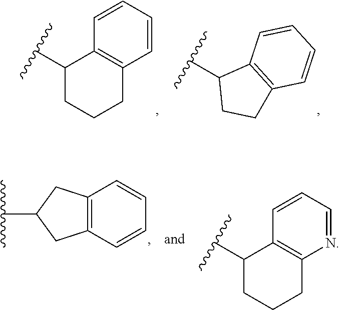

Non-limiting exemplary optionally substituted cycloalkyl groups include:

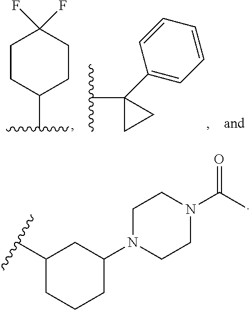

The term “heterocyclo” as used herein by itself or as part of another group refers to saturated and partially unsaturated, e.g., containing one or two double bonds, monocyclic, bicyclic, or tricyclic groups containing three to fourteen ring members, i.e., a 3- to 14-membered heterocyclo, comprising one, two, three, or four heteroatoms. Each heteroatom is independently oxygen, sulfur, or nitrogen. Each sulfur atom is independently oxidized to give a sulfoxide, i.e., S(═O), or sulfone, i.e., S(═O)₂.

The term heterocyclo includes groups wherein one or more —CH₂— groups is replaced with one or more —C(═O)— groups, including cyclic ureido groups such as imidazolidinyl-2-one, cyclic amide groups such as pyrrolidin-2-one or piperidin-2-one, and cyclic carbamate groups such as oxazolidinyl-2-one.

The term heterocyclo also includes groups having fused optionally substituted aryl or optionally substituted heteroaryl groups such as indoline, indolin-2-one, 2,3-dihydro-1H-pyrrolo[2,3-c]pyridine, 2,3,4,5-tetrahydro-1H-benzo[d]azepine, or 1,3,4,5-tetrahydro-2H-benzo[d]azepin-2-one.

In one embodiment, the heterocyclo group is a 4- to 8-membered cyclic group containing one ring and one or two oxygen atoms, e.g., tetrahydrofuran or tetrahydropyran, or one or two nitrogen atoms, e.g., pyrrolidine, piperidine, or piperazine, or one oxygen and one nitrogen atom, e.g., morpholine, and, optionally, one —CH₂— group is replaced with one —C(═O)— group, e.g., pyrrolidin-2-one or piperazin-2-one. In another embodiment, the heterocyclo group is a 5- to 8-membered cyclic group containing one ring and one or two nitrogen atoms and, optionally, one —CH₂— group is replaced with one —C(═O)— group. In another embodiment, the heterocyclo group is a 5- or 6-membered cyclic group containing one ring and one or two nitrogen atoms and, optionally, one —CH₂— group is replaced with one —C(═O)— group. In another embodiment, the heterocyclo group is a 8- to 12-membered cyclic group containing two rings and one or two nitrogen atoms. The heterocyclo can be linked to the rest of the molecule through any available carbon or nitrogen atom. Non-limiting exemplary heterocyclo groups include:

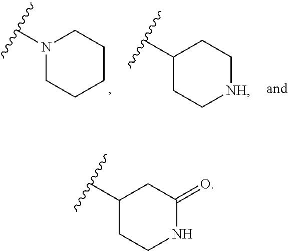

The term “optionally substituted heterocyclo” as used herein by itself or part of another group refers to a heterocyclo group that is either unsubstituted or substituted with one to four substituents, wherein each substituent is independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(Rsa)C(═O)R^{56b}, —N(R^{56c})S(═O)₂R^{56d}, —C(═O)R⁵⁷, —S(═O)R^{56e}, —S(═O)₂R⁵⁸, or —OR⁵⁹, wherein R^{56a}, R^{56b}, R^{56c}, R^{56d}, R^{56e}, R⁵⁷, R⁵⁸, and R⁵⁹ are as defined in connection with the term “optionally substituted cycloalkyl.” Substitution may occur on any available carbon or nitrogen atom of the heterocyclo group. Non-limiting exemplary optionally substituted heterocyclo groups include:

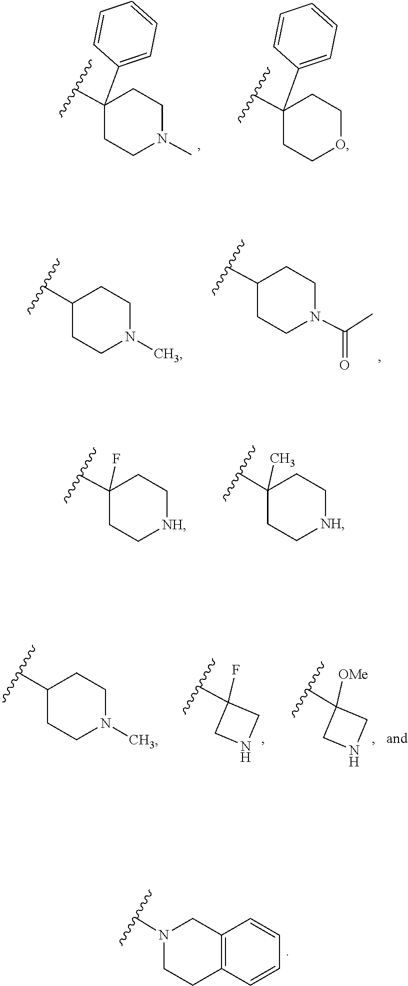

The term “aryl” as used herein by itself or as part of another group refers to an aromatic ring system having six to fourteen carbon atoms, i.e., C₆-C₁₄ aryl. Non-limiting exemplary aryl groups include phenyl (abbreviated as “Ph”), naphthyl, phenanthryl, anthracyl, indenyl, azulenyl, biphenyl, biphenylenyl, and fluorenyl groups. In one embodiment, the aryl group is phenyl or naphthyl. In another embodiment, the aryl group is phenyl.

The term “optionally substituted aryl” as used herein by itself or as part of another group refers to aryl that is either unsubstituted or substituted with one to five substituents, wherein the substituents are each independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, or —OR⁵⁹, wherein R^{56a}, R^{56b}, R^{56c}, R^{56d}, R^{56e}, R⁵⁷, R⁵⁸, and R⁵⁹ are as defined in connection with the term “optionally substituted cycloalkyl.”

In one embodiment, the optionally substituted aryl is an optionally substituted phenyl. In another embodiment, the optionally substituted phenyl has four substituents. In another embodiment, the optionally substituted phenyl has three substituents. In another embodiment, the optionally substituted phenyl has two substituents. In another embodiment, the optionally substituted phenyl has one substituent. Non-limiting exemplary optionally substituted aryl groups include 2-methylphenyl, 2-methoxyphenyl, 2-fluorophenyl, 2-chlorophenyl, 2-bromophenyl, 3-methylphenyl, 3-methoxyphenyl, 3-fluorophenyl, 3-chlorophenyl, 4-methylphenyl, 4-ethylphenyl, 4-methoxyphenyl, 4-fluorophenyl, 4-chlorophenyl, 2,6-di-fluorophenyl, 2,6-di-chlorophenyl, 2-methyl, 3-methoxyphenyl, 2-ethyl, 3-methoxyphenyl, 3,4-di-methoxyphenyl, 3,5-di-fluorophenyl 3,5-di-methylphenyl, 3,5-dimethoxy, 4-methylphenyl, 2-fluoro-3-chlorophenyl, 3-chloro-4-fluorophenyl, and 2-phenylpropan-2-amine. The term optionally substituted aryl includes aryl groups having fused optionally substituted cycloalkyl groups and fused optionally substituted heterocyclo groups. Non-limiting xamples include: 2,3-dihydro-1H-inden-1-yl, 1,2,3,4-tetrahydronaphthalen-1-yl, 1,3,4,5-tetrahydro-2H-benzo[c]azepin-2-yl, 1,2,3,4-tetrahydroisoquinolin-1-yl, and 2-oxo-2,3,4,5-tetrahydro-1H-benzo[d]azepin-1-yl.

The term “heteroaryl” as used herein by itself or as part of another group refers to monocyclic and bicyclic aromatic ring systems having five to 14 fourteen ring members, i.e., a 5- to 14-membered heteroaryl, comprising one, two, three, or four heteroatoms. Each heteroatom is independently oxygen, sulfur, or nitrogen. In one embodiment, the heteroaryl has three heteroatoms. In another embodiment, the heteroaryl has two heteroatoms. In another embodiment, the heteroaryl has one heteroatom. In another embodiment, the heteroaryl is a 5- to 10-membered heteroaryl. In another embodiment, the heteroaryl has 5 ring atoms, e.g., thienyl, a 5-membered heteroaryl having four carbon atoms and one sulfur atom. In another embodiment, the heteroaryl has 6 ring atoms, e.g., pyridyl, a 6-membered heteroaryl having five carbon atoms and one nitrogen atom. Non-limiting exemplary heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, β-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. In one embodiment, the heteroaryl is chosen from thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl) and isoxazolyl (e.g., isoxazol-3-yl, isoxazol-4-yl, and isoxazol-5-yl). The term heteroaryl also includes N-oxides. A non-limiting exemplary N-oxide is pyridyl N-oxide.

The term “optionally substituted heteroaryl” as used herein by itself or as part of another group refers to a heteroaryl that is either unsubstituted or substituted with one to four substituents, wherein the substituents are independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, or —OR⁵⁹, wherein R^{56a}, R^{56b}, R^{56c}, R^{56d}, R^{56e}, R⁵⁷, R⁵⁸, and R⁵⁹ are as defined in connection with the term “optionally substituted cycloalkyl.”

In one embodiment, the optionally substituted heteroaryl has two substituents. In another embodiment, the optionally substituted heteroaryl has one substituent. Any available carbon or nitrogen atom can be substituted.

The term “aryloxy” as used herein by itself or as part of another group refers to an optionally substituted aryl attached to a terminal oxygen atom. A non-limiting exemplary aryloxy group is PhO—.

The term “heteroaryloxy” as used herein by itself or as part of another group refers to an optionally substituted heteroaryl attached to a terminal oxygen atom. A non-limiting exemplary aryloxy group is pyridyl-O—.

The term “aralkyloxy” as used herein by itself or as part of another group refers to an aralkyl attached to a terminal oxygen atom. A non-limiting exemplary aralkyloxy group is PhCH₂O—.

The term “(cyano)alkyl” as used herein by itself or as part of another group refers to an alkyl substituted with one, two, or three cyano groups. In one embodiment, the alkyl is substituted with one cyano group. In another embodiment, the alkyl is a C₁-C₆ alkyl In another embodiment, the alkyl is a C₁-C₄ alkyl. Non-limiting exemplary (cyano)alkyl groups include —CH₂CH₂CN and —CH₂CH₂CH₂CN.

The term “(cycloalkyl)alkyl” as used herein by itself or as part of another group refers to an alkyl substituted with one or two optionally substituted cycloalkyl groups. In one embodiment, the cycloalkyl group(s) is an optionally substituted C₃-C₆ cycloalkyl. In another embodiment, the alkyl is a C₁-C₆ alkyl. In another embodiment, the alkyl is a C₁-C₄ alkyl. In another embodiment, the alkyl is a C₁ or C₂ alkyl. In another embodiment, the alkyl is substituted with one optionally substituted cycloalkyl group. In another embodiment, the alkyl is substituted with two optionally substituted cycloalkyl groups. Non-limiting exemplary (cycloalkyl)alkyl groups include:

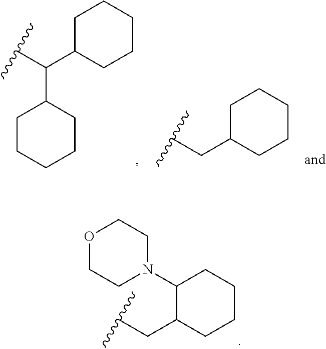 [SMILES: *C(C1CCCCC1)C1CCCCC1 ; *CC1CCCCC1 ; *CC1CCCCC1N1CCOCC1]

The term “sulfonamido” as used herein by itself or as part of another group refers to a radical of the formula —SO₂NR^{50a}R^{50b}, wherein R^{50a} and R^{50b} are each independently hydrogen, alkyl, optionally substituted cycloalkyl, optionally substituted heterocyclo, optionally substituted aryl, or optionally substituted heteroaryl; or R^{50a} and R^{50b} taken together with the nitrogen to which they are attached form a 3- to 8-membered optionally substituted heterocyclo group. Non-limiting exemplary sulfonamido groups include —SO₂NH₂, —SO₂N(H)CH₃, and —SO₂N(H)Ph.

The term “alkylcarbonyl” as used herein by itself or as part of another group refers to a carbonyl group, i.e., —C(═O)—, substituted by an alkyl group. In one embodiment, the alkyl is a C₁-C₄ alkyl. A non-limiting exemplary alkylcarbonyl group is —COCH₃.

The term “arylcarbonyl” as used herein by itself or as part of another group refers to a carbonyl group, i.e., —C(═O)—, substituted by an optionally substituted aryl group. A non-limiting exemplary arylcarbonyl group is —COPh.

The term “alkylsulfonyl” as used herein by itself or as part of another group refers to a sulfonyl group, i.e., —SO₂—, substituted by an alkyl group. A non-limiting exemplary alkylsulfonyl group is —SO₂CH₃.

The term “arylsulfonyl” as used herein by itself or as part of another group refers to a sulfonyl group, i.e., —SO₂—, substituted by an optionally substituted aryl group. A non-limiting exemplary arylsulfonyl group is —SO₂Ph.

The term “mercaptoalkyl” as used herein by itself or as part of another group refers to an alkyl substituted by a —SH group.

The term “carboxy” as used by itself or as part of another group refers to a radical of the formula —C(═O)OH.

The term “carboxamido” as used herein by itself or as part of another group refers to a radical of the formula —C(═O)NR^{50c}R^{50d}, wherein R^{50c} and R^{50d} are each independently hydrogen, alkyl, optionally substituted cycloalkyl, optionally substituted heterocyclo, optionally substituted aryl, optionally substituted heteroaryl, aralkyl, (heteroaryl)alkyl, or (heterocyclo)alkyl; or R^{50c} and R^{50d} taken together with the nitrogen to which they are attached form a 3- to 8-membered optionally substituted heterocyclo group. Non-limiting exemplary carboxamido groups include —C(═O)NH₂, —C(═O)N(H)CH₃, and —C(═O)N(H)Ph.

The term “(carboxamido)alkyl” as used herein by itself or as part of another group refers to an alkyl substituted with one carboxamido group. In one embodiment, the alkyl is a C₁-C₄ alkyl In another embodiment, the alkyl is a C₁-C₃ alkyl. Non-limiting exemplary (carboxamido)alkyl groups include —CH₂C(═O)NH₂ and —CH₂CH₂C(═O)NH₂.

The term “ureido” as used herein by itself or as part of another group refers to a radical of the formula —NR^{51a}—C(═O)—NR^{51b}R^{51c}, wherein R^{51a} is hydrogen or alkyl; and R^{51b} and R^{51c} are each independently hydrogen, alkyl, optionally substituted cycloalkyl, optionally substituted heterocyclo, optionally substituted aryl, or optionally substituted heteroaryl, or R^{51b} and R^{51c} taken together with the nitrogen to which they are attached form a 4- to 8-membered optionally substituted heterocyclo group. Non-limiting exemplary ureido groups include —NH—C(C═O)—NH₂ and —NH—C(C═O)—NHCH₃.

The term “guanidino” as used herein by itself or as part of another group refers to a radical of the formula —NR^{52a}—C(═NR⁵³)—NR^{52b}R^{52c}, wherein R^{52a} is hydrogen or alkyl; R^{52b} and R^{53c} are each independently hydrogen, alkyl, optionally substituted cycloalkyl, optionally substituted heterocyclo, optionally substituted aryl, or optionally substituted heteroaryl; or R^{52b} and R^{52c} taken together with the nitrogen to which they are attached form a 4- to 8-membered optionally substituted heterocyclo group; and R¹³ is hydrogen, alkyl, cyano, alkylsulfonyl, alkylcarbonyl, carboxamido, or sulfonamido. Non-limiting exemplary guanidino groups include —NH—C(C═NH)—NH₂, —NH—C(C═NCN)—NH₂, and —NH—C(C═NH)—NHCH₃.

The term “(heterocyclo)alkyl” as used herein by itself or as part of another group refers to an alkyl substituted with one, two, or three optionally substituted heterocyclo groups. In one embodiment, the alkyl is substituted with one optionally substituted 5- to 8-membered heterocyclo group. In another embodiment, alkyl is a C₁-C₆ alkyl. In another embodiment, alkyl is a C₁-C₄ alkyl. The heterocyclo group can be linked to the alkyl group through a carbon or nitrogen atom. Non-limiting exemplary (heterocyclo)alkyl groups include:

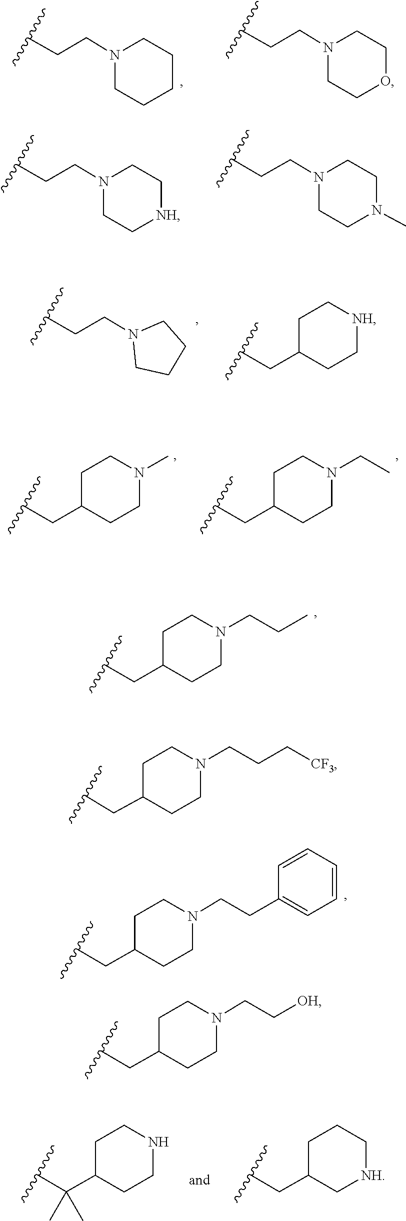

The term “carbamate” as used herein by itself or as part of another group refers to a radical of the formula —NR^{54a}—C(═O)—OR^{54b}, wherein R^{54a} is hydrogen or alkyl, and R⁵⁴¹ is hydrogen, alkyl, optionally substituted cycloalkyl, optionally substituted heterocyclo, optionally substituted aryl, or optionally substituted heteroaryl. A non-limiting exemplary carbamate group is —NH—(C═O)—OtBu.

The term “(heteroaryl)alkyl” as used herein by itself or as part of another group refers to an alkyl substituted with one or two optionally substituted heteroaryl groups. In one embodiment, the alkyl group is substituted with one optionally substituted 5- to 14-membered heteroaryl group. In another embodiment, the alkyl group is substituted with two optionally substituted 5- to 14-membered heteroaryl groups. In another embodiment, the alkyl group is substituted with one optionally substituted 5- to 9-membered heteroaryl group. In another embodiment, the alkyl group is substituted with two optionally substituted 5- to 9-membered heteroaryl groups. In another embodiment, the alkyl group is substituted with one optionally substituted 5- or 6-membered heteroaryl group. In another embodiment, the alkyl group is substituted with two optionally substituted 5- or 6-membered heteroaryl groups. In one embodiment, the alkyl group is a C₁-C₆ alkyl. In another embodiment, the alkyl group is a C₁-C₄ alkyl. In another embodiment, the alkyl group is a C₁ or C₂ alkyl. Non-limiting exemplary (heteroaryl)alkyl groups include:

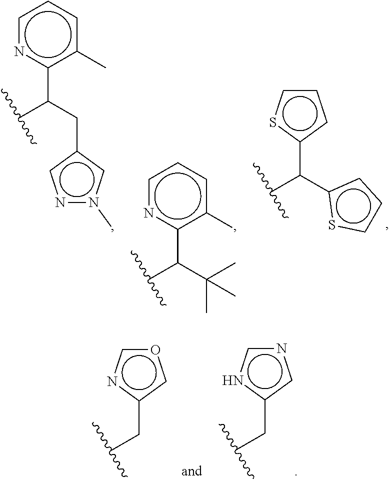

The terms “aralkyl” or “(aryl)alkyl” as used herein by themselves or as part of another group refers to an alkyl substituted with one, two, or three optionally substituted aryl groups. In one embodiment, the alkyl is substituted with one optionally substituted aryl group. In another embodiment, the alkyl is substituted with two optionally substituted aryl groups. In one embodiment, the aryl is an optionally substituted phenyl or optionally substituted naphthyl. In another embodiment, the aryl is an optionally substituted phenyl. In one embodiment, the alkyl is a C₁-C₆ alkyl. In another embodiment, the alkyl is a C₁-C₄ alkyl. In another embodiment, the alkyl is a C₁ or C₂ alkyl. Non-limiting exemplary (aryl)alkyl groups include benzyl, phenethyl, —CHPh₂, and —CH(4-F-Ph)₂.

The term “amido” as used herein by itself or as part of another group refers to a radical of formula —C(═O)NR^{60a}R^{60b}, wherein R^{60a} and R^{60b} are each independently hydrogen, optionally substituted alkyl, optionally substituted alkenyl, optionally substituted alkynyl, haloalkyl, (alkoxy)alkyl, (hydroxy)alkyl, (cyano)alkyl, optionally substituted cycloalkyl, optionally substituted heterocyclo, optionally substituted aryl, optionally substituted heteroaryl, (aryl)alkyl, (cycloalkyl)alkyl, (heterocyclo)alkyl, or (heteroaryl)alkyl; or R^{60a} and R^{60b} taken together with the nitrogen to which they are attached from a 4- to 8-membered optionally substituted heterocyclo group. In one embodiment, R^{60a} and R^{60b} are each independently hydrogen or C₁-C₆ alkyl.

The term “(amino)(aryl)alkyl” as used herein by itself or as part of another group refers to an alkyl group substituted with one amino group and one optionally substituted aryl group. In one embodiment, the amino group is —NH₂, alkylamino, or dialkylamino. In one embodiment, the aryl group is an optionally substituted phenyl. In one embodiment, the alkyl is a C₁-C₆ alkyl. In another embodiment, the alkyl is a C₁-C₄ alkyl. Non-limiting exemplary (amino)(aryl)alkyl groups include:

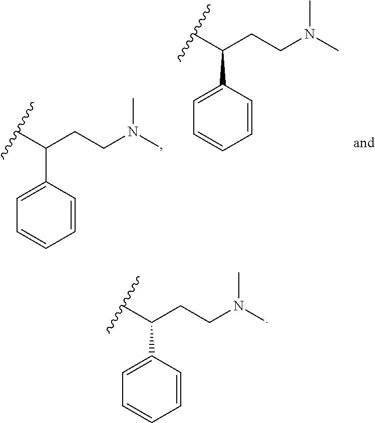

The term “amino” as used by itself or as part of another group refers to a radical of the formula —NR^{55a}R^{55b}, wherein R^{55a} and R^{55b} are independently hydrogen, optionally substituted alkyl, haloalkyl, (hydroxy)alkyl, (alkoxy)alkyl, (amino)alkyl, heteroalkyl, optionally substituted cycloalkyl, optionally substituted heterocyclo, optionally substituted aryl, optionally substituted heteroaryl, (aryl)alkyl, (cycloalkyl)alkyl, (heterocyclo)alkyl, or (heteroaryl)alkyl.

In one embodiment, the amino is —NH₂.

In another embodiment, the amino is an “alkylamino,” i.e., an amino group wherein R^{55a} is C₁₋₆ alkyl and R^{55b} is hydrogen. In one embodiment, R^{55a} is C₁-C₄ alkyl. Non-limiting exemplary alkylamino groups include —N(H)CH₃ and —N(H)CH₂CH₃.

In another embodiment, the amino is a “dialkylamino,” i.e., an amino group wherein R^{55a} and R^{55b} are each independently C₁₋₆ alkyl. In one embodiment, R^{55a} and R^{55b} are each independently C₁-C₄ alkyl. Non-limiting exemplary dialkylamino groups include —N(CH₃)₂ and —N(CH₃)CH₂CH(CH₃)₂.

In another embodiment, the amino is a “hydroxyalkylamino,” i.e., an amino group wherein R^{55a} is (hydroxyl)alkyl and R^{55b} is hydrogen or C₁-C₄ alkyl.

In another embodiment, the amino is a “cycloalkylamino,” i.e., an amino group wherein R^{55a} is optionally substituted cycloalkyl and R^{55b} is hydrogen or C₁-C₄ alkyl.

In another embodiment, the amino is a “aralkylamino,” i.e., an amino group wherein R^{55a} is aralkyl and R^{55b} is hydrogen or C₁-C₄ alkyl. Non-limiting exemplary aralkylamino groups include —N(H)CH₂Ph, —N(H)CHPh₂, and —N(CH₃)CH₂Ph.

In another embodiment, the amino is a “(heterocyclo)alkylamino,” i.e., an amino group wherein R^{55a} is (heterocyclo)alkyl and R^{55b} is hydrogen or C₁-C₄ alkyl. Non-limiting exemplary (heterocyclo)alkylamino groups include:

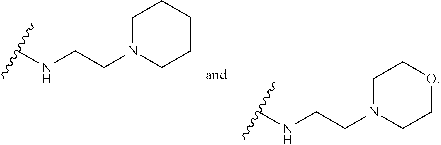 [SMILES: *NCCN1CCCCC1 ; *NCCN1CCOCC1]

The term “(amino)alkyl” as used herein by itself or as part of another group refers to an alkyl substituted with one amino group. In one embodiment, the amino group is —NH₂. In one embodiment, the amino group is an alkylamino. In another embodiment, the amino group is a dialkylamino. In another embodiment, the alkyl is a C₁-C₆ alkyl. In another embodiment, the alkyl is a C₁-C₄ alkyl. Non-limiting exemplary (amino)alkyl groups include —CH₂NH₂, CH₂CH₂N(H)CH₃, —CH₂CH₂N(CH₃)₂, CH₂N(H)cyclopropyl, —CH₂N(H)cyclobutyl, and —CH₂N(H)cyclohexyl, and —CH₂CH₂CH₂N(H)CH₂Ph and —CH₂CH₂CH₂N(H)CH₂ (4-CF₃-Ph).

The present disclosure encompasses any of the Compounds of the Disclosure being isotopically-labelled (i.e., radiolabeled) by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H (or deuterium (D)), ³H, ¹¹C, ¹³C, ¹⁴C ¹⁵N, ¹⁸O, ¹⁷O, ³¹P, ³²P, ³⁵S, ¹⁸F, and ³⁶Cl, respectively, e.g., ³H, ¹¹C, and ¹⁴C. In one embodiment, provided is a composition wherein substantially all of the atoms at a position within the Compound of the Disclosure are replaced by an atom having a different atomic mass or mass number. In another embodiment, provided is a composition wherein a portion of the atoms at a position within the Compound of the disclosure are replaced, i.e., the Compound of the Disclosure is enriched at a position with an atom having a different atomic mass or mass number.” Isotopically-labelled Compounds of the Disclosure can be prepared by methods known in the art.

Compounds of the Disclosure contain one or more asymmetric centers and may thus give rise to enantiomers, diastereomers, and other stereoisomeric forms. The present disclosure encompasses the use of all such possible forms, as well as their racemic and resolved forms and mixtures thereof. The individual enantiomers can be separated according to methods known in the art in view of the present disclosure. When the compounds described herein contain olefinic double bonds or other centers of geometric asymmetry, and unless specified otherwise, it is intended that they include both E and Z geometric isomers. All tautomers are also encompassed by the present disclosure.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive. In one embodiment, Compounds of the Disclosure are racemic.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem 68:2193 (1996), unless otherwise indicated.

The term “enantiomeric excess” or “ee” refers to a measure for how much of one enantiomer is present compared to the other. For a mixture of R and S enantiomers, the percent enantiomeric excess is defined as |R−S|*100, where R and S are the respective mole or weight fractions of enantiomers in a mixture such that R+S=1. With knowledge of the optical rotation of a chiral substance, the percent enantiomeric excess is defined as ([α]_{obs}/[α]ₘₐₓ)*100, where [α]_{obs} is the optical rotation of the mixture of enantiomers and [α]ₘₐₓ is the optical rotation of the pure enantiomer. Determination of enantiomeric excess is possible using a variety of analytical techniques, including NMR spectroscopy, chiral column chromatography or optical polarimetry.

### Example 1

### Preparation of Compounds 1a-1g, 2a-2g, 3a-3g, and 4a-4g

Compounds 1a-1g, 2a-2g, 3a-3g, and 4a-4g were prepared according to the following reaction Scheme.

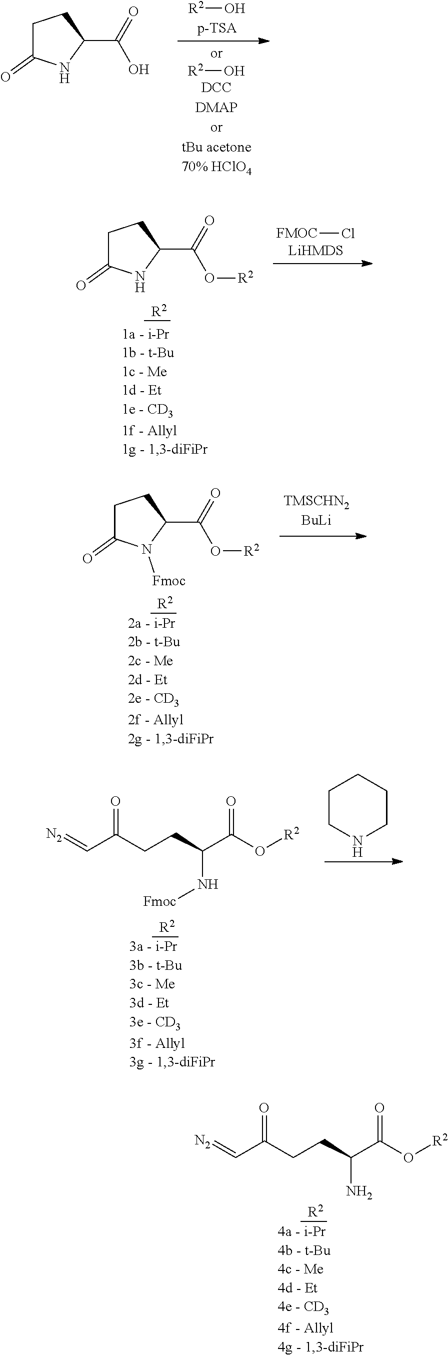

### Preparation of Isopropyl (S)-5-oxopyrrolidine-2-carboxylate (1a)

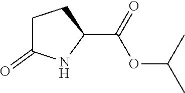

L-Pyroglutamic acid (12.9 g, 100 mmol, 1 equiv.) was suspended in anhydrous isopropylalcohol (26 mL), p-toluenesulfonic acid monohydrate (1.90 g, 10.0 mmol, 0.1 equiv.) and molecular sieves (20 pieces, 3 Å, activated) were added and the resulting mixture was heated to reflux for 13 h. The mixture was cooled to rt and Et₂O (400 mL) was added. Organic phase was washed with sat. NaHCO₃ (60 mL) and sat. NaCl (50 mL), combined water phases were then extracted with EtOAc (200 mL), combined organic phases were dried over anhydrous MgSO₄ and solvents were evaporated. Compound 1a was obtained as a colorless solid (12.7 g) in 74% yield. ¹H NMR (401 MHz, CDCl₃): 1.22 (dd, J=6.3, 0.8 Hz, 6H), 2.07-2.20 (m, 1H), 2.23-2.47 (m, 3H), 4.16 (ddd, J=8.7, 5.2, 0.8 Hz, 1H), 5.02 (hept, J=6.3 Hz, 1H), 6.85 (bs, 1H). ¹³C NMR (101 MHz, CDCl₃): 21.75 (2C), 24.88, 29.38, 55.70, 69.34, 171.66, 178.17.

### Preparation of tert-Butyl-(S)-5-oxopyrolidin-2-carboxylate (1b)

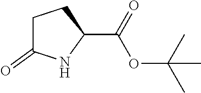

L-Pyroglutamic acid (6.46 g, 50 mmol, 1 equiv.) was suspended in t-butyl acetate (100 mL) in a pressure flask. 70% Perchloric acid (2.46 g, 1.50 mL, 17.5 mmol, 0.35 equiv.) was added and the resulting mixture was stirred at rt for 23 h. Then the mixture was slowly poured on sat. NaHCO₃ (200 mL) and stirred for 5 minutes. Phases were separated and water phase was extracted with DCM (2×100 mL), combined organic phases were dried over anhydrous MgSO₄ and solvents were evaporated. Compound 1b was obtained as a colorless solid (6.97 g) in 75% yield. ¹H NMR (401 MHz, CDCl₃): 1.45 (s, 9H), 2.09-2.19 (m, 1H), 2.22-2.48 (m, 3H), 4.06-4.14 (m, 1H), 6.56 (s, 1H). ¹³C NMR (101 MHz, CDCl₃): 24.9, 28.1 (3C), 29.5, 56.2, 82.4, 171.2, 178.1.

### Preparation of Methyl (S)-5-oxopyrrolidine-2-carboxylate (1c)

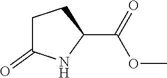

Commercially available compound, CAS: 98-79-3.

### Preparation of Ethyl (S)-5-oxopyrrolidine-2-carboxylate (1d)

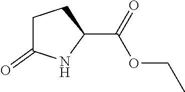

Commercially available compound, CAS: 7149-65-7.

### Preparation of Methyl-d₃ (S)-5-oxopyrrolidine-2-carboxylate (1e)

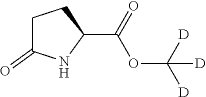 [SMILES: [2H]C([2H])([2H])OC(=O)[C@@H]1CCC(=O)N1]

L-Pyroglutamic acid (6.46 g, 50 mmol, 1 equiv.) was suspended in anhydrous DCM (20 mL), anhydrous CD₃OD (9.02 g, 10.2 mL, 250 mmol, 5 equiv.) was added and the mixture was cooled to 0° C. DCC (11.3 g, 55 mmol, 1.1 equiv.) and DMAP (611 mg, 5.0 mmol, 0.1 equiv.) were added in one portion. The resulting suspension was slowly heated to rt and stirred under inert atmosphere over weekend (90 h). The precipitate (DCU) was filtered off, washed with DCM (2×20 mL) and solvent was evaporated. The crude product was purified by LC on silica gel (EtOAc) and product 1e was obtained as a colorless oil (6.58 g) in 90% yield. ¹H NMR (401 MHz, CDCl₃): 2.17-2.30 (m, 1H), 2.32-2.41 (m, 2H), 2.43-2.53 (m, 1H), 4.25 (ddd, J=8.8, 5.1, 0.8 Hz, 1H), 6.33 (bs, 1H).

### Preparation of Allyl (S)-5-oxopyrrolidine-2-carboxylate (1f)

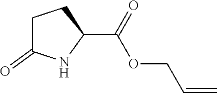

L-Pyroglutamic acid (11.6 g, 90 mmol, 1 equiv.) was suspended in anhydrous allylalcohol (40 mL), p-toluenesulfonic acid monohydrate (1.71 g, 9.0 mmol, 0.1 equiv.) and molecular sieves (20 pieces, 3 Å, activated) were added and the resulting mixture was heated to reflux for 18 h. The mixture was cooled to rt and EtOAc (350 mL) and Et₂O (350 mL) were added. Organic phase was washed with mixture of sat. NaHCO₃ (70 mL) and sat. NaCl (70 mL), water phases were then extracted with mixture of EtOAc (200 mL) and Et₂O (200 mL), combined organic phases were dried over anhydrous MgSO₄ and solvents were evaporated. Compound if was obtained as a colorless solid (12.9 g) in 85% yield. ¹H NMR (401 MHz, CDCl₃): 2.16-2.26 (m, 1H), 2.29-2.40 (m, 2H), 2.41-2.53 (m, 1H), 4.26 (ddd, J=8.6, 5.1, 2.5 Hz, 1H), 4.63 (ddq, J=5.6, 2.8, 1.3 Hz, 2H), 5.25 (ddt, J=10.4, 2.6, 1.6 Hz, 1H), 5.32 (ddt, J=17.2, 2.8, 1.6 Hz, 1H), 5.82-5.96 (m, 1H), 6.87 (bs, 1H). ¹³C NMR (101 MHz, CDCl₃): 24.89, 29.34, 55.57, 66.18, 119.25, 131.46, 171.86, 178.21.

### Preparation of 1,3-Difluoropropan-2-yl (S)-5-oxopyrrolidine-2-carboxylate (1g)

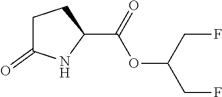 [SMILES: O=C1CC[C@@H](C(=O)OC(CF)CF)N1]

L-Pyroglutamic acid (6.46 g, 50 mmol, 1 equiv.) was suspended in 1,3-difluoropropan-2-ol (9 mL), p-toluenesulfonic acid monohydrate (951 mg, 5.0 mmol, 0.1 equiv.) and molecular sieves (15 pieces, 3 Å, activated) were added and the resulting mixture was heated to reflux (130° C.) for 22 h. The mixture was cooled to rt and EtOAc (300 mL) was added. Organic phase was washed with sat. NaHCO₃ (10 mL) and sat. NaCl (10 mL), combined water phases were then extracted with EtOAc (3×150 mL), combined organic phases were dried over anhydrous MgSO₄ and solvents were evaporated. The crude product was purified by LC on silica gel (EtOAc) and product 1g was obtained as a colorless oil (3.63 g) in 35% yield. ¹H NMR (401 MHz, CDCl₃): 2.17-2.27 (m, 1H), 2.29-2.43 (m, 2H), 2.44-2.56 (m, 1H), 4.32 (ddd, J=8.9, 4.8, 0.7 Hz, 1H), 4.54 (dt, J=4.4, 1.3 Hz, 2H), 4.65 (dt, J=4.4, 1.3 Hz, 2H), 5.28 (tp, J=19.6, 4.6 Hz, 1H), 6.94 (bs, 1H). ¹³C NMR (101 MHz, CDCl₃): 24.96, 29.20, 55.46, 71.63 (t, J=20.6 Hz), 79.42 (dd, J=7.3, 3.7 Hz), 81.14 (dd, J=7.3, 3.6 Hz), 171.66, 178.33.

General procedure for synthesis of compounds 2a-2g: Compounds 1a-1g (30 mmol, 1 equiv.) were dissolved in anhydrous THE (110 ml) and the mixture was cooled to −78° C. 1M solution of LiHMDS in THE (28.5 mL, 28.5 mmol, 0.95 equiv.) was added dropwise and the mixture was stirred for 30 minutes at the same temperature. Then it was transferred via cannula to a solution of Fmoc-Cl (23.3 g, 90 mmol, 3 equiv.) in anhydrous THE (160 mL) cooled to −78° C. The resulting mixture was stirred at −78° C. for 2 h and at rt overnight. Reaction was quenched with sat. NH₄Cl (200 mL) and the aqueous phase was extracted with EtOAc (2×200 mL). Combined organics were washed with brine (200 mL) and dried over anhydrous MgSO₄. Volatiles were evaporated in vacuo and the residue was chromatographed on silica gel (various mobile phases) to obtained colorless solids 2a-2g in yields 56-87%.

### Preparation of 1-((9H-Fluoren-9-yl)methyl) 2-isopropyl (S)-5-oxopyrrolidine-1,2-dicarboxylate (2a)

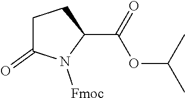

Starting material 1a (5.14 g); reaction time 17 h; mobile phase: cyclohexane/EtOAc, 2:1.

Product 2a (9.91 g), colorless solid, 84%. ¹H NMR (401 MHz, CDCl₃): 1.23 (d, J=6.3 Hz, 3H), 1.26 (d, J=6.3 Hz, 3H), 1.98-2.17 (m, 1H), 2.39 (ddt, J=13.3, 10.6, 9.3 Hz, 1H), 2.57 (ddd, J=17.5, 9.3, 3.2 Hz, 1H), 2.71 (ddd, J=17.5, 10.6, 9.4 Hz, 1H), 4.31 (t, J=7.5 Hz, 1H), 4.42 (dd, J=10.5, 7.5 Hz, 1H), 4.55 (dd, J=10.5, 7.4 Hz, 1H), 4.65 (dd, J=9.4, 2.6 Hz, 1H), 5.08 (hept, J=6.3 Hz, 1H), 7.33 (tt, J=7.5, 1.0 Hz, 2H), 7.41 (t, J=7.5 Hz, 2H), 7.70-7.74 (m, 1H), 7.72-7.80 (m, 3H). ¹³C NMR (101 MHz, CDCl₃): 21.69, 21.78, 22.01, 31.31, 46.64, 58.99, 69.20, 69.78, 120.06, 120.08, 125.43, 125.57, 127.32 (2C), 127.98 (2C), 141.31, 141.33, 143.39, 143.43, 151.56, 170.58, 172.92.

### Preparation of 1-((9H-Fluoren-9-yl)methyl) 2-(tert-butyl) (S)-5-oxopyrrolidine-1,2-dicarboxylate (2b)

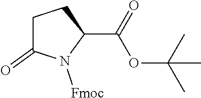

Starting material 1b (5.56 g); reaction time 21 h; mobile phase: cyclohexane/EtOAc, 2:1.

Product 2b (9.90 g), colorless solid, 81%. ¹H NMR (401 MHz, CDCl₃): 1.47 (s, 9H), 2.05-2.13 (m, 1H), 2.32-2.43 (m, 1H), 2.52-2.60 (m, 1H), 2.67-2.76 (m, 1H), 4.29-4.33 (t, J=7.5 Hz, 1H), 4.40-4.44 (m, 1H), 4.52-4.60 (m, 2H), 7.31-7.35 (m, 2H), 7.39-7.43 (m, 2H), 7.73-7.78 (m, 4H). ¹³C NMR (101 MHz, CDCl₃): 22.1, 28.1 (3C), 31.4, 46.7, 59.6, 69.2, 82.8, 120.1 (2C), 125.6, 125.7, 127.4 (2C), 128.0 (2C), 141.4 (2C), 143.5, 143.6, 151.6, 170.3, 173.0.

### Preparation of 1-((9H-Fluoren-9-yl)methyl) 2-methyl (S)-5-oxopyrrolidine-1,2-dicarboxylate (2c)

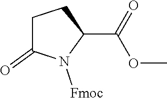

Starting material 1c (4.29 g); reaction time 19 h; mobile phase: cyclohexane/EtOAc, 3:1.

Product 2c (7.34 g), colorless solid, 67%. ¹H NMR (401 MHz, CDCl₃): 2.13 (dddd, J=13.4, 9.4, 3.1, 2.6 Hz, 1H), 2.40 (ddt, J=13.4, 10.8, 9.4 Hz, 1H), 2.58 (ddd, J=17.6, 9.3, 3.1 Hz, 1H), 2.73 (ddd, J=17.6, 10.7, 9.4 Hz, 1H), 3.76 (s, 3H), 4.33 (t, J=7.2 Hz, 1H), 4.48 (dd, J=10.6, 7.3 Hz, 1H), 4.60 (dd, J=10.6, 7.2 Hz, 1H), 4.67 (dd, J=9.4, 2.5 Hz, 1H), 7.36 (tt, J=7.4, 1.4 Hz, 2H), 7.44 (dddd, J=7.5, 6.8, 1.2, 0.7 Hz, 2H), 7.70-7.75 (m, 1H), 7.78 (ddt, J=8.3, 7.3, 1.0 Hz, 3H). ¹³C NMR (101 MHz, CDCl₃): 22.05, 31.26, 46.70, 52.91, 58.68, 69.10, 120.08, 120.11, 125.35, 125.50, 127.35 (2C), 127.99 (2C), 141.37, 141.41, 143.41, 143.49, 151.52, 171.54, 172.73.

### Preparation of 1-((9H-Fluoren-9-yl)methyl) 2-ethyl (S)-5-oxopyrrolidine-1,2-dicarboxylate (2d)

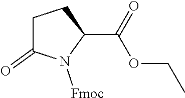

Starting material 1d (4.72 g); reaction time 19 h; mobile phase: cyclohexane/EtOAc, 3:1 to 1:1.

Product 2d (9.90 g), colorless solid, 87%. ¹H NMR (401 MHz, CDCl₃): 1.26 (t, J=7.2 Hz, 3H), 2.12 (ddt, J=13.3, 9.5, 2.9 Hz, 1H), 2.40 (ddt, J=13.4, 10.8, 9.4 Hz, 1H), 2.57 (ddd, J=17.6, 9.3, 3.1 Hz, 1H), 2.72 (ddd, J=17.5, 10.7, 9.4 Hz, 1H), 4.21 (q, J=7.2 Hz, 2H), 4.30 (t, J=7.3 Hz, 1H), 4.44 (dd, J=10.6, 7.4 Hz, 1H), 4.57 (dd, J=10.6, 7.3 Hz, 1H), 4.65 (dd, J=9.4, 2.5 Hz, 1H), 7.33 (tt, J=7.4, 1.1 Hz, 2H), 7.38-7.45 (m, 2H), 7.71 (dq, J=7.5, 0.9 Hz, 1H), 7.71-7.80 (m, 3H). ¹³C NMR (101 MHz, CDCl₃): 14.14, 21.96, 31.21, 46.62, 58.76, 61.92, 69.03, 119.99, 120.01, 125.30, 125.44, 127.26 (2C), 127.91 (2C), 141.27, 141.29, 143.35, 143.40, 151.44, 170.80, 172.99.

### Preparation of 1-((9H-Fluoren-9-yl)methyl) 2-(methyl-d₃) (S)-5-oxopyrrolidine-1,2-dicarboxylate (2e)

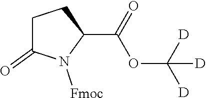

Starting material 1e (4.38 g); reaction time 23 h; mobile phase: cyclohexane/EtOAc, 4:1 or DCM/MeOH, 50:1.

Product 2e (9.62 g), colorless solid, 87%. ¹H NMR (401 MHz, CDCl₃): 2.11 (ddt, J=13.3, 9.4, 2.8 Hz, 1H), 2.38 (ddt, J=13.4, 10.7, 9.4 Hz, 1H), 2.56 (ddd, J=17.5, 9.3, 3.1 Hz, 1H), 2.70 (ddd, J=17.6, 10.7, 9.4 Hz, 1H), 4.30 (t, J=7.2 Hz, 1H), 4.45 (dd, J=10.6, 7.3 Hz, 1H), 4.58 (dd, J=10.5, 7.2 Hz, 1H), 4.64 (dd, J=9.5, 2.5 Hz, 1H), 7.33 (tt, J=7.4, 1.4 Hz, 2H), 7.41 (td, J=7.5, 1.1 Hz, 2H), 7.71 (dq, J=7.4, 1.0 Hz, 1H), 7.71-7.80 (m, 3H). ¹³C NMR (101 MHz, CDCl₃): 22.06, 31.27, 46.71, 51.59-52.85 (m), 58.68, 69.10, 120.08, 120.11, 125.35, 125.50, 127.35 (2C), 127.99 (2C), 141.38, 141.41, 143.41, 143.50, 151.53, 171.56, 172.74.

### Preparation of 1-((9H-Fluoren-9-yl)methyl) 2-allyl (S)-5-oxopyrrolidine-1,2-dicarboxylate (2f)

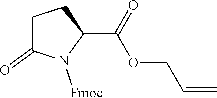

Starting material if (5.08 g); reaction time 19 h; mobile phase: cyclohexane/EtOAc, 2:1.

Product 2f (9.98 g), colorless solid, 85%. ¹H NMR (401 MHz, CDCl₃): 2.14 (ddt, J=13.4, 9.4, 2.9 Hz, 1H), 2.41 (ddt, J=13.4, 10.7, 9.4 Hz, 1H), 2.58 (ddd, J=17.6, 9.3, 3.1 Hz, 1H), 2.72 (ddd, J=17.5, 10.6, 9.4 Hz, 1H), 4.30 (t, J=7.3 Hz, 1H), 4.44 (dd, J=10.5, 7.4 Hz, 1H), 4.57 (dd, J=10.5, 7.3 Hz, 1H), 4.64 (dq, J=5.8, 1.4 Hz, 2H), 4.69 (dd, J=9.5, 2.5 Hz, 1H), 5.26 (dq, J=10.4, 1.2 Hz, 1H), 5.33 (dq, J=17.2, 1.5 Hz, 1H), 5.88 (ddt, J=17.1, 10.4, 5.8 Hz, 1H), 7.33 (tt, J=7.5, 1.1 Hz, 2H), 7.41 (tt, J=7.4, 0.8 Hz, 2H), 7.71 (dd, J=7.5, 1.0 Hz, 1H), 7.73-7.80 (m, 3H). ¹³C NMR (101 MHz, CDCl₃): 22.07, 31.30, 46.71, 58.81, 66.45, 69.21, 119.40, 120.09, 120.11, 125.42, 125.56, 127.37 (2C), 128.01 (2C), 131.31, 141.39, 141.41, 143.43, 143.51, 151.58, 170.77, 172.71.

### Preparation of 1-((9H-Fluoren-9-yl)methyl) 2-(1,3-difluoropropan-2-yl) (S)-5-oxopyrrolidine-1,2-dicarboxylate (2g)

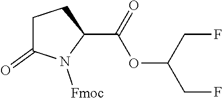

Starting material 1g (6.22 g); reaction time 23 h; mobile phase: DCM/EtOAc, 20:1.

Product 2g (7.21 g), colorless solid, 56%. ¹H NMR (401 MHz, CDCl₃): 2.13-2.22 (m, 1H), 2.47 (ddt, J=13.5, 10.4, 9.4 Hz, 1H), 2.63 (ddd, J=17.6, 9.3, 3.5 Hz, 1H), 2.76 (ddd, J=17.6, 10.3, 9.4 Hz, 1H), 4.34 (t, J=7.4 Hz, 1H), 4.45 (dd, J=10.5, 7.5 Hz, 1H), 4.55 (dt, J=4.4, 1.4 Hz, 2H), 4.60 (dd, J=10.5, 7.4 Hz, 1H), 4.66 (dt, J=4.5, 1.4 Hz, 2H), 4.76 (dd, J=9.5, 2.9 Hz, 1H), 5.22-5.40 (m, 1H), 7.36 (tt, J=7.5, 1.2 Hz, 2H), 7.40-7.48 (m, 2H), 7.74 (dd, J=7.5, 1.0 Hz, 1H), 7.76-7.82 (m, 3H). ¹³C NMR (101 MHz, CDCl₃): 21.93, 31.24, 46.66, 58.76, 69.39, 71.82 (t, J=20.7 Hz), 79.31 (dd, J=13.2, 7.2 Hz), 81.03 (dd, J=13.2, 7.2 Hz), 120.11, 120.13, 125.45, 125.59, 127.36, 127.37, 128.03 (2C), 141.38, 141.42, 143.40, 143.47, 151.64, 170.48, 172.43.

General procedure for synthesis of compounds 3a-3g: 2M solution of trimethylsilyl diazomethane (2.4 mL, 4.85 mmol, 1.25 equiv.) in diethyl ether was dissolved in absolute THE (30 mL) under argon and cooled to −98° C. A solution of n-butyllithium (2.5M in hexanes, 2.0 mL, 4.96 mmol, 1.28 equiv.) was added dropwise and the solution was stirred at −98° C. for 30 min. The resultant mixture was transferred via cannula to a solution of compound 2a-2g (3.88 mmol, 1 equiv.) in absolute THE (40 mL) at −116° C. during 60 minutes. The reaction mixture was slowly warmed to −78° C. (2 h) and then quenched with saturated NH₄Cl (10 mL) and H₂O (10 mL). The organic phase was separated and the water phase was extracted with ethyl acetate (50 ml), the combined organic layers were washed with H₂O (50 ml), sat. NaCl (50 ml) and dried over anhydrous MgSO₄. The organic solvent was evaporated in vacuo. The residue was chromatographed on silica gel (various mobile phases) to afford the desired product 3a-3g.

### Preparation of Isopropyl (S)-2-((((9H-fluoren-9-yl)methoxy)carbonyl)amino)-6-diazo-5-oxohexanoate (3a)

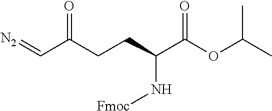

Starting material 2a (1.53 g); mobile phase: cyclohexane/EtOAc, 2:1.

Product 3a (1.37 g), light yellow solid, 81%. ¹H NMR (401 MHz, CDCl₃): 1.25 (d, J=5.7 Hz, 3H), 1.27 (d, J=5.7 Hz, 3H), 1.93-2.06 (m, 1H), 2.15-2.28 (m, 1H), 2.31-2.51 (m, 2H), 4.23 (t, J=7.0 Hz, 1H), 4.32 (tt, J=8.3, 4.2 Hz, 1H), 4.39 (dq, J=7.5, 3.6 Hz, 2H), 5.06 (hept, J=5.7 Hz, 1H), 5.26 (bs, 1H), 5.52 (d, J=8.2 Hz, 1H), 7.32 (tt, J=7.5, 1.4 Hz, 2H), 7.41 (tt, J=7.6, 1.6 Hz, 2H), 7.56-7.64 (m, 2H), 7.77 (dq, J=7.6, 1.0 Hz, 2H). ¹³C NMR (101 MHz, CDCl₃): 21.81, 21.84, 27.69, 36.56, 47.21, 53.67, 54.20, 67.10, 69.62, 120.08, 120.09, 125.18, 125.21, 127.16 (2C), 127.81 (2C), 141.35, 141.37, 143.75, 143.96, 156.16, 171.50, 193.67. ESI MS: 458.2 ([M+Na]+). HR ESI MS: calcd for C₂₄H₂₅O₅NNa 458.16873; found 458.16864.

### Preparation of tert-Butyl (S)-2-((((9H-fluoren-9-yl)methoxy)carbonyl)amino)-6-diazo-5-oxohexanoate (3b)

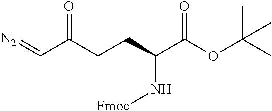

Starting material 2b (1.58 g); mobile phase: cyclohexane/EtOAc, 3:1.

Product 3b (1.17 g), yellow solid, 67%. ¹H NMR (401 MHz, CDC3): 1.50 (s, 9H), 1.94-2.07 (m, 1H), 2.18-2.29 (m, 1H), 2.31-2.53 (m, 2H), 4.21-4.32 (m, 2H), 4.41 (d, J=7.1 Hz, 2H), 5.29 (bs, 1H), 5.50 (d, J=8.1 Hz, 1H), 7.34 (tt, J=7.4, 1.4 Hz, 2H), 7.43 (t, J=7.4 Hz, 2H), 7.63 (dd, J=7.7, 4.0 Hz, 2H), 7.79 (d, J=7.3 Hz, 2H). ¹³C NMR (101 MHz, CDCl₃): 28.0 (3C), 28.1, 36.6, 47.3, 54.1, 54.9, 67.1, 82.7, 120.1, 120.1, 125.3 (2C), 127.2 (2C), 127.9 (2C), 141.4, 141.5, 143.9, 144.0, 156.2, 171.2, 193.8. ESI MS: 472.2 ([M+Na]+). HR ESI MS: calcd for C₂₅H₂₇O₅N₃Na 472.18429; found 472.18399.

### Preparation of Methyl (S)-2-((((9H-fluoren-9-yl)methoxy)carbonyl)amino)-6-diazo-5-oxohexanoate (3c)

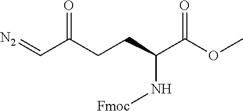

Starting material 2c (1.42 g); mobile phase: cyclohexane/EtOAc, 1:1.

Product 3c (854 mg), light yellow solid, 54%. ¹H NMR (401 MHz, CDCl₃): 1.92-2.01 (m, 1H), 2.16-2.32 (m, 1H), 2.31-2.54 (m, 2H), 3.76 (s, 3H), 4.23 (t, J=7.1 Hz, 1H), 4.34-4.45 (m, 3H), 5.26 (bs, 1H), 5.53 (d, J=8.1 Hz, 1H), 7.28-7.37 (m, 2H), 7.41 (dd, J=8.4, 6.6 Hz, 2H), 7.60 (t, J=7.2 Hz, 2H), 7.77 (d, J=7.5 Hz, 2H). ESI MS: 430.1 ([M+Na]+).

### Preparation of Ethyl (S)-2-((((9H-fluoren-9-yl)methoxy)carbonyl)amino)-6-diazo-5-oxohexanoate (3d)

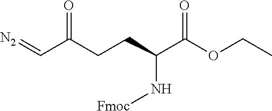

Starting material 2d (1.47 g); mobile phase: cyclohexane/EtOAc, 1:1.

Product 3d (1.31 g), light yellow solid, 80%. ¹H NMR (401 MHz, CDCl₃): 1.29 (t, J=7.2 Hz, 3H), 1.92-2.12 (m, 1H), 2.17-2.32 (m, 1H), 2.34-2.50 (m, 2H), 4.15-4.27 (m, 3H), 4.31-4.45 (m, 3H), 5.27 (bs, 1H), 5.56 (d, J=8.1 Hz, 1H), 7.32 (tt, J=7.4, 1.3 Hz, 2H), 7.36-7.45 (m, 2H), 7.60 (t, J=6.5 Hz, 2H), 7.77 (dq, J=7.6, 1.0 Hz, 2H). ¹³C NMR (101 MHz, CDCl₃): 14.28, 27.62, 36.56, 47.25, 53.63, 54.94, 61.88, 67.17, 120.12, 120.13, 125.22, 125.24, 127.20 (2C), 127.86 (2C), 141.40, 141.43, 143.79, 143.99, 156.18, 172.03, 193.65. ESI MS: 444.1 ([M+Na]+). HR ESI MS: calcd for C₂₃H₂₃O₅N₃Na 444.15299; found 444.15292.

### Preparation of Methyl-d₃ (S)-2-((((9H-fluoren-9-yl)methoxy)carbonyl)amino)-6-diazo-5-oxohexanoate (3e)

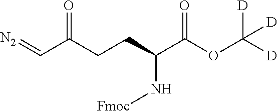

Starting material 2e (1.43 g); mobile phase: cyclohexane/EtOAc, 1:1.

Product 3e (812 mg), light yellow solid, 51%. ¹H NMR (401 MHz, CDCl₃): 1.95-2.08 (m, 1H), 2.16-2.29 (m, 1H), 2.32-2.51 (m, 2H), 4.22 (t, J=6.9 Hz, 1H), 4.32-4.46 (m, 3H), 5.25 (bs, 1H), 5.60 (d, J=8.9 Hz, 1H), 7.31 (tt, J=7.4, 1.3 Hz, 2H), 7.37-7.43 (m, 2H), 7.59 (q, J=6.3 Hz, 2H), 7.73-7.79 (m, 2H). ¹³C NMR (101 MHz, CDCl₃): 27.42, 36.42, 47.26, 53.26, 53.58, 54.86, 67.16, 120.09, 120.11, 125.19 (2C), 127.18 (2C), 127.84 (2C), 141.40, 141.42, 143.80, 143.97, 156.16, 172.48, 193.54. ESI MS: 433.2 ([M+Na]⁺). HR ESI MS: calcd for C₂₂H₁₈D₃O₅N₃Na 433.15617; found 433.15563.

### Preparation of Allyl (S)-2-((((9H-fluoren-9-yl)methoxy)carbonyl)amino)-6-diazo-5-oxohexanoate (3f)

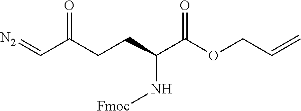

Starting material 2f (1.52 g); mobile phase: cyclohexane/EtOAc, 3:1.

Product 3f (1.41 g), light yellow solid, 84%. ¹H NMR (401 MHz, CDCl₃): 1.96-2.11 (m, 1H), 2.18-2.31 (m, 1H), 2.31-2.54 (m, 2H), 4.22 (t, J=7.0 Hz, 1H), 4.40 (tt, J=9.3, 6.9 Hz, 3H), 4.65 (d, J=5.8 Hz, 2H), 5.24-5.28 (m, 2H), 5.31-5.37 (m, 1H), 5.62 (d, J=8.2 Hz, 1H), 5.81-5.98 (m, 1H), 7.31 (tt, J=7.4, 1.3 Hz, 2H), 7.37-7.44 (m, 2H), 7.56-7.64 (m, 2H), 7.76 (dd, J=7.4, 1.1 Hz, 2H). ¹³C NMR (101 MHz, CDCl₃): 27.45, 36.53, 47.23, 53.66, 54.89, 66.29, 67.17, 119.22, 120.09, 120.10, 125.18, 125.21, 127.17 (2C), 127.83 (2C), 131.49, 141.38, 141.40, 143.77, 143.96, 156.17, 171.70, 193.55. ESI MS: 456.2 ([M+Na]⁺). HR ESI MS: calcd for C₂₄H₂₃O₅N₃Na 456.15299; found 456.15208.

### Preparation of 1,3-Difluoropropan-2-yl (S)-2-((((9H-fluoren-9-yl)methoxy)carbonyl)amino)-6-diazo-5-oxohexanoate (3g)

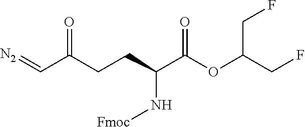

Starting material 2g (1.67 g); mobile phase: cyclohexane/EtOAc, 2:1.

Product 3g (457 mg), yellow solid, 25%. ¹H NMR (401 MHz, CDCl₃): 2.07-2.19 (m, 1H), 2.20-2.33 (m, 1H), 2.38-2.56 (m, 2H), 4.25 (t, J=7.0 Hz, 1H), 4.37-4.50 (m, 3H), 4.57 (t, J=5.0 Hz, 2H), 4.69 (t, J=5.0 Hz, 2H), 5.20-5.40 (m, 2H), 5.66 (d, J=7.9 Hz, 1H), 7.35 (tt, J=7.4, 1.3 Hz, 2H), 7.43 (dddd, J=8.6, 7.5, 1.8, 1.1 Hz, 2H), 7.62 (t, J=6.5 Hz, 2H), 7.79 (dq, J=7.5, 1.0 Hz, 2H). ¹³C NMR (101 MHz, CDCl₃): 27.04, 36.27, 47.30, 53.76, 53.91, 67.30, 71.27-71.97 (m), 79.38 (dd, J=15.2, 7.0 Hz), 81.10 (dd, J=14.8, 7.1 Hz), 120.15, 120.16, 125.23, 125.25, 127.23 (2C), 127.90 (2C), 141.45, 141.47, 143.79, 143.98, 156.15, 171.39, 194.70. ESI MS: 494.1 ([M+Na]⁺). HR ESI MS: calcd for C₂₄H₂₃O₅N₃F₂Na 494.14980; found 494.14966.

General procedure for synthesis of compounds 4a-4g: Compounds 3a-3g (13.4 mmol, 1 equiv.) was dissolved in anhydrous DCM (60 mL) and piperidine (5.68 g, 6.50 mL, 66.7 mmol, 5 equiv.) was added. The reaction mixture was stirred at room temperature under inert atmosphere for 2-5 h. Solvent was evaporated and the residue was purified by LC on silica gel (DCM/MeOH, 30:1) to afforded products 4a-4g.

### Preparation of Isopropyl (S)-2-amino-6-diazo-5-oxohexanoate (4a)

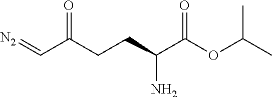

Starting material 3a (5.84 g); reaction time 5 h.

Product 4a (2.17 g), yellow oil, 76%. ¹H NMR (401 MHz, CDCl₃): 1.22 (d, J 6.3, 3H), 1.24 (d, J=6.3, 3H), 1.52 (bs, 2H), 1.74-1.85 (m, 1H), 2.02-2.10 (m, 1H), 2.36-2.53 (m, 2H), 3.37 (dd, J=8.4, 5.0, 1H) 5.00 (hept, J=6.3, 1H), 5.27 (bs, 1H). ¹³C NMR (101 MHz, CDCl₃): 21.87, 21.89, 29.65, 36.99, 53.94, 54.66, 68.64, 175.21, 194.25. ESI MS: 236.1 ([M+Na]⁺). HR ESI MS: calcd for C₉H₁₅O₃N₃Na 236.10056; found 236.10068.

### Preparation of tert-Butyl (S)-2-amino-6-diazo-5-oxohexanoate (4b)

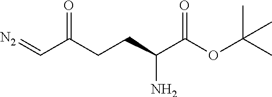

Starting material 3b (6.00 g); reaction time 3.5 h.

Product 4b (2.04 g), yellow oil, 67%. ¹H NMR (401 MHz, CDCl₃): 1.43 (s, 9H), 1.58 (bs, 2H), 1.72-1.81 (m, 1H), 1.99-2.09 (m, 1H), 2.38-2.50 (m, 2H), 3.30 (dd, J 8.3, 5.0, 1H), 5.27 (bs, 1H). ¹³C NMR (101 MHz, CDCl₃): 28.1 (3C), 29.8, 37.1, 54.4, 56.4, 81.4, 175.0, 194.4. ESI MS: 228.1 ([M+H]⁺). HR ESI MS: calcd for C₁₀H₁₈O₃N₃ 228.13427; found 228.13411.

### Preparation of Methyl (S)-2-amino-6-diazo-5-oxohexanoate (4c)

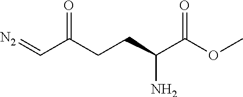

Starting material 3c (5.46 g); reaction time 2 h.

Product 4c (1.79 g), yellow oil, 72%. ¹H NMR (401 MHz, CDCl₃): 1.53 (bs, 2H), 1.76-1.87 (m, 1H), 2.03-2.15 (m, 1H), 2.42-2.55 (m, 2H), 3.44 (dd, J=8.3, 5.1, 1H), 3.64 (s, 3H), 5.26 (bs; 1H). ¹³C NMR (101 MHz, CDCl₃): 29.57, 36.44, 51.84, 53.80, 54.61, 175.62, 194.12. ESI MS: 186.1 ([M+H]⁺). HR ESI MS: calcd for C₇H₁₂O₃N₃ 186.08732; found 186.08745.

### Preparation of Ethyl (S)-2-amino-6-diazo-5-oxohexanoate (4d)

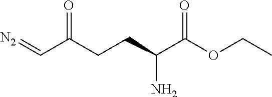

Starting material 3d (5.65 g); reaction time 4.5 h.

Product 4d (2.11 g), yellow oil, 79%. ¹H NMR (401 MHz, CDCl₃): 1.27 (t, J=7.1, 3H), 1.55 (bs, 2H), 1.78-1.88 (m, 1H), 2.06-2.17 (m, 1H), 2.40-2.54 (m, 2H), 3.44 (dd, J=8.3, 5.1, 1H), 4.17 (q, J=7.1, 2H), 5.27 (bs, 1H). ¹³C NMR (101 MHz, CDCl₃): 14.24, 29.56, 36.86, 53.77, 54.56, 61.05, 175.58, 194.15.

### Preparation of Methyl-d₃ (S)-2-amino-6-diazo-5-oxohexanoate (4e)

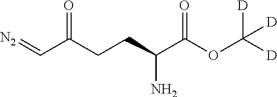

Starting material 3e (5.50 g); reaction time 2.5 h.

Product 4e (1.71 g), yellow oil, 68%. ¹H NMR (401 MHz, DMSO-d₆): 1.62 (dtd, J=13.5, 8.3, 6.4 Hz, 1H), 1.74-1.88 (m, 3H), 2.32-2.46 (m, 2H), 3.29 (dd, J=8.2, 5.3 Hz, 1H), 6.06 (bs, 1H). ¹³C NMR (101 MHz, DMSO-d₆): 29.40, 36.41, 50.74, 53.27, 53.74, 175.93, 194.70. ESI MS: 211.1 ([M+Na]⁺). HR ESI MS: calcd for C₇H₈D₃O₃N₃Na 211.08809; found 211.08844.

### Preparation of Allyl (S)-2-amino-6-diazo-5-oxohexanoate (4f)

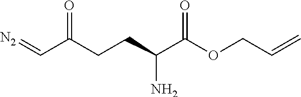

Starting material 3f (5.81 g); reaction time 4 h.

Product 4f (2.01 g), yellow oil, 71%. ¹H NMR (401 MHz, DMSO-d₆): 1.63 (dtd, J=13.5, 8.4, 6.4 Hz, 1H), 1.80 (bs, 2H), 1.86 (dddd, J=13.6, 8.4, 7.2, 5.2 Hz, 1H), 2.41 (q, J=8.4, 7.7 Hz, 2H), 3.32 (dd, J=8.3, 5.2 Hz, 1H), 4.56 (dq, J=5.5, 1.5 Hz, 2H), 5.22 (dq, J=10.5, 1.4 Hz, 1H), 5.31 (dq, J=17.3, 1.7 Hz, 1H), 5.92 (ddt, J=17.3, 10.7, 5.4 Hz, 1H), 6.06 (s, 1H). ¹³C NMR (101 MHz, DMSO-d₆): 29.44, 36.47, 53.37, 54.66, 64.49, 117.77, 132.66, 175.14, 194.69. ESI MS: 212.1 ([M+H]⁺). HR ESI MS: calcd for C₉H₁₄O₃N₃ 212.10297; found 212.10305.

### Preparation of 1,3-Difluoropropan-2-yl (S)-2-amino-6-diazo-5-oxohexanoate (4g)

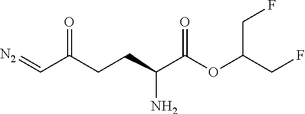

Starting material 3g (6.32 g); reaction time 2 h.

Product 4g (1.77 g), yellow oil, 53%. ¹H NMR (401 MHz, DMSO-d₆): 1.57-1.75 (m, 1H), 1.79-1.97 (m, 3H), 2.35-2.48 (m, 2H), 3.38 (dd, J=8.2, 5.2 Hz, 1H), 4.52-4.63 (m, 2H), 4.64-4.75 (m, 2H), 5.17-5.38 (m, 1H), 6.05 (bs, 1H). ESI MS: 272.1 ([M+Na]⁺). HR ESI MS: calcd for C₉H₁₃O₃N₃F₂Na 272.08172; found 272.08150.

### Example 2

Synthesis of Compounds 5a-5l

Compounds 5a-5l were prepared according to the following reaction Scheme.

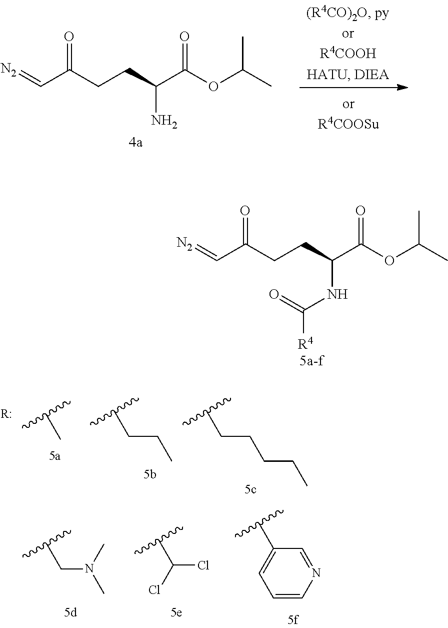

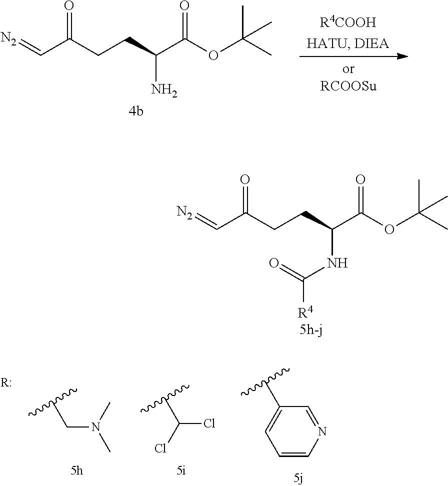

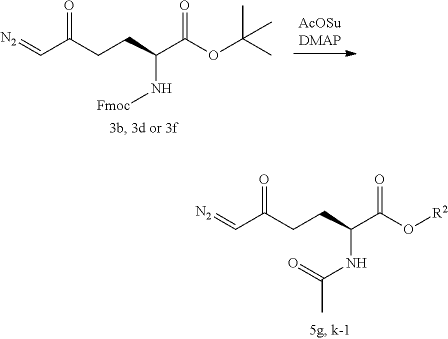

### Preparation of Isopropyl (S)-2-acetamido-6-diazo-5-oxohexanoate (5a)

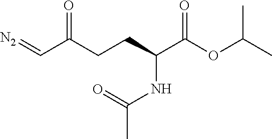

Compound 4a (300 mg, 1.41 mmol, 1 equiv.) was dissolved in anhydrous DMF (5 mL), the mixture was cooled to 0° C. and pyridine (222 mg, 227 μL, 2.81 mmol, 2 equiv.) followed by acetic anhydride (172 mg, 159 μL, 1.69 mmol, 1.2 equiv.) were added. The resulting mixture was stirred 30 minutes at 0° C. and 2.5 h at rt. DMF was evaporated and the crude product was purified by LC on silica gel (DCM/MeOH, 20:1) and product 5a was obtained as a light yellow solid (310 mg) in 86% yield. ¹H NMR (401 MHz, CDCl₃): 1.23 (d, J=6.3 Hz, 3H), 1.24 (d, J=6.3 Hz, 3H), 1.91-2.02 (m, 1H), 2.00 (s, 3H), 2.16 (dtd, J=14.5, 7.3, 4.7 Hz, 1H), 2.28-2.49 (m, 2H), 4.45-4.56 (m, 1H), 5.02 (hept, J=6.2 Hz, 1H), 5.30 (bs, 1H), 6.39 (d, J=7.8 Hz, 1H). ¹³C NMR (101 MHz, CDCl₃): 21.80, 21.81, 23.25, 27.54, 36.75, 52.06, 54.93, 69.55, 170.24, 171.59, 193.96. ESI MS: 278.1 ([M+Na]⁺). HR ESI MS: calcd for C₁₁H₁₇O₄N₃Na 278.11113; found 278.11125.

### Preparation of Isopropyl (S)-2-butyramido-6-diazo-5-oxohexanoate (5b)

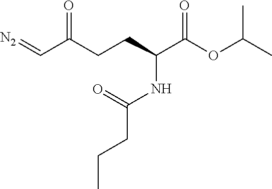

Compound 4a (100 mg, 0.469 mmol, 1 equiv.) was dissolved in anhydrous DMF (3 mL) and pyridine (74 mg, 76 μL, 0.938 mmol, 2 equiv.) followed by butyric anhydride (74 mg, 77 μL, 0.469 mmol, 1 equiv.) were added. The resulting mixture was stirred for 3 h at rt. DMF was evaporated and the crude product was purified by LC on silica gel (DCM/MeOH, 30:1) and product 5b was obtained as a light yellow solid (120 mg) in 90% yield. ¹H NMR (401 MHz, CDCl₃): 0.90 (t, J=7.4 Hz, 3H), 1.19 (d, J=6.2 Hz, 3H), 1.20 (d, J=6.2 Hz, 3H), 1.61 (h, J=7.4 Hz, 2H), 1.94 (ddd, J=12.8, 8.3, 6.5 Hz, 1H), 2.06-2.14 (m, 1H), 2.15 (t, J=7.5 Hz, 2H), 2.25-2.48 (m, 2H), 4.47 (td, J=8.3, 4.7 Hz, 1H), 4.98 (hept, J=6.4 Hz, 1H), 5.30 (bs, 1H), 6.41 (d, J=7.6 Hz, 1H). ¹³C NMR (101 MHz, CDCl₃): 13.72, 19.02, 21.71, 21.73, 27.34, 36.73, 38.40, 51.87, 54.87, 69.33, 171.54, 173.14, 193.97. ESI MS: 306.1 ([M+Na]⁺). HR ESI MS: calcd for C₁₃H₂₁O₄N₃Na 306.14243; found 306.14224.

### Preparation of Isopropyl (S)-6-diazo-2-hexanamido-5-oxohexanoate (5c)

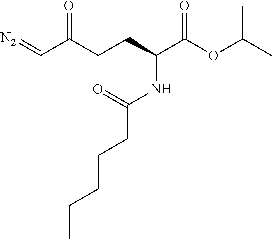

Hexanoic acid (55 mg, 0.469 mmol, 1 equiv.) and HATU (196 mg, 0.516 mmol, 1.1 equiv.) were dissolved in anhydrous DCM (3 mL), the mixture was cooled to 0° C. and DIEA (182 mg, 245 μL, 1.41 mmol, 3 equiv.) was added. After 5 minutes of stirring the solution of compound 4a (100 mg, 0.469 mmol, 1 equiv.) in anhydrous DCM (1 mL) was added. The resulting mixture was stirred for 30 minutes at 0° C. and 90 minutes at rt. DCM (50 mL) was added and the organic phase was washed with sat. NaHCO₃ (40 mL), H₂O (40 mL), 1M HCl (40 mL), H₂O (40 mL) and sat. NaCl (40 mL), dried over anhydrous MgSO₄ and DCM was evaporated. The crude product was purified by LC on silica gel (DCM/MeOH, 40:1) and product 5c was obtained as a light yellow solid (135 mg) in 93% yield. ¹H NMR (401 MHz, CDCl₃): 0.82 (t, J=7.0 Hz, 3H), 1.17 (d, J=6.2 Hz, 3H), 1.19 (d, J=6.2 Hz, 3H), 1.21-1.27 (m, 4H), 1.50-1.63 (m, 2H), 1.92 (dtd, J=14.4, 8.2, 6.6 Hz, 1H), 2.04-2.13 (m, 1H), 2.15 (t, J=7.4 Hz, 2H), 2.25-2.47 (m, 2H), 4.45 (td, J=8.2, 4.7 Hz, 1H), 4.96 (hept, J=6.2 Hz, 1H), 5.29 (bs, 1H), 6.42 (d, J=7.8 Hz, 1H). ¹³C NMR (101 MHz, CDCl₃): 13.89, 21.67, 21.69, 22.35, 25.24, 27.31, 31.37, 36.44, 36.72, 51.85, 54.77, 69.27, 171.51, 173.26, 193.91. ESI MS: 334.2 ([M+Na]⁺). HR ESI MS: calcd for C₁₅H₂₅O₄N₃Na 334.17373; found 334.17349.

### Preparation of Isopropyl (S)-6-diazo-2-(2-(dimethylamino)acetamido)-5-oxohexanoate (5d)

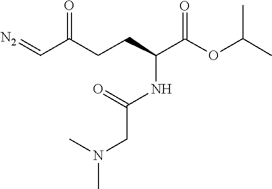

Dimethylglycine (97 mg, 0.938 mmol, 1 equiv.) and HATU (392 mg, 1.03 mmol, 1.1 equiv.) were dissolved in anhydrous DMF (7 mL), the mixture was cooled to 0° C. and DIEA (364 mg, 490 μL, 2.81 mmol, 3 equiv.) was added. After 5 minutes of stirring the solution of compound 4a (200 mg, 0.938 mmol, 1 equiv.) in anhydrous DMF (3 mL) was added. The resulting mixture was stirred for 30 minutes at 0° C. and 90 minutes at rt. DMF was evaporated, EtOAc (100 mL) was added and the organic phase was washed with sat. NaHCO₃ (50 mL), H₂O (50 mL) and sat. NaCl (50 mL), dried over MgSO₄ and solvent was evaporated. The crude product was purified by LC on silica gel (DCM/MeOH, 30:1) and product 5d was obtained as an yellow solid (233 mg) in 83% yield. ¹H NMR (401 MHz, CDCl₃): 1.23 (d, J=2.8 Hz, 3H), 1.25 (d, J=2.9 Hz, 3H), 1.98 (dtd, J=14.7, 8.7, 6.0 Hz, 1H), 2.22 (dddd, J=13.4, 8.5, 6.7, 4.7 Hz, 1H), 2.30 (s, 6H), 2.33-2.46 (m, 2H), 2.90 (d, J=16.3 Hz, 1H), 3.00 (d, J=16.3 Hz, 1H), 4.56 (td, J=8.9, 4.7 Hz, 1H), 5.02 (hept, J=6.3 Hz, 1H), 5.29 (bs, 1H), 7.64 (d, J=8.8 Hz, 1H). ¹³C NMR (101 MHz, CDCl₃): 21.83, 21.84, 27.88, 36.97, 46.07 (2C), 51.35, 54.81, 63.05, 69.47, 170.96, 171.32, 193.51. ESI MS: 299.2 ([M+H]⁺) HR ESI MS: calcd for C₁₃H₂₃O₄N₄ 299.17138; found 299.17121.

### Preparation of Isopropyl (S)-6-diazo-2-(2,2-dichloroacetamido)-5-oxohexanoate (5e)

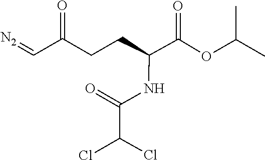

2,2-Dichloroacetic acid (133 mg, 85 μL, 1.03 mmol, 1.1 equiv.) and HATU (428 mg, 1.13 mmol, 1.2 equiv.) were dissolved in anhydrous DCM (8 mL), the mixture was cooled to 0° C. and DIEA (364 mg, 490 μL, 2.81 mmol, 3 equiv.) was added. After 5 minutes of stirring the solution of compound 4a (200 mg, 0.938 mmol, 1 equiv.) in anhydrous DCM (3 mL) was added. The resulting mixture was stirred for 30 minutes at 0° C. and 60 minutes at rt. DCM (50 mL) was added and the organic phase was washed with sat. NaHCO₃ (30 mL), H₂O (30 mL), 10% KHSO₄ (30 mL), H₂O (30 mL) and sat. NaCl (30 mL), dried over anhydrous MgSO₄ and DCM was evaporated. The crude product was purified by LC on silica gel (DCM/EtOAc, 5:1) and product 5e was obtained as an yellow solid (166 mg) in 55% yield. ¹H NMR (401 MHz, CDCl₃): 1.25 (d, J=6.2 Hz, 3H), 1.26 (d, J=6.2 Hz, 3H), 2.10 (ddt, J=14.1, 8.4, 6.9 Hz, 1H), 2.26 (dtd, J=14.3, 7.1, 4.7 Hz, 1H), 2.32-2.53 (m, 2H), 4.47 (td, J=8.0, 4.6 Hz, 1H), 5.05 (hept, J=6.3 Hz, 1H), 5.30 (bs, 1H), 5.94 (s, 1H), 7.46 (d, J=7.5 Hz, 1H). ¹³C NMR (101 MHz, CDCl₃): 21.77, 21.81, 26.69, 36.29, 52.93, 55.20, 66.26, 70.09, 164.30, 170.35, 193.58. ESI MS: 346.0 ([M+Na]⁺). HR ESI MS: calcd for C₁₁H₁₅O₄N₃Cl₂Na 346.03318; found 346.03293.

### Preparation of Isopropyl (S)-6-diazo-2-(nicotinamido)-5-oxohexanoate (5f)

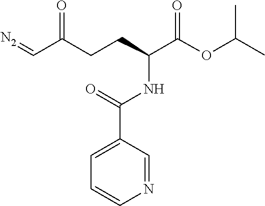

Compound 4a (200 mg, 0.937 mmol, 1 equiv.) and 2,5-dioxopyrrolidin-1-yl nicotinate (310 mg, 1.41 mmol, 1.5 equiv.) were dissolved in anhydrous DCM (5 mL) and the mixture was stirred at rt for 2 h. DCM was evaporated and the crude product was purified by LC on silica gel (DCM/MeOH, 40:1) and product 5f was obtained as a light yellow-brown oil (132 mg) in 44% yield. ¹H NMR (401 MHz, CDCl₃): 1.26 (d, J=6.3 Hz, 3H), 1.28 (d, J=6.3 Hz, 3H), 2.11-2.24 (m, 1H), 2.23-2.32 (m, 1H), 2.41-2.62 (m, 2H), 4.66 (ddd, J=8.4, 7.1, 4.4 Hz, 1H), 5.07 (hept, J=6.3 Hz, 1H), 5.31 (bs, 1H), 7.39 (ddd, J=8.0, 4.9, 0.9 Hz, 1H), 7.62 (s, 1H), 8.14 (dt, J=8.0, 2.0 Hz, 1H), 8.73 (dd, J=4.9, 1.7 Hz, 1H), 9.07 (d, J=2.7 Hz, 1H). ¹³C NMR (101 MHz, CDCl₃): 21.86, 21.88, 26.66, 36.71, 52.99, 55.47, 69.71, 123.56, 129.51, 135.15, 148.58, 152.54, 165.53, 171.22, 194.55. ESI MS: 341.1 ([M+Na]⁺). HR ESI MS: calcd for C₁₅H₁₈O₄N₄Na 341.12203; found 341.12188.

### Preparation of tert-Butyl (S)-2-acetamido-6-diazo-5-oxohexanoate (5g)

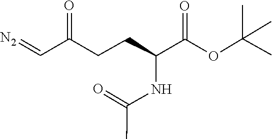

Compound 3b (300 mg, 0.667 mmol, 1 equiv.), DMAP (815 mg, 6.67 mmol, 10 equiv.) and AcOSu (157 mg, 1.00 mmol, 1.5 equiv.) were dissolved in anhydrous DCM (3 mL) and the resulting mixture was stirred at rt for 26 h. DCM was evaporated, the crude product was purified by LC on silica gel (DCM/MeOH, 30:1) and product 5g was obtained as a light yellow oil (139 mg) in 77% yield. ¹H NMR (401 MHz, CDCl₃): 1.45 (s, 9H), 1.89-2.00 (m, 1H), 2.00 (s, 3H), 2.15 (dddd, J=14.7, 8.0, 6.8, 4.6 Hz, 1H), 2.27-2.54 (m, 2H), 4.45 (td, J=8.2, 4.6 Hz, 1H), 5.30 (bs, 1H), 6.27 (d, J=7.9 Hz, 1H). ¹³C NMR (101 MHz, CDCl₃): 23.33, 27.82, 28.09 (3C), 36.82, 52.42, 54.88, 82.59, 170.16, 171.27, 194.01. ESI MS: 292.2 ([M+Na]⁺). HR ESI MS: calcd for C₁₂H₁₉O₄N₃Na 292.12678; found 292.12650.

### Preparation of tert-Butyl (S)-6-diazo-2-(2-(dimethylamino)acetamido)-5-oxohexanoate (5h)

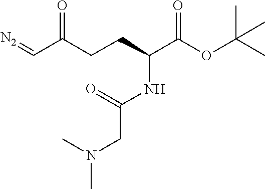

Dimethylglycine (68 mg, 0.660 mmol, 1 equiv.) and HATU (276 mg, 0.726 mmol, 1.1 equiv.) were dissolved in anhydrous DMF (5 mL), the mixture was cooled to 0° C. and DIEA (256 mg, 345 μL, 1.98 mmol, 3 equiv.) was added. After 5 minutes of stirring the solution of compound 4b (150 mg, 0.660 mmol, 1 equiv.) in anhydrous DMF (2 mL) was added. The resulting mixture was stirred for 30 minutes at 0° C. and 90 minutes at rt. DMF was evaporated, EtOAc (70 mL) was added and the organic phase was washed with sat. NaHCO₃ (30 mL), H₂O (30 mL) and sat. NaCl (30 mL), dried over anhydrous MgSO₄ and solvent was evaporated. The crude product was purified by LC on silica gel (DCM/MeOH, 30:1) and product 5h was obtained as a light yellow oil (138 mg) in 67% yield. ¹H NMR (401 MHz, CDCl₃): 1.45 (s, 9H), 1.91-2.02 (m, 1H), 2.12-2.25 (m, 1H), 2.30 (s, 6H), 2.32-2.46 (m, 2H), 2.90 (d, J=16.3 Hz, 1H), 2.99 (d, J=16.3 Hz, 1H), 4.49 (td, J=8.7, 4.7 Hz, 1H), 5.30 (bs, 1H), 7.62 (d, J=8.8 Hz, 1H). ¹³C NMR (101 MHz, CDCl₃): 28.09 (3C), 29.79, 36.98, 46.09 (2C), 51.76, 54.78, 63.07, 82.44, 170.84, 170.92, 193.65. ESI MS: 335.2 ([M+Na]⁺). HR ESI MS: calcd for C₁₄H₂₄O₄N₄Na 335.16898; found 335.16875.

### Preparation of tert-Butyl (S)-6-diazo-2-(2,2-dichloroacetamido)-5-oxohexanoate (5i)

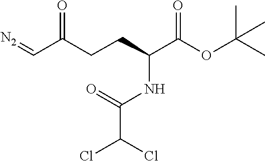

2,2-Dichloroacetic acid (94 mg, 60 μL, 0.726 mmol, 1.1 equiv.) and HATU (301 mg, 0.792 mmol, 1.2 equiv.) were dissolved in anhydrous DCM (6 mL), the mixture was cooled to 0° C. and DIEA (256 mg, 345 μL, 1.98 mmol, 3 equiv.) was added. After 5 minutes of stirring the solution of compound 4b (150 mg, 0.660 mmol, 1 equiv.) in anhydrous DCM (2 mL) was added. The resulting mixture was stirred for 30 minutes at 0° C. and 60 minutes at rt. DCM (50 mL) was added and the organic phase was washed with sat. NaHCO₃ (30 mL), H₂O (30 mL), 10% KHSO₄ (30 mL), H₂O (30 mL) and sat. NaCl (30 mL), dried over anhydrous MgSO₄ and DCM was evaporated. The crude product was purified by LC on silica gel (DCM/EtOAc, 5:1) and product 5i was obtained as an yellow amorphous oil (137 mg) in 61% yield. ¹H NMR (401 MHz, CDCl₃): 1.48 (s, 9H), 2.05-2.13 (m, 1H), 2.27 (dtd, J=14.4, 7.0, 4.5 Hz, 1H), 2.32-2.53 (m, 2H), 4.37-4.46 (m, 1H), 5.29 (bs, 1H), 5.93 (s, 1H), 7.37 (d, J=7.5 Hz, 1H). ¹³C NMR (101 MHz, CDCl₃). 26.96, 28.09 (3C), 36.34, 53.28, 55.15, 66.34, 83.30, 164.20, 169.97, 193.59. ESI MS: 360.0 ([M+Na]⁺). HR ESI MS: calcd for C₁₂H₁₇O₄N₃Cl₂Na 360.04883; found 360.04858.

### Preparation of tert-Butyl (S)-6-diazo-2-(nicotinamido)-5-oxohexanoate (5j)

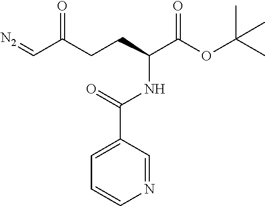

Compound 4b (100 mg, 0.440 mmol, 1 equiv.) and 2,5-dioxopyrrolidin-1-yl nicotinate (102 mg, 0.462 mmol, 1.05 equiv.) were dissolved in anhydrous DCM (4 mL) and the mixture was stirred at rt for 18 h. DCM was evaporated and the crude product was purified by LC on silica gel (DCM/MeOH, 40:1) and product 5j was obtained as a light yellow-brown oil (64 mg) in 44% yield. ¹H NMR (401 MHz, CDCl₃): 1.46 (s, 9H), 2.13 (dq, J=14.5, 7.2 Hz, 1H), 2.25 (dtd, J=14.1, 7.0, 4.8 Hz, 1H), 2.38-2.60 (m, 2H), 4.57-4.63 (m, 1H), 5.31 (bs, 1H), 7.36 (ddd, J=7.9, 4.9, 0.9 Hz, 1H), 7.54 (d, J=6.9 Hz, 1H), 8.11 (dt, J=8.0, 2.0 Hz, 1H), 8.70 (dd, J=4.8, 1.7 Hz, 1H), 9.04 (d, J=2.2 Hz, 1H). ¹³C NMR (101 MHz, CDCl₃): 26.92, 28.08 (3C), 36.73, 53.23, 55.31, 82.70, 123.50, 129.59, 135.09, 148.50, 152.43, 165.44, 170.85, 194.46. ESI MS: 355.1 ([M+Na]⁺). HR ESI MS: calcd for C₁₆H₂₀O₄N₄Na 355.13768; found 355.13739.

### Preparation of Ethyl (S)-2-acetamido-6-diazo-5-oxohexanoate (5k)

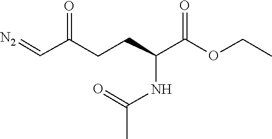

Compound 3d (400 mg, 0.949 mmol, 1 equiv.), DMAP (1.16 g, 9.49 mmol, 10 equiv.) and AcOSu (224 mg, 1.42 mmol, 1.5 equiv.) were dissolved in anhydrous DCM (4 mL) and the resulting mixture was stirred at rt for 20 h. DCM was evaporated, the crude product was purified by LC on silica gel (DCM/MeOH, 30:1) and product 5k was obtained as an yellow solid (220 mg) in 96% yield. ¹H NMR (401 MHz, CDCl₃): 1.23 (t, J=7.1 Hz, 3H), 1.90-2.02 (m, 1H), 1.97 (s, 3H), 2.14 (dtd, J=14.5, 7.3, 4.9 Hz, 1H), 2.27-2.48 (m, 2H), 4.14 (qd, J=7.1, 1.3 Hz, 2H), 4.50 (td, J=8.2, 4.8 Hz, 1H), 5.30 (bs, 1H), 6.56 (d, J=7.8 Hz, 1H). ¹³C NMR (101 MHz, CDCl₃): 14.16, 23.10, 27.26, 36.66, 51.92, 54.89, 61.64, 170.26, 172.03, 193.92. ESI MS: 264.1 ([M+Na]⁺). HR ESI MS: calcd for C₁₀H₁₅O₄N₃Na 264.09548; found 264.09523.

### Preparation of Allyl (S)-2-acetamido-6-diazo-5-oxohexanoate (51)

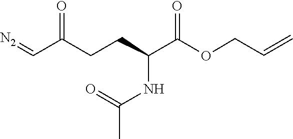

Compound 3f (300 mg, 0.692 mmol, 1 equiv.), DMAP (846 mg, 6.92 mmol, 10 equiv.) and AcOSu (163 mg, 1.04 mmol, 1.5 equiv.) were dissolved in anhydrous DCM (3 mL) and the resulting mixture was stirred at rt for 17.5 h. DCM was evaporated, the crude product was purified by LC on silica gel (DCM/MeOH, 30:1) and product 51 was obtained as an yellow oil (170 mg) in 97% yield. ¹H NMR (401 MHz, CDCl₃): 1.99 (s, 3H), 1.95-2.05 (m, 1H), 2.11-2.23 (m, 1H), 2.30-2.52 (m, 2H), 4.51-4.60 (m, 1H), 4.60 (dt, J=5.8, 1.4 Hz, 2H), 5.23 (dq, J=10.4, 1.2 Hz, 1H), 5.30 (bs, 1H), 5.31 (dq, J=17.2, 1.5 Hz, 1H), 5.87 (ddt, J=17.1, 10.4, 5.8 Hz, 1H), 6.47-6.59 (m, 1H). ¹³C NMR (101 MHz, CDCl₃): 23.15, 27.18, 36.68, 52.01, 54.98, 66.17, 119.09, 131.50, 170.29, 171.73, 193.92. ESI MS: 276.1 ([M+Na]⁺). HR ESI MS: calcd for C₁₁H₁₅O₄N₃Na 276.09548; found 276.09524.

### Example 3

### Synthesis of Compound 5m

Compound 5m was prepared according to the following reaction Scheme.

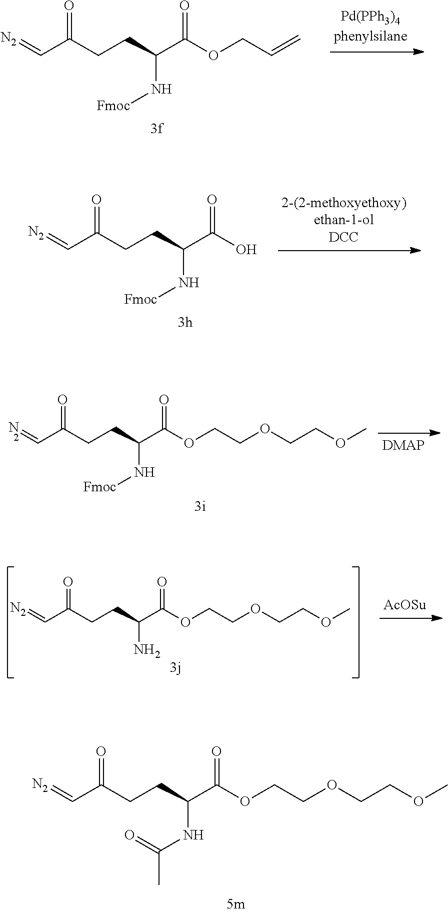

### Preparation of (S)-2-((((9H-Fluoren-9-yl)methoxy)carbonyl)amino)-6-diazo-5-oxohexanoic acid (3h)

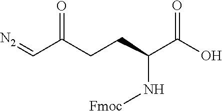

Compound 3f (5.00 g, 11.5 mmol, 1 equiv.) was dissolved in anhydrous DCM (50 mL) and phenylsilane (2.50 g, 2.84 mL, 23.1 mmol, 2 equiv.) followed by Pd(PPh₃)₄ (267 mg, 0.231 mmol, 0.02 equiv.) were added. The resulting mixture was stirred for 2 h at rt under inert atmosphere. DCM and phenylsilane were evaporated. The crude product was purified by LC on silica gel (CHCl₃/MeOH, 5:1) and compound 3h was obtained as a light yellow-brown solid (3.35 g) in 74% yield. ¹H NMR (401 MHz, CDCl₃): 1.98-2.15 (m, 1H), 2.15-2.31 (m, 1H), 2.38-2.72 (m, 2H), 4.21 (t, J=6.9 Hz, 1H), 4.37 (dt, J=12.4, 6.2 Hz, 3H), 5.32 (bs, 1H), 5.84 (d, J=7.3 Hz, 1H), 7.31 (td, J=7.4, 1.2 Hz, 2H), 7.37-7.43 (m, 2H), 7.59 (t, J=6.6 Hz, 2H), 7.74-7.78 (m, 2H). ¹³C NMR (101 MHz, CDCl₃): 25.87, 34.42, 47.14, 53.05, 53.49, 67.23, 120.09 (2C), 125.19 (2C), 127.19 (2C), 127.84 (2C), 141.36 (2C), 143.70 (2C), 156.54, 174.77, 195.08. ESI MS: 416.1 ([M+Na]⁺). HR ESI MS: calcd for C₂₁H₁₉O₅N₃Na 416.12169; found 416.12140.

### Preparation of 2-(2-Methoxyethoxy)ethyl (S)-2-((((9H-fluoren-9-yl)methoxy)carbonyl)amino)-6-diazo-5-oxohexanoate (3i)

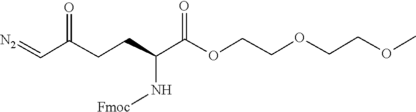

Compound 3h (500 mg, 1.27 mmol, 1 equiv.) was dissolved in anhydrous DCM (10 mL) and 2-(2-methoxyethoxy)ethan-1-ol (183 mg, 180 μL, 1.53 mmol, 1.2 equiv.) followed by solution of DCC (289 mg, 1.40 mmol, 1.1 equiv.) in anhydrous DCM (5 mL) were added. The resulting mixture was stirred under inert atmosphere at rt for 18 h. The precipitate (DCU) was filtered off and washed with DCM (10 mL) and solvent was evaporated. The residue was purified by LC on silica gel (DCM/EtOAc, 1:1) and product 3h was obtained as a yellow amorphous compound (231 mg) in 37% yield. ¹H NMR (401 MHz, CDCl₃): 1.97-2.11 (m, 1H), 2.16-2.28 (m, 1H), 2.32-2.53 (m, 2H), 3.35 (s, 3H), 3.47-3.55 (m, 2H), 3.58-3.65 (m, 2H), 3.67-3.73 (m, 2H), 4.21 (t, J=7.0 Hz, 1H), 4.25-4.43 (m, 5H), 5.31 (bs, 1H), 5.64 (d, J=8.1 Hz, 1H), 7.31 (tt, J=7.5, 1.2 Hz, 2H), 7.39 (t, J=7.3 Hz, 2H), 7.55-7.63 (m, 2H), 7.76 (d, J=7.5 Hz, 2H). ¹³C NMR (101 MHz, CDCl₃): 27.55, 36.49, 47.24, 53.60, 54.83, 59.10, 64.59, 67.12, 68.91, 70.55, 71.95, 120.09 (2C), 125.19 (2C), 127.18 (2C), 127.82 (2C), 141.38, 141.40, 143.80, 143.97, 156.10, 171.97, 193.69. ESI MS: 518.2 ([M+Na]⁺). HR ESI MS: calcd for C₂₆H₂₉O₇N₃Na 518.18977; found 518.18927.

### Preparation of 2-(2-Methoxyethoxy)ethyl (S)-2-acetamido-6-diazo-5-oxohexanoate (5m)

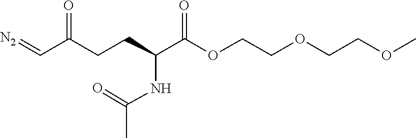

Compound 3i (90 mg, 0.182 mmol, 1 equiv.), AcOSu (34 mg, 0.218 mmol, 1.2 equiv.) and DMAP (221 mg, 1.81 mmol, 10 equiv.) were dissolved in anhydrous DCM (0.6 mL) and the mixture was stirred at rt for 18 h. DCM was evaporated and the crude product was purified by LC on silica gel (DCM/MeOH, 20:1) and compound 5m was isolated as an yellow oil (35 mg) in 61% yield. ¹H NMR (401 MHz, CDCl₃): 1.99 (s, 3H), 2.01-2.09 (m, 1H), 2.10-2.22 (m, 1H), 2.31-2.52 (m, 2H), 3.36 (s, 3H), 3.49-3.55 (m, 2H), 3.59-3.64 (m, 2H), 3.67-3.71 (m, 2H), 4.22-4.39 (m, 2H), 4.53-4.61 m, 1H), 5.34 (s, 1H), 6.46 (d, J=7.7 Hz, 1H). ¹³C NMR (101 MHz, CDCl₃): 23.0, 23.3, 27.2, 36.6, 55.0, 59.1, 64.5, 68.9, 70.5, 71.9, 170.2, 172.0, 194.1. ESI MS: 338.2 ([M+Na]⁺). HR ESI MS: calcd for C₁₃H₂₁O₆N₃Na 338.13281; found: 338.13290.

### Example 4

### Synthesis of Compounds 8a-8n

Compounds 8a-8n were prepared according to the following Scheme.

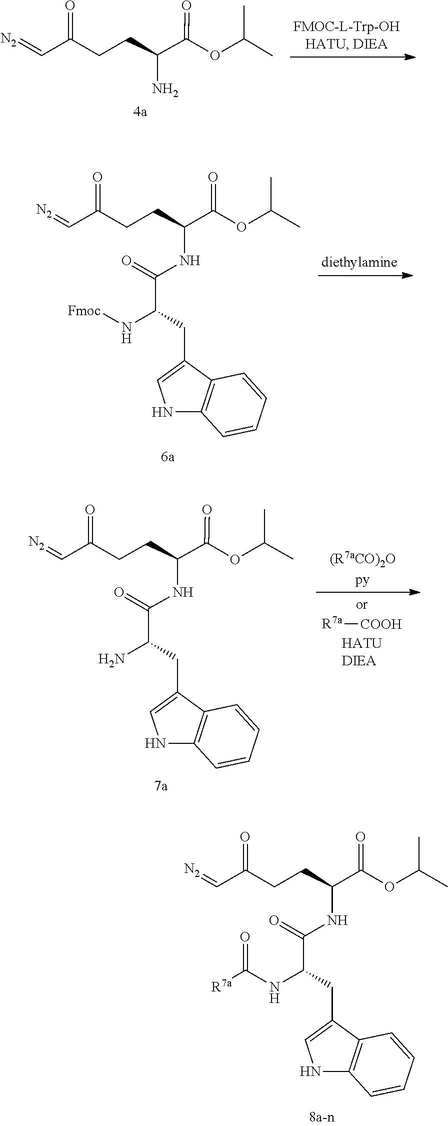

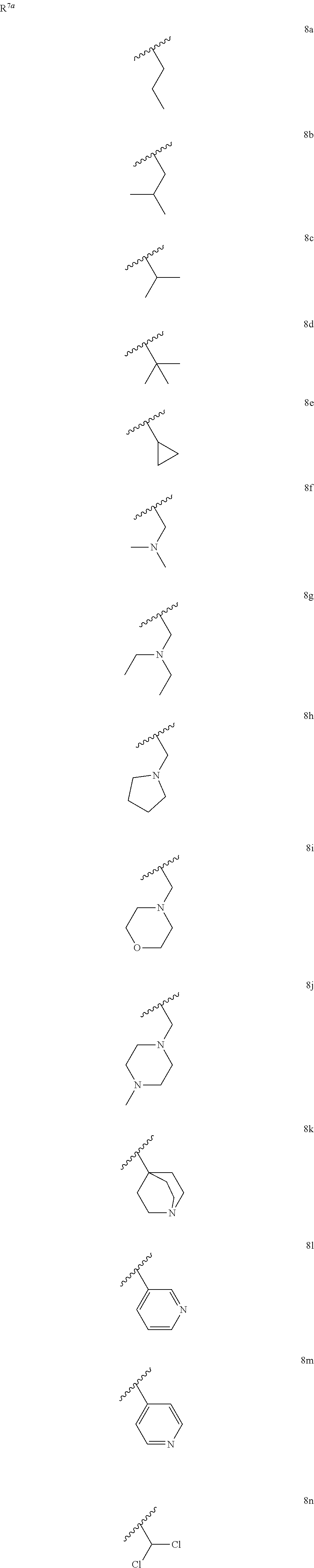

### Preparation of Isopropyl (S)-2-((S)-2-((((9H-fluoren-9-yl)methoxy)carbonyl)amino)-3-(1H-indol-3-yl)propanamido)-6-diazo-5-oxohexanoate (6a)

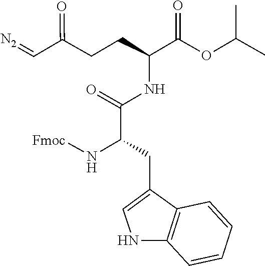

Fmoc-L-Trp-OH (9.72 g, 22.8 mmol, 1.1 equiv.) and HATU (9.06 g, 23.8 mmol, 1.15 equiv.) were dissolved in anhydrous DCM (150 mL), the mixture was cooled to 0° C. and DIEA (8.04 g, 10.8 mL, 62.2 mmol, 3 equiv.) was added. After 5 minutes of stirring the solution of compound 4a (4.42 g, 20.7 mmol, 1 equiv.) in anhydrous DCM (30 mL) was added. The resulting mixture was stirred for 30 minutes at 0° C. and 90 minutes at rt. The crude reaction mixture containing product 6a was used to the following step without any purification. ¹H NMR (401 MHz, CDCl₃): 1.19 (d, J=6.3 Hz, 3H), 1.23 (t, J=6.3 Hz, 3H), 1.81-1.92 (m, 1H), 2.01-2.28 (m, 3H), 3.18 (dd, J=14.5, 7.1, 1H), 3.39 (dd, J=14.2, 5.2 Hz, 1H), 4.20 (t, J=7.1 Hz, 1H), 4.30-4.48 (m, 3H), 4.54 (q, J=6.9 Hz, 1H), 4.88-4.99 (m, 1H), 5.07 (bs, 1H), 5.50 (d, J=7.9 Hz, 1H), 6.59 (d, J=7.4 Hz, 1H), 7.07 (bs, 1H), 7.14 (t, J=7.0 Hz, 1H), 7.20 (t, J=7.2 Hz, 1H), 7.30 (tdd, J=7.5, 2.5, 1.1 Hz, 2H), 7.36 (d, J=8.0 Hz, 1H), 7.40 (t, J=7.5 Hz, 2H), 7.56 (t, J=6.6 Hz, 2H), 7.67 (d, J=7.9 Hz, 1H), 7.77 (d, J=7.6 Hz, 2H), 8.23 (bs, 1H). ¹³C NMR (101 MHz, CDCl₃): 21.8, 21.8, 27.2, 28.5, 36.2, 47.2, 52.2, 55.8, 67.2, 69.2, 110.3, 111.4, 118.9, 120.0, 120.1, 120.1 (2C), 122.5, 123.7, 125.3, 125.3, 127.2 (2C), 127.6, 127.9 (2C), 136.4, 141.4 (2C), 143.9, 144.0, 156.1, 170.8, 171.4, 193.9. ESI MS: 644.2 ([M+Na]⁺). HR ESI MS: calcd for C₃₅H₃₅O₆N₅Na 644.24795; found 644.24811.

### Preparation of Isopropyl (S)-2-((S)-2-amino-3-(1H-indol-3-yl)propanamido)-6-diazo-5-oxohexanoate (7a)

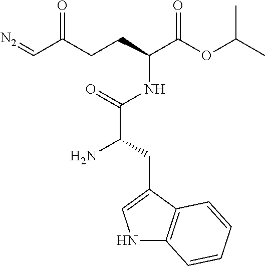

To the solution of compound 6a in DCM was added diethylamine (17.7 g, 20.3 mL, 207 mmol, 10 equiv.) and the resulting mixture was stirred at rt under inert atmosphere for 2 h. DCM was evaporated and the crude mixture was purified by LC on silica gel (DCM/MeOH, 30:1) and compound 7a was obtained as an yellow solid (5.96 g) in 72% yield over 2 steps. ¹H NMR (401 MHz, CDCl₃): 1.24 (d, J=6.0 Hz, 3H), 1.25 (d, J=6.0 Hz, 3H), 1.50 (bs, 2H), 1.88-2.00 (m, 1H), 2.04-2.27 (m, 3H), 3.04 (dd, J=14.4, 8.2 Hz, 1H), 3.30 (ddd, J=14.2, 4.2, 0.9 Hz, 1H), 3.74 (dd, J=8.1, 4.2 Hz, 1H), 4.51 (td, J=8.3, 4.0 Hz, 1H), 5.02 (hept, J=6.0 Hz, 1H), 5.11 (bs, 1H), 7.08 (d, J=2.3 Hz, 1H), 7.11 (ddd, J=8.0, 7.1, 1.0 Hz, 1H), 7.19 (ddd, J=8.1, 7.1, 1.1 Hz, 1H), 7.36 (d, J=8.1 Hz, 1H), 7.67 (d, J=7.9 Hz, 1H), 7.88 (d, J=8.2 Hz, 1H), 8.45 (bs, 1H). ¹³C NMR (101 MHz, CDCl₃): 21.8, 21.8, 27.7, 30.8, 36.6, 51.6, 54.8, 55.5, 69.5, 111.4 (2C), 119.2, 119.7, 122.3, 123.5, 127.7, 136.5, 171.4, 175.1, 193.9. ESI MS: 422.2 ([M+Na]⁺). HR ESI MS: calcd for C₂₀H₂₅O₄N₅Na 422.17988; found 422.17992.

### Preparation of Isopropyl (S)-2-((S)-2-butyramido-3-(1H-indol-3-yl)propanamido)-6-diazo-5-oxohexanoate (8a)

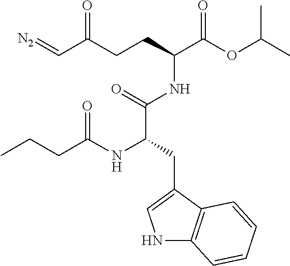

Compound 7a (100 mg, 0.250 mmol, 1 equiv.) was dissolved in anhydrous DMF (3 mL) and pyridine (40 mg, 40 μL, 0.501 mmol, 2 equiv.) followed by butyric anhydride (48 mg, 49 μL, 0.300 mmol, 1.2 equiv.) were added. The resulting mixture was stirred at rt for 5 h and DMF was evaporated. The residue was purified by LC on silica gel (DCM/MeOH, 35:1) and the product 8a was obtained as a light yellow solid (89 mg) in 76% yield. ¹H NMR (401 MHz, CDCl₃): 0.90 (t, J=7.4 Hz, 3H), 1.20 (d, J=6.3 Hz, 3H), 1.24 (d, J=6.3 Hz, 3H), 1.58-1.68 (m, 2H), 1.81-1.95 (m, 1H), 2.05-2.20 (m, 3H), 2.19-2.35 (m, 2H), 3.16 (dd, J=14.6, 7.4 Hz, 1H), 3.35 (dd, J=14.5, 5.4 Hz, 1H), 4.37 (td, J=7.6, 4.3 Hz, 1H), 4.76 (td, J=7.5, 5.4 Hz, 1H), 4.95 (hept, J=6.3 Hz, 1H), 5.15 (bs, 1H), 6.13 (d, J=7.6 Hz, 1H), 6.52 (d, J=7.4 Hz, 1H), 7.10-7.16 (m, 2H), 7.16-7.22 (m, 1H), 7.33-7.39 (m, 1H), 7.69 (d, J=7.8 Hz, 1H), 8.16 (bs, 1H). ESI MS: 492.2 ([M+Na]⁺). HR ESI MS: calcd for C₂₄H₃₁O₅N₅Na 492.22174; found 492.22129.

### Preparation of Isopropyl (S)-2-((S)-3-(1H-indol-3-yl)-2-(3-methylbutanamido)propanamido)-6-diazo-5-oxohexanoate (8b)

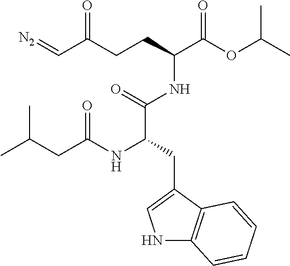

Compound 7a (100 mg, 0.250 mmol, 1 equiv.) was dissolved in anhydrous DMF (3 mL) and pyridine (40 mg, 40 μL, 0.501 mmol, 2 equiv.) followed by isovaleric anhydride (56 mg, 60 μL, 0.300 mmol, 1.2 equiv.) were added. The resulting mixture was stirred at rt for 5 h and DMF was evaporated. The residue was purified by LC on silica gel (DCM/MeOH, 40:1) and the product 8b was obtained as an yellow solid (100 mg) in 83% yield. ¹H NMR (401 MHz, CDCl₃): 0.90 (d, J=5.7 Hz, 3H), 0.92 (d, J=5.7 Hz, 3H), 1.20 (d, J=6.2 Hz, 3H), 1.23 (d, J=6.3 Hz, 3H), 1.79-1.95 (m, 1H), 2.00-2.32 (m, 6H), 3.15 (dd, J=14.6, 7.4 Hz, 1H), 3.35 (dd, J=14.6, 5.5 Hz, 1H), 4.37 (td, J=7.7, 4.4 Hz, 1H), 4.76 (td, J=7.5, 5.5 Hz, 1H), 4.95 (hept, J=6.3 Hz, 1H), 5.15 (bs, 1H), 6.11 (d, J=7.6 Hz, 1H), 6.50 (d, J=7.3 Hz, 1H), 7.09-7.17 (m, 2H), 7.19 (ddd, J=8.2, 7.0, 1.3 Hz, 1H), 7.31-7.40 (m, 1H), 7.70 (d, J=7.8 Hz, 1H), 8.18 (bs, 1H). ESI MS: 506.2 ([M+Na]⁺). HR ESI MS: calcd for C₂₅H₃₃O₅N₅Na 506,23739; found 506,23692.

### Preparation of Isopropyl (S)-2-((S)-3-(1H-indol-3-yl)-2-isobutyramidopropanamido)-6-diazo-5-oxohexanoate (8c)

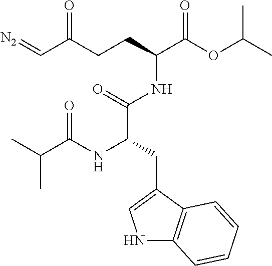

Compound 7a (100 mg, 0.250 mmol, 1 equiv.) was dissolved in anhydrous DMF (3 mL) and pyridine (40 mg, 40 μL, 0.501 mmol, 2 equiv.) followed by isobutyric anhydride (48 mg, 50 μL, 0.300 mmol, 1.2 equiv.) were added. The resulting mixture was stirred at rt for 5 h and DMF was evaporated. The residue was purified by LC on silica gel (DCM/MeOH, 30:1) and the product 8c was obtained as a light yellow solid (103 mg) in 88% yield. ¹H NMR (401 MHz, CDCl₃): 1.11 (d, J=3.3 Hz, 3H), 1.13 (d, J=3.3 Hz, 3H), 1.21 (d, J=6.3 Hz, 3H), 1.24 (d, J=6.3 Hz, 3H), 1.81-1.96 (m, 1H), 2.03-2.29 (m, 3H), 2.35 (hept, J=6.9 Hz, 1H), 3.16 (dd, J=14.5, 7.4 Hz, 1H), 3.36 (dd, J=14.5, 5.4 Hz, 1H), 4.38 (td, J=7.6, 4.4 Hz, 1H), 4.74 (td, J=7.4, 5.3 Hz, 1H), 4.95 (hept, J=6.2 Hz, 1H), 5.15 (bs, 1H), 6.16 (d, J=7.6 Hz, 1H), 6.49 (d, J=7.3 Hz, 1H), 7.09-7.17 (m, 2H), 7.19 (td, J=8.1, 7.6, 1.3 Hz, 1H), 7.36 (dt, J=8.1, 1.0 Hz, 1H), 7.71 (d, J=7.8 Hz, 1H), 8.16 (bs, 1H). ESI MS: 492.2 ([M+Na]⁺). HR ESI MS: calcd for C₂₄H₃₁O₅N₅Na 492.22174; found 492.22135.

### Preparation of Isopropyl (S)-2-((S)-3-(1H-indol-3-yl)-2-pivalamidopropanamido)-6-diazo-5-oxohexanoate (8d)

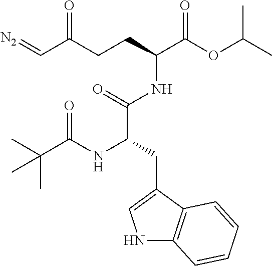

Compound 7a (280 mg, 0.701 mmol, 1 equiv.) was dissolved in anhydrous DMF (12 mL) and DIEA (181 mg, 244 μL, 1.40 mmol, 2 equiv.) followed by trimethylacetyl chloride (110 mg, 112 μL, 0.911 mmol, 1.3 equiv.) were added. The resulting mixture was stirred at rt for 3 h and DMF was evaporated. The residue was purified by LC on silica gel (DCM/MeOH, 30:1) and the product 8d was obtained as an yellow solid (303 mg) in 89% yield. ¹H NMR (401 MHz, CDCl₃): 1.12 (s, 9H), 1.20 (d, J=6.2 Hz, 3H), 1.23 (d, J=6.2 Hz, 3H), 1.84-1.94 (m, 1H), 2.01-2.31 (m, 3H), 3.17 (dd, J=14.6, 7.1 Hz, 1H), 3.34 (dd, J=14.6, 5.7 Hz, 1H), 4.37 (td, J=7.6, 4.3 Hz, 1H), 4.73 (td, J=7.2, 5.8 Hz, 1H), 4.96 (hept, J=6.3 Hz, 1H), 5.16 (bs, 1H), 6.36 (d, J=7.3 Hz, 1H), 6.67 (d, J=7.1 Hz, 1H), 7.07-7.13 (m, 2H), 7.18 (ddd, J=8.1, 7.0, 1.2 Hz, 1H), 7.35 (dt, J=8.1, 1.0 Hz, 1H), 7.69 (dd, J=7.8, 1.1 Hz, 1H), 8.40 (bs, 1H). ¹³C NMR (101 MHz, CDCl₃): 21.79, 21.83, 27.06, 27.48 (3C), 28.05, 36.24, 38.85, 52.25, 53.82, 54.82, 69.51, 110.51, 111.38, 119.00, 119.85, 122.37, 123.53, 127.66, 136.37, 170.85, 171.72, 178.71, 193.89. ESI MS: 506.3 ([M+Na]⁺). HR ESI MS: calcd for C₂₅H₃₃O₅N₅Na 506.23739; found 506.23743.

### Preparation of Isopropyl (S)-2-((S)-2-(cyclopropanecarboxamido)-3-(1H-indol-3-yl)propanamido)-6-diazo-5-oxohexanoate (8e)

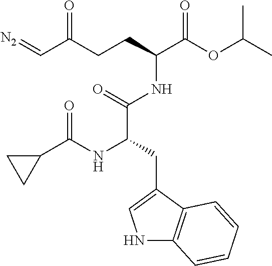

Compound 7a (100 mg, 0.250 mmol, 1 equiv.) was dissolved in anhydrous DMF (3 mL) and pyridine (40 mg, 40 μL, 0.501 mmol, 2 equiv.) followed by cyclopropanecarboxylic anhydride (45 mg, 46 μL, 0.300 mmol, 1.2 equiv.) were added. The resulting mixture was stirred at rt for 3 h and DMF was evaporated. The residue was purified by LC on silica gel (CHCl₃/MeOH, 25:1) and then recrystallized (MeOH) and the product 8e was obtained as a light yellow solid (47 mg) in 40% yield. ¹H NMR (401 MHz, CDCl₃): 0.71-0.83 (m, 2H), 0.93-1.05 (m, 2H), 1.20 (d, J=6.3 Hz, 3H), 1.24 (d, J=6.3 Hz, 3H), 1.36 (ddd, J=11.4, 7.6, 4.1 Hz, 1H), 1.80-1.94 (m, 1H), 2.06-2.31 (m, 3H), 3.16 (dd, J=14.6, 7.6 Hz, 1H), 3.38 (dd, J=14.5, 5.2 Hz, 1H), 4.38 (td, J=7.7, 4.2 Hz, 1H), 4.76 (td, J=7.5, 5.0 Hz, 1H), 4.94 (hept, J=6.3 Hz, 1H), 5.15 (bs, 1H), 6.33 (d, J=7.7 Hz, 1H), 6.44 (d, J=7.4 Hz, 1H), 7.11-7.16 (m, 2H), 7.20 (td, J=8.1, 7.6, 1.3 Hz, 1H), 7.36 (d, J=8.1 Hz, 1H), 7.71 (d, J=7.9 Hz, 1H), 8.16 (bs, 1H). ESI MS: 490.2 ([M+Na]⁺). HR ESI MS: calcd for C₂₄H₂₉O₅N₅Na 490.20609; found 490.20570.

### Preparation of Isopropyl (S)-6-diazo-2-((S)-2-(2-(dimethylamino)acetamido)-3-(1H-indol-3-yl)propanamido)-5-oxohexanoate (8f)

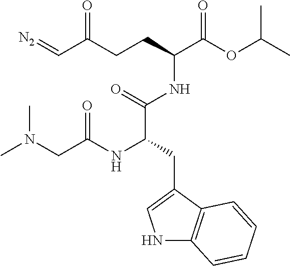

Dimethylglycine (28 mg, 0.275 mmol, 1.1 equiv.) and HATU (114 mg, 0.300 mmol, 1.2 equiv.) were dissolved in anhydrous DMF (4 mL), the mixture was cooled to 0° C. and DIEA (97 mg, 131 μL, 0.751 mmol, 3 equiv.) was added. After 5 minutes of stirring the solution of compound 7a (100 mg, 0.250 mmol, 1 equiv.) in anhydrous DMF (1.5 mL) was added. The resulting mixture was stirred for 60 minutes at 0° C. and 120 minutes at rt. DMF was evaporated, EtOAc (70 mL) was added and the organic phase was washed with 10% NaHCO₃ (50 mL), H₂O (50 mL) and sat. NaCl (50 mL), dried over anhydrous MgSO₄ and solvent was evaporated. The crude product was purified by LC on silica gel (CHCl₃/MeOH, 15:1) and product 8f was obtained as a light yellow solid (73 mg) in 60% yield. ¹H NMR (401 MHz, CDCl₃): 1.19 (d, J=6.3 Hz, 3H), 1.21 (d, J=6.3 Hz, 3H), 1.82-1.96 (m, 1H), 2.04-2.29 (m, 3H), 2.10 (s, 6H), 2.81 (d, J=16.3 Hz, 1H), 2.94 (d, J=16.2 Hz, 1H), 3.18-3.32 (m, 2H), 4.37 (td, J=7.8, 4.6 Hz, 1H), 4.74 (q, J=6.9 Hz, 1H), 4.95 (hept, J=6.3 Hz, 1H), 5.16 (bs, 1H), 6.96 (d, J=7.4 Hz, 1H), 7.04-7.10 (m, 2H), 7.14 (ddd, J=8.1, 7.0, 1.2 Hz, 1H), 7.32 (dt, J=8.1, 0.9 Hz, 1H), 7.62 (dd, J=7.8, 1.1 Hz, 1H), 7.73 (d, J=7.9 Hz, 1H), 8.70 (bs, 1H). ¹³C NMR (101 MHz, CDCl₃): 21.72, 21.76, 26.98, 27.97, 36.26, 45.80 (2C), 52.18, 53.72, 54.84, 62.92, 69.42, 110.42, 111.35, 118.75, 119.52, 122.09, 123.34, 127.58, 136.35, 170.88, 171.26, 171.61, 194.03. ESI MS: 507.2 ([M+Na]⁺). HR ESI MS: calcd for C₂₄H₃₂O₅N₆Na 507.23264; found 507.23212.

### Preparation of Isopropyl (S)-6-diazo-2-((S)-2-(2-(diethylamino)acetamido)-3-(1H-indol-3-yl)propanamido)-5-oxohexanoate (8g)

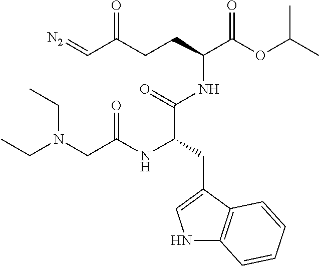

Diethylglycine hydrochloride (69 mg, 0.413 mmol, 1.1 equiv.) and HATU (171 mg, 0.451 mmol, 1.2 equiv.) were dissolved in anhydrous DMF (6 mL), the mixture was cooled to 0° C. and DIEA (194 mg, 262 μL, 1.50 mmol, 4 equiv.) was added. After 5 minutes of stirring the solution of compound 7a (150 mg, 0.376 mmol, 1 equiv.) in anhydrous DMF (3 mL) was added. The resulting mixture was stirred for 30 minutes at 0° C. and 150 minutes at rt. DMF was evaporated, EtOAc (100 mL) was added and the organic phase was washed with sat. NaHCO₃ (50 mL), H₂O (50 mL) and sat. NaCl (50 mL), dried over anhydrous MgSO₄ and solvent was evaporated. The crude product was purified by LC on silica gel (DCM/MeOH, 20:1) and product 8g was obtained as an yellow-orange solid (152 mg) in 79% yield. ¹H NMR (401 MHz, CDCl₃): 0.88 (t, J=7.2 Hz, 6H), 1.19 (d, J=6.3 Hz, 3H), 1.22 (d, J=6.3 Hz, 3H), 1.82-1.95 (m, 1H), 2.03-2.29 (m, 3H), 2.48 (q, J=7.2 Hz, 4H), 3.09 (bs, 2H), 3.25 (d, J=6.6 Hz, 2H), 4.40 (td, J=7.8, 4.6 Hz, 1H), 4.75 (q, J=6.9 Hz, 1H), 4.95 (hept, J=6.2 Hz, 1H), 5.22 (bs, 1H), 6.95 (d, J=7.3 Hz, 1H), 7.02-7.11 (m, 2H), 7.14 (ddd, J=8.2, 7.0, 1.2 Hz, 1H), 7.33 (dt, J=8.1, 1.0 Hz, 1H), 7.61 (d, J=7.8 Hz, 1H), 7.97 (d, J=7.9 Hz, 1H), 8.54 (bs, 1H). ¹³C NMR (101 MHz, CDCl₃): 11.51 (2C), 21.76, 21.80, 27.19, 27.98, 36.16, 48.58 (2C), 52.12, 53.97, 56.78, 69.54, 70.31, 110.32, 111.40, 118.78, 119.62, 122.17, 123.58, 127.60, 136.35, 171.00, 171.51 (2C), 194.15. ESI MS: 513.3 ([M+H]⁺). HR ESI MS: calcd for C₂₆H₃₇O₅N₆ 513.28199; found 513.28172.

### Preparation of Isopropyl (S)-2-((S)-3-(1H-indol-3-yl)-2-(2-(pyrrolidin-1-yl)acetamido)propanamido)-6-diazo-5-oxohexanoate (8h)

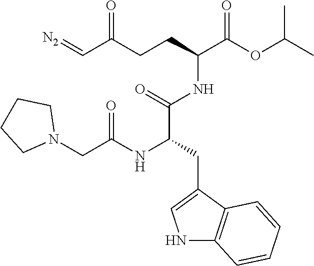

2-(Pyrrolidin-1-yl)acetic acid hydrochloride (46 mg, 0.275 mmol, 1.1 equiv.) and HATU (110 mg, 0.288 mmol, 1.15 equiv.) were dissolved in anhydrous DMF (4 mL), the mixture was cooled to 0° C. and DIEA (129 mg, 175 μL, 1.00 mmol, 4 equiv.) was added. After 5 minutes of stirring the solution of compound 7a (100 mg, 0.250 mmol, 1 equiv.) in anhydrous DMF (2 mL) was added. The resulting mixture was stirred for 30 minutes at 0° C. and 120 minutes at rt. DMF was evaporated, EtOAc (100 mL) was added and the organic phase was washed with sat. NaHCO₃ (50 mL), H₂O (50 mL) and sat. NaCl (50 mL), dried over anhydrous MgSO₄ and solvent was evaporated. The crude product was purified by LC on silica gel (DCM/MeOH, 10:1) and product 8h was obtained as a light yellow solid (53 mg) in 41% yield. ¹H NMR (401 MHz, CDCl₃): 1.20 (d, J=6.3 Hz, 3H), 1.23 (d, J=6.3 Hz, 3H), 1.62-1.75 (m, 4H), 1.90 (ddt, J=14.2, 8.6, 4.3 Hz, 1H), 2.03-2.31 (m, 3H), 2.43-2.64 (m, 4H), 3.12-3.33 (m, 4H), 4.38 (td, J=7.9, 4.7 Hz, 1H), 4.78 (q, J=6.9 Hz, 1H), 4.96 (hept, J=6.3 Hz, 1H), 5.23 (bs, 1H), 7.02-7.10 (m, 2H), 7.10-7.19 (m, 2H), 7.33 (d, J=8.1 Hz, 1H), 7.58 (d, J=7.9 Hz, 1H), 7.77 (d, J=8.0 Hz, 1H), 8.69 (bs, 1H). ¹³C NMR (101 MHz, CDCl₃): 21.76, 21.80, 23.82, 26.97, 28.02 (2C), 36.21, 52.27, 53.88, 54.57 (2C), 55.00, 58.34, 69.57, 110.17, 111.48, 118.68, 119.57, 122.13, 123.73, 127.59, 136.32, 169.96, 171.07, 171.71, 194.33. ESI MS: 511.2 ([M+H]⁺). HR ESI MS: calcd for C₂₆H₃₅O₅N₆ 511.18262; found 511.18265.

### Preparation of Isopropyl (S)-2-((S)-3-(1H-indol-3-yl)-2-(2-morpholinoacetamido)propanamido)-6-diazo-5-oxohexanoate (8i)

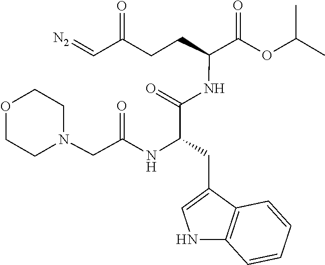

2-Morpholinoacetic acid hydrochloride (50 mg, 0.275 mmol, 1.1 equiv.) and HATU (110 mg, 0.288 mmol, 1.15 equiv.) were dissolved in anhydrous DMF (4 mL), the mixture was cooled to 0° C. and DIEA (129 mg, 175 μL, 1.00 mmol, 4 equiv.) was added. After 5 minutes of stirring the solution of compound 7a (100 mg, 0.250 mmol, 1 equiv.) in anhydrous DMF (2 mL) was added. The resulting mixture was stirred for 30 minutes at 0° C. and overnight (17.5 h) at rt. DMF was evaporated, EtOAc (100 mL) was added and the organic phase was washed with sat. NaHCO₃ (50 mL), H₂O (50 mL) and sat. NaCl (50 mL), dried over anhydrous MgSO₄ and solvent was evaporated. The crude product was purified by LC on silica gel (DCM/MeOH, 20:1) and product 8i was obtained as a light yellow solid (93 mg) in 71% yield. ¹H NMR (401 MHz, CDCl₃): 1.22 (d, J=6.3 Hz, 3H), 1.25 (d, J=6.3 Hz, 3H), 1.88-2.02 (m, 1H), 2.09-2.46 (m, 7H), 3.23-3.36 (m, 2H), 3.36-3.51 (m, 6H), 4.43 (td, J=7.7, 4.5 Hz, 1H), 4.72-4.81 (m, 1H), 4.99 (hept, J=6.3 Hz, 1H), 5.23 (bs, 1H), 6.83-6.89 (m, 1H), 7.08-7.15 (m, 2H), 7.18 (ddd, J=8.2, 7.1, 1.2 Hz, 1H), 7.35 (dt, J=8.1, 0.9 Hz, 1H), 7.64 (dt, J=7.9, 1.0 Hz, 1H), 7.65 (bs, 1H), 8.36 (bs, 1H). ¹³C NMR (101 MHz, CDCl₃): 21.83, 21.86, 27.09, 27.74 (2C), 36.31, 52.23, 53.59, 53.63 (2C), 55.02, 61.78, 66.70, 69.61, 110.42, 111.42, 118.80, 119.92, 122.45, 123.40, 127.71, 136.30, 171.00, 171.48 (2C), 194.03. ESI MS: 549.2 ([M+Na]⁺). HR ESI MS: calcd for C₂₆H₃₄O₆N₆Na 549.24320; found 549.24243.

### Preparation of Isopropyl (S)-2-((S)-3-(1H-indol-3-yl)-2-(2-(4-methylpiperazin-1-yl)acetamido)propanamido)-6-diazo-5-oxohexanoate (8j)

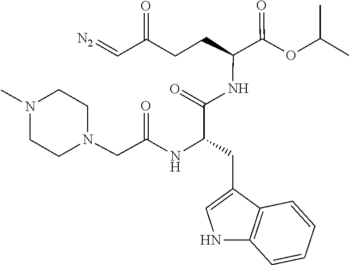

2-(4-Methylpiperazin-1-yl)acetic acid (65 mg, 0.413 mmol, 1.1 equiv.) and HATU (171 mg, 0.451 mmol, 1.2 equiv.) were dissolved in anhydrous DMF (6 mL), the mixture was cooled to 0° C. and DIEA (145 mg, 196 μL, 1.13 mmol, 3 equiv.) was added. After 5 minutes of stirring the solution of compound 7a (150 mg, 0.376 mmol, 1 equiv.) in anhydrous DMF (3 mL) was added. The resulting mixture was stirred for 30 minutes at 0° C. and 90 minutes at rt. DMF was evaporated. The crude product was purified by LC on silica gel (DCM/MeOH, 30:1+1% Et₃N) and product 8j was obtained as an yellow solid (140 mg) in 69% yield. ¹H NMR (401 MHz, CDCl₃): 1.22 (d, J=6.2 Hz, 3H), 1.24 (d, J=6.3 Hz, 3H), 1.88-2.01 (m, 1H), 2.10-2.25 (m, 6H), 2.25-2.35 (m, 3H), 2.34-2.51 (m, 2H), 2.81-3.05 (m, 5H), 3.19-3.38 (m, 2H), 4.43 (td, J=7.7, 4.6 Hz, 1H), 4.73 (q, J=6.9 Hz, 1H), 4.98 (hept, J=6.2 Hz, 1H), 5.22 (bs, 1H), 6.68 (d, J=7.4 Hz, 1H), 7.08-7.15 (m, 2H), 7.19 (ddd, J=8.2, 7.1, 1.3 Hz, 1H), 7.36 (dt, J=8.1, 1.0 Hz, 1H), 7.61-7.70 (m, 2H), 8.23 (bs, 1H). ESI MS: 540.3 ([M+H]⁺). HR ESI MS: calcd for C₂₇H₃₈O₅N₇ 540.29289; found 540.29246.

### Preparation of Isopropyl (S)-2-((S)-3-(1H-indol-3-yl)-2-(quinuclidine-4-carboxamido)propanamido)-6-diazo-5-oxohexanoate (8k)

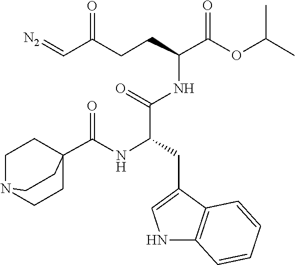

Quinuclidine-4-carboxylic acid hydrochloride (79 mg, 0.413 mmol, 1.1 equiv.) and HATU (171 mg, 0.451 mmol, 1.2 equiv.) were dissolved in anhydrous DMF (6 mL), the mixture was cooled to 0° C. and DIEA (194 mg, 262 μL, 1.50 mmol, 4 equiv.) was added. After 5 minutes of stirring the solution of compound 7a (150 mg, 0.376 mmol, 1 equiv.) in anhydrous DMF (3 mL) was added. The resulting mixture was stirred for 30 minutes at 0° C. and 60 minutes at rt. DMF was evaporated. The crude product was purified by LC on silica gel (DCM/MeOH, 5:1+1% Et₃N) and product 8k was obtained as a light yellow solid (148 mg) in 73% yield. ¹H NMR (401 MHz, DMSO-d₆): 1.18 (d, J=2.7 Hz, 3H), 1.20 (d, J=2.7 Hz, 3H), 1.41-1.51 (m, 6H), 1.77-1.90 (m, 1H), 1.92-2.06 (m, 1H), 2.36-2.44 (m, 2H), 2.67-2.75 (m, 6H), 3.01 (dd, J=14.7, 9.5 Hz, 1H), 3.13 (dd, J=14.6, 4.3 Hz, 1H), 4.14-4.27 (m, 1H), 4.55 (td, J=8.9, 4.2 Hz, 1H), 4.91 (hept, J=6.2 Hz, 1H), 6.03 (bs, 1H), 6.97 (ddd, J=8.0, 7.0, 1.1 Hz, 1H), 7.05 (ddd, J=8.1, 6.9, 1.2 Hz, 1H), 7.13 (d, J=2.4 Hz, 1H), 7.17 (d, J=8.1 Hz, 1H), 7.31 (dt, J=8.1, 0.9 Hz, 1H), 7.60 (d, J=7.9 Hz, 1H), 8.29 (d, J=7.5 Hz, 1H), 10.77 (bs, 1H). ESI MS: 559.3 ([M+Na]⁺). HR ESI MS: calcd for C₂₈H₃₆O₅N₆Na 559.26394; found 559.26353.

### Preparation of Isopropyl (S)-2-((S)-3-(1H-indol-3-yl)-2-(nicotinamido)propanamido)-6-diazo-5-oxohexanoate (81)

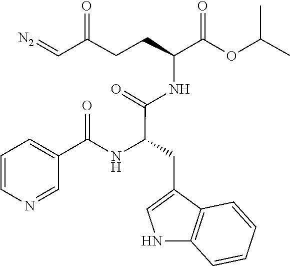

Nicotinic acid (34 mg, 0.275 mmol, 1.1 equiv.) and HATU (114 mg, 0.300 mmol, 1.2 equiv.) were dissolved in anhydrous DMF (4 mL), the mixture was cooled to 0° C. and DIEA (97 mg, 131 μL, 0.751 mmol, 3 equiv.) was added. After 5 minutes of stirring the solution of compound 7a (100 mg, 0.250 mmol, 1 equiv.) in anhydrous DMF (1.5 mL) was added. The resulting mixture was stirred for 30 minutes at 0° C. and 3.5 h at rt. DMF was evaporated, EtOAc (60 mL) was added and the organic phase was washed with 10% KHSO₄ (60 mL), H₂O (60 mL), sat. NaHCO₃ (60 mL), H₂O (60 mL) and sat. NaCl (60 mL). Water phases were extracted with EtOAc (60 mL), combined organic phases were dried over anhydrous MgSO₄ and solvent was evaporated. The crude product was purified by LC on silica gel (CHCl₃/MeOH, 25:1) and product 81 was obtained as a light yellow solid (98 mg) in 78% yield. ¹H NMR (401 MHz, CDCl₃): 1.22 (d, J=6.3 Hz, 3H), 1.25 (d, J=6.3 Hz, 3H), 1.83-1.98 (m, 1H), 2.04-2.36 (m, 3H), 3.29 (dd, J=14.2, 7.4 Hz, 1H), 3.51 (dd, J=14.6, 5.0 Hz, 1H), 4.39 (dt, J=11.8, 5.7 Hz, 1H), 4.87-5.03 (m, 2H), 5.14 (bs, 1H), 6.73 (d, J=7.0 Hz, 1H), 7.03 (d, J=7.4 Hz, 1H), 7.08-7.18 (m, 1H), 7.16-7.25 (m, 2H), 7.32-7.43 (m, 2H), 7.70-7.77 (m, 1H), 8.09 (d, J=7.3 Hz, 1H), 8.20 (bs, 1H), 8.72 (d, J=4.9 Hz, 1H), 8.98 (bs, 1H). ¹³C NMR (101 MHz, CDCl₃): 21.76, 21.79, 26.82, 28.26, 36.28, 52.48, 54.51, 55.04, 69.54, 110.14, 111.50, 118.72, 119.73, 122.27, 123.47, 123.72, 127.59, 129.61, 135.30, 136.40, 148.29, 152.28, 165.52, 170.90, 171.57, 194.19. ESI MS: 527.2 ([M+Na]⁺). HR ESI MS: calcd for C₂₆H₂₈O₅N₆Na 527.20134; found 527.20115.

### Preparation of Isopropyl (S)-2-((S)-3-(1H-indol-3-yl)-2-(isonicotinamido)propanamido)-6-diazo-5-oxohexanoate (8m)

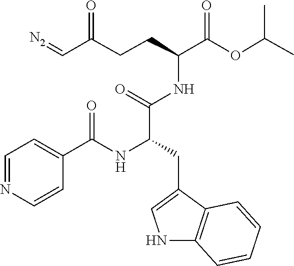

Isonicotinic acid (34 mg, 0.275 mmol, 1.1 equiv.) and HATU (114 mg, 0.300 mmol, 1.2 equiv.) were dissolved in anhydrous DMF (4 mL), the mixture was cooled to 0° C. and DIEA (97 mg, 131 μL, 0.751 mmol, 3 equiv.) was added. After 5 minutes of stirring the solution of compound 7a (100 mg, 0.250 mmol, 1 equiv.) in anhydrous DMF (1.5 mL) was added. The resulting mixture was stirred for 30 minutes at 0° C. and 2 h at rt. DMF was evaporated, EtOAc (60 mL) was added and the organic phase was washed with 10% KHSO₄ (60 mL), H₂O (60 mL), sat. NaHCO₃ (60 mL), H₂O (60 mL) and sat. NaCl (60 mL). Water phases were extracted with EtOAc (2×60 mL), combined organic phases were dried over anhydrous MgSO₄ and solvent was evaporated. The crude product was purified by LC on silica gel (CHCl₃/MeOH, 25:1) and product 8m was obtained as a light yellow solid (86 mg) in 68% yield. ¹H NMR (401 MHz, CDCl₃): 1.22 (d, J=6.2 Hz, 3H), 1.26 (d, J=6.1 Hz, 3H), 1.85-1.98 (m, 1H), 2.03-2.36 (m, 3H), 3.29 (dd, J=14.6, 7.5 Hz, 1H), 3.50 (dd, J=14.6, 7.5 Hz, 1H), 4.38 (dq, J=7.9, 4.5 Hz, 1H), 4.89-5.03 (m, 2H), 5.13 (bs, 1H), 6.77 (bs, 1H), 7.09-7.14 (m, 2H), 7.17-7.23 (m, 2H), 7.37 (d, J=8.1 Hz, 1H), 7.59-7.67 (m, 2H), 7.72 (d, J=7.9 Hz, 1H), 8.23 (bs, 1H), 8.70 (d, J=5.3 Hz, 2H). ESI MS: 527.2 ([M+Na]⁺). HR ESI MS: calcd for C₂₆H₂₈O₅N₆Na 527.20134; found 527.20056.

### Preparation of Isopropyl (S)-6-diazo-2-((S)-2-(2,2-dichloroacetamido)-3-(1H-indol-3-yl)propanamido)-5-oxohexanoate (8n)

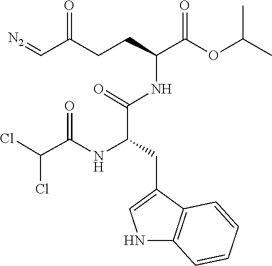

Dichloroacetic acid (53 mg, 34 μL, 0.376 mmol, 1.1 equiv.) and HATU (171 mg, 0.451 mmol, 1.2 equiv.) were dissolved in anhydrous DCM (6 mL), the mixture was cooled to 0° C. and DIEA (146 mg, 196 μL, 1.13 mmol, 3 equiv.) was added. After 5 minutes of stirring the solution of compound 7a (150 mg, 0.376 mmol, 1 equiv.) in anhydrous DCM (2 mL) was added. The resulting mixture was stirred for 30 minutes at 0° C. and 90 minutes at rt. DCM (70 mL) was added and the organic phase was washed with 10% KHSO₄ (50 mL), H₂O (50 mL), sat. NaHCO₃ (50 mL), H₂O (50 mL) and sat. NaCl (50 mL), dried over anhydrous MgSO₄ and solvent was evaporated. The crude product was purified by LC on silica gel (DCM/MeOH, 40:1) and product 8n was obtained as a light yellow solid (132 mg) in 69% yield. ¹H NMR (401 MHz, DMSO-d₆): 1.18 (d, J=3.8 Hz, 3H), 1.20 (d, J=3.8 Hz, 3H), 1.77-1.90 (m, 1H), 1.94-2.05 (m, 1H), 2.36-2.42 (m, 2H), 2.96 (dd, J=14.8, 8.9 Hz, 1H), 3.18 (dd, J=14.9, 4.6 Hz, 1H), 4.21 (ddd, J=9.1, 7.3, 5.2 Hz, 1H), 4.62 (td, J=8.5, 4.5 Hz, 1H), 4.91 (hept, J=6.3 Hz, 1H), 6.02 (bs, 1H), 6.47 (s, 1H), 6.97 (t, J=7.8 Hz, 1H), 7.06 (ddd, J=8.1, 6.9, 1.2 Hz, 1H), 7.11 (d, J=2.4 Hz, 1H), 7.31 (d, J=8.1 Hz, 1H), 7.59 (d, J=7.8 Hz, 1H), 8.66 (d, J=7.4 Hz, 1H), 8.76 (d, J=8.1 Hz, 1H), 10.85 (d, J=2.5 Hz, 1H). ESI MS: 532.1 ([M+Na]⁺). HR ESI MS: calcd for C₂₂H₂₅O₅N₅Cl₂Na 532.11194; found 532.11250.

### Example 5

### Synthesis of Compounds 8n-8o

Compounds 8n-8o were prepared according to the following reaction Scheme.

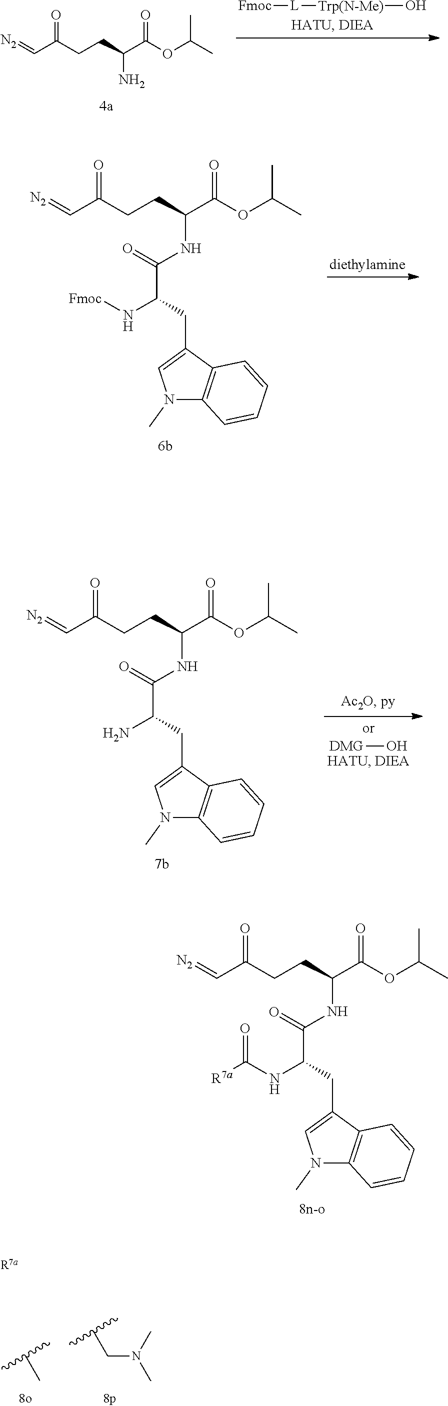

### Preparation of Isopropyl (S)-2-((S)-2-((((9H-fluoren-9-yl)methoxy)carbonyl)amino)-3-(1-methyl-1H-indol-3-yl)propanamido)-6-diazo-5-oxohexanoate (6b)

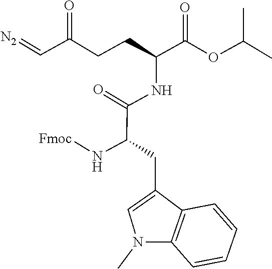

Fmoc-L-Trp(N-Me)-OH (600 mg, 1.36 mmol, 1 equiv.) and HATU (570 mg, 1.50 mmol, 1.1 equiv.) were dissolved in anhydrous DMF (12 mL), the mixture was cooled to 0° C. and DIEA (528 mg, 712 μL, 4.09 mmol, 3 equiv.) was added. After 5 minutes of stirring the solution of compound 4a (291 mg, 1.36 mmol, 1 equiv.) in anhydrous DMF (3 mL) was added. The resulting mixture was stirred for 30 minutes at 0° C. and overnight (16 h) at rt. DMF was evaporated and the crude product was purified by LC on silica gel (DCM/EtOAc, 4:1) and product 6b was obtained as a light yellow solid (545 mg) in 63% yield. ¹H NMR (401 MHz, CDCl₃): 1.19 (d, J=6.3 Hz, 3H), 1.23 (d, J=6.3 Hz, 3H), 1.71-1.85 (m, 1H), 1.92-2.17 (m, 3H), 3.23 (dd, J=14.5, 7.4 Hz, 1H), 3.28-3.39 (m, 1H), 3.73 (s, 3H), 4.19 (t, J=7.1 Hz, 1H), 4.28-4.47 (m, 3H), 4.50-4.64 (m, 1H), 4.97 (hept, J=6.3 Hz, 1H), 5.02-5.17 (m, 1H), 5.52 (d, J=7.3 Hz, 1H), 6.67 (d, J=7.6 Hz, 1H), 6.95 (bs, 1H), 7.10-7.19 (m, 1H), 7.19-7.28 (m, 1H), 7.25-7.33 (m, 3H), 7.39 (ddd, J=9.0, 5.3, 1.9 Hz, 2H), 7.53 (t, J=7.7 Hz, 2H), 7.65 (d, J=7.9 Hz, 1H), 7.76 (d, J=7.5 Hz, 2H). ¹³C NMR (101 MHz, CDCl₃): 21.72, 21.77, 26.98, 28.35, 32.76, 36.06, 47.16, 52.03, 54.66, 55.89, 67.28, 69.48, 108.68, 109.47, 118.86, 119.47, 120.06 (2C), 121.96, 125.22 (2C), 127.17 (2C), 127.81 (2C), 128.05, 137.05 (2C), 141.34, 141.35, 143.80, 143.90, 156.11, 170.95, 171.43, 193.61. ESI MS: 658.3 ([M+Na]⁺). HR ESI MS: calcd for C₃₆H₃₇O₆N₅Na 658.26361; found 658.26275.

### Preparation of Isopropyl (S)-2-((S)-2-amino-3-(1-methyl-1H-indol-3-yl)propanamido)-6-diazo-5-oxohexanoate (7b)

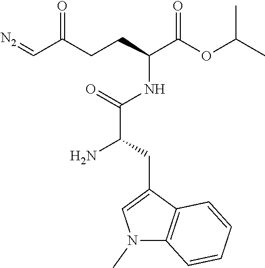

Compound 6b (545 mg, 0.857 mmol, 1 equiv.) was dissolved in anhydrous DMF (10 mL) and diethylamine (627 mg, 887 μL, 8.57 mmol, 10 equiv.) was added. The mixture was stirred at rt for 4 h and DMF was evaporated. The crude product was purified by LC on silica gel (DCM/MeOH, 30:1) and product 7b was obtained as an yellow amorphous compound (280 mg) in 79% yield. ¹H NMR (401 MHz, CDCl₃): 1.22 (d, J=6.2 Hz, 3H), 1.24 (d, J=6.2 Hz, 3H), 1.91 (ddt, J=16.4, 13.4, 8.0 Hz, 1H), 2.05-2.32 (m, 3H), 2.81 (bs, 2H), 3.04 (dd, J=14.5, 8.0 Hz, 1H), 3.30 (dd, J=14.5, 4.5 Hz, 1H), 3.72 (s, 3H), 3.81 (dd, J=8.0, 4.6 Hz, 1H), 4.47 (td, J=8.4, 4.3 Hz, 1H), 5.00 (hept, J=6.2 Hz, 1H), 5.17 (bs, 1H), 6.96 (s, 1H), 7.09 (ddd, J=7.9, 6.8, 1.2 Hz, 1H), 7.20 (ddd, J=8.2, 6.9, 1.2 Hz, 1H), 7.23-7.29 (m, 1H), 7.66 (dt, J=8.0, 1.0 Hz, 1H), 7.88 (d, J=8.2 Hz, 1H). ¹³C NMR (101 MHz, CDCl₃): 21.73, 21.75, 27.53, 30.20, 32.68, 36.57, 51.67, 54.72, 55.32, 69.33, 109.30, 109.37, 119.18, 119.23, 121.85, 127.98, 128.22, 137.16, 171.27, 174.11, 193.85. ESI MS: 436.2 ([M+Na]⁺). HR ESI MS: calcd for C₂₁H₂₇O₄N₅Na 436.19553; found 436.19523.

### Preparation of Isopropyl (S)-2-((S)-2-acetamido-3-(1-methyl-1H-indol-3-yl)propanamido)-6-diazo-5-oxohexanoate (80)

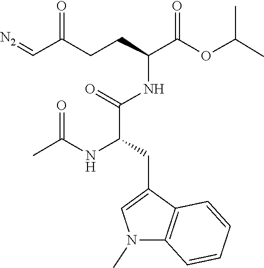

Compound 7b (130 mg, 0.314 mmol, 1 equiv.) was dissolved in anhydrous DCM (6 mL) and pyridine (50 mg, 51 μL, 0.629 mmol, 2 equiv.) followed by acetic anhydride (39 mg, 36 μL, 0.377 mmol, 1.2 equiv.) were added. The resulting mixture was stirred at rt for 3 h and DCM was evaporated. The mixture was purified by LC on silica gel (CHCl₃/MeOH, 30:1) and the product 8o was obtained as a light yellow solid (120 mg) in 84% yield. ¹H NMR (401 MHz, CDCl₃): 1.19 (d, J=6.2 Hz, 3H), 1.23 (d, J=6.3 Hz, 3H), 1.72-1.85 (m, 1H), 1.89-1.97 (m, 1H), 1.98 (s, 3H), 2.04-2.16 (m, 2H), 3.16 (dd, J=14.6, 7.8 Hz, 1H), 3.27-3.38 (m, 1H), 3.75 (s, 3H), 4.34 (td, J=7.8, 4.4 Hz, 1H), 4.77 (td, J=7.6, 5.3 Hz, 1H), 4.97 (hept, J=6.2 Hz, 1H), 5.08 (bs, 1H), 6.11 (d, J=7.5 Hz, 1H), 6.63 (d, J=7.4 Hz, 1H), 6.97 (bs, 1H), 7.12 (ddd, J=8.0, 6.9, 1.1 Hz, 1H), 7.22 (ddd, J=8.2, 6.9, 1.2 Hz, 1H), 7.25-7.31 (m, 1H), 7.63 (dt, J=7.9, 1.0 Hz, 1H). ¹³C NMR (101 MHz, CDCl₃): 21.67, 21.71, 23.24, 26.52, 28.06, 32.66, 35.95, 52.09, 54.13, 54.62, 69.27, 108.94, 109.34, 118.82, 119.25, 121.76, 127.98, 128.13, 136.88, 170.29, 170.91, 171.59, 193.76. ESI MS: 478.2 ([M+Na]⁺). HR ESI MS: calcd for C₂₃H₂₉O₅N₅Na 478.20609; found 478.20660.

### Preparation of Isopropyl (S)-6-diazo-2-((S)-2-(2-(dimethylamino)acetamido)-3-(1-methyl-1H-indol-3-yl)propanamido)-5-oxohexanoate (8p)

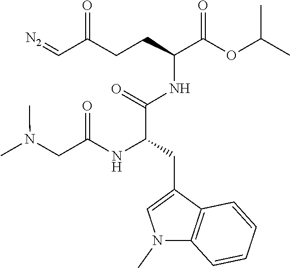

Dimethylglycine (17 mg, 0.160 mmol, 1.1 equiv.) and HATU (66 mg, 0.174 mmol, 1.2 equiv.) were dissolved in anhydrous DMF (3 mL), the mixture was cooled to 0° C. and DIEA (56 mg, 76 μL, 0.435 mmol, 3 equiv.) was added. After 5 minutes of stirring the solution of compound 7b (60 mg, 0.145 mmol, 1 equiv.) in anhydrous DMF (1.5 mL) was added. The resulting mixture was stirred for 30 minutes at 0° C. and 3.5 h at rt. DMF was evaporated and EtOAc (70 mL) was added and the organic phase was washed with sat. NaHCO₃ (40 mL), H₂O (40 mL) and sat. NaCl (40 mL), dried over anhydrous MgSO₄ and solvent was evaporated. The crude product was purified by LC on silica gel (CHCl₃/MeOH, 15:1) and product 8p was obtained as a light yellow solid (50 mg) in 69% yield. ¹H NMR (401 MHz, CDCl₃): 1.20 (d, J=6.3 Hz, 3H), 1.23 (d, J=6.3 Hz, 3H), 1.89 (dtd, J=14.3, 8.0, 5.7 Hz, 1H), 2.04-2.32 (m, 3H), 2.18 (s, 6H), 2.92 (d, J=16.1 Hz, 1H), 3.01 (d, J=16.1 Hz, 1H), 3.25 (qd, J=14.7, 6.8 Hz, 2H), 3.73 (s, 3H), 4.39 (td, J=7.8, 4.6 Hz, 1H), 4.68 (q, J=7.1 Hz, 1H), 4.96 (hept, J=6.3 Hz, 1H), 5.19 (bs, 1H), 6.76 (d, J=7.6 Hz, 1H), 6.96 (s, 1H), 7.09 (ddd, J=8.0, 6.9, 1.2 Hz, 1H), 7.20 (ddd, J=8.2, 6.8, 1.2 Hz, 1H), 7.26 (d, J=8.2 Hz, 1H), 7.65 (dt, J=8.0, 1.0 Hz, 1H), 7.78 (d, J=7.7 Hz, 1H). ¹³C NMR (101 MHz, CDCl₃): 21.76, 21.82, 27.20, 27.79, 32.75, 36.33, 45.74 (2C), 52.14, 53.95, 54.76, 62.75, 69.43, 109.11, 109.30, 119.08, 119.23, 121.87, 128.01, 128.04, 137.11, 170.71, 170.91, 171.49, 193.90. ESI MS: 499.2 ([M+H]⁺). HR ESI MS: calcd for C₂₅H₃₅O₅N₆ 499.26634; found 499.26672.

### Example 6

### Synthesis of Compounds 11a-11d

Compounds 11a-11d were prepared according to the following reaction Scheme.

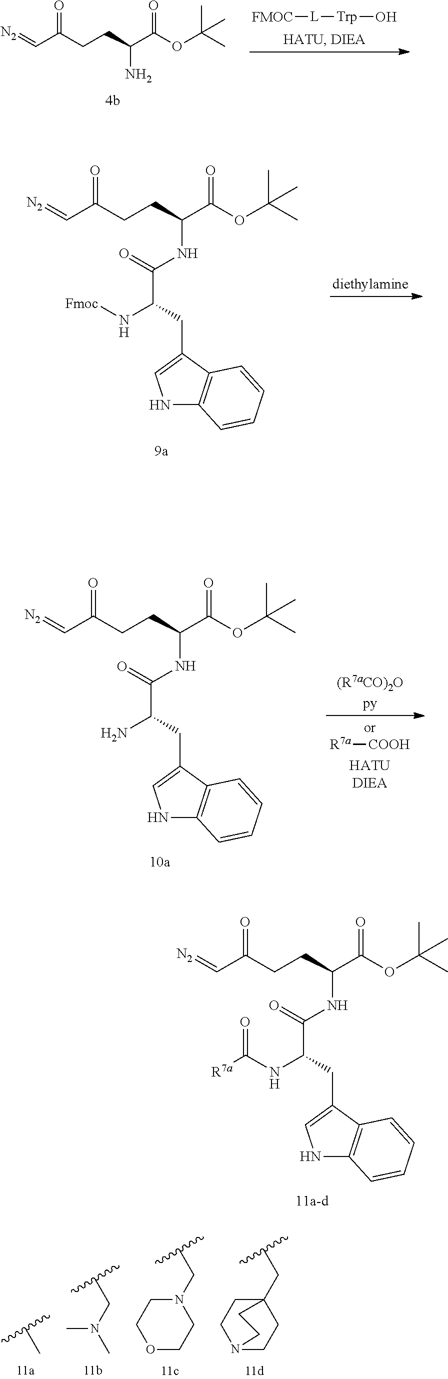

### Preparation of tert-Butyl (S)-2-((S)-2-acetamido-3-(1H-indol-3-yl)propanamido)-6-diazo-5-oxohexanoate (11a)

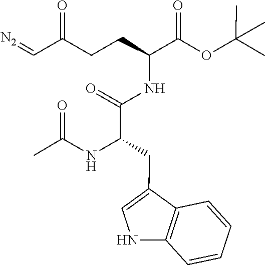

Compound 10a (100 mg, 0.242 mmol, 1 equiv.) was dissolved in anhydrous DMF (1 mL) and pyridine (38 mg, 39 μL, 0.484 mmol, 2 equiv.) followed by acetic anhydride (27 mg, 25 μL, 0.266 mmol, 1.1 equiv.) were added. The resulting mixture was stirred at rt for 2 h and DMF was evaporated. The mixture was purified by LC on silica gel (CHCl₃/MeOH, 30:1) and the product 11a was obtained as an yellow solid (101 mg) in 92% yield. ¹H NMR (401 MHz, CDCl₃): 1.42 (s, 9H), 1.81-1.92 (m, 1H), 1.94 (s, 3H), 2.01-2.17 (m, 2H), 2.18-2.30 (m, 1H), 3.19 (dd, J=14.7, 6.6 Hz, 1H), 3.29 (dd, J=14.7, 5.7 Hz, 1H), 4.31 (td, J=7.7, 4.5 Hz, 1H), 4.77 (q, J=6.4 Hz, 1H), 5.16 (bs, 1H), 6.35 (d, J=7.7 Hz, 1H), 6.82 (d, J=7.4 Hz, 1H), 7.04-7.12 (m, 2H), 7.16 (ddd, J=8.2, 7.0, 1.3 Hz, 1H), 7.33 (dt, J=8.1, 1.0 Hz, 1H), 7.63 (d, J=7.9 Hz, 1H), 8.56 (bs, 1H). ¹³C NMR (101 MHz, CDCl₃): 23.33, 27.11, 28.04 (3C), 28.16, 36.29, 52.70, 53.99, 54.92, 82.49, 110.30, 111.45, 118.75, 119.76, 122.25, 123.57, 127.72, 136.35, 170.34, 170.48, 171.51, 194.22. ESI MS: 478.2 ([M+Na]⁺). HR ESI MS: calcd for C₂₃H₂₉O₅N₅Na 478.20609; found 478.20567.

### Preparation of tert-Butyl (S)-6-diazo-2-((S)-2-(2-(dimethylamino)acetamido)-3-(1H-indol-3-yl)propanamido)-5-oxohexanoate (11b)

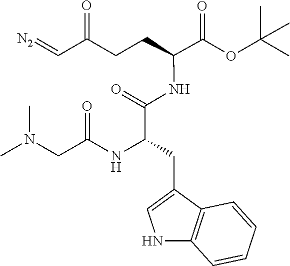

Dimethylglycine (137 mg, 1.33 mmol, 1.1 equiv.) and HATU (552 mg, 1.45 mmol, 1.2 equiv.) were dissolved in anhydrous DMF (15 mL), the mixture was cooled to 0° C. and DIEA (469 mg, 632 μL, 3.63 mmol, 3 equiv.) was added. After 5 minutes of stirring the solution of compound 10a (500 mg, 1.21 mmol, 1 equiv.) in anhydrous DMF (4 mL) was added. The resulting mixture was stirred for 30 minutes at 0° C. and 2 h at rt. DMF was evaporated and EtOAc (200 mL) was added and the organic phase was washed with sat. NaHCO₃ (100 mL), H₂O (100 mL) and sat. NaCl (100 mL), dried over anhydrous MgSO₄ and solvent was evaporated. The crude product was purified by LC on silica gel (DCM/MeOH, 15:1) and product 11b was obtained as a light yellow solid (533 mg) in 88% yield. ¹H NMR (401 MHz, CDCl₃): 1.40 (s, 9H), 1.84-1.94 (m, 1H), 2.02-2.12 (m, 1H), 2.11 (s, 6H), 2.17-2.30 (m, 2H), 2.87 (d, J=16.1 Hz, 1H), 2.96 (d, J=16.1 Hz, 1H), 3.26 (d, J=6.9 Hz, 2H), 4.32 (q, J=7.2 Hz, 1H), 4.76 (q, J=6.9 Hz, 1H), 5.21 (bs, 1H), 7.01-7.17 (m, 4H), 7.32 (d, J=8.1 Hz, 1H), 7.61 (d, J=7.8 Hz, 1H), 7.85 (d, J=8.0 Hz, 1H), 8.93 (bs, 1H). ¹³C NMR (101 MHz, CDCl₃): 27.08, 27.98 (3C), 29.71, 36.32, 45.50 (2C), 52.64, 53.82, 54.83, 62.37, 82.31, 110.29, 111.43, 118.71, 119.43, 122.01, 123.45, 127.53, 136.34, 170.52, 171.53, 171.54, 194.26. ESI MS: 499.3 ([M+H]⁺). HR ESI MS: calcd for C₂₅H₃₅O₅N₆ 499.26634; found 499.26585.

### Preparation of tert-Butyl (S)-2-((S)-3-(1H-indol-3-yl)-2-(2-morpholinoacetamido)propanamido)-6-diazo-5-oxohexanoate (11c)

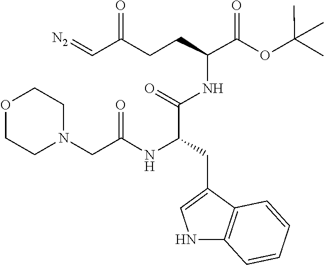

2-Morpholinoacetic acid hydrochloride (48 mg, 0.266 mmol, 1.1 equiv.) and HATU (106 mg, 0.278 mmol, 1.15 equiv.) were dissolved in anhydrous DMF (4 mL), the mixture was cooled to 0° C. and DIEA (125 mg, 168 μL, 0.967 mmol, 4 equiv.) was added. After 5 minutes of stirring the solution of compound 10a (100 mg, 0.242 mmol, 1 equiv.) in anhydrous DMF (2 mL) was added. The resulting mixture was stirred for 30 minutes at 0° C. and overnight (19.5 h) at rt. DMF was evaporated, EtOAc (100 mL) was added and the organic phase was washed with sat. NaHCO₃ (50 mL), H₂O (50 mL) and sat. NaCl (50 mL), dried over anhydrous MgSO₄ and solvent was evaporated. The crude product was purified by LC on silica gel (DCM/MeOH, 20:1) and product 11c was obtained as a light yellow solid (107 mg) in 82% yield. ¹H NMR (401 MHz, CDCl₃): 1.44 (s, 9H), 1.86-2.02 (m, 1H), 2.08-2.40 (m, 7H), 2.85 (d, J=16.4 Hz, 1H), 2.98 (d, J=16.4 Hz, 1H), 3.29 (t, J=7.1 Hz, 2H), 3.38 (dtd, J=14.0, 8.0, 6.6, 3.0 Hz, 4H), 4.38 (td, J=7.5, 4.6 Hz, 1H), 4.77 (q, J=6.8 Hz, 1H), 5.23 (bs, 1H), 6.84 (d, J=7.4 Hz, 1H), 7.07-7.13 (m, 2H), 7.17 (ddd, J=8.1, 7.0, 1.2 Hz, 1H), 7.34 (dt, J=8.1, 1.0 Hz, 1H), 7.58 (d, J=7.8 Hz, 1H), 7.63 (dd, J=8.0, 1.1 Hz, 1H), 8.45 (bs, 1H). ¹³C NMR (101 MHz, CDCl₃): 27.29, 27.65, 28.09 (3C), 36.33, 52.60, 53.44, 53.66 (2C), 54.90, 61.88, 66.80 (2C), 82.57, 110.35, 111.43, 118.76, 119.87, 122.41, 123.34, 127.71, 136.30, 170.50, 170.57, 171.39, 194.09. ESI MS: 541.3 ([M+H]⁺). HR ESI MS: calcd for C₂₇H₃₇O₆N₆ 541.27691; found 541.27637.

### Preparation of tert-Butyl (S)-2-((S)-3-(1H-indol-3-yl)-2-(quinuclidine-4-carboxamido)propanamido)-6-diazo-5-oxohexanoate (11d)

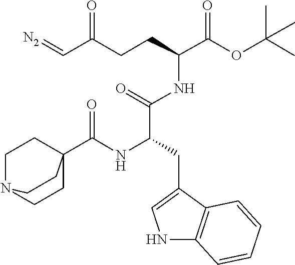

Quinuclidine-4-carboxylic acid hydrochloride (51 mg, 0.266 mmol, 1.1 equiv.) and HATU (106 mg, 0.278 mmol, 1.15 equiv.) were dissolved in anhydrous DMF (4 mL), the mixture was cooled to 0° C. and DIEA (125 mg, 168 μL, 0.967 mmol, 4 equiv.) was added. After 5 minutes of stirring the solution of compound 10a (100 mg, 0.242 mmol, 1 equiv.) in anhydrous DMF (2 mL) was added. The resulting mixture was stirred for 30 minutes at 0° C. and 90 minutes at rt. DMF was evaporated. The crude product was purified by LC on silica gel (DCM/MeOH, 5:1+1% Et₃N) and product 11d was obtained as a light yellow-orange solid (118 mg) in 89% yield. ¹H NMR (401 MHz, CDCl₃): 1.42 (s, 9H), 1.55-1.67 (m, 6H), 1.82-1.94 (m, 1H), 2.00-2.17 (m, 2H), 2.17-2.32 (m, 1H), 2.82-2.89 (m, 6H), 3.17 (dd, J=14.7, 6.8 Hz, 1H), 3.33 (dd, J=14.7, 6.8 Hz, 1H), 4.29 (td, J=7.5, 4.4 Hz, 1H), 4.74 (td, J=7.0, 5.6 Hz, 1H), 5.17 (bs, 1H), 6.24 (d, J=7.4 Hz, 1H), 6.72 (d, J=7.2 Hz, 1H), 7.07-7.13 (m, 2H), 7.17 (ddd, J=8.1, 6.9, 1.2 Hz, 1H), 7.35 (d, J=8.1 Hz, 1H), 7.66 (d, J=7.8 Hz, 1H), 8.69 (bs, 1H). ¹³C NMR (101 MHz, CDCl₃): 27.03, 28.07 (3C), 28.12 (3C), 29.81, 36.30, 45.97, 47.29 (3C), 52.78, 53.64, 54.85, 82.49, 110.29, 111.47, 119.01, 119.75, 122.37, 123.61, 127.66, 136.40, 170.46, 171.43, 176.21, 194.13. ESI MS: 551.3 ([M+H]⁺). HR ESI MS: calcd for C₂₉H₃₉O₅N₆ 551.29764; found 551.29730.

### Example 7

### Synthesis of Compounds 12a-12k

Compounds 12a-12k were prepared according to the following reaction Scheme.

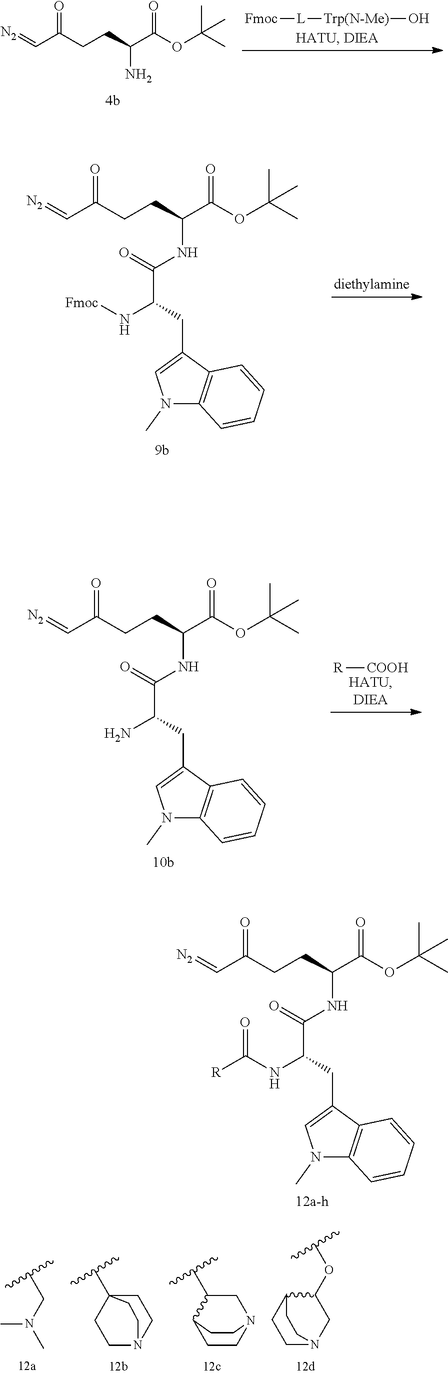

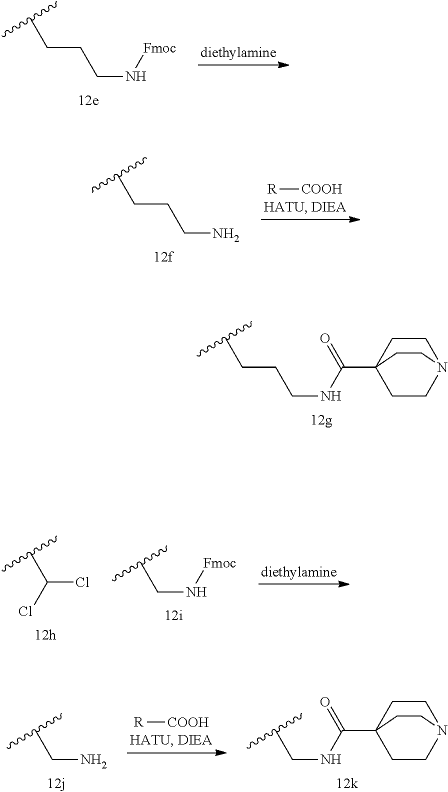

### Preparation of tert-Butyl (S)-6-diazo-2-((S)-2-(2-(dimethylamino)acetamido)-3-(1-methyl-1H-indol-3-yl)propanamido)-5-oxohexanoate (12a)

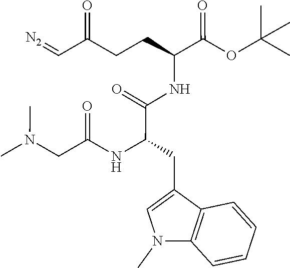

Dimethylglycine (21 mg, 0.206 mmol, 1.1 equiv.) and HATU (85 mg, 0.225 mmol, 1.2 equiv.) were dissolved in anhydrous DMF (3 mL), the mixture was cooled to 0° C. and DIEA (73 mg, 98 μL, 0.561 mmol, 3 equiv.) was added. After 5 minutes of stirring the solution of compound 10b (80 mg, 0.187 mmol, 1 equiv.) in anhydrous DMF (2 mL) was added. The resulting mixture was stirred for 30 minutes at 0° C. and 90 minutes at rt. DMF was evaporated, EtOAc (70 mL) was added and the organic phase was washed with sat. NaHCO₃ (50 mL), H₂O (50 mL) and sat. NaCl (50 mL), dried over anhydrous MgSO₄ and solvent was evaporated. The crude product was purified by LC on silica gel (DCM/MeOH, 12:1) and product 12a was obtained as a light yellow solid (70 mg) in 73% yield. ¹H NMR (401 MHz, CDCl₃): 1.39 (s, 9H), 1.85 (dtd, J=14.3, 8.4, 5.8 Hz, 1H), 2.01-2.30 (m, 3H), 2.09 (s, 6H), 2.80 (d, J=16.2 Hz, 1H), 2.92 (d, J=16.2 Hz, 1H), 3.21 (d, J=6.8 Hz, 2H), 3.69 (s, 3H), 4.30 (td, J=7.9, 4.7 Hz, 1H), 4.61-4.67 (m, 1H), 5.22 (bs, 1H), 6.92 (s, 1H), 7.00 (d, J=7.6 Hz, 1H), 7.06 (ddd, J=8.0, 6.9, 1.1 Hz, 1H), 7.16 (ddd, J=8.2, 6.9, 1.2 Hz, 1H), 7.22 (dt, J=8.3, 1.0 Hz, 1H), 7.60 (dt, J=8.0, 1.0 Hz, 1H), 7.70 (d, J=7.9 Hz, 1H). ¹³C NMR (101 MHz, CDCl₃): 27.18, 27.66, 27.91 (3C), 32.61, 36.26, 45.71 (2C), 52.36, 53.51, 54.83, 62.78, 82.29, 108.83, 109.21, 118.87, 119.06, 121.72, 127.95, 136.97 (2C), 170.46, 171.26, 171.42, 194.28. ESI MS: 535.3 ([M+Na]⁺). HR ESI MS: calcd for C₂₆H₃₆O₅N₆Na 535.26394; found 535.26373.

### Preparation of tert-Butyl (S)-6-diazo-2-((S)-3-(1-methyl-1H-indol-3-yl)-2-(quinuclidine-4-carboxamido)propanamido)-5-oxohexanoate (12b)

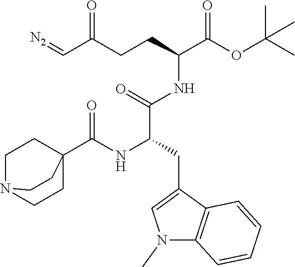

Quinuclidine-4-carboxylic acid hydrochloride (37 mg, 0.193 mmol, 1.1 equiv.) and HATU (80 mg, 0.211 mmol, 1.2 equiv.) were dissolved in anhydrous DMF (3 mL), the mixture was cooled to 0° C. and DIEA (91 mg, 122 μL, 0.702 mmol, 4 equiv.) was added. After 5 minutes of stirring the solution of compound 10b (75 mg, 0.175 mmol, 1 equiv.) in anhydrous DMF (2 mL) was added. The resulting mixture was stirred for 30 minutes at 0° C. and 60 minutes at rt. DMF was evaporated. The crude product was purified by LC on silica gel (DCM/MeOH, 15:1+1% Et₃N) and product 12b was obtained as an yellow solid (57 mg) in 58% yield. ¹H NMR (401 MHz, CDCl₃): 1.43 (s, 9H), 1.69-1.79 (m, 6H), 1.83-1.94 (m, 1H), 1.99-2.11 (m, 1H), 2.11-2.37 (m, 2H), 2.95-3.03 (m, 6H), 3.17 (dd, J=14.6, 7.0 Hz, 1H), 3.28 (dd, J=14.6, 5.3 Hz, 1H), 3.71 (s, 3H), 4.29 (td, J=7.6, 4.7 Hz, 1H), 4.71 (td, J=7.2, 5.3 Hz, 1H), 5.17 (bs, 1H), 6.41 (d, J=7.3 Hz, 1H), 6.95 (bs, 2H), 7.07 (ddd, J=7.9, 6.9, 1.1 Hz, 1H), 7.19 (ddd, J=8.1, 6.9, 1.1 Hz, 1H), 7.26 (d, J=8.1 Hz, 1H), 7.62 (d, J=7.9 Hz, 1H). ¹³C NMR (101 MHz, CDCl₃): 26.89 (3C), 27.97 (2C), 28.04 (3C), 32.76, 35.96, 36.36, 46.17 (3C), 52.74, 53.74, 54.83, 82.37, 108.57, 109.43, 119.04, 119.20, 121.92, 128.16, 128.38, 136.99, 170.50, 171.18, 174.54, 194.04. ESI MS: 565.3 ([M+H]⁺). HR ESI MS: calcd for C₃₀H₄₁O₅N₆ 565.31329; found 565.31258.

### Preparation of tert-Butyl (2S)-6-diazo-2-((2S)-3-(1-methyl-1H-indol-3-yl)-2-(quinuclidine-3-carboxamido)propanamido)-5-oxohexanoate (12c)

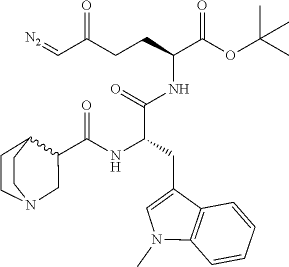

Quinuclidine-3-carboxylic acid hydrochloride (racemic, 25 mg, 0.128 mmol, 1.1 equiv.) and HATU (51 mg, 0.134 mmol, 1.15 equiv.) were dissolved in anhydrous DCM (2 mL), the mixture was cooled to 0° C. and DIEA (45 mg, 61 μL, 0.351 mmol, 3 equiv.) was added. After 5 minutes of stirring the solution of compound 10b (50 mg, 0.117 mmol, 1 equiv.) in anhydrous DCM (2 mL) was added. The resulting mixture was stirred for 30 minutes at 0° C. and 60 minutes at rt. DCM was evaporated. The crude product was purified by LC on silica gel (DCM/MeOH, 15:1+1% Et₃N) and product 12c was obtained as an yellow solid (62 mg) in 97% yield. ¹H NMR (401 MHz, DMSO-d₆): 1.40 (s, 9H), 1.52 (d, J=31.6 Hz, 2H), 1.66 (q, J=3.1 Hz, 1H), 1.77-1.87 (m, 1H), 1.91-2.02 (m, 2H), 2.29-2.39 (m, 4H), 2.57-2.68 (m, 4H), 2.88 (dt, J=14.6, 10.8 Hz, 2H), 2.99-3.18 (m, 1H), 3.70 (d, J=10.2 Hz, 3H), 4.12 (td, J=8.5, 5.3 Hz, 1H), 4.55 (td, J=8.9, 4.5 Hz, 1H), 4.65 (td, J=9.5, 4.1 Hz, 1H), 6.04 (d, J=18.3 Hz, 1H), 6.95-7.17 (m, 3H), 7.35 (t, J=7.9 Hz, 1H), 7.63 (t, J=7.4 Hz, 1H), 7.86 (dd, J=8.4, 2.1 Hz, 1H), 8.28 (dd, J=14.0, 7.5 Hz, 1H). ¹³C NMR (101 MHz, CDCl₃): [22.1, 22.2], 25.6, 25.8, [27.0, 27.1], 28.1 (3C), [28.1, 28.2], [32.8, 32.8], 36.3, [42.8, 42.9], 47.1, [47.2, 47.3], 49.2, 49.5, [52.6, 52.7], [53.9, 53.9], 82.4, [108.8, 108.9], [109.4, 109.4], 119.0, [119.3, 119.4], [121.9, 121.9], [128.1, 128.1], [128.2, 128.3], 137.1, [170.4, 170.5], [171.5, 171.5], [174.3, 174.4], 194.0. ESI MS: 565.3 ([M+H]⁺). HR ESI MS: calcd for C₃₀H₄₁O₅N₆ 565.31329; found 565.31305.

### Preparation of tert-Butyl (2S)-6-diazo-2-((2S)-3-(1-methyl-1H-indol-3-yl)-2-(((quinuclidin-3-yloxy)carbonyl)amino)propanamido)-5-oxohexanoate (12d)

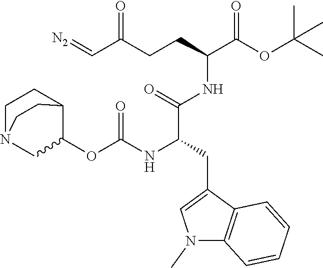

Compound 10b (52 mg, 0.122 mmol, 1 equiv.) was dissolved in anhydrous DCM (3 mL) and the mixture was cooled to 0° C. Triethylamine (49 mg, 68 μL, 0.487 mmol, 4 equiv.) followed by suspension of quinuclidin-3-yl carbonochloridate hydrochloride (racemic, 55 mg, 0.243 mmol, 2 equiv.) in anhydrous DCM (2 mL) were added. The resulting mixture was stirred 60 minutes at 0° C. and 60 minutes at rt. DCM was evaporated. The crude product was purified by LC on silica gel (DCM/MeOH, 5:1+1% Et₃N) and product 12d was obtained as a light yellow solid (52 mg) in 74% yield. ¹H NMR (401 MHz, CDCl₃): 1.43 (s, 9H), 1.50-1.62 (m, 1H), 1.63-1.78 (m, 1H), 1.81-2.02 (m, 2H), 2.01-2.13 (m, 1H), 2.14-2.36 (m, 3H), 2.97-3.02 (m, 3H), 3.43-3.48 (m, 4H), 3.63 (d, J=2.6 Hz, 1H), 3.74 (d, J=6.6 Hz, 3H), 4.32 (q, J=7.3 Hz, 1H), 4.43-4.51 (m, 1H), 4.80-4.90 (m, 1H), 5.15-5.25 (m, 1H), 5.55 (dd, J=25.9, 8.0 Hz, 1H), 6.76 (bs, 1H), 6.96 (d, J=1.7 Hz, 1H), 7.09 (q, J=8.3, 7.7 Hz, 1H), 7.21 (q, J=7.3 Hz, 1H), 7.27-7.35 (m, 2H), 7.62 (dd, J=7.9, 3.3 Hz, 1H). ¹³C NMR (101 MHz, CDCl₃): [18.4, 18.4], [22.8, 22.8], 25.0, 25.1, 27.2, 28.5 (3C), 28.5, [32.8, 32.8], 36.4, 45.5, 46.4, 52.7, 54.1, [55.6, 55.6], 69.9, 82.5, 108.6, [109.5, 109.5], [119.0, 119.0], 119.4, [122.0, 122.0], 128.1, 128.3, 137.1, 155.4, 170.6, [171.2, 171.3], 194.0. ESI MS: 581.3 ([M+H]⁺). HR ESI MS: calcd for C₃₀H₄₁O₆N₆ 581.30821; found 581.30798.

### Preparation of tert-Butyl (10S,13S)-13-(4-diazo-3-oxobutyl)-1-(9H-fluoren-9-yl)-10-((1-methyl-1H-indol-3-yl)methyl)-3,8,11-trioxo-2-oxa-4,9,12-triazatetradecan-14-oate (12e)

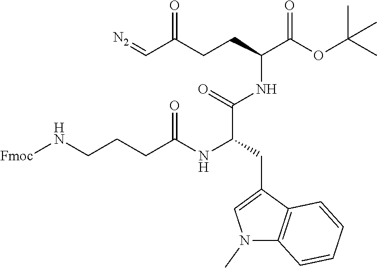

Fmoc-GABA-OH (167 mg, 0.515 mmol, 1.1 equiv.) and HATU (213 mg, 0.561 mmol, 1.2 equiv.) were dissolved in anhydrous DMF (7 mL), the mixture was cooled to 0° C. and DIEA (181 mg, 245 μL, 1.40 mmol, 3 equiv.) was added. After 5 minutes of stirring the solution of compound 10b (200 mg, 0.468 mmol, 1 equiv.) in anhydrous DMF (3 mL) was added. The resulting mixture was stirred for 30 minutes at 0° C. and overnight (23.5 h) at rt. DMF was evaporated, EtOAc (150 mL) was added and the organic phase was washed with sat. NaHCO₃ (50 mL), H₂O (50 mL) and sat. NaCl (50 mL), dried over anhydrous MgSO₄ and solvent was evaporated. The crude product was purified by LC on silica gel (DCM/MeOH, 30:1) and product 12e was obtained as a light yellow solid (321 mg) in 93% yield. ¹H NMR (401 MHz, CDCl₃): 1.43 (s, 9H), 1.71-1.93 (m, 3H), 2.00-2.30 (m, 3H), 2.19 (t, J=6.9 Hz, 2H), 3.07-3.22 (m, 3H), 3.32 (dd, J=14.6, 5.7 Hz, 1H), 3.68 (s, 3H), 4.18 (t, J=6.9 Hz, 1H), 4.35 (dq, J=8.6, 4.2 Hz, 3H), 4.75 (q, J=6.9 Hz, 1H), 5.04-5.18 (m, 2H), 6.36 (d, J=7.6 Hz, 1H), 6.74 (d, J=7.4 Hz, 1H), 6.93 (s, 1H), 7.06-7.12 (m, 1H), 7.17 (t, J=7.4 Hz, 1H), 7.23 (d, J=8.1 Hz, 1H), 7.29 (tdd, J=7.5, 5.0, 1.2 Hz, 2H), 7.39 (tt, J=7.5, 1.6 Hz, 2H), 7.53-7.61 (m, 2H), 7.65 (dt, J=7.9, 1.0 Hz, 1H), 7.76 (d, J=7.5 Hz, 2H). ¹³C NMR (101 MHz, CDCl₃): 25.84, 27.22, 27.91 (3C), 28.08, 32.74, 33.42, 36.36, 40.17, 47.37, 52.62, 53.98, 54.81, 66.63, 82.41, 108.84, 109.42, 119.06, 119.38, 120.08 (2C), 121.93, 125.18 (2C), 127.16 (2C), 127.80 (2C), 128.16, 128.29, 137.09, 141.43 (2C), 144.06, 144.13, 156.85, 170.56, 171.38, 172.49, 194.06. ESI MS: 757.3 ([M+Na]⁺). HR ESI MS: calcd for C₄₁H₄₆O₇N₆Na 757.33202; found 757.33106.

### Preparation of tert-Butyl (S)-2-((S)-2-(4-aminobutanamido)-3-(1-methyl-1H-indol-3-yl)propanamido)-6-diazo-5-oxohexanoate (12f)

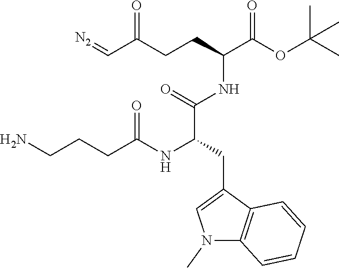

Compound 12e (300 mg, 0.408 mmol, 1 equiv.) was dissolved in anhydrous DCM/DMF 3:1 (3+1 mL) and diethylamine (299 mg, 422 μL, 4.08 mmol, 10 equiv.) was added. The mixture was stirred at rt for 3 h and solvents were evaporated. The crude product was purified by LC on silica gel (DCM/MeOH, 3:1+1% Et₃N) and product 12f was obtained as an yellow amorphous compound (150 mg) in 72% yield. ¹H NMR (401 MHz, CDCl₃): 1.40 (s, 9H), 1.61-1.73 (m, 2H), 1.79-1.90 (m, 1H), 2.00-2.12 (m, 5H), 2.20 (t, J=7.2 Hz, 2H), 2.55-2.68 (m, 2H), 3.15 (dd, J=14.7, 6.8 Hz, 1H), 3.25 (dd, J=14.6, 5.9 Hz, 1H), 3.68 (s, 3H), 4.31 (td, J=7.9, 4.6 Hz, 1H), 4.75 (q, J=6.7 Hz, 1H), 5.18 (bs, 1H), 6.82 (d, J=7.7 Hz, 1H), 6.92 (s, 1H), 7.01-7.08 (m, 2H), 7.16 (ddd, J=8.2, 6.9, 1.2 Hz, 1H), 7.23 (dt, J=8.3, 1.0 Hz, 1H), 7.60 (dt, J=7.9, 1.0 Hz, 1H). ¹³C NMR (101 MHz, CDCl₃): 27.16, 27.84, 27.95 (3C), 28.47, 32.63, 33.89, 36.29, 41.22, 52.47, 53.84, 54.62, 82.17, 108.92, 109.26, 118.93, 119.12, 121.71, 128.10, 128.13, 136.94, 170.52, 171.63, 172.92, 193.91. ESI MS: 513.3 ([M+H]⁺). HR ESI MS: calcd for C₂₆H₃₇O₅N₆ 513.28199; found 513.28135.

### Preparation of tert-Butyl (S)-6-diazo-2-((S)-3-(1-methyl-1H-indol-3-yl)-2-(4-(quinuclidine-4-carboxamido)butanamido)propanamido)-5-oxohexanoate (12g)

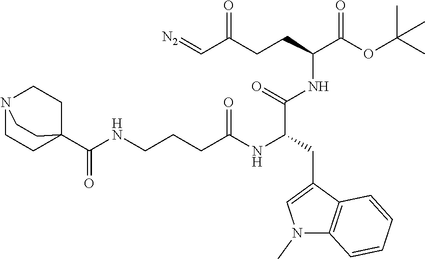

Quinuclidine-4-carboxylic acid hydrochloride (21 mg, 0.107 mmol, 1.1 equiv.) and HATU (45 mg, 0.117 mmol, 1.2 equiv.) were dissolved in anhydrous DMF (2 mL), the mixture was cooled to 0° C. and DIEA (50 mg, 68 μL, 0.390 mmol, 4 equiv.) was added. After 5 minutes of stirring the solution of compound 12f (50 mg, 0.098 mmol, 1 equiv.) in anhydrous DMF (1 mL) was added. The resulting mixture was stirred for 60 minutes at 0° C. and 90 minutes at rt. DMF was evaporated. The crude product was purified by LC on silica gel (DCM/MeOH, 5:1+1% Et₃N) and product 12g was obtained as a light yellow solid (43 mg) in 68% yield. ¹H NMR (401 MHz, CDCl₃): 1.42 (s, 9H), 1.54-1.67 (m, 6H), 1.70-1.91 (m, 3H), 2.01-2.31 (m, 5H), 2.79-2.94 (m, 6H), 3.11-3.34 (m, 4H), 3.72 (s, 3H), 4.31 (td, J=7.8, 4.6 Hz, 1H), 4.74 (td, J=7.2, 5.5 Hz, 1H), 5.18 (bs, 1H), 6.41 (t, J=5.6 Hz, 1H), 6.67 (d, J=7.7 Hz, 1H), 6.94 (d, J=10.3 Hz, 1H), 6.97 (bs, 1H), 7.08 (ddd, J=8.0, 6.9, 1.1 Hz, 1H), 7.19 (ddd, J=8.1, 6.8, 1.1 Hz, 1H), 7.26 (d, J=8.1 Hz, 1H), 7.64 (d, J=7.9 Hz, 1H). ¹³C NMR (101 MHz, CDCl₃): 25.03, 27.08, 28.04 (3C), 28.51 (3C), 32.76, 33.84, 36.42, 39.06, 46.22, 47.59 (3C), 52.62, 54.01, 54.75, 63.87, 82.28, 108.82, 109.40, 118.97, 119.29, 121.84, 128.13, 128.38, 137.08, 170.52, 171.39, 172.83, 176.91, 194.07. ESI MS: 672.3 ([M+Na]⁺). HR ESI MS: calcd for C₃₄H₄₇O₆N₇Na 672.34800; found 672.34699.

### Preparation of tert-Butyl (S)-6-diazo-2-((S)-2-(2,2-dichloroacetamido)-3-(1-methyl-1H-indol-3-yl)propanamido)-5-oxohexanoate (12h)

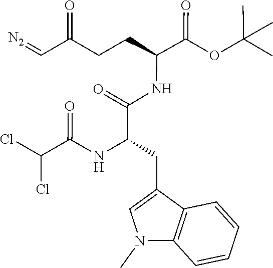

Dichloroacetic acid (17 mg, 11 μL, 0.129 mmol, 1.1 equiv.) and HATU (53 mg, 0.140 mmol, 1.2 equiv.) were dissolved in anhydrous DCM (2 mL), the mixture was cooled to 0° C. and DIEA (45 mg, 61 μL, 0.351 mmol, 3 equiv.) was added. After 5 minutes of stirring the solution of compound 10b (50 mg, 0.117 mmol, 1 equiv.) in anhydrous DCM (2 mL) was added. The resulting mixture was stirred for 30 minutes at 0° C. and 3 h at rt. DCM (70 mL) was added and the organic phase was washed with sat. NaHCO₃ (50 mL), H₂O (50 mL) and sat. NaCl (50 mL), dried over anhydrous MgSO₄ and solvent was evaporated. The crude product was purified by LC on silica gel (DCM/EtOAc, 3:1) and product 12h was obtained as a light yellow solid (60 mg) in 95% yield. ¹H NMR (401 MHz, CDCl₃): 1.44 (s, 9H), 1.88 (dtd, J=14.3, 8.4, 7.7, 5.5 Hz, 1H), 2.06-2.14 (m, 1H), 2.14-2.32 (m, 2H), 3.20 (dd, J=14.6, 7.4 Hz, 1H), 3.34 (ddd, J=14.6, 5.4, 2.6 Hz, 1H), 3.72 (s, 3H), 4.33 (td, J=7.7, 4.5 Hz, 1H), 4.65-4.74 (m, 1H), 5.16 (bs, 1H), 5.97 (bs, 1H), 6.70 (s, 1H), 6.97 (d, J=1.3 Hz, 1H), 7.10 (tt, J=6.9, 1.3 Hz, 1H), 7.21 (ddt, J=8.2, 6.9, 1.2 Hz, 1H), 7.27 (dd, J=7.9, 1.4 Hz, 1H), 7.46 (t, J=10.0 Hz, 1H), 7.68 (ddt, J=8.0, 1.9, 1.0 Hz, 1H). ¹³C NMR (101 MHz, CDCl₃): 27.04, 28.05 (3C), 28.12, 32.76, 36.22, 52.75, 54.54, 54.87, 66.40, 82.55, 108.05, 109.41, 119.09, 119.39, 121.97, 127.84, 128.56, 137.11, 163.92, 170.26, 170.34, 193.91. ESI MS: 560.1 ([M+Na]⁺). HR ESI MS: calcd for C₂₄H₂₉O₅N₅Cl₂Na 560.14380; found 560.14301.

### Preparation of tert-Butyl (8S,11S)-11-(4-diazo-3-oxobutyl)-1-(9H-fluoren-9-yl)-8-((1-methyl-1H-indol-3-yl)methyl)-3,6,9-trioxo-2-oxa-4,7,10-triazadodecan-12-oate (12i)

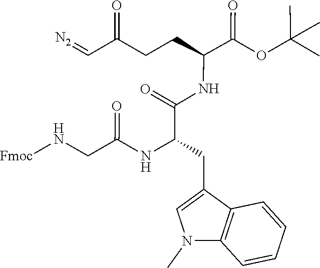

Fmoc-Gly-OH (76 mg, 0.257 mmol, 1.1 equiv.) and HATU (98 mg, 0.257 mmol, 1.1 equiv.) were dissolved in anhydrous DCM (1.5 mL), the mixture was cooled to 0° C. and DIEA (91 mg, 122 μL, 0.702 mmol, 3 equiv.) was added. After 5 minutes of stirring the solution of compound 10b (100 mg, 0.234 mmol, 1 equiv.) in anhydrous DCM (1 mL) was added. The resulting mixture was stirred for 30 minutes at 0° C. and 60 minutes at rt. DCM (40 mL) was added and the organic phase was washed with sat. NaHCO₃ (30 mL), H₂O (30 mL) and sat. NaCl (30 mL), dried over anhydrous MgSO₄ and solvent was evaporated. The crude product was purified by LC on silica gel (DCM/EtOAc, 3:1+1% Et₃N) and product 12i was obtained as a light yellow solid (161 mg) in 98% yield. ¹H NMR (401 MHz, CDCl₃): 1.41 (s, 9H), 1.76-1.92 (m, 1H), 1.97-2.08 (m, 1H), 2.19-2.28 (m, 2H), 3.14-3.26 (m, 2H), 3.66 (s, 3H), 3.82 (ddd, J=58.2, 17.1, 5.1 Hz, 2H), 4.16 (t, J=7.2 Hz, 1H), 4.25-4.42 (m, 3H), 4.67 (q, J=6.8 Hz, 1H), 5.21 (bs, 1H), 6.03 (bs, 1H), 6.92 (d, J=15.0 Hz, 2H), 7.01-7.10 (m, 2H), 7.11-7.25 (m, 2H), 7.29 (d, J=7.7 Hz, 2H), 7.37 (t, J=7.5 Hz, 2H), 7.53-7.58 (m, 3H), 7.74 (d, J=7.5 Hz, 2H). ¹³C NMR (101 MHz, d₆-DMSO): 26.0, 27.6 (3C), 27.9, 36.2, 38.3, 43.3, 44.4, 53.1, 53.9, 58.0, 65.7, 80.7, 109.0, 109.8, 118.3, 118.6, 120.0, 120.1, 120.9, 121.4, 124.2, 125.2, 127.1, 127.3, 127.6 (2C), 127.7, 136.4, 139.4, 140.7, 142.6, 143.8, 156.5, 168.9, 170.8, 171.6, 194.1. ESI MS: 729.4 ([M+Na]⁺). HR ESI MS: calcd for C₃₉H₄₂O₇N₆Na 729.30072; found 729.30080.

### Preparation of tert-Butyl (S)-2-((S)-2-(2-aminoacetamido)-3-(1-methyl-1H-indol-3-yl)propanamido)-6-diazo-5-oxohexanoate (12j)

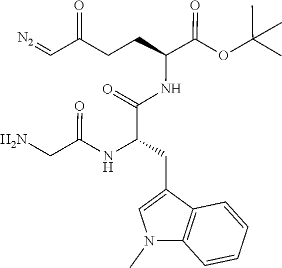

Compound 12i (150 mg, 0.212 mmol, 1 equiv.) was dissolved in anhydrous DCM/DMF 1:1 (1.5+1.5 mL) and diethylamine (155 mg, 220 μL, 2.12 mmol, 10 equiv.) was added. The mixture was stirred at rt for 3 h and solvents were evaporated. The crude product was purified by LC on silica gel (DCM/MeOH, 10:1+1% Et₃N) and product 12j was obtained as an yellow amorphous compound (72 mg) in 70% yield. ¹H NMR (401 MHz, d₆-DMSO): 1.41 (s, 9H), 1.80 (dtd, J=14.7, 8.9, 6.0 Hz, 1H), 1.91-2.02 (m, 1H), 2.25-2.42 (m, 2H), 2.87-2.98 (m, 1H), 3.08-3.21 (m, 3H), 3.71 (s, 3H), 4.12 (ddd, J=9.1, 7.4, 5.2 Hz, 1H), 4.30 (bs, 2H), 4.61 (td, J=7.9, 4.6 Hz, 1H), 6.05 (bs, 1H), 7.01 (td, J=7.4, 6.9, 1.0 Hz, 1H), 7.06-7.16 (m, 2H), 7.36 (d, J=8.2 Hz, 1H), 7.61 (d, J=7.9 Hz, 1H), 8.16 (d, J=8.2 Hz, 1H), 8.44 (d, J=7.6 Hz, 1H). ¹³C NMR (101 MHz, CDCl₃): 27.1, 28.0, 28.1 (3C), 32.8, 36.5, 43.7, 52.9, 54.1, 55.0, 82.4, 109.0, 109.4, 119.2 (2C), 121.8, 128.1, 128.5, 137.1, 170.7 (2C), 171.7, 194.6. ESI MS: 485.3 ([M+H]). HR ESI MS: calcd for C₂₄H₃₃O₅N₆ 485.25069; found 485.25060.

### Preparation of tert-Butyl (S)-6-diazo-2-((S)-3-(1-methyl-1H-indol-3-yl)-2-(2-(quinuclidine-4-carboxamido)acetamido)propanamido)-5-oxohexanoate (12k)

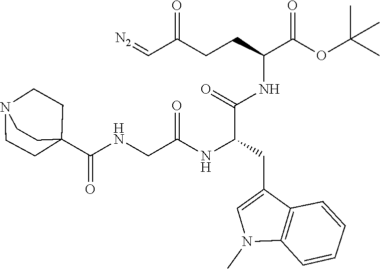

Quinuclidine-4-carboxylic acid hydrochloride (26 mg, 0.136 mmol, 1.1 equiv.) and HATU (52 mg, 0.136 mmol, 1.1 equiv.) were dissolved in anhydrous DCM/DMF 4:1 (2+0.5 mL), the mixture was cooled to 0° C. and DIEA (48 mg, 65 μL, 0.371 mmol, 3 equiv.) was added. After 5 minutes of stirring the solution of compound 12j (60 mg, 0.123 mmol, 1 equiv.) in anhydrous DCM (1 mL) was added. The resulting mixture was stirred for 30 minutes at 0° C. and 120 minutes at rt. Solvents were evaporated. The crude product was purified by LC on silica gel (DCM/MeOH, 10:1+1% Et₃N) and product 12k was obtained as an yellow solid (38 mg) in 49% yield. ¹H NMR (401 MHz, CDCl₃): 1.45 (s, 9H), 1.50 (q, J=7.1, 6.6 Hz, 4H), 1.83-1.94 (m, 1H), 2.00-2.36 (m, 4H), 2.68-2.76 (m, 1H), 2.86 (t, J=7.7 Hz, 6H), 3.15 (dd, J=14.6, 6.9 Hz, 1H), 3.39 (dd, J=14.6, 4.7 Hz, 1H), 3.75 (s, 3H), 3.90 (dd, J=5.2, 3.8 Hz, 2H), 4.33 (td, J=7.8, 4.4 Hz, 1H), 4.75 (td, J=7.2, 4.6 Hz, 1H), 5.21 (bs, 1H), 6.19 (t, J=5.2 Hz, 1H), 6.46 (d, J=7.8 Hz, 1H), 6.68 (d, J=7.5 Hz, 1H), 6.97 (s, 1H), 7.12 (ddd, J=8.0, 7.0, 1.1 Hz, 1H), 7.22 (dd, J=8.2, 1.2 Hz, 1H), 7.30 (dt, J=8.1, 1.0 Hz, 1H), 7.62 (dt, J=7.9, 1.0 Hz, 1H). ¹³C NMR (101 MHz, CDCl₃): 27.6, 28.1 (3C), 28.2 (3C), 29.8, 32.8, 36.1, 43.4, 46.2, 47.5 (3C), 52.7, 53.8, 54.8, 82.3, 108.4, 109.4, 118.9, 119.4, 121.8, 128.2, 128.6, 137.1, 168.9, 170.5, 171.0, 177.1, 194.1. ESI MS: 622.3 ([M+H]⁺). HR ESI MS: calcd for C₃₂H₄₄O₆N₇ 622.33476; found 622.33496.

### Example 8

### Preparation of Compounds 9a-9n, 10a-10b, 10d-10f, 10h-10o, 13a, 14a-d, 15a-15c, 16a, 17a, 17b, 18a, 18b, 19a, 20a, 21a, 22a, 23a, 24a, and 25a

Compounds 9a-9n, 10a-10b, 10d-10f, 10h-10o, 13a, 14a-d, 15a-15c, 16a, 17a, 17b, 18a, 18b, 19a, 20a, 21a, 22a, 23a, and 24a were prepared according to the following reaction Scheme.

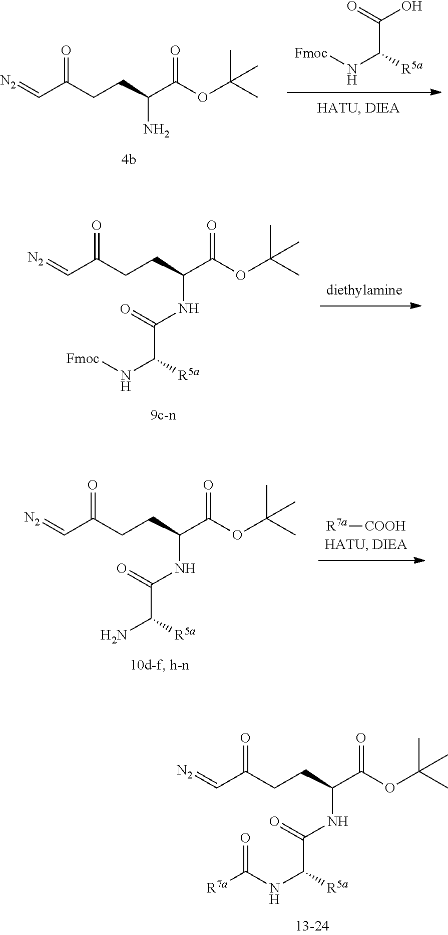

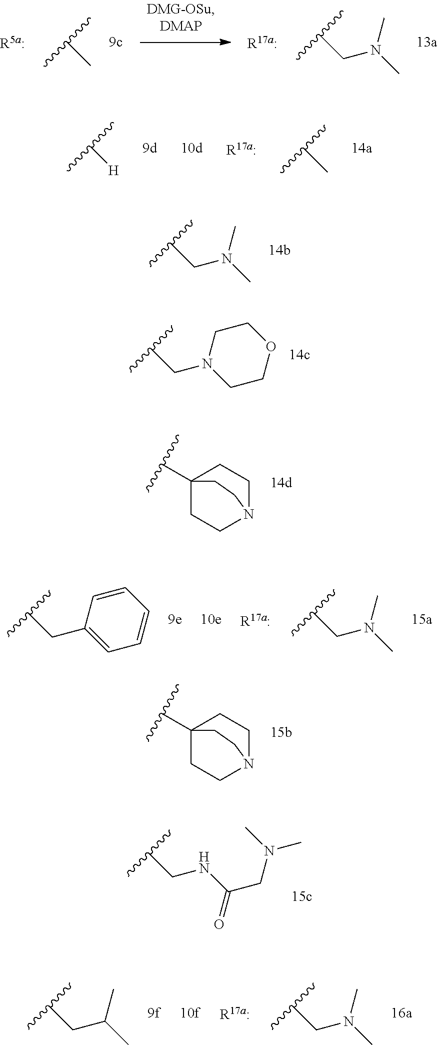

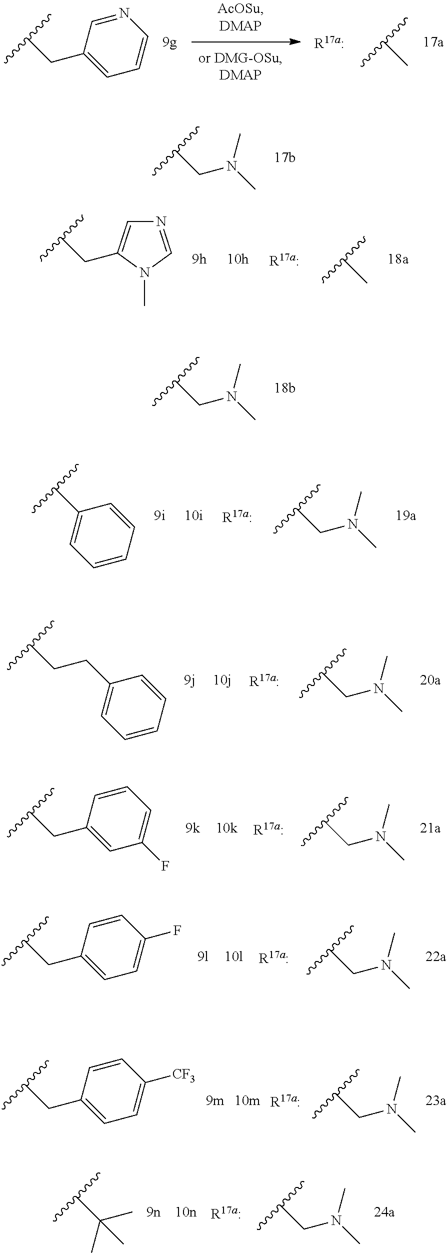

Compound 25a was prepared according to the following reaction Scheme.

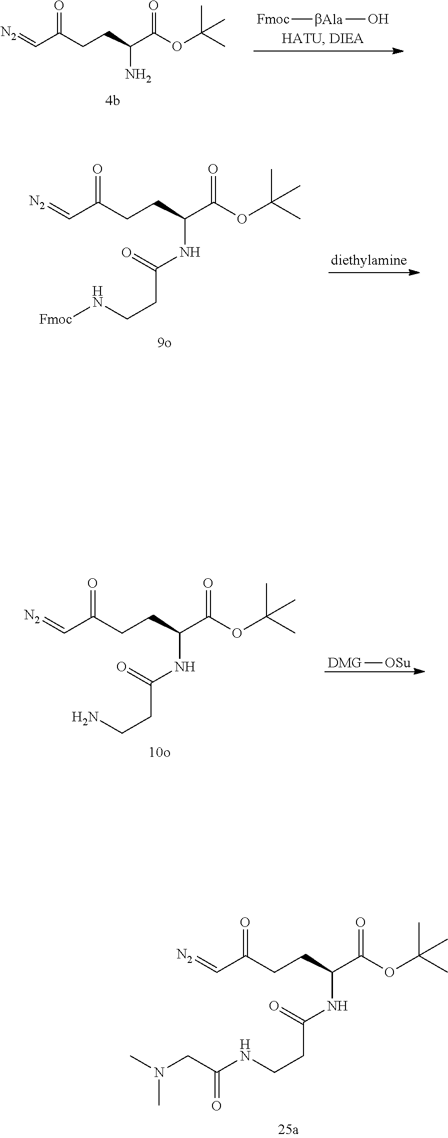

General procedure for synthesis of compounds 9a-9n: Fmoc-L-AA-OH (4.84 mmol, 1.1 equiv.) and HATU (1.92 g, 5.06 mmol, 1.15 equiv.) were suspended in anhydrous DCM (20 mL) and the reaction mixture was cooled to 0° C. DIEA (1.71 g, 2.30 mL, 13.2 mmol, 3 equiv.) was added and the mixture was stirred for 5 minutes under inert atmosphere. Finally solution of compound 4b (1.00 g, 4.40 mmol, 1 equiv.) in anhydrous DCM (10 mL) was slowly added during 5 minutes. The resulting mixture was stirred for 30 minutes at 0° C. and then 1-16.5 h at room temperature. DCM was evaporated, EtOAc (100 mL) was added and the organic phase was washed with sat. NaHCO₃ (50 mL), H₂O (50 mL), 10% KHSO₄ (50 mL), H₂O (50 mL), sat. NaCl (50 mL) and dried over anhydrous MgSO₄. EtOAc was evaporated and the residue was purified by LC on silica gel (various mobile phases).

### Preparation of tert-Butyl (S)-2-((S)-2-((((9H-fluoren-9-yl)methoxy)carbonyl)amino)-3-(1H-indol-3-yl)propanamido)-6-diazo-5-oxohexanoate (9a)

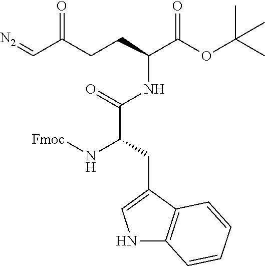

Fmoc-L-Trp-OH (2.06 g), reaction time: 17 h, mobile phase: DCM/EtOAc, 3:1. Product 9a: light yellow solid (2.35 g), 84%. H NMR (401 MHz, DMSO-d₆): 1.41 (s, 9H), 1.78-1.88 (m, 1H), 2.04-2.14 (m, 1H), 2.32-2.44 (m, 2H), 2.95 (dd, J=14.7, 10.5 Hz, 1H), 3.12 (dd, J=14.6, 4.0 Hz, 1H), 4.11-4.19 (m, 4H), 4.34 (ddd, J=10.0, 8.3, 3.9 Hz, 1H), 6.02 (bs, 1H), 6.99 (t, J=7.3 Hz, 1H), 7.07 (t, J=7.2 Hz, 1H), 7.21 (d, J=2.3 Hz, 1H), 7.25 (td, J=7.5, 1.1 Hz, 1H), 7.30-7.36 (m, 2H), 7.36-7.45 (m, 2H), 7.53 (d, J=8.5 Hz, 1H), 7.62 (d, J=7.5 Hz, 1H), 7.66 (d, J=7.4 Hz, 1H), 7.70 (d, J=7.8 Hz, 1H), 7.88 (d, J=7.5 Hz, 2H), 8.38 (d, J=7.5 Hz, 1H), 10.83 (bs, 1H). ¹³C NMR (101 MHz, DMSO-d₆): 25.94, 27.61 (3C), 27.78, 36.32, 46.56, 52.16, 52.19, 55.18, 65.63, 80.66, 110.19, 111.31, 118.18, 118.57, 120.07 (2C), 120.84, 123.94, 125.27, 125.36, 127.05 (2C), 127.24, 127.60 (2C), 136.09, 140.64 (2C), 143.74, 143.79, 155.81, 170.80, 172.24, 194.15. ESI MS: 658.3 ([M+Na]⁺). HR ESI MS: calcd for C₃₆H₃₇O₆N₅Na 658.26361; found 658.26300.

### Preparation of tert-Butyl (S)-2-((S)-2-((((9H-fluoren-9-yl)methoxy)carbonyl)amino)-3-(1-methyl-1H-indol-3-yl)propanamido)-6-diazo-5-oxohexanoate (9b)

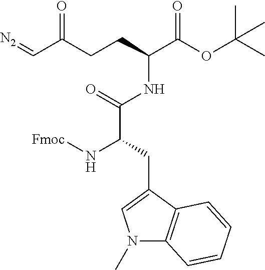

Fmoc-L-Trp(N-Me)-OH (2.13 g), reaction time: 3 h, mobile phase: DCM/EtOAc, 3:1. Product 9b: yellow solid (2.26 g), 79%. ¹H NMR (401 MHz, CDCl₃): 1.43 (s, 9H), 1.81-1.93 (m, 1H), 2.05-2.25 (m, 3H), 3.16 (dd, J=14.6, 7.1 Hz, 1H), 3.32-3.45 (m, 1H), 3.73 (s, 3H), 4.21 (t, J=7.1 Hz, 1H), 4.33-4.40 (m, 2H), 4.44 (dd, J=10.5, 7.3 Hz, 1H), 4.53 (d, J=7.1 Hz, 1H), 5.04 (bs, 1H), 5.49 (d, J=7.7 Hz, 1H), 6.55 (d, J=7.6 Hz, 1H), 6.91 (bs, 1H), 7.13 (td, J=7.4, 6.9, 1.2 Hz, 1H), 7.23 (ddd, J=8.2, 6.8, 1.1 Hz, 1H), 7.27-7.33 (m, 3H), 7.40 (tdd, J=7.6, 2.3, 1.3 Hz, 2H), 7.52-7.62 (m, 2H), 7.68 (d, J=8.0 Hz, 1H), 7.74-7.79 (m, 2H). ¹³C NMR (101 MHz, CDCl₃): 27.36, 28.05 (3C), 28.38, 32.80, 36.33, 47.26, 52.57, 54.68, 55.61, 67.20, 82.50, 108.63, 109.43, 119.13, 119.48, 120.08, 120.09, 122.00, 125.25, 125.30, 127.22 (2C), 127.84 (2C), 128.03, 128.34, 137.17, 141.39 (2C), 143.89, 143.98, 156.09, 170.48, 171.30, 193.79. ESI MS: 672.3 ([M+Na]⁺). HR ESI MS: calcd for C₃₇H₃₉O₆N₅Na 672.27926; found 672.27867.

### Preparation of tert-Butyl (S)-2-((S)-2-((((9H-fluoren-9-yl)methoxy)carbonyl)amino)propanamido)-6-diazo-5-oxohexanoate (9c)

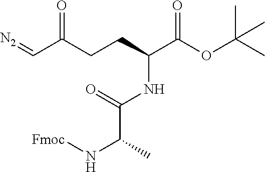

Fmoc-L-Ala-OH monohydrate (1.59 g), reaction time: 4 h, mobile phase: DCM/EtOAc, 3:1. Product 9c: light yellow solid (2.24 g), 98%. ¹H NMR (401 MHz, CDCl₃): 1.41 (d, J=7.4 Hz, 3H), 1.44 (s, 9H), 1.97 (tt, J=14.6, 7.2 Hz, 1H), 2.18 (ddd, J=14.8, 7.1, 2.5 Hz, 1H), 2.25-2.48 (m, 2H), 4.21 (t, J=7.1 Hz, 1H), 4.28 (t, J=7.2 Hz, 1H), 4.37 (dd, J=7.4, 3.1 Hz, 2H), 4.43 (td, J=8.2, 4.6 Hz, 1H), 5.21 (bs, 1H), 5.59 (d, J=7.5 Hz, 1H), 6.91 (d, J=7.8 Hz, 1H), 7.30 (td, J=7.5, 1.0 Hz, 2H), 7.39 (dd, J=8.2, 6.7 Hz, 2H), 7.59 (d, J=7.5 Hz, 2H), 7.75 (d, J=7.5 Hz, 2H). ¹³C NMR (101 MHz, CDCl₃): 18.93, 27.30, 28.04 (3C), 36.52, 47.18, 50.61, 52.56, 54.96, 67.19, 82.56, 120.07, 120.08, 125.19, 125.22, 127.19 (2C), 127.83 (2C), 141.36 (2C), 143.90 (2C), 155.99, 170.66, 172.37, 194.04. ESI MS: 543.2 ([M+Na]⁺). HR ESI MS: calcd for C₂₈H₃₂O₆N₄Na 543.22141; found 543.22096.

### Preparation of tert-Butyl (S)-2-(2-((((9H-fluoren-9-yl)methoxy)carbonyl)amino)acetamido)-6-diazo-5-oxohexanoate (9d)

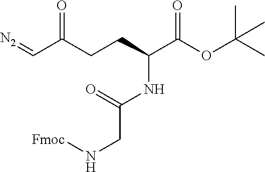

Fmoc-Gly-OH (1.44 g), reaction time: 2 h, mobile phase: EtOAc. Product 9d: light yellow solid (2.05 g), 92%. ¹H NMR (401 MHz, CDCl₃): 1.44 (s, 9H), 1.97 (dt, J=14.5, 7.5 Hz, 1H), 2.14-2.25 (m, 1H), 2.25-2.45 (m, 2H), 3.91 (d, J=5.7 Hz, 2H), 4.21 (t, J=7.2 Hz, 1H), 4.38 (d, J=7.0 Hz, 2H), 4.48 (td, J=8.1, 4.6 Hz, 1H), 5.27 (bs, 1H), 5.84 (t, J=5.7 Hz, 1H), 7.06 (d, J=7.8 Hz, 1H), 7.28 (t, J=7.6 Hz, 2H), 7.37 (t, J=7.4 Hz, 2H), 7.58 (d, J=7.5 Hz, 2H), 7.73 (d, J=7.6 Hz, 2H). ¹³C NMR (101 MHz, CDCl₃): 27.32, 27.97 (3C), 36.44, 44.43, 47.09, 52.41, 54.87, 59.71, 67.30, 82.57, 120.00 (2C), 125.13, 125.15, 127.12 (2C), 127.76 (2C), 141.28, 141.28, 143.81, 143.83, 156.68, 169.16, 170.78, 193.89. ESI MS: 529.2 ([M+Na]⁺). HR ESI MS: calcd for C₂₇H₃₀O₆N₄Na 529.20576; found 529.20604.

### Preparation of tert-Butyl (S)-2-((S)-2-((((9H-fluoren-9-yl)methoxy)carbonyl)amino)-3-phenylpropanamido)-6-diazo-5-oxohexanoate (9e)

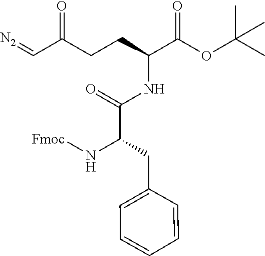

Fmoc-L-Phe-OH (1.88 g), reaction time: 16 h, mobile phase: DCM/EtOAc, 5:1. Product 9e: light yellow solid (2.00 g), 76%. ¹H NMR (401 MHz, DMSO-d₆): 1.40 (s, 9H), 1.76-1.87 (m, 1H), 1.93-2.06 (m, 1H), 2.35-2.43 (m, 2H), 2.79 (dd, J=13.8, 10.9 Hz, 1H), 3.02 (dd, J=13.8, 3.6 Hz, 1H), 4.05-4.21 (m, 4H), 4.29 (ddd, J=10.9, 8.8, 3.6 Hz, 1H), 6.04 (bs, 1H), 7.15-7.22 (m, 1H), 7.22-7.44 (m, 8H), 7.63 (dd, J=10.6, 7.5 Hz, 3H), 7.84-7.90 (m, 2H), 8.37 (d, J=7.5 Hz, 1H). ¹³C NMR (101 MHz, DMSO-d₆): 26.0, 27.6 (3C), 36.3, 37.4, 46.5, 52.1, 55.9, 56.6, 65.6, 80.7, 120.1 (2C), 125.3 (2C), 126.4, 127.0 (2C), 127.6 (2C), 128.0 (2C), 129.2 (2C), 138.1 (2C), 140.6, 143.7, 143.8, 155.8, 170.7, 171.8, 194.1. ESI MS: 619.3 ([M+Na]⁺). HR ESI MS: calcd for C₃₄H₃₆O₆N₄Na 619.25271; found 619.25162.

### Preparation of tert-Butyl (S)-2-((S)-2-((((9H-fluoren-9-yl)methoxy)carbonyl)amino)-4-methylpentanamido)-6-diazo-5-oxohexanoate (9f)

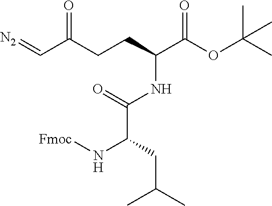

Fmoc-L-Leu-OH (1.71 g), reaction time: 2 h, mobile phase: cyclohexane/EtOAc, 1:1. Product 9f: light yellow solid (1.88 g), 76%. ¹H NMR (401 MHz, CDCl₃): 0.82-1.00 (m, 7H), 1.45 (s, 9H), 1.51-1.74 (m, 2H), 1.96 (dq, J=14.8, 7.7 Hz, 1H), 2.12-2.26 (m, 1H), 2.24-2.45 (m, 2H), 4.14-4.26 (m, 2H), 4.31-4.47 (m, 3H), 5.18 (bs, 1H), 5.27 (d, J=8.3 Hz, 1H), 6.68 (d, J=7.8 Hz, 1H), 7.31 (tt, J=7.4, 1.0 Hz, 2H), 7.40 (tt, J=7.5, 1.5 Hz, 2H), 7.59 (d, J=7.5 Hz, 2H), 7.71-7.81 (m, 2H). ¹³C NMR (101 MHz, CDCl₃): 22.07, 23.11, 24.80, 27.46, 28.09 (3C), 36.51, 41.83, 47.27, 52.55, 53.71, 54.92, 67.20, 82.63, 120.11, 120.14, 125.18, 125.25, 127.24 (2C), 127.87, 127.88, 141.42 (2C), 143.90, 143.93, 156.27, 170.66, 172.24, 193.98. ESI MS: 585.3 ([M+Na]⁺). HR ESI MS: calcd for C₃₁H₃₈O₆N₄Na 585.26836; found 585.26795.

### Preparation of tert-Butyl (S)-2-((S)-2-((((9H-fluoren-9-yl)methoxy)carbonyl)amino)-3-(pyridin-3-yl)propanamido)-6-diazo-5-oxohexanoate (9g)

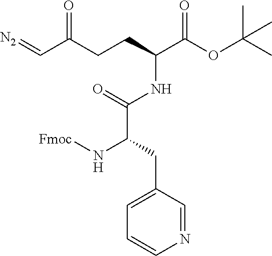

Fmoc-L-3-Pal-OH (1.88 g), reaction time: 2 h, mobile phase: DCM/MeOH, 30:1. Product 9g: light yellow solid (2.29 g), 87%. ¹H NMR (401 MHz, CDCl₃): 1.45 (s, 9H), 1.85-2.01 (m, 1H), 2.08-2.21 (m, 1H), 2.21-2.44 (m, 2H), 3.09 (q, J=6.5 Hz, 2H), 4.18 (t, J=6.9 Hz, 1H), 4.29-4.53 (m, 4H), 5.20 (bs, 1H), 5.54 (d, J=8.3 Hz, 1H), 6.96 (bs, 1H), 7.18 (dd, J=7.8, 4.8 Hz, 1H), 7.30 (tdd, J=7.5, 2.6, 1.2 Hz, 2H), 7.39 (tt, J=7.6, 1.1 Hz, 2H), 7.48-7.60 (m, 3H), 7.75 (dt, J=7.6, 1.0 Hz, 2H), 8.44 (bs, 1H), 8.47 (dd, J=4.8, 1.6 Hz, 1H). ¹³C NMR (101 MHz, CDCl₃): 27.12, 28.09 (3C), 35.86, 36.39, 47.21, 52.68, 55.03, 55.69, 67.28, 82.74, 120.12, 120.14, 123.55, 125.12, 125.22, 127.25 (2C), 127.91 (2C), 132.03, 137.10, 141.42 (2C), 143.82 (2C), 148.58, 150.77, 155.93, 170.30, 170.43, 193.95. ESI MS: 598.3 ([M+H]⁺). HR ESI MS: calcd for C₃₃H₃₆O₆N₅ 598.26601; found 598.26525.

### Preparation of tert-Butyl (S)-2-((S)-2-((((9H-fluoren-9-yl)methoxy)carbonyl)amino)-3-(1-methyl-1H-imidazol-5-yl)propanamido)-6-diazo-5-oxohexanoate (9h)

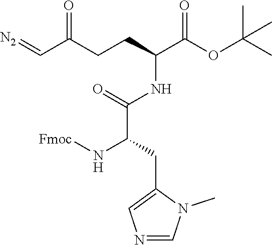

Fmoc-L-His(N-Me)-OH (1.89 g), reaction time: 2.5 h, mobile phase: DCM/MeOH, 20:1+1% Et₃N. Product 9h: yellow solid (2.19 g), 83%. ¹H NMR (401 MHz, CDCl₃): 1.46 (s, 9H), 1.88-2.03 (m, 1H), 2.08-2.19 (m, 1H), 2.20-2.40 (m, 2H), 2.97-3.16 (m, 2H), 3.57 (s, 3H), 4.21 (t, J=6.8 Hz, 1H), 4.31-4.51 (m, 4H), 5.24 (bs, 1H), 5.55 (d, J=8.1 Hz, 1H), 6.80-6.94 (m, 2H), 7.32 (tdd, J=7.4, 2.0, 1.2 Hz, 2H), 7.36-7.44 (m, 3H), 7.58 (dd, J=7.6, 1.1 Hz, 2H), 7.77 (dt, J=7.5, 0.9 Hz, 2H). ESI MS: 601.3 ([M+H]⁺). HR ESI MS: calcd for C₃₂H₃₇O₆N₆ 601.27691; found 601.27641.

### Preparation of tert-Butyl (S)-2-((S)-2-((((9H-fluoren-9-yl)methoxy)carbonyl)amino)-2-phenylacetamido)-6-diazo-5-oxohexanoate (9i)

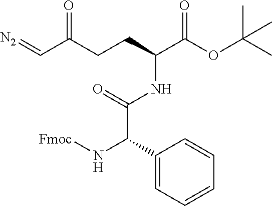

Fmoc-L-Phg-OH (1.81 g), reaction time: 1.5 h, mobile phase: DCM/EtOAc, 10:1. Product 9i: yellow solid (1.74 g), 68%. ¹H NMR (401 MHz, DMSO-d₆): 1.26 (s, 9H), 1.74-1.85 (m, 1H), 1.94 (dq, J=14.2, 7.3 Hz, 1H), 2.30-2.42 (m, 2H), 4.08-4.16 (m, 1H), 4.23 (q, J=5.7 Hz, 3H), 5.30 (d, J=8.5 Hz, 1H), 6.02 (bs, 1H), 7.32 (ddd, J=17.8, 8.0, 5.1 Hz, 5H), 7.38-7.49 (m, 4H), 7.77 (d, J=7.5 Hz, 2H), 7.88 (d, J=7.5 Hz, 2H), 8.07 (d, J=8.5 Hz, 1H), 8.52 (d, J=7.4 Hz, 1H). ¹³C NMR (101 MHz, CDCl₃): 27.2, 27.9 (3C), 29.8, 36.5, 47.2, 53.1, 55.0, 59.0, 82.6, 120.0 (2C), 125.2, 125.2, 127.2 (2C), 127.4, 127.8 (2C), 128.7, 129.2 (2C), 129.3, 137.6, 141.4 (2C), 143.9, 144.0, 155.8, 169.8, 170.1, 194.0. ESI MS: 605.2 ([M+Na]⁺). HR ESI MS: calcd for C₃₃H₃₄O₆N₄Na 605.23706; found 605.23743.

### Preparation of tert-Butyl (S)-2-((S)-2-((((9H-fluoren-9-yl)methoxy)carbonyl)amino)-4-phenylbutanamido)-6-diazo-5-oxohexanoate (9j)

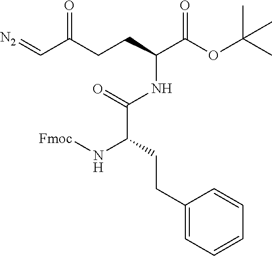

Fmoc-L-Homophe-OH (1.94 g), reaction time: 1.5 h, mobile phase: DCM/EtOAc, 10:1. Product 9j: yellow solid (2.31 g), 86%. ¹H NMR (401 MHz, CDCl₃): 1.46 (s, 9H), 1.90-2.02 (m, 2H), 2.14-2.24 (m, 2H), 2.28-2.44 (m, 2H), 2.69 (d, J=8.2 Hz, 2H), 4.21 (dt, J=11.4, 6.8 Hz, 2H), 4.33-4.51 (m, 3H), 5.18 (bs, 1H), 5.37 (d, J=8.1 Hz, 1H), 6.66 (d, J=7.7 Hz, 1H), 7.16-7.23 (m, 3H), 7.27-7.35 (m, 4H), 7.40 (tdd, J=7.5, 6.0, 2.6 Hz, 2H), 7.60 (d, J=7.4 Hz, 2H), 7.73-7.79 (m, 2H). ¹³C NMR (101 MHz, CDCl₃): 27.1, 28.1 (3C), 31.7, 34.5, 36.5, 47.2, 52.6, 54.7, 59.8, 67.2, 82.6, 120.1, 120.1, 125.2, 125.2, 126.3, 127.3 (2C), 127.9 (2C), 128.5 (2C), 128.6 (2C), 140.9, 141.4 (2C), 143.9, 143.9, 156.2, 170.6, 171.6, 194.0. ESI MS: 633.3 ([M+Na]⁺). HR ESI MS: calcd for C₃₅H₃₈O₆N₄Na 633.26836; found 633.26825.

### Preparation of tert-Butyl (S)-2-((S)-2-((((9H-fluoren-9-yl)methoxy)carbonyl)amino)-3-(3-fluorophenyl)propanamido)-6-diazo-5-oxohexanoate (9k)

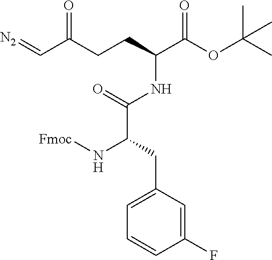

Fmoc-L-Phe(3-F)—OH (1.96 g), reaction time: 16 h, mobile phase: DCM/MeOH, 40:1. Product 9k: yellow solid (2.43 g), 90%. ¹H NMR (401 MHz, CDCl₃): 1.44 (s, 9H), 1.72-2.01 (m, 2H), 2.09-2.42 (m, 2H), 3.01-3.15 (m, 2H), 4.19 (t, J=6.8 Hz, 1H), 4.26-4.53 (m, 4H), 5.18 (bs, 1H), 5.45 (s, 1H), 6.67-7.04 (m, 4H), 7.19-7.28 (m, 1H), 7.30 (t, J=7.4 Hz, 2H), 7.40 (dd, J=8.3, 6.9 Hz, 2H), 7.50-7.60 (m, 2H), 7.70-7.83 (m, 2H). ¹³C NMR (101 MHz, CDCl₃): 27.3, 28.0 (3C), 36.4, 38.3, 47.2, 52.6, 53.5, 54.9, 55.9, 67.2, 82.7, 114.0, 114.2, 116.4, 116.6, 120.1, 125.1, 125.2, 127.2, 127.9, 130.2, 130.2, 138.9, 141.4, 143.9, 155.9, 161.7, 164.2, 170.4, 170.5, 193.9. ESI MS: 637.2 ([M+Na]). HR ESI MS: calcd for C₃₄H₃₅O₆N₄FNa 637.24328; found 637.24253.

### Preparation of tert-Butyl (S)-2-((S)-2-((((9H-fluoren-9-yl)methoxy)carbonyl)amino)-3-(4-fluorophenyl)propanamido)-6-diazo-5-oxohexanoate (91)

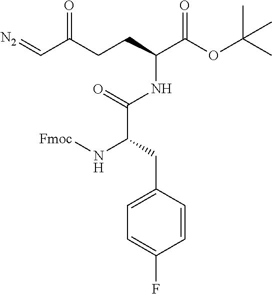

Fmoc-L-Phe(4-F)—OH (1.96 g), reaction time: 1.5 h, mobile phase: DCM/EtOAc, 5:1. Product 91: light yellow solid (2.08 g), 77%. ¹H NMR (401 MHz, DMSO-d₆): 1.39 (s, 9H), 1.81 (dtd, J=14.7, 9.0, 6.1 Hz, 1H), 1.92-2.05 (m, 1H), 2.30-2.44 (m, 2H), 2.77 (dd, J=13.8, 10.9 Hz, 1H), 3.00 (dd, J=13.7, 3.7 Hz, 1H), 4.05-4.21 (m, 4H), 4.27 (ddt, J=10.8, 8.7, 3.7 Hz, 1H), 6.04 (bs, 1H), 7.08 (dd, J=10.1, 7.7 Hz, 2H), 7.23-7.45 (m, 6H), 7.62 (dd, J=8.2, 4.3 Hz, 3H), 7.88 (d, J=7.5 Hz, 2H), 8.36 (d, J=7.5 Hz, 1H). ¹³C NMR (101 MHz, CDCl₃): 26.0, 27.6 (3C), 33.7, 36.6, 46.5, 52.1, 55.9, 59.0, 65.6, 80.7, 114.6, 114.8, 120.0 (2C), 125.2, 125.3, 127.0 (2C), 127.6 (2C), 131.0, 131.0, 134.2, 140.6, 143.7, 143.8, 155.8, 159.8, 162.2, 170.7, 171.7, 194.1. ESI MS: 637.3 ([M+Na]⁺). HR ESI MS: calcd for C₃₄H₃₅O₆N₄FNa 637.24328; found 637.24402.

### Preparation of tert-Butyl (S)-2-((S)-2-((((9H-fluoren-9-yl)methoxy)carbonyl)amino)-3-(4-(trifluoromethyl)phenyl)propanamido)-6-diazo-5-oxohexanoate (9m)

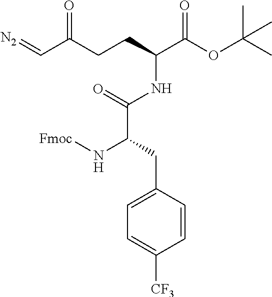

Fmoc-L-Phe(4-CF₃)—OH (2.20 g), reaction time: 3.5 h, without purification to the following step (low solubility). Product 9m: light yellow solid (2.92 g), quant. ¹H NMR (401 MHz, CDCl₃): 1.40 (s, 9H), 1.77-1.89 (m, 1H), 1.93-2.06 (m, 1H), 2.36-2.45 (m, 2H), 2.84-2.94 (m, 2H), 4.10-4.22 (m, 4H), 4.30-4.39 (m, 1H), 6.03 (bs, 1H), 7.23-7.34 (m, 2H), 7.40 (dtd, J=8.6, 4.6, 2.4 Hz, 3H), 7.56 (t, J=8.7 Hz, 2H), 7.59-7.65 (m, 3H), 7.68 (d, J=8.8 Hz, 1H), 7.85-7.91 (m, 2H), 8.40 (d, J=7.5 Hz, 1H). ESI MS: 687.3 ([M+Na]⁺). HR ESI MS: calcd for C₃₅H₃₅O₆N₄F₃Na 687.24009; found 687.23944.

### Preparation of tert-Butyl (S)-2-((S)-2-((((9H-fluoren-9-yl)methoxy)carbonyl)amino)-4,4-dimethylpentanamido)-6-diazo-5-oxohexanoate (9n)

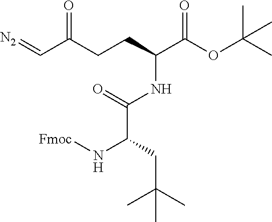

Fmoc-L-Ala(t-Bu)-OH (1.78 g), reaction time: 16 h, mobile phase: DCM/MeOH, 50:1. Product 9n: yellow solid (2.28 g), 90%. ¹H NMR (401 MHz, CDCl₃): 0.97 (s, 9H), 1.44 (s, 9H), 1.78-2.01 (m, 2H), 2.09-2.44 (m, 2H), 4.15-4.36 (m, 2H), 4.36-4.50 (m, 2H), 5.16 (s, 1H), 5.34 (d, J=8.5 Hz, 1H), 6.78 (d, J=7.9 Hz, 1H), 7.29 (td, J=7.5, 1.2 Hz, 2H), 7.34-7.42 (m, 2H), 7.54-7.62 (m, 2H), 7.71-7.77 (m, 2H). ¹³C NMR (101 MHz, CDCl₃): 28.0 (3C), 29.8 (3C), 30.6, 36.5, 46.0, 47.2, 52.5, 53.0, 53.5, 54.8, 67.2, 82.5, 120.1, 120.1, 125.1, 125.2, 127.2, 127.2, 127.8, 141.3, 143.8, 143.9, 156.0, 170.6, 172.7, 193.9. ESI MS: 577.3 ([M+H]⁺). HR ESI MS: calcd for C₃₂H₄₁O₆N₄ 577.30206; found 577.30234.

### Preparation of tert-Butyl (S)-2-(3-((((9H-fluoren-9-yl)methoxy)carbonyl)amino)propanamido)-6-diazo-5-oxohexanoate (9o)

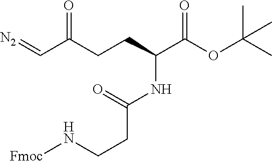

Fmoc-β-Ala-OH (1.51 g), reaction time: 2 h, mobile phase: DCM/EtOAc, 1:1. Product 9o: light yellow solid (1.60 g), 70%. ¹H NMR (401 MHz, CDCl₃): 1.47 (s, 9H), 1.91-2.06 (m, 1H), 2.11-2.25 (m, 1H), 2.29-2.41 (m, 2H), 2.45 (t, J=5.9 Hz, 2H), 3.43-3.58 (m, 2H), 4.20 (t, J=7.2 Hz, 1H), 4.31-4.49 (m, 3H), 5.21 (bs, 1H), 5.63 (t, J=6.2 Hz, 1H), 6.45-6.53 (m, 1H), 7.30 (t, J=7.5 Hz, 2H), 7.39 (t, J=7.5 Hz, 2H), 7.59 (d, J=7.5 Hz, 2H), 7.75 (d, J=7.6 Hz, 2H). ¹³C NMR (101 MHz, CDCl₃): 27.13, 28.10 (3C), 36.12, 36.65, 37.22, 47.34, 52.64, 54.99, 66.83, 82.68, 120.07 (2C), 125.26 (2C), 127.15 (2C), 127.78 (2C), 141.40 (2C), 144.08, 144.10, 156.57, 171.06, 171.61, 193.96. ESI MS: 543.2 ([M+Na]⁺). HR ESI MS: calcd for C₂₈H₃₂O₆N₄Na 543.22141; found 543.22076.

General procedure for synthesis of compounds 10a-10b, 10d-10f, and 10h-10o: Compound 9a-9b, 9d-9f, and 9h-9o (3.00 mmol, 1 equiv.) was dissolved in anhydrous DCM (27 mL) and diethylamine (2.19 g, 3.10 mL, 30.0 mmol, 10 equiv.) was added. The reaction mixture was stirred for 1.5-7 h under inert atmosphere. Solvent and excess of secondary amine were evaporated and the residue was purified by LC on silica gel (various mobile phases).

### Preparation of tert-Butyl (S)-2-((S)-2-amino-3-(1H-indol-3-yl)propanamido)-6-diazo-5-oxohexanoate (10a)

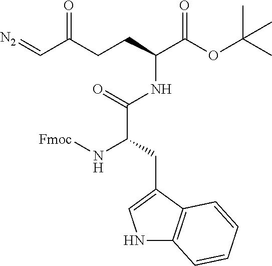

Starting material 9a (1.91 g); reaction time: 3 h; mobile phase: DCM/MeOH, 30:1. Product 10a (844 mg), light yellow solid, 68%. H NMR (401 MHz, CDCl₃): 1.48 (s, 9H), 1.88-2.00 (m, 1H), 2.01-2.28 (m, 5H), 3.07 (dd, J=14.5, 8.1 Hz, 1H), 3.33 (dd, J=14.5, 3.9 Hz, 1H), 3.79 (dd, J=8.2, 4.1 Hz, 1H), 4.47 (td, J=8.3, 4.2 Hz, 1H), 5.16 (bs, 1H), 7.09-7.16 (m, 2H), 7.21 (ddd, J=8.1, 7.0, 1.2 Hz, 1H), 7.38 (dt, J=8.1, 0.9 Hz, 1H), 7.69 (dd, J=7.9, 1.0 Hz, 1H), 7.92 (d, J=8.2 Hz, 1H), 8.51 (bs, 1H). ¹³C NMR (101 MHz, CDCl₃): 27.84, 28.10 (3C), 30.60, 36.61, 52.04, 54.81, 55.41, 82.49, 111.11, 111.40, 119.15, 119.71, 122.31, 123.63, 127.66, 136.51, 171.06, 174.59, 194.12. ESI MS: 436.2 ([M+Na]⁺). HR ESI MS: calcd for C₂₁H₂₇O₄N₅Na 436.19553; found 436.19511.

### Preparation of tert-Butyl (S)-2-((S)-2-amino-3-(1-methyl-1H-indol-3-yl)propanamido)-6-diazo-5-oxohexanoate (10b)

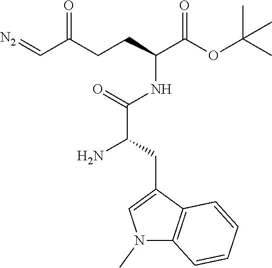

Starting material 9b (1.95 g); reaction time: 7 h; mobile phase: DCM/MeOH, 30:1. Product 10b (1.15 g), light yellow amorphous compound, 90%. ¹H NMR (401 MHz, CDCl₃): 1.46 (s, 9H), 1.55 (bs, 2H), 1.86-2.00 (m, 1H), 2.07-2.32 (m, 3H), 2.98 (dd, J=14.4, 8.5 Hz, 1H), 3.31 (dd, J=14.0, 3.8 Hz, 1H), 3.72 (dd, J=8.5, 4.1 Hz, 1H), 3.76 (s, 3H), 4.41-4.51 (m, 1H), 5.12 (bs, 1H), 6.94 (s, 1H), 7.12 (ddd, J=8.0, 6.9, 1.1 Hz, 1H), 7.23 (ddd, J=8.2, 6.9, 1.1 Hz, 1H), 7.29 (dt, J=8.2, 1.0 Hz, 1H), 7.68 (dt, J=8.0, 1.0 Hz, 1H), 7.87 (d, J=8.4 Hz, 1H). ¹³C NMR (101 MHz, CDCl₃): 27.95, 28.11 (3C), 30.76, 32.81, 36.74, 51.96, 54.66, 55.63, 82.40, 109.38, 110.07, 119.26, 119.41, 121.97, 128.08, 137.27 (2C), 171.15, 174.92, 193.85. ESI MS: 450.2 ([M+Na]⁺). HR ESI MS: calcd for C₂₂H₂₉O₄N₅Na 450.21118; found 450.21112.

### Preparation of tert-Butyl (S)-2-(2-aminoacetamido)-6-diazo-5-oxohexanoate (10d)

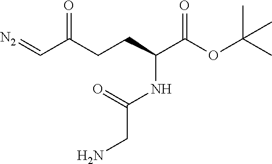

Starting material 9d (1.52 g); reaction time: 3 h; mobile phase: DCM/MeOH, 10:1. Product 10d (768 mg), yellow-orange oil, 90%. ¹H NMR (401 MHz, CDCl₃): 1.43 (s, 9H), 1.73 (bs, 2H), 1.95 (dtd, J=14.5, 8.6, 6.1 Hz, 1H), 2.17 (dddd, J=13.4, 8.5, 6.7, 4.7 Hz, 1H), 2.26-2.48 (m, 2H), 3.34 (s, 2H), 4.47 (td, J=8.4, 4.7 Hz, 1H), 5.31 (bs, 1H), 7.75 (d, J=8.4 Hz, 1H). ¹³C NMR (101 MHz, CDCl₃): 27.97, 28.04 (3C), 36.82, 44.75, 51.87, 54.82, 82.48, 171.04, 172.92, 193.85. ESI MS: 307.1 ([M+Na]). HR ESI MS: calcd for C₁₂H₂₀O₄N₄Na 307.13768; found 307.13744.

### Preparation of tert-Butyl (S)-2-((S)-2-amino-3-phenylpropanamido)-6-diazo-5-oxohexanoate (10e)

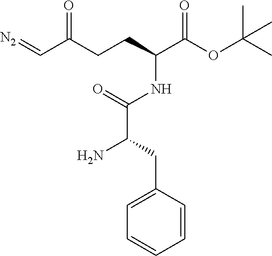

Starting material 9e (1.79 g); reaction time: 2 h; mobile phase: DCM/MeOH, 30:1. Product 10e (1.08 g), yellow amorphous compound, 96%. ¹H NMR (401 MHz, DMSO-d₆): 1.40 (s, 9H), 1.71 (bs, 2H), 1.75-1.85 (m, 1H), 1.90-1.99 (m, 1H), 2.24-2.38 (m, 2H), 2.59 (dd, J=13.4, 8.4 Hz, 1H), 2.95 (dd, J=13.4, 4.5 Hz, 1H), 3.43 (dd, J=8.4, 4.5 Hz, 1H), 4.07-4.17 (m, 1H), 6.05 (bs, 1H), 7.17-7.29 (m, 5H), 8.13 (d, J=7.9 Hz, 1H). ¹³C NMR (101 MHz, CDCl₃): 27.8, 28.1 (3C), 36.8, 41.1, 50.8, 54.8, 56.5, 82.5, 127.0, 128.8 (2C), 129.5 (2C), 137.8, 171.0, 174.4, 193.7. ESI MS: 397.2 ([M+Na]⁺). HR ESI MS: calcd for C₁₉H₂₆O₄N₄Na 397.18463; found 397.18427.

### Preparation of tert-Butyl (S)-2-((S)-2-amino-4-methylpentanamido)-6-diazo-5-oxohexanoate (10f)

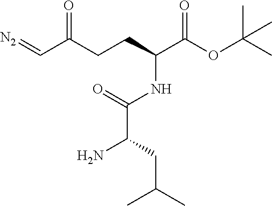

Starting material 9f (1.69 g); reaction time: 2 h; mobile phase: DCM/MeOH, 15:1. Product 10f (950 mg), yellow amorphous compound, 93%. ¹H NMR (401 MHz, CDCl₃): 0.91 (d, J=6.3 Hz, 3H), 0.95 (d, J=6.3 Hz, 3H), 1.27-1.36 (m, 1H), 1.44 (s, 9H), 1.47 (bs, 2H), 1.58-1.80 (m, 2H), 1.95 (dtd, J=14.5, 8.6, 6.1 Hz, 1H), 2.10-2.24 (m, 1H), 2.25-2.47 (m, 2H), 3.37 (dd, J=10.0, 3.8 Hz, 1H), 4.43 (td, J=8.5, 4.7 Hz, 1H), 5.30 (bs, 1H), 7.82 (d, J=8.4 Hz, 1H). ¹³C NMR (101 MHz, CDCl₃): 21.36, 23.58, 24.99, 27.90, 28.09 (3C), 36.91, 44.31, 51.91, 53.64, 54.81, 82.39, 171.17, 175.92, 193.84. ESI MS: 341.2 ([M+H]⁺). HR ESI MS: calcd for C₁₆H₂₉O₄N₄ 341.21833; found 341.21816.

### Preparation of tert-butyl (S)-2-((S)-2-amino-3-(1-methyl-1H-imidazol-5-yl)propanamido)-6-diazo-5-oxohexanoate (10h)

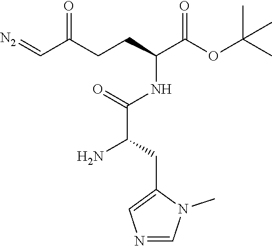

Starting material 9h (1.80 g); reaction time: 3 h; mobile phase: DCM/MeOH, 5:1+1% Et₃N. Product 10h (1.08 g), yellow amorphous compound, 95%. ¹H NMR (401 MHz, CDCl₃): 1.43 (s, 9H), 1.87-2.00 (m, 1H), 2.10-2.20 (m, 1H), 2.22-2.36 (m, 2H), 2.87 (dd, J=15.4, 8.6 Hz, 1H), 3.08-3.13 (m, 1H), 3.24 (bs, 2H), 3.60 (s, 3H), 3.67 (dd, J=8.7, 3.9 Hz, 1H), 4.39 (td, J=8.1, 4.6 Hz, 1H), 5.42 (bs, 1H), 6.92 (bs, 1H), 7.50 (bs, 1H), 8.06 (d, J=8.2 Hz, 1H). ¹³C NMR (101 MHz, CDCl₃): 27.56, 28.05 (3C), 28.92, 31.83, 36.62, 52.18, 53.88, 54.95, 82.47, 127.49, 127.80, 138.34, 170.79, 173.14, 193.89. ESI MS: 379.2 ([M+H]⁺). HR ESI MS: calcd for C₁₇H₂₇O₄N₆ 379.20883; found 379.20869.

### Preparation of tert-Butyl (S)-2-((S)-2-amino-2-phenylacetamido)-6-diazo-5-oxohexanoate (10i)

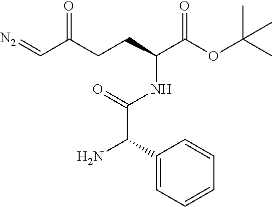

Starting material 9i (1.75 g); reaction time: 3 h; mobile phase: DCM/MeOH, 30:1. Product 10i (822 mg), yellow solid, 76%. ¹H NMR (401 MHz, CDCl₃): 1.45 (s, 9H), 1.64 (bs, 2H), 1.93 (ddt, J=11.7, 8.3, 3.6 Hz, 1H), 2.10-2.35 (m, 3H), 4.44 (td, J=8.5, 4.0 Hz, 1H), 4.55 (s, 1H), 5.04 (bs, 1H), 7.27-7.45 (m, 5H), 7.80 (d, J=8.3 Hz, 1H). ¹³C NMR (101 MHz, CDCl₃): 27.9, 28.0 (3C), 36.5, 51.9, 54.6, 60.0, 82.4, 126.7 (2C), 128.0, 128.8 (2C), 140.9, 170.9, 173.2, 193.7. ESI MS: 361.2 ([M+H]⁺). HR ESI MS: calcd for C₁₈H₂₅O₄N₄ 361.18703; found 361.18675.

### Preparation of tert-Butyl (S)-2-((S)-2-amino-4-phenylbutanamido)-6-diazo-5-oxohexanoate (10j) (KNM201)

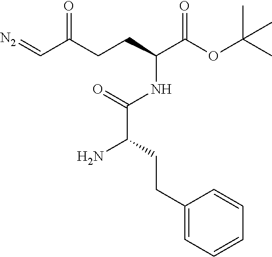

Starting material 9j (1.83 g); reaction time: 3 h; mobile phase: DCM/MeOH, 30:1. Product 10j (886 mg), yellow amorphous compound, 76%. ¹H NMR (401 MHz, CDCl₃): 1.47 (s, 9H), 1.62 (bs, 2H), 1.79 (dtd, J=14.3, 8.9, 6.0 Hz, 1H), 1.98 (dtd, J=14.5, 8.5, 6.1 Hz, 1H), 2.15-2.25 (m, 2H), 2.36 (t, J=21.3 Hz, 2H), 2.66-2.81 (m, 2H), 3.38 (dd, J=8.4, 4.4 Hz, 1H), 4.46 (td, J=8.4, 4.7 Hz, 1H), 5.28 (bs, 1H), 7.16-7.24 (m, 3H), 7.26-7.32 (m, 2H), 7.80 (d, J=8.4 Hz, 1H). ¹³C NMR (101 MHz, CDCl₃): 28.0, 28.1 (3C), 32.4, 37.0, 52.0, 53.5, 54.7, 55.0, 82.4, 126.2, 128.5 (2C), 128.6 (2C), 141.2, 171.1, 175.1, 193.7. ESI MS: 389.2 ([M+H]⁺). HR ESI MS: calcd for C₂₀H₂₉O₄N₄ 389.21833; found 389.21798.

### Preparation of tert-Butyl (S)-2-((S)-2-amino-3-(3-fluorophenyl)propanamido)-6-diazo-5-oxohexanoate (10k)

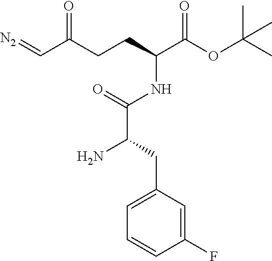

Starting material 9k (1.22 g); reaction time: 2 h; mobile phase: DCM/MeOH, 20:1. Product 10k (1.04 g), yellow solid, 88%. ¹H NMR (401 MHz, CDCl₃): 1.42 (d, J=1.3 Hz, 9H), 1.82-2.00 (m, 1H), 2.06-2.44 (m, 3H), 2.76 (dd, J=13.7, 8.7 Hz, 1H), 3.15 (dd, J=13.7, 4.0 Hz, 1H), 3.59 (ddd, J=8.7, 4.1, 1.1 Hz, 1H), 4.41 (dtd, J=8.3, 4.6, 2.3 Hz, 1H), 5.27 (bs, 1H), 6.84-7.04 (m, 3H), 7.18-7.32 (m, 1H), 7.82 (d, J=8.3 Hz, 1H). ¹³C NMR (101 MHz, CDCl₃): 28.0 (3C), 36.7, 40.7, 40.7, 52.0, 54.7, 56.2, 82.4, 113.7, 113.9, 116.1, 116.3, 125.1, 125.2, 130.1, 130.2, 140.2, 140.3, 161.7, 164.2, 170.9, 174.0, 193.7. ¹⁹F NMR (377 MHz, CDCl₃): −112.95-−112.78 (m). ESI MS: 393.2 ([M+H]⁺). HR ESI MS: calcd for C₁₉H₂₆O₄N₄F 393.19326; found 393.19334.

### Preparation of tert-Butyl (S)-2-((S)-2-amino-3-(4-fluorophenyl)propanamido)-6-diazo-5-oxohexanoate (101)

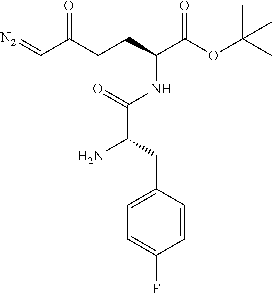

Starting material 91 (1.22 g); reaction time: 3 h; mobile phase: DCM/MeOH, 30:1. Product 10l (977 mg), yellow amorphous compound, 83%. ¹H NMR (401 MHz, CDCl₃). 1.41 (bs, 2H), 1.46 (s, 9H), 1.97 (dtd, J=14.3, 8.4, 5.6 Hz, 1H), 2.17 (td, J=13.5, 5.7 Hz, 1H), 2.30 (d, J=28.2 Hz, 2H), 2.75 (dd, J=13.8, 8.9 Hz, 1H), 3.17 (dd, J=13.8, 4.0 Hz, 1H), 3.61 (dd, J=8.9, 4.1 Hz, 1H), 4.45 (td, J=8.2, 4.6 Hz, 1H), 5.26 (bs, 1H), 6.96-7.05 (m, 2H), 7.15-7.22 (m, 2H), 7.80 (d, J=8.2 Hz, 1H). ¹³C NMR (101 MHz, CDCl₃): 27.8, 28.1 (3C), 36.7, 40.2, 52.0, 54.8, 56.5, 82.5, 115.5, 115.7, 130.9, 131.0, 133.4, 133.4, 160.8, 171.0, 174.2, 193.7. ESI MS: 393.2 ([M+H]⁺). HR ESI MS: calcd for C₁₉H₂₆O₄N₄F 393.19326; found 393.19330.

### Preparation of tert-Butyl (S)-2-((S)-2-amino-3-(4-(trifluoromethyl)phenyl)propanamido)-6-diazo-5-oxohexanoate (10m)

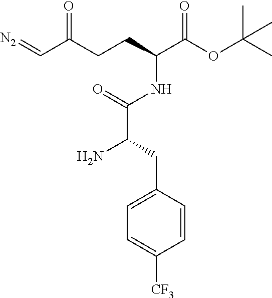

Starting material 9m (1.99 g); reaction time: 1.5 h; mobile phase: DCM/MeOH, 30:1. Product 10m (1.01 g), yellow solid, 76% over 2 steps. ¹H NMR (401 MHz, CDCl₃): 1.41 (bs, 2H), 1.44 (s, 9H), 1.88-2.05 (m, 1H), 2.11-2.42 (m, 3H), 2.82 (dd, J=13.7, 8.9 Hz, 1H), 3.25 (dd, J=13.7, 4.1 Hz, 1H), 3.64 (dd, J=8.9, 4.1 Hz, 1H), 4.43 (td, J=8.1, 4.4 Hz, 1H), 5.24 (bs, 1H), 7.34 (d, J=7.9 Hz, 2H), 7.56 (d, J=7.9 Hz, 2H), 7.82 (d, J=8.2 Hz, 1H). ¹³C NMR (101 MHz, CDCl₃): 27.55, 27.97 (3C), 36.57, 40.77, 52.03, 54.69, 56.20, 82.45, 125.58 (q, J=3.7 Hz, 2C), 129.20 (q, J=32.4 Hz), 129.74 (2C), 141.95 (2C), 170.84, 173.73, 193.50. ESI MS: 443.2 ([M+H]⁺). HR ESI MS: calcd for C₂₀H₂₆O₄N₄F₃ 443.19007; found 443.19016.

### Preparation of tert-Butyl (S)-2-((S)-2-amino-4,4-dimethylpentanamido)-6-diazo-5-oxohexanoate (10n)

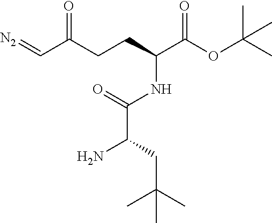

Starting material 9n (1.73 g); reaction time: 3 h; mobile phase: DCM/MeOH, 15:1. Product 10n (1.01 g), yellow oil, 95%. ¹H NMR (401 MHz, CDCl₃): 0.96 (s, 9H), 1.18 (dd, J=14.3, 8.7 Hz, 1H), 1.43 (s, 9H), 1.87 (dd, J=14.3, 2.5 Hz, 1H), 1.90-2.01 (m, 1H), 2.09-2.22 (m, 1H), 2.24-2.47 (m, 2H), 3.37 (dd, J=8.6, 2.5 Hz, 1H), 4.40 (td, J=8.5, 4.7 Hz, 1H), 5.30 (bs, 1H), 7.88 (d, J=8.4 Hz, 1H). ¹³C NMR (101 MHz, CDCl₃): 28.1 (3C), 30.1 (3C), 30.8, 36.9, 49.6, 52.0, 53.1, 54.8, 82.3, 171.1, 176.4, 193.8. ESI MS: 355.3 ([M+H]⁺). HR ESI MS: calcd for C₁₇H₃₁O₄N₄ 355.23398; found 355.23361.

### Preparation of tert-Butyl (S)-2-(3-aminopropanamido)-6-diazo-5-oxohexanoate (100)

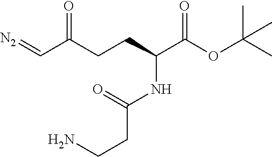

Starting material 9o (1.56 g); reaction time: 4.5 h; mobile phase: DCM/MeOH, 5:1+1% Et₃N. Product 10o (752 mg), light yellow-brown oil, 84%. ¹H NMR (401 MHz, CDCl₃): 1.38 (s, 9H), 1.83-1.95 (m, 1H), 2.00 (bs, 2H), 2.03-2.14 (m, 1H), 2.25-2.30 (m, 2H), 2.30-2.42 (m, 2H), 2.89-3.01 (m, 2H), 4.39 (td, J=8.1, 4.8 Hz, 1H), 5.30 (bs, 1H), 7.65 (d, J=8.0 Hz, 1H). ¹³C NMR (101 MHz, CDCl₃): 27.49, 27.94 (3C), 36.76, 38.13, 38.52, 52.20, 54.73, 82.16, 171.16, 172.46, 193.94. ESI MS: 299.2 ([M+H]⁺). HR ESI MS: calcd for C₁₃H₂₃O₄N₄ 299.17138; found 299.17109.

### Preparation of tert-Butyl (S)-6-diazo-2-((S)-2-(2-(dimethylamino)acetamido)propanamido)-5-oxohexanoate (13a)

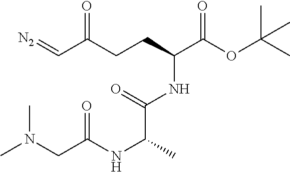

Compound 9c (150 mg, 0.288 mmol, 1 equiv.), dimethylglycine OSu (87 mg, 0.432 mmol, 1.5 equiv.) and DMAP (352 mg, 2.88 mmol, 10 equiv.) were dissolved in anhydrous DCM (1.2 mL). The resulting mixture was stirred at rt for 20 h. The crude product was purified by LC on silica gel (DCM/MeOH, 20:1) and the compound 13a was obtained as a light yellow solid (100 mg) in 91% yield. ¹H NMR (401 MHz, DMSO-d₆): 1.24 (d, J=7.0 Hz, 3H), 1.39 (s, 9H), 1.78 (ddd, J=14.3, 9.4, 6.0 Hz, 1H), 1.96 (dq, J=14.0, 7.1 Hz, 1H), 2.21 (s, 6H), 2.33-2.42 (m, 2H), 2.79-2.93 (m, 2H), 4.09 (ddd, J=9.1, 7.3, 5.1 Hz, 1H), 4.36 (p, J=7.1 Hz, 1H), 6.06 (bs, 1H), 7.73 (d, J=7.9 Hz, 1H), 8.26 (d, J=7.6 Hz, 1H). ¹³C NMR (101 MHz, CDCl₃): 18.2, 27.3, 28.1 (3C), 36.5, 46.1 (2C), 48.5, 52.6, 63.1, 70.6, 82.5, 170.7, 171.0, 172.2, 194.2. ESI MS: 384.2 ([M+H]⁺). HR ESI MS: calcd for C₁₇H₃₀O₅N₅ 384.22415; found 384.22401.

### Preparation of tert-Butyl (S)-2-(2-acetamidoacetamido)-6-diazo-5-oxohexanoate

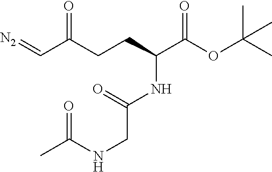

Compound 9d (150 mg, 0.296 mmol, 1 equiv.), AcOSu (70 mg, 0.444 mmol, 1.5 equiv.) and DMAP (362 mg, 2.96 mmol, 10 equiv.) were dissolved in anhydrous DCM (1.5 mL). The resulting mixture was stirred at rt for 17 h. The crude product was purified by LC on silica gel (DCM/MeOH, 20:1) and the compound 14a was obtained as a yellow amorphous oil (84 mg) in 87% yield. ¹H NMR (401 MHz, CDCl₃): 1.42 (s, 9H), 1.88-2.00 (m, 1H), 2.01 (s, 3H), 2.08-2.21 (m, 1H), 2.26-2.49 (m, 2H), 3.92 (d, J=5.4 Hz, 2H), 4.39 (td, J=8.1, 4.7 Hz, 1H), 5.35 (bs, 1H), 6.78 (t, J=5.4 Hz, 1H), 7.23 (d, J=7.7 Hz, 1H). ¹³C NMR (101 MHz, CDCl₃): 22.98, 27.05, 28.01 (3C), 36.46, 43.18, 52.55, 55.01, 82.52, 169.26, 170.77, 170.94, 194.09. ESI MS: 349.1 ([M+Na]⁺). HR ESI MS: calcd for C₁₄H₂₂O₅N₄Na 349.14824; found 349.14843.

### Preparation of tert-Butyl (S)-6-diazo-2-(2-(2-(dimethylamino)acetamido)acetamido)-5-oxohexanoate (14b)

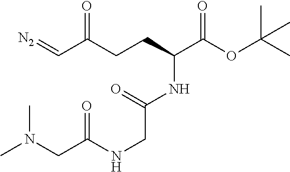

Dimethylglycine (116 mg, 1.12 mmol, 1.2 equiv.) and HATU (427 mg, 1.12 mmol, 1.2 equiv.) were dissolved in anhydrous DMF (4 mL), the mixture was cooled to 0° C. and DIEA (363 mg, 489 μL, 2.81 mmol, 3 equiv.) was added. After 5 minutes of stirring the solution of compound 10d (266 mg, 0.936 mmol, 1 equiv.) in anhydrous DMF (4 mL) was added. The resulting mixture was stirred for 30 minutes at 0° C. and 60 minutes at rt. DMF was evaporated, EtOAc (100 mL) was added and the organic phase was washed with sat. NaHCO₃ (50 mL), H₂O (50 mL) and sat. NaCl (50 mL), dried over anhydrous MgSO₄ and solvent was evaporated. The crude product was purified by LC on silica gel (DCM/MeOH, 10:1 to 5:1), dissolved in acetonitril/H₂O (4:1, 50 mL), lyophilized and product 14b was obtained as a light yellow solid (223 mg) in 64% yield. ¹H NMR (401 MHz, CDCl₃): 1.46 (s, 9H), 1.99 (dtd, J=14.3, 8.0, 6.5 Hz, 1H), 2.19 (dtd, J=15.7, 8.5, 7.9, 5.2 Hz, 1H), 2.33 (s, 6H), 2.34-2.46 (m, 2H), 2.99 (d, J=16.3 Hz, 1H), 3.06 (d, J=16.3 Hz, 1H), 3.93 (dd, J=16.6, 5.9 Hz, 1H), 4.02 (dd, J=16.6, 5.9 Hz, 1H), 4.44 (td, J=8.0, 4.6 Hz, 1H), 5.31 (bs, 1H), 6.76 (d, J=7.6 Hz, 1H), 7.72 (bs, 1H). ¹³C NMR (101 MHz, CDCl₃): 27.25, 28.05 (3C), 36.54, 42.75, 46.15 (2C), 52.52, 54.98, 62.99, 82.57, 169.00, 170.74, 171.73, 194.01. ESI MS: 392.2 ([M+Na]⁺). HR ESI MS: calcd for C₁₆H₂₇O₅N₅Na 392.19044; found 392.19016.

### Preparation of tert-Butyl (S)-6-diazo-2-(2-(2-morpholinoacetamido)acetamido)-5-oxohexanoate (14c)

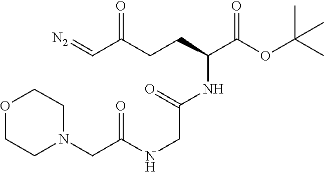

2-Morpholinoacetic acid hydrochloride (53 mg, 0.290 mmol, 1.1 equiv.) and HATU (115 mg, 0.303 mmol, 1.15 equiv.) were dissolved in anhydrous DCM (3 mL), the mixture was cooled to 0° C. and DIEA (136 mg, 184 μL, 1.06 mmol, 4 equiv.) was added. After 5 minutes of stirring the solution of compound 10d (75 mg, 0.264 mmol, 1 equiv.) in anhydrous DCM (2 mL) was added. The resulting mixture was stirred for 30 minutes at 0° C. and overnight (19.5 h) at rt. DCM (60 mL) was added and the organic phase was washed with sat. NaHCO₃ (50 mL), H₂O (50 mL) and sat. NaCl (50 mL), dried over anhydrous MgSO₄ and solvent was evaporated. The crude product was purified by LC on silica gel (DCM/MeOH, 30:1+1% Et₃N) and product 14c was obtained as a light yellow amorphous solid (63 mg) in 58% yield. ¹H NMR (401 MHz, CDCl₃): 1.41 (s, 9H), 1.94 (dtd, J=14.4, 8.0, 6.4 Hz, 1H), 2.12 (dtd, J=14.5, 7.3, 4.7 Hz, 1H), 2.26-2.46 (m, 2H), 2.49-2.58 (m, 4H), 3.00 (d, J=16.4 Hz, 1H), 3.06 (d, J=16.4 Hz, 1H), 3.64-3.75 (m, 4H), 3.92 (dd, J=16.8, 5.8 Hz, 1H), 3.99 (dd, J=16.8, 5.8 Hz, 1H), 4.38 (td, J=8.0, 4.7 Hz, 1H), 5.30 (bs, 1H), 7.02 (d, J=7.7 Hz, 1H), 7.70 (t, J=5.7 Hz, 1H). ¹³C NMR (101 MHz, CDCl₃): 27.05, 27.99 (3C), 36.51, 42.50, 52.50, 53.88 (2C), 54.94, 61.86, 67.00 (2C), 82.49, 168.82, 170.70, 170.75, 193.93. ESI MS: 412.2 ([M+H]⁺). HR ESI MS: calcd for C₁₈H₃₀O₆N₅ 412.21906; found 412.21881.

### Preparation of tert-Butyl (S)-6-diazo-5-oxo-2-(2-(quinuclidine-4-carboxamido)acetamido)hexanoate (14d)

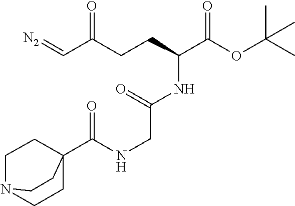

Quinuclidine-4-carboxylic acid hydrochloride (56 mg, 0.290 mmol, 1.1 equiv.) and HATU (115 mg, 0.303 mmol, 1.15 equiv.) were dissolved in anhydrous DMF (3 mL), the mixture was cooled to 0° C. and DIEA (136 mg, 184 μL, 1.06 mmol, 4 equiv.) was added. After 5 minutes of stirring the solution of compound 10d (75 mg, 0.264 mmol, 1 equiv.) in anhydrous DMF (2 mL) was added. The resulting mixture was stirred for 30 minutes at 0° C. and 60 minutes at rt. DMF was evaporated. The crude product was purified by HPLC (acetonitril/H₂O, 0.1% formic acid), lyophilized and product 14d was obtained as a light yellow foam (35 mg) in 32% yield. ¹H NMR (401 MHz, CDCl₃): 1.42 (s, 9H), 1.88-2.00 (m, 1H), 2.00-2.08 (m, 6H), 2.08-2.18 (m, 1H), 2.29-2.52 (m, 2H), 3.13-3.26 (m, 6H), 3.92 (d, J=5.1 Hz, 2H), 4.37 (td, J=7.8, 4.8 Hz, 1H), 5.34 (bs, 1H), 7.23 (t, J=5.1 Hz, 1H), 7.35 (d, J=7.5 Hz, 1H). ¹³C NMR (101 MHz, CDCl₃): 26.31 (3C), 27.03, 28.06 (3C), 35.80, 36.56, 43.19, 45.76 (3C), 52.60, 55.07, 82.61, 168.93, 170.72, 174.55, 194.18. ESI MS: 422.2 ([M+H]⁺). HR ESI MS: calcd for C₂₀H₃₂O₅N₅ 422.23980; found 422.23920.

### Preparation of tert-Butyl (S)-6-diazo-2-((S)-2-(2-(dimethylamino)acetamido)-3-phenylpropanamido)-5-oxohexanoate (15a)

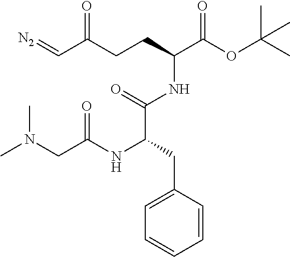

Dimethylglycine (91 mg, 0.881 mmol, 1.1 equiv.) and HATU (350 mg, 0.921 mmol, 1.15 equiv.) were dissolved in anhydrous DCM (15 mL), the mixture was cooled to 0° C. and DIEA (311 mg, 419 μL, 2.40 mmol, 3 equiv.) was added. After 5 minutes of stirring the solution of compound 10e (300 mg, 0.801 mmol, 1 equiv.) in anhydrous DCM (5 mL) was added. The resulting mixture was stirred for 30 minutes at 0° C. and 2 h at rt. DCM was evaporated, EtOAc (100 mL) was added and the organic phase was washed with sat. NaHCO₃ (50 mL), H₂O (50 mL) and sat. NaCl (50 mL), dried over anhydrous MgSO₄ and solvent was evaporated. The crude product was purified by LC on silica gel (DCM/MeOH, 15:1) and product 15a was obtained as an yellow amorphous compound (247 mg) in 67% yield. ¹H NMR (401 MHz, CDCl₃): 1.40 (s, 9H), 1.82-1.97 (m, 1H), 2.06-2.17 (m, 1H), 2.10 (s, 6H), 2.17-2.39 (m, 2H), 2.76 (d, J=16.3 Hz, 1H), 2.93 (d, J=16.3 Hz, 1H), 2.97 (dd, J=14.0, 8.5 Hz, 1H), 3.15 (dd, J=14.0, 5.9 Hz, 1H), 4.32 (td, J=7.9, 4.7 Hz, 1H), 4.66 (td, J=8.3, 5.9 Hz, 1H), 5.30 (bs, 1H), 7.02 (d, J=8.6 Hz, 1H), 7.13-7.19 (m, 3H), 7.19-7.25 (m, 2H), 7.55 (d, J=8.1 Hz, 1H). ¹³C NMR (101 MHz, CDCl₃): 27.22, 27.95 (3C), 36.34, 37.82, 45.83 (2C), 52.45, 53.93, 54.72, 62.86, 82.29, 126.92, 128.57 (2C), 129.21 (2C), 136.58, 170.39, 170.98, 171.10, 193.84. ESI MS: 460.3 ([M+H]⁺). HR ESI MS: calcd for C₂₃H₃₄O₅N₅ 460.25545; found 460.25482.

### Preparation of tert-Butyl (S)-6-diazo-5-oxo-2-((S)-3-phenyl-2-(quinuclidine-4-carboxamido)propanamido)hexanoate (15b)

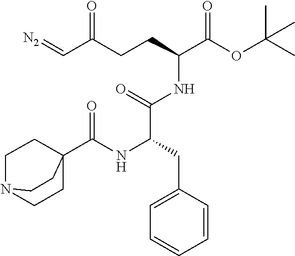

Quinuclidine-4-carboxylic acid hydrochloride (40 mg, 0.206 mmol, 1.1 equiv.) and HATU (82 mg, 0.215 mmol, 1.15 equiv.) were dissolved in anhydrous DMF (2 mL), the mixture was cooled to 0° C. and DIEA (97 mg, 130 μL, 0.742 mmol, 4 equiv.) was added. After 5 minutes of stirring the solution of compound 10e (70 mg, 0.188 mmol, 1 equiv.) in anhydrous DMF (2 mL) was added. The resulting mixture was stirred for 30 minutes at 0° C. and 60 minutes at rt. DMF was evaporated. The crude product was purified by LC on silica gel (DCM/MeOH, 20:1+1% Et₃N) and product 15b was obtained as an light yellow solid (42 mg) in 33% yield. ¹H NMR (401 MHz, CDCl₃): 1.46 (s, 9H), 1.79-1.88 (m, 6H), 1.97 (dq, J=14.4, 7.1 Hz, 1H), 2.06-2.17 (m, 1H), 2.34 (d, J=25.8 Hz, 2H), 3.01-3.17 (m, 8H), 4.34 (td, J=7.5, 4.7 Hz, 1H), 4.69 (dt, J=7.7, 6.6 Hz, 1H), 5.26 (bs, 1H), 6.17 (d, J=7.6 Hz, 1H), 6.80 (bs, 1H), 7.14-7.32 (m, 5H). ¹³C NMR (101 MHz, CDCl₃): 27.2, 27.9 (3C), 28.0 (3C), 36.2, 38.2, 47.2 (3C), 52.6, 54.0, 55.7, 70.6, 82.5, 127.1, 128.6 (2C), 129.5 (2C), 136.4, 170.4, 170.9, 175.7, 194.0. ESI MS: 512.3 ([M+H]⁺). HR ESI MS: calcd for C₂₇H₃₈O₅N₅ 512.28675; found 512.28634.

### Preparation of tert-Butyl (9S,12S)-9-benzyl-12-(4-diazo-3-oxobutyl)-2-methyl-4,7,10-trioxo-2,5,8,11-tetraazatridecan-13-oate (15c)

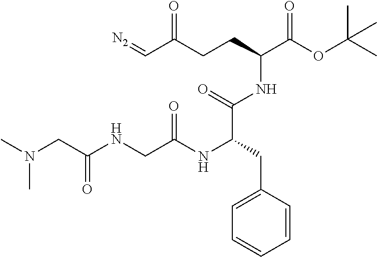

Dimethylglycylglycine (50 mg, 0.312 mmol, 2 equiv.) and HATU (119 mg, 0.312 mmol, 2 equiv.) were dissolved in anhydrous solvents DCM/DMF 2:1 (2+1 mL), the mixture was cooled to 0° C. and DIEA (81 mg, 109 μL, 0.624 mmol, 4 equiv.) was added. After 5 minutes of stirring the solution of compound 10e (58 mg, 0.156 mmol, 1 equiv.) in anhydrous DCM (2 mL) was added. The resulting mixture was stirred for 30 minutes at 0° C. and 60 minutes at rt. Solvents were evaporated. EtOAc (50 mL) was added and the organic phase was washed with sat. NaHCO₃ (30 mL), H₂O (30 mL) and sat. NaCl (30 mL), dried over anhydrous MgSO₄ and solvent was evaporated. The crude product was purified by LC on silica gel (DCM/MeOH, 20:1) and product 15c was obtained as a yellow amorphous compound (31 mg) in 38% yield. ¹H NMR (401 MHz, CDCl₃): 1.44 (s, 9H), 1.93 (tt, J=14.7, 6.9 Hz, 1H), 2.12 (tdd, J=12.0, 6.4, 3.1 Hz, 1H), 2.20-2.26 (m, 2H), 2.34 (s, 6H), 2.97-3.15 (m, 4H), 3.84-4.00 (m, 2H), 4.35 (td, J=7.8, 4.6 Hz, 1H), 4.68 (q, J=7.0 Hz, 1H), 5.32-5.41 (bs, 1H), 6.83 (dd, J=7.7, 3.4 Hz, 2H), 7.13-7.32 (m, 5H), 7.82 (t, J=5.9 Hz, 1H). ¹³C NMR (101 MHz, CDCl₃): 27.3, 28.1 (3C), 36.4, 38.0, 43.1, 45.9 (2C), 52.6, 54.5, 55.0, 62.4, 82.4, 127.1, 128.8 (2C), 129.5 (2C), 136.4, 169.0, 170.5, 170.7, 171.2, 194.3. ESI MS: 517.6 ([M+H]⁺). HR ESI MS: calcd for C₂₅H₃₇O₆N₆ 517.27691; found 517.27631.

### Preparation of tert-Butyl (S)-6-diazo-2-((S)-2-(2-(dimethylamino)acetamido)-4-methylpentanamido)-5-oxohexanoate (16a)

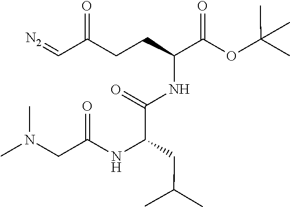

Compound 10f (58 mg, 0.170 mmol, 1 equiv.) and dimethylglycine OSu were dissolved in anhydrous DCM (1.5 mL) and the resulting mixture was stirred at rt for 5 h under inert atmosphere. DCM was evaporated, EtOAc (70 mL) was added and the organic phase was washed with sat. NaHCO₃ (2×50 mL) and sat. NaCl (50 mL), dried over anhydrous MgSO₄ and solvent was evaporated. The residue was purified by LC on silica gel (DCM/MeOH, 10:1) to obtained product 16a as an yellow amorphous compound (52 mg) in 72% yield. ¹H NMR (401 MHz, CDCl₃): 0.89 (d, J=6.1 Hz, 3H), 0.92 (d, J=6.1 Hz, 3H), 1.42 (s, 9H), 1.51-1.71 (m, 3H), 1.91 (dtd, J=14.4, 8.4, 6.1 Hz, 1H), 2.08-2.21 (m, 1H), 2.28 (s, 6H), 2.20-2.45 (m, 2H), 2.96 (d, J=3.1 Hz, 2H), 4.33-4.46 (m, 2H), 5.34 (bs, 1H), 6.90 (d, J=7.7 Hz, 1H), 7.47 (d, J=8.5 Hz, 1H). ¹³C NMR (101 MHz, CDCl₃): 21.90, 23.08, 24.87, 27.38, 28.03 (3C), 29.74, 36.46, 41.00, 45.99, 51.39, 52.44, 54.85, 62.98, 82.36, 170.63, 170.93, 172.02, 194.00. ESI MS: 426.3 ([M+H]⁺). HR ESI MS: calcd for C₂₀H₃₆O₅N₅ 426.27110; found 426.27057.

### Preparation of tert-Butyl (S)-2-((S)-2-acetamido-3-(pyridin-3-yl)propanamido)-6-diazo-5-oxohexanoate (17a)

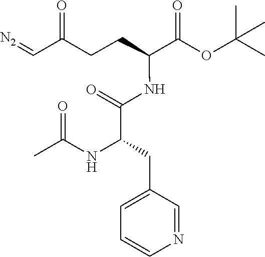

Compound 9g (150 mg, 0.251 mmol, 1 equiv.), AcOSu (59 mg, 0.376 mmol, 1.5 equiv.) and DMAP (307 mg, 2.51 mmol, 10 equiv.) were dissolved in anhydrous DCM (1.2 mL). The resulting mixture was stirred at rt for 20 h. The crude product was purified by LC on silica gel (DCM/MeOH, 10:1) and the compound 17a was obtained as a light yellow solid (89 mg) in 85% yield. ¹H NMR (401 MHz, CDCl₃): 1.41 (s, 9H), 1.83-1.96 (m, 1H), 1.90 (s, 3H), 2.03-2.19 (m, 1H), 2.21-2.44 (m, 2H), 2.93 (dd, J=14.0, 7.4 Hz, 1H), 3.09 (dd, J=14.0, 5.9 Hz, 1H), 4.34 (td, J=7.9, 4.8 Hz, 1H), 4.78 (td, J=7.6, 5.9 Hz, 1H), 5.33 (bs, 1H), 7.03 (d, J=8.0 Hz, 1H), 7.09-7.19 (m, 1H), 7.52 (dt, J=7.9, 2.0 Hz, 1H), 7.65 (d, J=7.6 Hz, 1H), 8.35 (d, J=2.3 Hz, 1H), 8.40 (dd, J=4.8, 1.7 Hz, 1H). ¹³C NMR (101 MHz, CDCl₃): 22.97, 26.95, 27.99 (3C), 35.58, 36.38, 52.56, 53.83, 54.92, 82.36, 123.47, 132.45, 137.16, 148.11, 150.46, 170.38, 170.44, 170.97, 193.93. ESI MS: 440.2 ([M+Na]⁺). HR ESI MS: calcd for C₂₀H₂₇O₅N₅Na 440.19044; found 440.19033.

### Preparation of tert-Butyl (S)-6-diazo-2-((S)-2-(2-(dimethylamino)acetamido)-3-(pyridin-3-yl)propanamido)-5-oxohexanoate (17b)

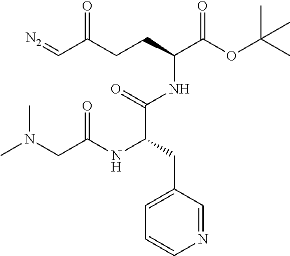

Compound 9g (150 mg, 0.251 mmol, 1 equiv.), dimethylglycine OSu (75 mg, 0.376 mmol, 1.5 equiv.) and DMAP (307 mg, 2.51 mmol, 10 equiv.) were dissolved in anhydrous DCM (1.2 mL). The resulting mixture was stirred at rt for 23 h. The crude product was purified by LC on silica gel (DCM/MeOH, 5:1) and the product 17b was obtained as a yellow amorphous compound (84 mg) in 73% yield. ¹H NMR (401 MHz, CDCl₃): 1.39 (s, 9H), 1.89 (dtd, J=14.4, 8.2, 6.3 Hz, 1H), 2.03-2.11 (m, 1H), 2.13 (s, 6H), 2.21-2.38 (m, 2H), 2.80 (d, J=16.3 Hz, 1H), 2.92 (d, J=16.3 Hz, 1H), 2.96 (dd, J=14.2, 5.9 Hz, 1H), 3.16 (dd, J=14.2, 5.9 Hz, 1H), 4.31 (td, J=7.8, 4.8 Hz, 1H), 4.72 (td, J=8.2, 5.8 Hz, 1H), 5.31 (bs, 1H), 7.15 (ddd, J=7.8, 4.8, 0.8 Hz, 1H), 7.37 (d, J=7.5 Hz, 1H), 7.51 (dt, J=8.0, 1.9 Hz, 1H), 7.64 (d, J=8.4 Hz, 1H), 8.39 (d, J=2.9 Hz, 1H), 8.40 (dd, J=4.9, 1.6 Hz, 1H). ¹³C NMR (101 MHz, CDCl₃): 26.95, 27.96 (3C), 35.31, 36.36, 45.87 (2C), 52.55, 53.29, 54.83, 62.77, 82.32, 123.43, 132.32, 136.85, 148.32, 150.54, 170.38, 170.53, 170.97, 193.86. ESI MS: 461.2 ([M+H]⁺). HR ESI MS: calcd for C₂₂H₃₃O₅N₆ 461.25069; found 461.25099.

### Preparation of tert-Butyl (S)-2-((S)-2-acetamido-3-(1-methyl-1H-imidazol-5-yl)propanamido)-6-diazo-5-oxohexanoate (18a)

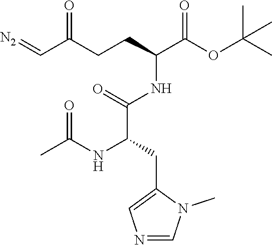

Compound 10h (84 mg, 0.222 mmol, 1 equiv.) and AcOSu (42 mg, 0.266 mmol, 1.2 equiv.) were dissolved in anhydrous DCM (2 mL). The resulting mixture was stirred at rt for 17 h. The crude product was purified by LC on silica gel (DCM/MeOH, 10:1+1% Et₃N) and the compound 18a was obtained as a light yellow solid (54 mg) in 58% yield. ¹H NMR (401 MHz, CDCl₃): 1.34 (s, 9H), 1.80-1.93 (m, 1H), 1.90 (s, 3H), 1.95-2.11 (m, 1H), 2.21-2.36 (m, 2H), 2.95 (dd, J=15.4, 7.5 Hz, 1H), 3.04 (dd, J=15.4, 7.5 Hz, 1H), 3.52 (s, 3H), 4.26 (td, J=7.8, 4.6 Hz, 1H), 4.64 (q, J=7.5 Hz, 1H), 5.45 (bs, 1H), 6.77 (bs, 1H), 7.40 (bs, 1H), 7.51 (d, J=8.1 Hz, 1H), 7.72 (d, J=7.5 Hz, 1H). ¹³C NMR (101 MHz, CDCl₃): 22.91, 26.32, 26.82, 27.85 (3C), 31.59, 36.35, 52.21, 52.54, 54.86, 82.04, 127.01, 127.78, 137.66, 170.28, 170.48, 170.67, 194.25. ESI MS: 421.2 ([M+H]⁺). HR ESI MS: calcd for C₁₉H₂₉O₅N₆ 421.21939; found 421.21923.

### Preparation of tert-Butyl (S)-6-diazo-2-((S)-2-(2-(dimethylamino)acetamido)-3-(1-methyl-1H-imidazol-5-yl)propanamido)-5-oxohexanoate (18b)

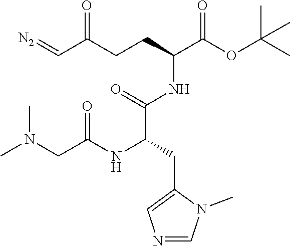

Compound 10h (94 mg, 0.248 mmol, 1 equiv.) and dimethylglycine OSu (60 mg, 0.298 mmol, 1.2 equiv.) were dissolved in anhydrous DCM (2 mL). The resulting mixture was stirred at rt for 18 h. The crude product was purified by LC on silica gel (DCM/MeOH, 10:1+1% Et₃N) and by HPLC (acetonitril/H₂O, 0.1% formic acid), lyophilized and the compound 18b was obtained as a light yellow foam (43 mg) in 37% yield. ¹H NMR (401 MHz, CDCl₃): 1.44 (s, 9H), 1.90-2.03 (m, 1H), 2.03-2.21 (m, 1H), 2.22-2.40 (m, 2H), 2.29 (s, 6H), 2.90-3.21 (m, 4H), 3.66 (s, 3H), 4.33 (td, J=7.2, 4.5 Hz, 1H), 4.66 (q, J=7.2 Hz, 1H), 5.33 (bs, 1H), 6.94 (bs, 1H), 7.38 (bs, 1H), 7.59 (bs, 1H), 7.93 (d, J=8.1 Hz, 1H). ¹³C NMR (101 MHz, CDCl₃): 26.65, 27.92, 28.10 (3C), 32.02, 36.27, 45.92 (2C), 51.84, 52.84, 55.17, 62.71, 82.54, 125.61, 126.77, 127.63, 170.23, 170.31, 170.82, 194.22. ESI MS: 464.1 ([M+H]⁺). HR ESI MS: calcd for C₂₁H₃₄O₅N₇ 464.26159; found 464.26169.

### Preparation of tert-Butyl (S)-6-diazo-2-((S)-2-(2-(dimethylamino)acetamido)-2-phenylacetamido)-5-oxohexanoate (19a)

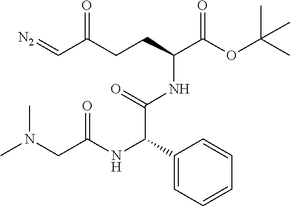

Compound 10i (70 mg, 0.194 mmol, 1 equiv.) and dimethylglycineOSu (43 mg, 0.214 mmol, 1.1 equiv.) were dissolved in anhydrous DCM (1 mL). The resulting mixture was stirred at rt for 20 h. DCM (30 mL) was added and the organic phase was washed with sat. NaHCO₃ (20 mL), H₂O (20 mL) and sat. NaCl (20 mL), dried over anhydrous MgSO₄ and solvent was evaporated. The crude product was purified by LC on silica gel (DCM/MeOH, 15:1) and the product 19a was obtained as an yellow amorphous compound (59 mg) in 68% yield. ¹H NMR (401 MHz, CDCl₃): 1.36 (s, 9H), 1.97 (dtd, J=14.4, 8.0, 6.4 Hz, 1H), 2.11-2.25 (m, 1H), 2.30 (s, 6H), 2.32-2.51 (m, 2H), 2.92-3.04 (m, 2H), 4.39 (td, J=7.9, 4.7 Hz, 1H), 5.31 (bs, 1H), 5.45 (d, J=7.5 Hz, 1H), 6.52 (d, J=7.5 Hz, 1H), 7.30-7.42 (m, 5H), 8.09 (d, J=7.6 Hz, 1H). ¹³C NMR (101 MHz, CDCl₃): 26.1, 27.1 (3C), 28.9, 45.3 (2C), 52.0, 55.2, 62.4, 69.8, 80.9, 126.7 (2C), 127.3, 128.0 (2C), 137.6, 169.3 (2C), 169.6, 193.4. ESI MS: 446.2 ([M+H]⁺). HR ESI MS: calcd for C₂₂H₃₂O₅N₅ 446.23980; found 446.23917.

### Preparation of tert-Butyl (S)-6-diazo-2-((S)-2-(2-(dimethylamino)acetamido)-4-phenylbutanamido)-5-oxohexanoate (20a)

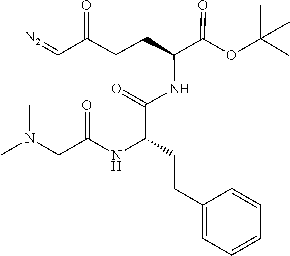

Compound 10j (70 mg, 0.180 mmol, 1 equiv.) and dimethylglycineOSu (43 mg, 0.198 mmol, 1.1 equiv.) were dissolved in anhydrous DCM (1 mL). The resulting mixture was stirred at rt for 2 h. The crude product was purified by LC on silica gel (DCM/MeOH, 30:1 to 15:1) and the product 20a was obtained as an yellow amorphous compound (43 mg) in 51% yield. ¹H NMR (401 MHz, CDCl₃): 1.46 (s, 9H), 1.67-1.85 (m, 2H), 1.91-2.05 (m, 2H), 2.12-2.27 (m, 2H), 2.31 (s, 6H), 2.70 (t, J=7.8 Hz, 2H), 2.98 (d, J=1.2 Hz, 2H), 4.37-4.45 (m, 2H), 5.32 (bs, 1H), 6.76 (d, J=7.7 Hz, 1H), 7.16-7.22 (m, 3H), 7.27-7.31 (m, 2H), 7.62 (d, J=8.3 Hz, 1H). ¹³C NMR (101 MHz, CDCl₃): 27.3, 28.1 (3C), 32.0, 34.0, 46.0 (2C), 52.6, 52.8, 55.1, 63.0, 70.7, 82.5, 126.3, 128.5 (2C), 128.6 (2C), 140.9, 170.6, 171.0, 171.4, 194.0. ESI MS: 474.4 ([M+H]⁺). HR ESI MS: calcd for C₂₄H₃₆O₅N₅ 474.27110; found 474.27011.

### Preparation of tert-Butyl (S)-6-diazo-2-((S)-2-(2-(dimethylamino)acetamido)-3-(3-fluorophenyl)propanamido)-5-oxohexanoate (21a)

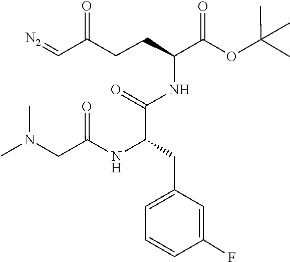

Compound 10k (75 mg, 0.191 mmol, 1 equiv.) and dimethylglycineOSu (42 mg, 0.210 mmol, 1.1 equiv.) and DIEA (74 mg, 99 μL, 0.573 mmol, 3 equiv.) were dissolved in anhydrous DCM (3 mL). The resulting mixture was stirred at rt for 16 h. The crude product was purified by LC on silica gel (DCM/MeOH, 20:1) and the compound 21a was obtained as an yellow oil (75 mg) in 82% yield. ¹H NMR (401 MHz, CDCl₃): 1.44 (s, 9H), 1.87-2.01 (m, 1H), 2.16 (s, 6H), 2.19-2.42 (m, 2H), 2.82 (d, J=16.3 Hz, 1H), 2.98 (d, J=16.2 Hz, 1H), 3.00-3.08 (m, 1H), 3.17 (dd, J=14.0, 6.2 Hz, 1H), 4.32-4.39 (m, 1H), 4.65 (td, J=8.1, 6.1 Hz, 1H), 5.28-5.33 (bs, 1H), 6.85-6.95 (m, 3H), 6.99 (d, J=7.8 Hz, 1H), 7.19-7.26 (m, 1H), 7.59 (d, J=8.2 Hz, 1H). ¹³C NMR (101 MHz, CDCl₃): 27.3, 28.1 (3C), 36.4, 37.5, 37.6, 46.0 (2C), 52.6, 53.6, 53.9, 54.9, 63.0, 82.6, 113.9, 114.1, 116.2, 116.5, 125.0, 125.0, 130.1, 130.2, 139.2, 139.3, 161.7, 164.2, 170.4, 170.6, 171.2, 193.9. ¹⁹F NMR (377 MHz, CDCl₃): −113.12-−113.02 (m). ESI MS: 478.3 ([M+H]⁺). HR ESI MS: calcd for C₂₃H₃₃O₅N₅F 478.24602; found 478.24527.

### Preparation of tert-Butyl (S)-6-diazo-2-((S)-2-(2-(dimethylamino)acetamido)-3-(4-fluorophenyl)propanamido)-5-oxohexanoate (22a)

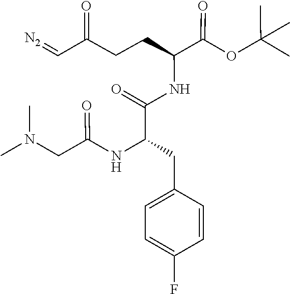

Compound 10l (70 mg, 0.178 mmol, 1 equiv.) and dimethylglycineOSu (39 mg, 0.196 mmol, 1.1 equiv.) were dissolved in anhydrous DMF (1 mL). The resulting mixture was stirred at rt for 16 h. DMF was evaporated, DCM (50 mL) was added and the organic phase was washed with sat. NaHCO₃ (30 mL), H₂O (30 mL) and sat. NaCl (30 mL), dried over anhydrous MgSO₄ and solvent was evaporated. The crude product was purified by LC on silica gel (DCM/MeOH, 20:1) and the compound 22a was obtained as an yellow solid (62 mg) in 73% yield. ¹H NMR (401 MHz, CDCl₃): 1.45 (s, 9H), 1.94 (dtd, J=14.2, 8.0, 6.3 Hz, 1H), 2.09-2.16 (m, 1H), 2.18 (s, 6H), 2.23-2.43 (m, 2H), 2.80-2.90 (m, 1H), 2.94-3.06 (m, 2H), 3.14 (dd, J=14.1, 6.6 Hz, 1H), 4.35 (td, J=7.6, 4.8 Hz, 1H), 4.60 (td, J=8.0, 6.6 Hz, 1H), 5.29 (bs, 1H), 6.73 (d, J=7.4 Hz, 1H), 6.92-7.01 (m, 2H), 7.13-7.23 (m, 2H), 7.57 (d, J=8.1 Hz, 1H). ¹³C NMR (101 MHz, CDCl₃): 27.3, 28.1 (3C), 37.2, 46.0 (2C), 52.6, 54.2, 54.9, 63.0, 70.1, 82.6, 115.4, 115.6, 130.9, 130.9, 132.4, 132.4, 163.2, 170.4, 170.7, 171.2, 193.9. ESI MS: 478.3 ([M+H]⁺). HR ESI MS: calcd for C₂₃H₃₃O₅N₅F 478.24602; found 478.24526.

### Preparation of tert-Butyl (S)-6-diazo-2-((S)-2-(2-(dimethylamino)acetamido)-3-(4-(trifluoromethyl)phenyl)propanamido)-5-oxohexanoate (23a)

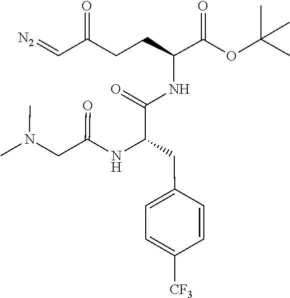

Compound 10m (75 mg, 0.170 mmol, 1 equiv.) and dimethylglycineOSu (37 mg, 0.186 mmol, 1.1 equiv.) were dissolved in anhydrous DCM (2 mL). The resulting mixture was stirred at rt for 2 h. DCM (50 mL) was added and the organic phase was washed with sat. NaHCO₃ (30 mL) and sat. NaCl (30 mL), dried over anhydrous MgSO₄ and solvent was evaporated. The crude product was purified by LC on silica gel (DCM/MeOH, 30:1) and the compound 23a was obtained as an yellow solid (68 mg) in 77% yield. ¹H NMR (401 MHz, CDCl₃): 1.42 (s, 9H), 1.93 (dtd, J=14.3, 8.0, 6.3 Hz, 1H), 2.14 (s, 6H), 2.06-2.18 (m, 1H), 2.20-2.42 (m, 2H), 2.81 (d, J=16.3 Hz, 1H), 2.96 (d, J=16.3 Hz, 1H), 3.07 (dd, J=14.1, 8.0 Hz, 1H), 3.24 (dd, J=14.1, 6.3 Hz, 1H), 4.34 (td, J=7.7, 4.8 Hz, 1H), 4.72 (td, J=8.1, 6.3 Hz, 1H), 5.28 (bs, 1H), 7.03 (d, J=7.4 Hz, 1H), 7.32 (d, J=8.4 Hz, 2H), 7.51 (d, J=7.4 Hz, 2H), 7.59 (d, J=8.3 Hz, 1H). ¹³C NMR (101 MHz, CDCl₃): 27.05, 28.01 (3C), 36.35, 37.71, 45.93 (2C), 52.61, 53.61, 54.94, 62.96, 82.53, 125.51 (q, J=3.7 Hz, 2C), 129.35 (q, J=32.6 Hz), 129.78 (2C), 140.92, 140.93, 170.38, 170.52, 171.20, 193.89. ESI MS: 528.3 ([M+H]⁺). HR ESI MS: calcd for C₂₄H₃₃O₅N₅F₃ 528.24283; found 528.24252.

### Preparation of tert-Butyl (S)-6-diazo-2-((S)-2-(2-(dimethylamino)acetamido)-4,4-dimethylpentanamido)-5-oxohexanoate (24a)

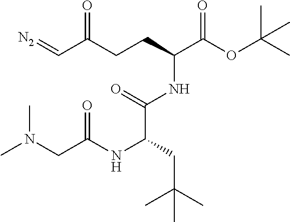

Compound 10n (70 mg, 0.205 mmol, 1 equiv.), dimethylglycineOSu (42 mg, 0.226 mmol, 1.1 equiv.) and DIEA (80 mg, 107 μL, 0.617 mmol, 3 equiv.) were dissolved in anhydrous DCM (3 mL). The resulting mixture was stirred at rt for 2.5 h. The crude product was purified by LC on silica gel (DCM/MeOH, 20:1 to 15:1) and the compound 24a was obtained as an yellow oil (90 mg) in 92% yield. ¹H NMR (401 MHz, CDCl₃): 0.90 (d, J=1.7 Hz, 9H), 1.33-1.50 (m, 10H), 1.79-1.95 (m, 2H), 2.05-2.17 (m, 1H), 2.19-2.38 (m, 8H), 2.90 (s, 2H), 4.24-4.46 (m, 2H), 5.33 (bs, 1H), 6.83-6.94 (m, 1H), 7.43 (d, J=8.3 Hz, 1H). ¹³C NMR (101 MHz, CDCl₃): 28.0 (3C), 29.7 (3C), 30.5, 45.4, 46.1 (2C), 50.6, 52.4, 53.5, 63.0, 82.2, 82.3, 170.5, 170.8, 172.3, 193.9. ESI MS: 440.3 ([M+H]⁺). HR ESI MS: calcd for C₂₁H₃₈O₅N₅ 440.28675; found 440.28632.

### Preparation of tert-Butyl (S)-6-diazo-2-(3-(2-(dimethylamino)acetamido)propanamido)-5-oxohexanoate (25a)

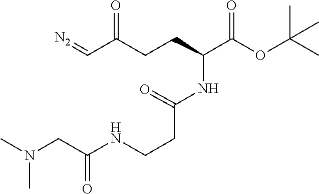

Compound 10o (63 mg, 0.211 mmol, 1 equiv.) and dimethylglycineOSu (47 mg, 0.232 mmol, 1.1 equiv.) were dissolved in anhydrous DCM (2 mL). The resulting mixture was stirred at rt for 3 h. DCM was evaporated and the crude product was purified by HPLC (acetonitril/H₂O, 0.1% formic acid), lyophilized and the compound 25a was obtained as an yellow oil (40 mg) in 49% yield. ¹H NMR (401 MHz, CDCl₃): 1.42 (s, 9H), 1.83-2.01 (m, 1H), 2.05-2.20 (m, 1H), 2.29 (s, 6H), 2.25-2.41 (m, 2H), 2.40-2.48 (m, 2H), 2.99 (s, 2H), 3.53 (dtt, J=20.0, 13.3, 6.2 Hz, 2H), 4.40 (td, J=8.1, 4.7 Hz, 1H), 5.33 (bs, 1H), 6.79 (d, J=7.9 Hz, 1H), 7.66 (d, J=6.3 Hz, 1H). ¹³C NMR (101 MHz, CDCl₃): 27.35, 28.03 (3C), 35.19, 35.94, 36.70, 45.68 (2C), 52.45, 54.89, 62.56, 82.44, 170.33, 171.05, 171.41, 193.94. ESI MS: 384.2 ([M+H]⁺). HR ESI MS: calcd for C₁₇H₃₀O₅N₅ 384.22415; found 384.22351.

### Example 9

### Preparation of Compound 26a

Compound 26a was prepared according to the following reaction Scheme.

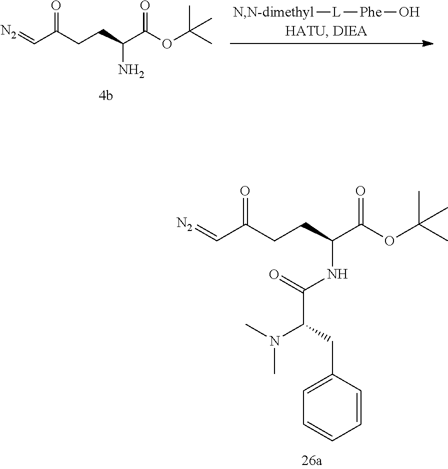

### Preparation of tert-Butyl (S)-6-diazo-2-((S)-2-(dimethylamino)-3-phenylpropanamido)-5-oxohexanoate (26a)

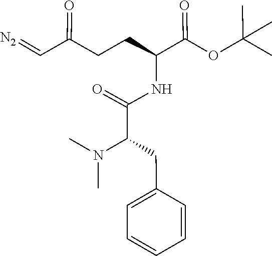

N,N-Dimethyl-L-Phe-OH (85 mg, 0.440 mmol, 1 equiv.) and HATU (184 mg, 0.484 mmol, 1.1 equiv.) were dissolved in anhydrous DCM (4 mL), the mixture was cooled to 0° C. and DIEA (170 mg, 230 μL, 1.32 mmol, 3 equiv.) was added. After 5 minutes of stirring the solution of compound 4b (100 mg, 0.440 mmol, 1 equiv.) in anhydrous DCM (2 mL) was added. The resulting mixture was stirred for 30 minutes at 0° C. and 90 minutes at rt. DCM (50 mL) was added and the organic phase was washed with sat. NaHCO₃ (50 mL), H₂O (50 mL) and sat. NaCl (50 mL), dried over anhydrous MgSO₄ and solvent was evaporated. The crude product was purified by LC on silica gel (DCM/MeOH, 30:1) and product 26a was obtained as an yellow oil (143 mg) in 81% yield. ¹H NMR (401 MHz, CDCl₃): 1.42 (s, 9H), 1.88 (ddt, J=13.8, 8.5, 4.2 Hz, 1H), 2.11 (dddd, J=13.8, 9.0, 6.3, 4.8 Hz, 1H), 2.18-2.30 (m, 2H), 2.31 (s, 6H), 2.91 (dd, J=14.1, 6.1 Hz, 1H), 3.14 (dd, J=14.1, 6.8 Hz, 1H), 3.21-3.30 (m, 1H), 4.37 (td, J=8.3, 4.7 Hz, 1H), 5.24 (bs, 1H), 7.13-7.20 (m, 1H), 7.25 (d, J=4.4 Hz, 4H), 7.32 (d, J=8.3 Hz, 1H). ¹³C NMR (101 MHz, CDCl₃): 27.86, 28.06 (3C), 33.48, 36.83, 42.66 (2C), 52.00, 54.70, 71.12, 82.37, 126.27, 128.43 (2C), 129.29 (2C), 139.68, 171.00, 172.53, 193.62. ESI MS: 403.3 ([M+H]⁺). HR ESI MS: calcd for C₂₁H₃₁O₄N₄ 403.23398; found 403.23389.

### Example 10

### Preparation of Compounds 27a-27c and 28a-28c

Compounds 27a-27c and 28a-28c were prepared according to the following reaction Scheme.

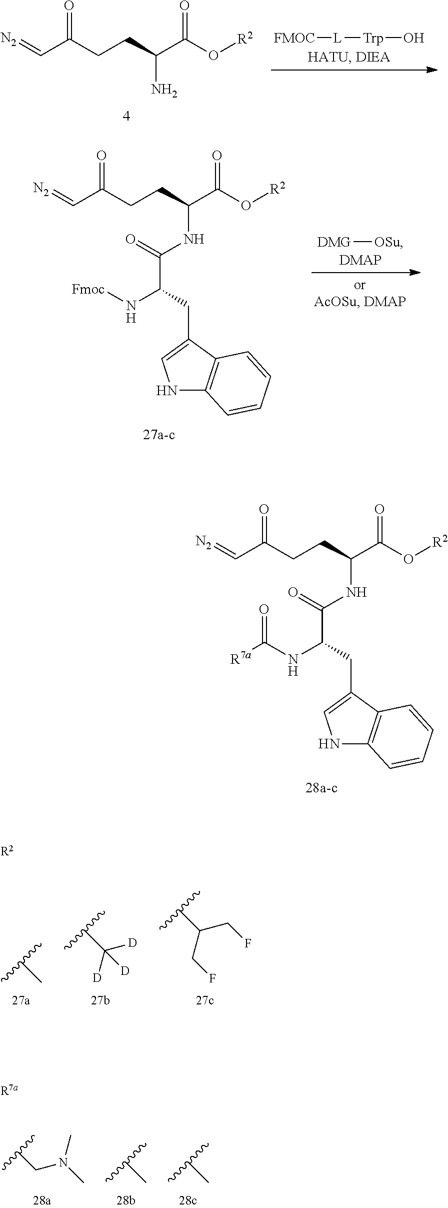

### Preparation of Methyl (S)-2-((S)-2-((((9H-fluoren-9-yl)methoxy)carbonyl)amino)-3-(1H-indol-3-yl)propanamido)-6-diazo-5-oxohexanoate (27a)

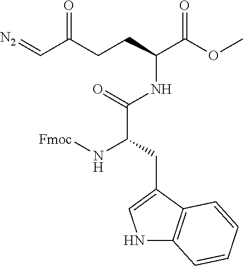

Fmoc-L-Trp-OH (1.21 g, 2.84 mmol, 1.05 equiv.) and HATU (1.13 g, 2.97 mmol, 1.1 equiv.) were dissolved in anhydrous DCM (15 mL), the mixture was cooled to 0° C. and DIEA (1.05 g, 1.48 mL, 8.10 mmol, 3 equiv.) was added. After 5 minutes of stirring the solution of compound 4c (500 mg, 2.70 mmol, 1 equiv.) in anhydrous DCM (8 mL) was added. The resulting mixture was stirred for 30 minutes at 0° C. and overnight (17.5 h) at rt. DCM (70 mL) was added and the organic phase was washed with sat. NaHCO₃ (50 mL), H₂O (50 mL), 10% KHSO₄ (50 mL), H₂O (50 mL), sat. NaCl (50 mL) and dried over anhydrous MgSO₄. DCM was evaporated and the crude product was purified by LC (DCM/EtOAc, 1:1) to obtained compound 27a as a light yellow solid (1.28 g) in 80% yield. ¹H NMR (401 MHz, CDCl₃): 1.85-1.97 (m, 1H), 2.08-2.18 (m, 2H), 2.18-2.28 (m, 1H), 3.19 (dd, J=14.5, 7.4 Hz, 1H), 3.41 (d, J=8.6 Hz, 1H), 3.67 (s, 3H), 4.24 (t, J=7.1 Hz, 1H), 4.34-4.51 (m, 3H), 4.52-4.60 (m, 1H), 5.08 (bs, 1H), 5.50 (d, J=7.8 Hz, 1H), 6.57 (d, J=7.2 Hz, 1H), 7.10 (bs, 1H), 7.16 (t, J=7.4 Hz, 1H), 7.23 (ddd, J=8.2, 7.0, 1.2 Hz, 1H), 7.33 (tdd, J=7.5, 2.4, 1.1 Hz, 2H), 7.37-7.40 (m, 1H), 7.41-7.46 (m, 2H), 7.59 (dd, J=7.5, 5.0 Hz, 2H), 7.70 (d, J=7.8 Hz, 1H), 7.79 (d, J=7.5 Hz, 2H), 8.19 (bs, 1H). ESI MS: 616.2 ([M+Na]⁺). HR ESI MS: calcd for C₃₃H₃₁O₆N₅Na 616.21665; found 616.21622.

### Preparation of Methyl-d₃ (S)-2-((S)-2-((((9H-fluoren-9-yl)methoxy)carbonyl)amino)-3-(1H-indol-3-yl)propanamido)-6-diazo-5-oxohexanoate (27b)

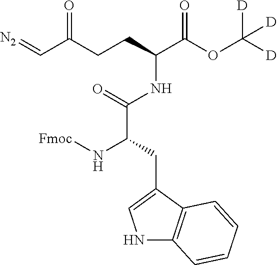

Fmoc-L-Trp-OH (900 mg, 2.11 mmol, 1.05 equiv.) and HATU (840 mg, 2.21 mmol, 1.1 equiv.) were dissolved in anhydrous DCM (10 mL), the mixture was cooled to 0° C. and DIEA (779 mg, 1.10 mL, 6.03 mmol, 3 equiv.) was added. After 5 minutes of stirring the solution of compound 4e (395 mg, 2.01 mmol, 1 equiv.) in anhydrous DCM (5 mL) was added. The resulting mixture was stirred for 30 minutes at 0° C. and 90 minutes at rt. DCM (70 mL) was added and the organic phase was washed with sat. NaHCO₃ (50 mL), H₂O (50 mL), 10% KHSO₄ (50 mL), H₂O (50 mL), sat. NaCl (50 mL) and dried over anhydrous MgSO₄. DCM was evaporated and the crude product was purified by LC (DCM/EtOAc, 1:1) to obtained compound 27c as a light yellow solid (935 mg) in 78% yield. ¹H NMR (401 MHz, CDCl₃): 1.80-1.94 (m, 1H), 2.06-2.26 (m, 3H), 3.17 (dd, J=14.6, 7.3 Hz, 1H), 3.31-3.44 (m, 1H), 4.20 (t, J=7.1 Hz, 1H), 4.29-4.48 (m, 3H), 4.49-4.59 (m, 1H), 5.05 (bs, 1H), 5.52 (d, J=7.9 Hz, 1H), 6.62 (d, J=6.8 Hz, 1H), 7.04-7.09 (m, 1H), 7.10-7.15 (m, 1H), 7.19 (ddd, J=8.1, 7.0, 1.2 Hz, 1H), 7.30 (tdd, J=7.5, 2.3, 1.2 Hz, 2H), 7.35 (dt, J=8.1, 0.9 Hz, 1H), 7.37-7.43 (m, 2H), 7.52-7.60 (m, 2H), 7.66 (d, J=7.8 Hz, 1H), 7.76 (d, J=7.5 Hz, 2H), 8.28 (s, 1H). ¹³C NMR (101 MHz, CDCl₃): 26.97, 28.52, 36.16, 47.12-47.37 (m), 52.04, 52.09, 54.90, 55.75, 67.27, 110.30, 111.39, 118.89, 120.02, 120.11, 120.12, 122.43, 123.65, 125.25, 125.29, 127.23 (2C), 127.61, 127.87 (2C), 136.40, 141.41 (2C), 143.87, 143.97, 156.10, 171.51, 171.76, 193.84. ESI MS: 619.2 ([M+Na]⁺). HR ESI MS: calcd for C₃₃H₂₈D₃O₆N₅Na 619.23549; found 619.23531.

### Preparation of 1,3-Difluoropropan-2-yl (S)-2-((S)-2-((((9H-fluoren-9-yl)methoxy)carbonyl)amino)-3-(1H-indol-3-yl)propanamido)-6-diazo-5-oxohexanoate (27c)

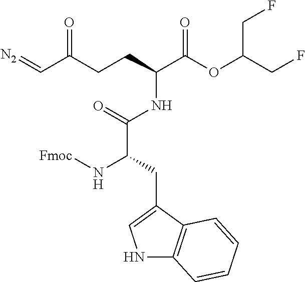

Fmoc-L-Trp-OH (94 mg, 0.221 mmol, 1.1 equiv.) and HATU (92 mg, 0.241 mmol, 1.2 equiv.) were dissolved in anhydrous DCM (4 mL), the mixture was cooled to 0° C. and DIEA (78 mg, 105 μL, 0.602 mmol, 3 equiv.) was added. After 5 minutes of stirring the solution of compound 4g (50 mg, 0.200 mmol, 1 equiv.) in anhydrous DCM (2 mL) was added. The resulting mixture was stirred for 30 minutes at 0° C. and 120 minutes at rt. DCM (60 mL) was added and the organic phase was washed with sat. NaHCO₃ (50 mL), H₂O (50 mL), 10% KHSO₄ (50 mL), H₂O (50 mL), sat. NaCl (50 mL) and dried over anhydrous MgSO₄. DCM was evaporated and the crude product 27c was obtained as light yellow solid (100 mg) in 76% yield and was used to the following step without any purification. ¹H NMR (401 MHz, DMSO-d₆): 1.89 (dtd, J=14.5, 8.9, 6.1 Hz, 1H), 2.04 (dq, J=14.1, 7.0 Hz, 1H), 2.37-2.46 (m, 2H), 2.94 (dd, J=14.7, 10.5 Hz, 1H), 3.10 (dd, J=14.7, 4.0 Hz, 1H), 4.09-4.20 (m, 3H), 4.28-4.39 (m, 2H), 4.50-4.63 (m, 2H), 4.63-4.74 (m, 2H), 5.19-5.35 (m, 1H), 5.99 (bs, 1H), 6.96-7.01 (m, 1H), 7.04-7.09 (m, 1H), 7.20 (d, J=2.2 Hz, 1H), 7.24 (td, J=7.5, 1.1 Hz, 1H), 7.28-7.35 (m, 2H), 7.39 (td, J=7.6, 1.1 Hz, 2H), 7.56 (d, J=8.4 Hz, 1H), 7.61 (d, J=7.6 Hz, 1H), 7.65 (d, J=7.5 Hz, 1H), 7.69 (d, J=7.9 Hz, 1H), 7.84-7.89 (m, 2H), 8.58 (d, J=7.2 Hz, 1H), 10.83 (d, J=2.4 Hz, 1H). ESI MS: 680.2 ([M+Na]⁺). HR ESI MS: calcd for C₃₅H₃₃O₆N₅F₂Na 680.22911; found 680.22877.

### Preparation of Methyl (S)-6-diazo-2-((S)-2-(2-(dimethylamino)acetamido)-3-(1H-indol-3-yl)propanamido)-5-oxohexanoate (28a)

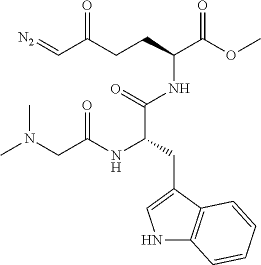

Dimethylglycine (31 mg, 0.296 mmol, 1.1 equiv.) and HATU (118 mg, 0.309 mmol, 1.15 equiv.) were dissolved in anhydrous DMF (4 mL), the mixture was cooled to 0° C. and DIEA (104 mg, 140 μL, 0.807 mmol, 3 equiv.) was added. After 5 minutes of stirring the solution of compound 27a (100 mg, 0.269 mmol, 1 equiv.) in anhydrous DMF (3 mL) was added. The resulting mixture was stirred for 30 minutes at 0° C. and overnight (19.5 h) at rt. DMF was evaporated, EtOAc (70 mL) was added and the organic phase was washed with sat. NaHCO₃ (50 mL), H₂O (50 mL) and sat. NaCl (50 mL), dried over anhydrous MgSO₄ and solvent was evaporated. The crude product was purified by LC on silica gel (DCM/MeOH, 12:1) and product 28a was obtained as an yellow solid (75 mg) in 61% yield. ¹H NMR (401 MHz, CDCl₃): 1.82-1.95 (m, 1H), 2.01-2.14 (m, 1H), 2.15-2.26 (m, 2H), 2.19 (s, 6H), 2.93 (d, J=16.2 Hz, 1H), 3.03 (d, J=16.1 Hz, 1H), 3.24 (d, J=6.7 Hz, 2H), 3.65 (s, 3H), 4.40 (dd, J=8.4, 4.9 Hz, 1H), 4.68 (t, J=6.8 Hz, 1H), 5.26 (bs, 1H), 7.04-7.12 (m, 2H), 7.15 (ddd, J=8.2, 7.1, 1.2 Hz, 1H), 7.21 (d, J=7.7 Hz, 1H), 7.34 (dt, J=8.1, 1.0 Hz, 1H), 7.62 (dt, J=7.8, 1.0 Hz, 1H), 7.76 (d, J=7.9 Hz, 1H), 8.92 (bs, 1H). ¹³C NMR (101 MHz, CDCl₃): 26.79, 27.94, 36.09, 45.55 (2C), 51.90, 52.51, 53.70, 53.79, 62.32, 109.91, 111.32, 118.63, 119.45, 122.02, 123.49, 127.48, 136.28, 170.69, 171.67, 171.93, 193.77. ESI MS: 479.2 ([M+Na]⁺). HR ESI MS: calcd for C₂₂H₂₈O₅N₆Na 479.20134; found 479.20095.

### Preparation of Methyl-d₃ (S)-2-((S)-2-acetamido-3-(1H-indol-3-yl)propanamido)-6-diazo-5-oxohexanoate (28b)

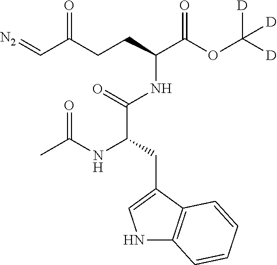

Compound 27b (150 mg, 0.251 mmol, 1 equiv.), AcOSu (198 mg, 1.26 mmol, 5 equiv.) and DMAP (307 mg, 2.51 mmol, 10 equiv.) were dissolved in anhydrous DCM (1.5 mL). The resulting mixture was stirred at rt for 20 h. The crude product was purified by LC on silica gel (DCM/MeOH, 10:1) and the compound 28b was obtained as an yellow solid (84 mg) in 80% yield. ¹H NMR (401 MHz, CDCl₃): 1.82-1.95 (m, 1H), 1.98 (s, 3H), 2.04-2.13 (m, 1H), 2.14-2.34 (m, 2H), 3.17 (dd, J=14.6, 7.3 Hz, 1H), 3.32 (dd, J=14.6, 5.4 Hz, 1H), 4.40 (td, J=7.7, 4.6 Hz, 1H), 4.76 (td, J=7.5, 5.3 Hz, 1H), 5.15 (bs, 1H), 6.27 (d, J=7.7 Hz, 1H), 6.72 (d, J=7.2 Hz, 1H), 7.07-7.14 (m, 2H), 7.18 (ddd, J=8.1, 7.0, 1.2 Hz, 1H), 7.35 (d, J=7.9 Hz, 1H), 7.65 (d, J=7.8 Hz, 1H), 8.38 (bs, 1H). ¹³C NMR (101 MHz, CDCl₃): 23.47, 26.82, 28.32, 36.21, 52.13, 54.01, 54.16, 54.89, 110.45, 111.39, 118.86, 119.90, 122.35, 123.60, 127.74, 136.37, 170.24, 171.58, 171.80, 193.96. ESI MS: 439.2 ([M+Na]⁺). HR ESI MS: calcd for C₂₀H₂₀D₃O₅N₅Na 439.17740; found 439.17797.

### Preparation of 1,3-Difluoropropan-2-yl (S)-2-((S)-2-acetamido-3-(1H-indol-3-yl)propanamido)-6-diazo-5-oxohexanoate (28c)

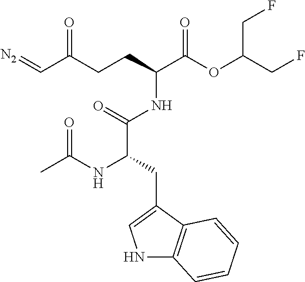

Compound 27c (50 mg, 0.076 mmol, 1 equiv.), AcOSu (60 mg, 0.380 mmol, 5 equiv.) and DMAP (93 mg, 0.760 mmol, 10 equiv.) were dissolved in anhydrous DCM (0.5 mL). The resulting mixture was stirred at rt for 26 h. The crude product was purified by LC on silica gel (DCM/MeOH, 30:1) and the compound 28c was obtained as an light yellow solid (24 mg) in 67% yield. ¹H NMR (401 MHz, CDCl₃): 1.82-1.94 (m, 1H), 1.91 (s, 3H), 2.00-2.11 (m, 1H), 2.12-2.26 (m, 2H), 3.15 (d, J=6.6 Hz, 2H), 4.34 (td, J=7.7, 4.7 Hz, 1H), 4.45 (t, J=4.1 Hz, 2H), 4.57 (t, J=4.1 Hz, 2H), 4.61-4.70 (m, 1H), 5.15 (tt, J=19.5, 4.6 Hz, 1H), 5.22 (bs, 1H), 6.78 (d, J=7.9 Hz, 1H), 7.00-7.08 (m, 2H), 7.12 (t, J=7.5 Hz, 1H), 7.31 (d, J=8.0 Hz, 1H), 7.47 (d, J=7.3 Hz, 1H), 7.56 (d, J=7.8 Hz, 1H), 9.06 (s, 1H). ¹³C NMR (101 MHz, CDCl₃): 22.85, 26.34, 27.94, 35.84, 51.94-52.22 (m, 2C), 53.82, 53.85, 53.94, 80.20 (dt, J=173.1, 6.5 Hz), 109.74, 111.36, 118.48, 119.42, 121.94, 123.50, 127.52, 136.25, 170.58, 170.99, 172.19, 194.55. ESI MS: 500.2 ([M+Na]⁺). HR ESI MS: calcd for C₂₂H₂₅O₅N₅F₂Na 500.17160; found 500.17202.

### Example 11

### Preparation of Compounds 30a and 30b

Compounds 30a and 30b were prepared according to the following reaction Scheme.

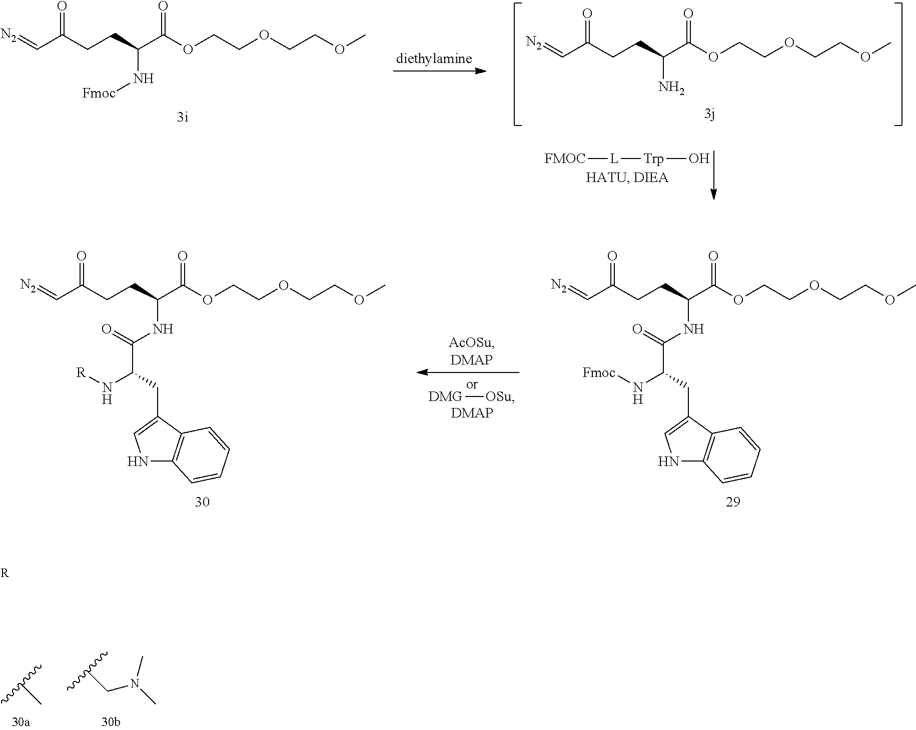

### Preparation of 2-(2-Methoxyethoxy)ethyl (S)-2-((S)-2-((((9H-fluoren-9-yl)methoxy)carbonyl)amino)-3-(1H-indol-3-yl)propanamido)-6-diazo-5-oxohexanoate (29)

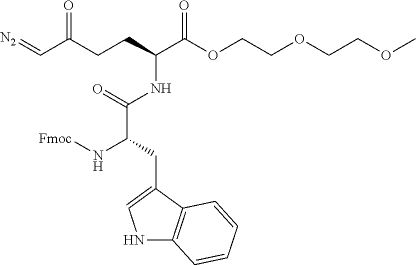

Compound 3i (373 mg, 0.753 mmol, 1 equiv.) was dissolved in anhydrous DCM (3 mL) and piperidine (321 mg, 372 μL, 3.76 mmol, 5 equiv.) was added. The reaction mixture was stirred for 2 h at rt under inert atmosphere. Solvent and excess of secondary amine were evaporated and the residue was purified by LC on silica gel (DCM/MeOH, 20:1) and the intermediate 3j (yellow amorphous compound, 97 mg, 47%) was immediately used to the following step. Compound 3j (97 mg, 0.355 mmol, 1 equiv.) was dissolved in anhydrous DCM (2 mL), Fmoc-L-Trp-OSu (279 mg, 0.532 mmol, 1.5 equiv.) was added and the resulting mixture was stirred at rt for 45 minutes. DCM was evaporated and the residue was purified by LC (DCM/MeOH, 40:1) and the product 29 was obtained as a light yellow solid (206 mg) in 85% yield. ¹H NMR (401 MHz, CDCl₃): 1.80-1.92 (m, 1H), 1.93-2.13 (m, 1H), 2.35-2.43 (m, 2H), 2.86-3.15 (m, 2H), 3.19 (s, 3H), 3.39 (dd, J=5.8, 3.7 Hz, 2H), 3.47-3.54 (m, 2H), 3.55-3.64 (m, 2H), 4.07-4.25 (m, 5H), 4.24-4.38 (m, 2H), 5.98 (bs, 1H), 6.94-7.11 (m, 2H), 7.15-7.27 (m, 1H), 7.27-7.36 (m, 2H), 7.36-7.45 (m, 2H), 7.52 (t, J=8.4 Hz, 2H), 7.57-7.72 (m, 1H), 7.83-7.90 (m, 3H), 8.48 (d, J=7.5 Hz, 2H), 10.82 (s, 1H). ¹³C NMR (101 MHz, DMSO-d₆): 25.9, 27.7, 33.8, 46.6, 51.4, 55.2, 58.0, 58.3, 63.9, 65.6, 68.2, 69.6, 71.2, 110.2, 111.3, 118.2, 118.6, 120.1 (2C), 120.8, 123.9, 125.3, 125.4, 127.0 (2C), 127.2, 127.6 (2C), 136.1, 140.6 (2C), 143.7, 143.8, 155.8, 171.6, 172.3, 194.1. ESI MS: 704.3 ([M+Na]⁺). HR ESI MS: calcd for C₃₇H₃₉O₈N₅Na 704.26908; found 704.26856.

### Preparation of 2-(2-Methoxyethoxy)ethyl (S)-2-((S)-2-acetamido-3-(1H-indol-3-yl)propanamido)-6-diazo-5-oxohexanoate (30a)

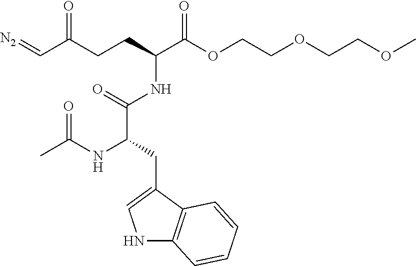

Compound 29 (100 mg, 0.147 mmol, 1 equiv.), AcOSu (46 mg, 0.294 mmol, 2 equiv.) and DMAP (179 mg, 1.47 mmol, 10 equiv.) were dissolved in anhydrous DCM (1 mL). The resulting mixture was stirred at rt for 24 h. The crude product was purified by LC on silica gel (DCM/MeOH, 30:1) and the product 30a was obtained as an yellow amorphous compound (33 mg) in 45% yield. ¹H NMR (401 MHz, CDCl₃): 1.88 (dt, J=14.1, 7.0 Hz, 1H), 1.97 (s, 3H), 2.06 (p, J=5.6, 4.2 Hz, 1H), 2.12-2.30 (m, 2H), 3.22 (ddd, J=56.3, 14.5, 6.4 Hz, 2H), 3.36 (s, 3H), 3.50-3.71 (m, 6H), 4.21 (dtd, J=21.1, 12.2, 10.8, 5.8 Hz, 2H), 4.41 (q, J=7.2, 6.7 Hz, 1H), 4.75 (q, J=6.9 Hz, 1H), 5.20 (bs, 1H), 6.44 (d, J=7.6 Hz, 1H), 6.73 (d, J=7.4 Hz, 1H), 7.03-7.18 (m, 3H), 7.33 (d, J=8.1 Hz, 1H), 7.65 (d, J=7.9 Hz, 1H), 8.83 (s, 1H). ¹³C NMR (101 MHz, CDCl₃): 23.3, 27.0, 28.5, 36.1, 52.1, 54.1, 54.9, 59.0, 64.6, 69.0, 70.5, 71.9, 110.0, 111.4, 118.7, 119.6, 122.1, 123.8, 127.6, 136.4, 170.2, 171.2, 171.6, 194.0. ESI MS: 524.2 ([M+Na]⁺). HR ESI MS: calcd for C₂₄H₃₁O₇N₅Na 524.21157; found 524.21106.

### Preparation of 2-(2-Methoxyethoxy)ethyl (S)-6-diazo-2-((S)-2-(2-(dimethylamino)acetamido)-3-(1H-indol-3-yl)propanamido)-5-oxohexanoate (30b)

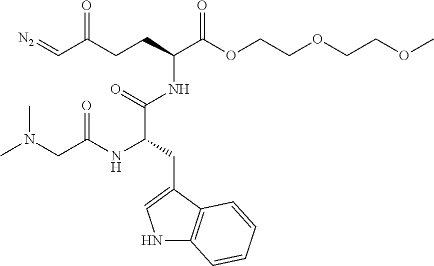

Compound 29 (100 mg, 0.147 mmol, 1 equiv.), dimethylglycineOSu (44 mg, 0.221 mmol, 1.5 equiv.) and DMAP (179 mg, 1.47 mmol, 10 equiv.) were dissolved in anhydrous DCM (1 mL). The resulting mixture was stirred at rt for 24 h. The crude product was purified by LC on silica gel (DCM/MeOH, 15:1) and the product 30b was obtained as an yellow amorphous compound (53 mg) in 66% yield. ¹H NMR (401 MHz, DMSO-d₆): 1.81-1.90 (m, 1H), 1.94-2.08 (m, 7H), 2.35-2.44 (m, 2H), 2.75 (dt, J=31.2, 15.8 Hz, 2H), 3.14-3.19 (m, 2H), 3.20 (s, 3H), 3.40 (dd, J=5.8, 3.7 Hz, 2H), 3.52 (dd, J=5.9, 3.6 Hz, 2H), 3.60 (t, J=4.9 Hz, 2H), 4.04-4.17 (m, 1H), 4.17-4.25 (m, 1H), 4.25-4.31 (m, 1H), 4.61-4.69 (m, 1H), 6.02 (bs, 1H), 6.97 (t, J=7.4 Hz, 1H), 7.05 (t, J=7.5 Hz, 1H), 7.12-7.16 (m, 1H), 7.31 (d, J=8.1 Hz, 1H), 7.56-7.67 (m, 2H), 8.52 (d, J=7.5 Hz, 1H), 10.81 (s, 1H). ¹³C NMR (101 MHz, CDCl₃): 27.9, 29.6, 36.0, 45.4, 51.7, 53.6, 55.1, 58.8 (2C), 62.4, 64.3, 68.7, 70.2, 71.7, 109.4, 111.2, 118.3, 119.1, 121.7, 123.5, 127.3, 136.3, 171.1, 171.2, 171.8, 194.8. ESI MS: 545.3 ([M+H]⁺). HR ESI MS: calcd for C₂₆H₃₇O₇N₆ 545.27182; found 545.27157.

### Example 12

### Preparation of Compounds 31a and 31b

Compounds 31a and 31b were prepared according to the following reaction Scheme.

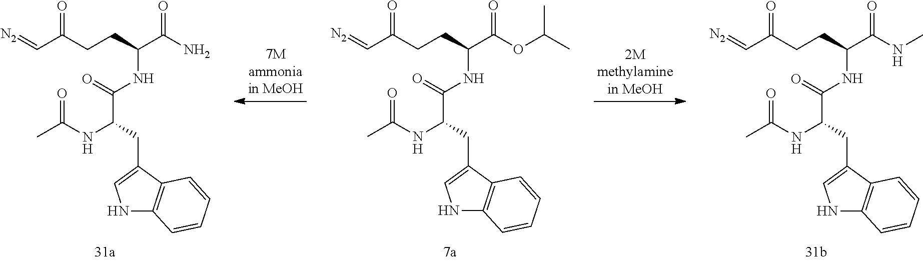

### Preparation of (S)-2-((S)-2-Acetamido-3-(1H-indol-3-yl)propanamido)-6-diazo-5-oxohexanamide (31a)

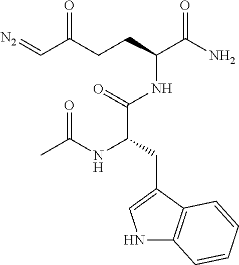

Compound 7a (200 mg, 0.453 mmol, 1 equiv.) was dissolved in 7M NH₃ in MeOH (15 mL) and the reaction mixture was heated to 60° C. for 40 h. Solvent was evaporated and the residue was purified by LC on silica gel (CHCl₃/MeOH, 10:1+1% Et₃N). Compound 31a was obtained as a light yellow solid (144 mg) in 80% yield. ¹H NMR (401 MHz, DMSO-d₆): 1.68-1.78 (m, 1H), 1.79 (s, 3H), 1.87-2.00 (m, 1H), 2.20-2.32 (m, 2H), 2.91 (dd, J=14.7, 9.1 Hz, 1H), 3.12 (dd, J=14.7, 4.7 Hz, 1H), 4.15 (td, J=8.6, 5.1 Hz, 1H), 4.49 (ddd, J=9.1, 7.5, 4.7 Hz, 1H), 5.99 (bs, 1H), 6.97 (t, J=7.4 Hz, 1H), 7.02-7.10 (m, 2H), 7.12-7.22 (m, 2H), 7.32 (d, J=8.0 Hz, 1H), 7.60 (d, J=7.9 Hz, 1H), 7.99 (d, J=8.0 Hz, 1H), 8.05 (d, J=7.6 Hz, 1H), 10.80 (bs, 1H). ¹³C NMR (101 MHz, DMSO-d₆): 22.59, 27.03, 27.40, 29.01, 51.84, 53.72, 69.80, 110.15, 111.29, 118.20, 118.49, 120.86, 123.62, 127.31, 136.07, 169.51, 171.73, 173.02, 194.44. ESI MS: 421.2 ([M+Na]⁺). HR ESI MS: calcd for C₁₉H₂₂O₄N₆Na 421.15947; found 421.15918.

### Preparation of (S)-2-((S)-2-Acetamido-3-(1H-indol-3-yl)propanamido)-6-diazo-N-methyl-5-oxohexanamide (31b)

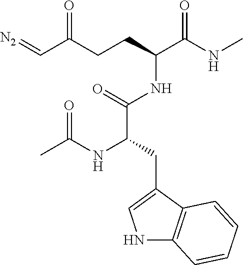

Starting material 7a (200 mg, 0.453 mmol, 1 equiv.) was dissolved in the solution of 2M methylamine in MeOH (12 mL) and the reaction mixture was heated to 60° C. for 20 h. Solvent was evaporated and the residue was purified by LC on silica gel (CHCl₃/MeOH, 10:1+1% Et₃N). Compound 31b was obtained as an yellow solid (122 mg) in 65% yield.

¹H NMR (401 MHz, DMSO-d₆): 1.66-1.77 (m, 1H), 1.81 (s, 3H), 1.88-2.01 (m, 1H), 2.20-2.31 (m, 2H), 2.53 (d, J=4.6 Hz, 3H), 2.92 (dd, J=14.6, 8.7 Hz, 1H), 3.12 (dd, J=14.6, 5.2 Hz, 1H), 4.15 (td, J=8.6, 5.2, 1H), 4.48-4.52 (m, 1H), 5.97 (bs, 1H), 6.98 (t, J=7.3 Hz, 1H), 7.06 (t, J=7.1 Hz, 1H), 7.16 (d, J=2.1 Hz, 1H), 7.33 (d, J=8.0 Hz, 1H), 7.45 (d, J=4.5 Hz, 1H), 7.59 (d, J=7.7 Hz, 1H), 8.03-8.09 (m, 1H), 8.22 (d, J=7.0 Hz, 1H), 10.83 (d, J=2.7 Hz, 1H). ¹³C NMR (101 MHz, DMSO-d₆): 22.59, 25.59, 27.04, 27.46, 36.40, 51.98, 53.70, 54.44, 110.05, 111.30, 118.23, 118.52, 120.89, 123.66, 127.31, 136.05, 169.50, 171.24, 171.75, 194.34. ESI MS: 435.2 ([M+Na]⁺). HR ESI MS: calcd for C₂₀H₂₄O₄N₆Na 435.17512; found 435.17489.

### Example 13

### Preparation of Compounds 8f, 31c-31f, and 33a-33c

Compounds 8f, 31c-31f, and 33a-33c were prepared according to the following reaction Scheme.

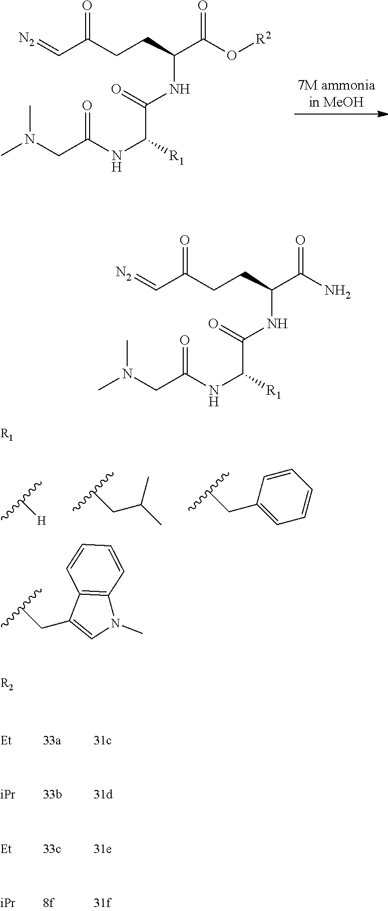

### Preparation of (S)-6-Diazo-2-(2-(2-(dimethylamino)acetamido)acetamido)-5-oxohexanamide (31c)

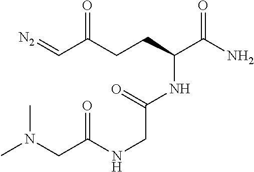

Compound 33a (75 mg, 0.220 mmol, 1 equiv.) was dissolved in the solution of 7M NH₃ in MeOH (6 mL) and the reaction mixture was stirred at rt for 24 h. Solvent was evaporated and the residue was purified by LC on silica gel (DCM/MeOH, 1:1). Compound 31c was obtained as a light yellow solid (47 mg) in 68% yield. ¹H NMR (401 MHz, CDCl₃): 2.10 (q, J=6.2 Hz, 2H), 2.35 (s, 6H), 2.41-2.70 (m, 2H), 2.95-3.12 (m, 2H), 3.94 (d, J=5.8 Hz, 2H), 4.45 (dt, J=7.2, 6.0 Hz, 1H), 5.34 (bs, 1H), 5.45 (bs, 1H), 6.86-6.91 (m, 1H), 7.69 (bs, 1H), 7.79-7.84 (m, 1H). ¹³C NMR (101 MHz, CDCl₃): 26.9, 36.8, 43.0, 46.2 (2C), 52.8, 55.4, 63.0, 169.6, 172.4, 174.1, 195.5. ESI MS: 313.2 ([M+H]⁺). HR ESI MS: calcd for C₁₂H₂₁O₄N₆ 313.16188; found 313.16211.

### (S)-6-Diazo-2-((S)-2-(2-(dimethylamino)acetamido)-4-methylpentanamido)-5-oxohexanamide (31d)

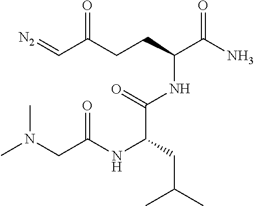

Compound 33b (168 mg, 0.408 mmol, 1 equiv.) was dissolved in the solution of 7M NH₃ in MeOH (13.5 mL) and the reaction mixture was stirred at rt for 48 h. Solvent was evaporated and the residue was purified by LC on silica gel (DCM/MeOH, 10:1). Compound 31d was obtained as a light yellow solid (66 mg) in 44% yield. ¹H NMR (401 MHz, CDCl₃): 0.94 (d, J=6.2 Hz, 3H), 0.99 (d, J=6.2 Hz, 3H), 1.55-1.83 (m, 5H), 2.12 (tdd, J=13.7, 11.2, 6.6 Hz, 2H), 2.35 (s, 6H), 2.43-2.66 (m, 2H), 4.26 (ddd, J=10.4, 6.1, 4.5 Hz, 1H), 4.43 (td, J=7.3, 4.4 Hz, 1H), 5.34 (bs, 1H), 5.40 (bs, 1H), 6.94 (bs, 1H), 7.62 (d, J=6.1 Hz, 1H), 7.82 (bs, 1H). ¹³C NMR (101 MHz, CDCl₃): 21.5, 23.3, 25.2, 26.4, 36.8, 40.5, 46.2 (2C), 52.7, 52.7, 55.4, 63.1, 172.3, 172.5, 173.7, 196.0. ESI MS: 369.2 ([M+H]⁺). HR ESI MS: calcd for C₁₆H₂₉O₄N₆ 369.22448; found 369.22470.

### Preparation of (S)-6-Diazo-2-((S)-2-(2-(dimethylamino)acetamido)-3-phenylpropanamido)-5-oxohexanamide (31e)

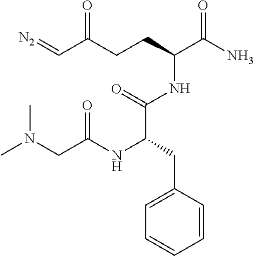

Compound 33c (50 mg, 0.116 mmol, 1 equiv.) was dissolved in the solution of 7M NH₃ in MeOH (5 mL) and the reaction mixture was stirred at rt for 20 h. Solvent was evaporated and the residue was purified by LC on silica gel (DCM/MeOH, 10:1). Compound 31e was obtained as a light yellow solid (29 mg) in 62% yield. ¹H NMR (401 MHz, DMSO-d₆): 1.67-1.82 (m, 1H), 1.87-2.00 (m, 1H), 2.06 (s, 6H), 2.27-2.35 (m, 2H), 2.65-3.12 (m, 4H), 4.14-4.23 (m, 1H), 4.55-4.63 (m, 1H), 6.04 (bs, 1H), 7.09 (bs, 1H), 7.14-7.28 (m, 6H), 7.76 (d, J=8.4 Hz, 1H), 8.12 (d, J=8.1 Hz, 1H). ¹³C NMR (101 MHz, DMSO-d₆): 27.2, 37.3, 45.4 (2C), 51.6, 53.2, 63.0, 69.8, 72.4, 126.3, 128.1 (2C), 129.6 (2C), 137.6, 169.3, 170.8, 172.8, 194.3. ESI MS: 403.2 ([M+H]⁺). HR ESI MS: calcd for C₁₉H₂₇O₄N₆ 403.20883; found 403.20898.

### Preparation of (S)-6-Diazo-2-((S)-2-(2-(dimethylamino)acetamido)-3-(1-methyl-1H-indol-3-yl)propanamido)-5-oxohexanamide (31f)

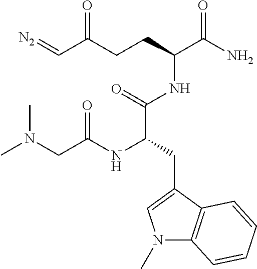

Compound 8f (40 mg, 0.080 mmol, 1 equiv.) was dissolved in the solution of 7M NH₃ in MeOH (4 mL) and the reaction mixture was heated to 50° C. for 72 h. Solvent was evaporated and the residue was purified by LC on silica gel (DCM/MeOH, 10:1+1% Et₃N). Compound 31f was obtained as an yellow solid (11 mg) in 30% yield. ¹H NMR (401 MHz, CDCl₃): 1.86-2.07 (m, 2H), 2.17 (s, 6H), 2.20-2.51 (m, 2H), 2.84 (d, J=16.3 Hz, 1H), 3.17 (d, J=16.4 Hz, 1H), 3.30 (d, J=6.6 Hz, 2H), 3.76 (s, 3H), 4.32 (td, J=7.3, 4.2 Hz, 1H), 4.50 (q, J=6.3 Hz, 1H), 5.11 (bs, 1H), 5.34 (bs, 1H), 6.79 (s, 1H), 6.96 (s, 1H), 7.12 (t, J=7.9 Hz, 1H), 7.20-7.31 (m, 2H), 7.64 (d, J=7.9 Hz, 2H), 7.91 (d, J=5.3 Hz, 1H). ESI MS: 456.2 ([M+H]⁺). HR ESI MS: calcd for C₂₂H₃₀O₄N₇ 456.23538; found 456.23512.

### Example 14

### Preparation of Compounds 32a-32c and 33a-33c

Compounds 32a-32c and 33a-33c were prepared according to the following reaction Scheme.

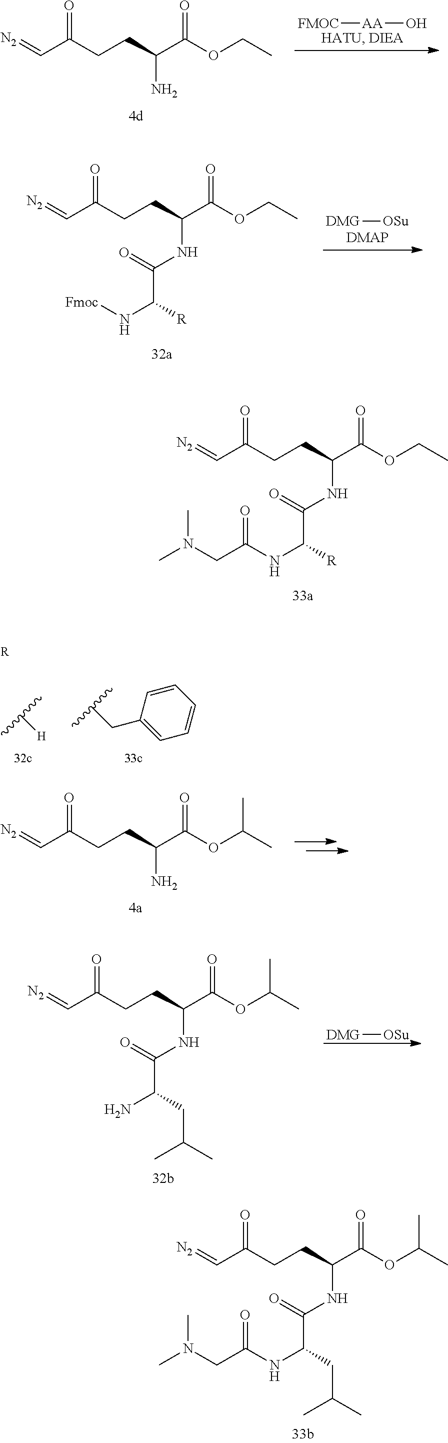

### Preparation of Ethyl (S)-2-(2-((((9H-fluoren-9-yl)methoxy)carbonyl)amino)acetamido)-6-diazo-5-oxohexanoate (32a)

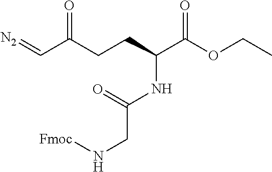

Fmoc-Gly-OH (561 mg, 2.01 mmol, 1 equiv.) and HATU (840 mg, 2.21 mmol, 1.1 equiv.) were suspended in anhydrous DCM (10 mL) and reaction mixture was cooled to 0° C. DIEA (779 mg, 1.05 mL, 6.02 mmol, 3 equiv.) was added and the mixture was stirred for 5 minutes under inert atmosphere. Finally solution of compound 4d (400 mg, 2.01 mmol, 1 equiv.) in anhydrous DCM (5 mL) was slowly added during 5 minutes. The resulting mixture was stirred for 30 minutes at 0° C. and overnight (18.5 h) at room temperature. DCM was evaporated, EtOAc (100 mL) was added and the organic phase was washed with sat. NaHCO₃ (50 mL), H₂O (50 mL), 10% KHSO₄ (50 mL), H₂O (50 mL), sat. NaCl (50 mL) and dried over anhydrous MgSO₄. EtOAc was evaporated and the residue was purified by LC on silica gel (DCM/EtOAc, 5:1) and product 32a was obtained as an yellow solid (716 mg) in 75% yield. ¹H NMR (401 MHz, CDCl₃): 1.28 (t, J=7.1 Hz, 3H), 1.95-2.10 (m, 1H), 2.16-2.30 (m, 1H), 2.30-2.58 (m, 2H), 3.84-4.00 (m, 2H), 4.16-4.27 (m, 3H), 4.42 (d, J=7.0 Hz, 2H), 4.56 (td, J=8.0, 4.6 Hz, 1H), 5.26 (bs, 1H), 5.42 (bs, 1H), 6.87 (d, J=7.5 Hz, 1H), 7.32 (td, J=7.5, 1.2 Hz, 2H), 7.40 (tt, J=7.5, 1.0 Hz, 2H), 7.60 (d, J=7.5 Hz, 2H), 7.77 (dt, J=7.6, 1.0 Hz, 2H).

### Preparation of Ethyl (S)-6-diazo-2-(2-(2-(dimethylamino)acetamido)acetamido)-5-oxohexanoate (33a)

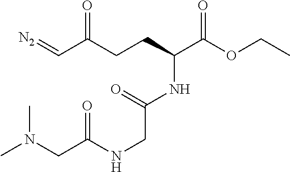

Compound 32a (716 mg, 1.50 mmol, 1 equiv.), dimethylglycineOSu (449 mg, 2.24 mmol, 1.5 equiv.) and DMAP (1.83 g, 15.0 mmol, 10 equiv.) were dissolved in anhydrous DCM (5 mL). The resulting mixture was stirred at rt for 17 h. The crude product was purified by LC on silica gel (DCM/MeOH, 5:1) and the compound 33a was obtained as a light yellow oil (281 mg) in 55% yield. ¹H NMR (401 MHz, CDCl₃): 1.28 (t, J=7.2 Hz, 3H), 2.04 (tt, J=14.7, 7.1 Hz, 1H), 2.21 (dtd, J=13.1, 6.5, 6.0, 4.2 Hz, 1H), 2.30-2.53 (m, 2H), 2.34 (s, 6H), 3.01 (d, J=16.3 Hz, 1H), 3.08 (d, J=16.3 Hz, 1H), 3.93 (dd, J=16.7, 6.0 Hz, 1H), 4.04 (dd, J=16.6, 6.0 Hz, 1H), 4.19 (q, J=7.2 Hz, 2H), 4.52 (td, J=8.0, 4.6 Hz, 1H), 5.31 (bs, 1H), 6.92 (d, J=7.7 Hz, 1H), 7.76 (bs, 1H). ¹³C NMR (101 MHz, CDCl₃): 14.29, 26.88, 36.54, 42.86, 46.17 (2C), 52.19, 54.19, 61.88, 62.98, 169.14, 171.66, 171.71, 194.57. ESI MS: 342.2 ([M+H]⁺). HR ESI MS: calcd for C₁₄H₂₄O₅N₅ 342.17720; found 342.17704.

### Preparation of Isopropyl (S)-6-diazo-2-((S)-2-(2-(dimethylamino)acetamido)-4-methylpentanamido)-5-oxohexanoate (33b)

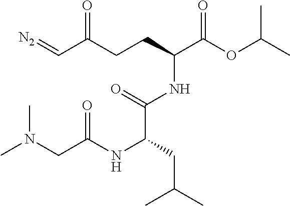

Compound 32b (synthesized according to the published procedure: WO2017/23774 A1) (250 mg, 0.766 mmol, 1 equiv.) and dimethylglycineOSu (153 mg, 0.766 mmol, 1 equiv.) were dissolved in anhydrous DCM (5 mL). The resulting mixture was stirred at rt for 3 h. DCM was evaporated. EtOAc (100 mL) was added and the organic phase was washed with sat. NaHCO₃ (2×50 mL) and sat. NaCl (50 mL), dried over anhydrous MgSO₄ and solvent was evaporated. The crude product was purified by LC on silica gel (DCM/MeOH, 10:1) and the compound 33b was obtained as an yellow oil (261 mg) in 83% yield. ¹H NMR (401 MHz, CDCl₃): 0.92 (d, J=6.2 Hz, 3H), 0.95 (d, J=6.2 Hz, 3H), 1.23 (d, J=6.2 Hz, 3H), 1.24 (d, J=6.2 Hz, 3H), 1.51-1.61 (m, 1H), 1.60-1.74 (m, 2H), 1.95 (dtd, J=14.4, 8.2, 6.2 Hz, 1H), 2.11-2.23 (m, 1H), 2.28 (s, 6H), 2.30-2.44 (m, 2H), 2.95 (d, J=3.9 Hz, 2H), 4.37-4.50 (m, 2H), 5.01 (hept, J=6.2 Hz, 1H), 5.33 (bs, 1H), 6.90 (d, J=7.7 Hz, 1H), 7.45 (d, J=8.3 Hz, 1H). ¹³C NMR (101 MHz, CDCl₃): 21.81, 21.83, 22.01, 23.06, 24.90, 27.21, 36.43, 41.04, 46.09 (2C), 51.42, 52.07, 54.94, 63.10, 69.48, 70.67, 171.10, 172.13, 193.91. ESI MS: 412.3 ([M+H]⁺). HR ESI MS: calcd for C₁₉H₃₄O₅N₅ 412.25545; found 412.25545.

### Preparation of Ethyl (S)-2-((S)-2-((((9H-fluoren-9-yl)methoxy)carbonyl)amino)-3-phenylpropanamido)-6-diazo-5-oxohexanoate (32c)

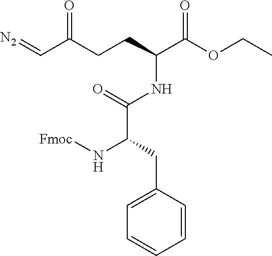

Fmoc-L-Phe-OH (389 mg, 1.00 mmol, 1 equiv.) and HATU (420 mg, 1.10 mmol, 1.1 equiv.) were suspended in anhydrous DCM (4 mL) and reaction mixture was cooled to 0° C. DIEA (389 mg, 525 μL, 3.01 mmol, 3 equiv.) was added and the mixture was stirred for 5 minutes under inert. Finally solution of compound 4d (200 mg, 1.00 mmol, 1 equiv.) in anhydrous DCM (2 mL) was slowly added during 5 minutes. The resulting mixture was stirred for 30 minutes at 0° C. and overnight (17.5 h) at room temperature. DCM was evaporated, EtOAc (100 mL) was added and the organic phase was washed with sat. NaHCO₃ (50 mL), H₂O (50 mL), 10% KHSO₄ (50 mL), H₂O (50 mL), sat. NaCl (50 mL) and dried over anhydrous MgSO₄. EtOAc was evaporated and the residue was purified by LC on silica gel (DCM/EtOAc, 5:1) and product 32c was obtained as a light yellow solid (390 mg) in 68% yield. ¹H NMR (401 MHz, CDCl₃): 1.26 (t, J=7.1 Hz, 3H), 1.88-2.02 (m, 1H), 2.10-2.22 (m, 1H), 2.23-2.41 (m, 2H), 3.06-3.12 (m, 2H), 4.07-4.23 (m, 3H), 4.25-4.36 (m, 1H), 4.38-4.53 (m, 3H), 5.16 (bs, 1H), 5.37 (d, J=7.9 Hz, 1H), 6.71 (d, J=7.3 Hz, 1H), 7.16-7.26 (m, 3H), 7.26-7.33 (m, 4H), 7.40 (tq, J=7.6, 1.0 Hz, 2H), 7.51-7.59 (m, 2H), 7.76 (dd, J=7.5, 0.9 Hz, 2H). ¹³C NMR (101 MHz, CDCl₃): 14.27, 27.10, 36.32, 38.51, 47.26, 52.17, 54.13, 56.29, 61.86, 67.28, 120.15, 120.16, 125.18, 125.28, 125.31, 127.01, 127.26 (2C), 127.91 (2C), 128.85 (2C), 129.54 (2C), 141.45 (2C), 143.87, 143.90, 157.32, 170.87, 171.28, 196.26. ESI MS: 591.3 ([M+Na]⁺). HR ESI MS: calcd for C₃₂H₃₂O₆N₄Na 591.22141; found 591.22107.

### Preparation of Ethyl (S)-6-diazo-2-((S)-2-(2-(dimethylamino)acetamido)-3-phenylpropanamido)-5-oxohexanoate (33c)

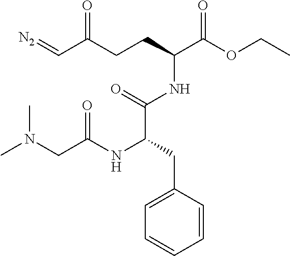

Compound 32c (200 mg, 0.352 mmol, 1 equiv.), dimethylglycineOSu (141 mg, 0.703 mmol, 2 equiv.) and DMAP (430 mg, 3.52 mmol, 10 equiv.) were dissolved in anhydrous DCM (1.5 mL). The resulting mixture was stirred at rt for 18 h. The crude product was purified by LC on silica gel (DCM/MeOH, 20:1+1% Et₃N) and the compound 33c was obtained as a light yellow solid (127 mg) in 84% yield. ¹H NMR (401 MHz, CDCl₃): 1.19 (t, J=7.1 Hz, 3H), 1.83 (dtd, J=14.7, 8.7, 6.1 Hz, 1H), 1.95-2.04 (m, 1H), 2.06 (s, 6H), 2.35-2.44 (m, 2H), 2.71 (d, J=15.5 Hz, 1H), 2.82 (d, J=15.5 Hz, 1H), 2.82-2.90 (m, 1H), 3.05 (dd, J=13.8, 4.6 Hz, 1H), 4.09 (q, J=7.1 Hz, 2H), 4.24 (ddd, J=9.2, 7.5, 5.3 Hz, 1H), 4.63 (td, J=9.0, 4.5 Hz, 1H), 6.05 (bs, 1H), 7.13-7.33 (m, 5H), 7.69 (d, J=8.6 Hz, 1H), 8.49 (d, J=7.5 Hz, 1H). ¹³C NMR (101 MHz, CDCl₃): 14.23, 27.07, 36.35, 37.76, 45.95 (2C), 52.08, 54.12, 54.96, 61.71, 62.98, 127.05, 128.71 (2C), 129.31 (2C), 136.66, 171.12, 171.29, 171.35, 193.33. ESI MS: 432.2 ([M+H]⁺). HR ESI MS: calcd for C₂₁H₃₀O₅N₅ 432.22415; found 432.22432.

### Example 15

### Compound Profiling

### Metabolic Stability

The representative Compounds of the Disclosure were screened for metabolic stability in swine jejunum/liver tissue homogenates, following our previously reported methods (Zimmermann et al., J. Med. Chem., 61(9):3918-29 (2018); Tenora et al., J. Med. Chem., 62(7):3524-38 (2019)). In brief, freshly collected tissues were homogenized by probe sonication in 9× volume of 0.1 M potassium phosphate buffer over ice. Post homogenization, prodrugs were spiked at a concentration of 20 μM (in triplicate) and incubated in an orbital shaker at 37° C. for 60 min. Final concentration of DMSO in the incubations was 0.2% v/v. At each time point (0, 30, 60 min), 100 μL of sample was precipitated with 300 μL of methanol containing internal standard (IS; losartan: 0.5 μM). Precipitated samples were thoroughly vortexed and centrifuged at 10000×g for 10 min at 4° C. After centrifugation, 50 μL of supernatant was diluted with 50 μL of water, vortexed and submitted for analysis. Prodrug disappearance over time was measured using liquid chromatography tandem mass spectrometry (LC-MS/MS).

The processed supernatants were analyzed on a Thermo Scientific Vanquish UPLC system (equipped with Vanquish autosampler, pumps, and column compartment) hyphenated to TSQ Altis mass spectrometer (Thermo Fisher Scientific Inc., Waltham MA). Samples were ionized in positive mode using heated electrospray probe and peak area counts were measured with selected reaction monitoring (SRM). Chromatographic separation was achieved using Waters XBridge C18 column (100×2.1 mm, 1.8 μm particle size; maintained at 35° C.). The autosampler was operated at 4° C. The mobile phase consisted of 0.1% formic acid in water and acetonitrile as aqueous and organic modifiers respectively. Pumps were operated at a flow rate of 0.4 mL/min with gradient elution [time(min)/% B=0.00/5, 0.50/5, 2.50/95, 3.50/95, 3.60/5, 5.00/5] spanning over a run time of 5 min. Peak area ratios obtained from peak area counts of analyte and internal standard were used to measure the disappearance of prodrugs.

### Human Tumor Cell-to-Plasma Partitioning Assay

The human plasma-to-tumor cell partitioning assays were conducted using P493B lymphoma following our previously reported method. Gao et al. 2009; Tenora et al., 2019. In brief, cells were cultured in 150 cm² T-flasks (Falcon™, USA, Cat. #08-772-48) with RPMI Medium 1640 medium 1× containing L-glutamine (RPMI-1640, Corning®, USA, Cat. #10-040-CV), supplemented with 10% v/v Fetal Bovine Serum (FBS, Gibco™, USA, Cat. #26140079), and 1% v/v antimycotic/antibiotic (Gibco™, USA, Cat. #15240062). Cells were grown at 37° C., in a humidified atmosphere with 5% CO₂. Cells were harvested after achieving>80% confluency and centrifuged at 200×g for 5 min at 25° C. The obtained cell pellet was re-suspended in 20 mL of Dulbecco's phosphate-buffered saline (DPBS, Gibco™, USA, Cat. #14-190-144) maintained at 37° C. and cell count was determined using an automated cell counter (Bio-Rad, USA). Cell suspension in DPBS was further centrifuged at 200×g for 5 min at 25° C. and cell pellet was resuspended in human plasma (Innovative Research, USA) for partitioning assessment. Final cell density after resuspending in plasma was 10 million cells/mL of plasma. Preincubated (37° C. for 5 min) cell-plasma suspension was spiked with prodrug at a final concentration of 20 μM and incubated at 37° C. for 1 h (in triplicate) and was constantly stirred on an orbital shaker. Following incubation, 1 mL aliquot of cell-plasma suspension was centrifuged at 1000×g for 5 min at 4° C. and supernatant plasma was collected and stored at −80° C. until bioanalysis. The cell pellet was washed with ice cold DPBS buffer, and centrifuged to remove plasma traces. Supernatant was removed and left-over cell pellet was stored at −80° C. Both plasma and cell pellet fractions were analyzed for intact prodrug and DON levels.

Frozen plasma and cell pellets were thawed on wet ice. Once thawed, cell pellet was resuspended in water and the total weight of cells was noted and plasma was processed as such. The calibration curves (0.03 to 100 nmol/mL) were prepared in both human plasma and untreated P493B cells. Fifty microliter of cell suspension/plasma was precipitated with 250 μL of methanol containing internal standards (glutamate d5:5 μM and losartan:0.5 μM). Samples were briefly vortexed for 30 s and centrifuged at 10000×g for 10 min at 4° C. For DON analysis, 200 μL of supernatant was dried at 45° C. under vacuum for 1 h. Post evaporation, derivatization was initiated by adding 50 μL of 0.2 M sodium bicarbonate buffer (pH 9.0) and 100 μL of 10 mM dabsyl chloride. Samples were mixed well and incubated at 60° C. for 15 min, followed by centrifugation at 16000×g for 5 min at 4° C. After centrifugation, 100 μL of the supernatant was diluted with 400 μL of water, and analyzed in LC-MS/MS. For intact prodrug analysis, 50 μL of supernatant obtained after centrifugation was diluted with 50 μL water and injected into LC-MS/MS.

The underivatized supernatants/derivatized samples were analyzed on a LC-MS/MS system consisting of Dionex ultra high-performance LC system hyphenated with Q Exactive Focus Orbitrap mass spectrometer (Thermo Fisher Scientific Inc., Waltham, MA). Derivatized samples were ionized in positive mode using heated electrospray probe and peak area counts were measured with parallel reaction monitoring (PRM). Abundant and informative molecular ions of DON (m/z 459.1445) and glutamate-d5 (m/z 440.1647) were fragmented at a collision energy of 10 and 30 eV respectively, to pick the relevant and sensitive fragment ions (DON-m/z 403.1434/431.1384; glutamate d5-m/z 156.9955/394.1585). Chromatographic separation was achieved using Waters XBridge C18 column (100×2.1 mm, 1.8 μm particle size; maintained at 35° C.). The autosampler was operated at 10° C. The mobile phase consisted of 0.1% formic acid in water and acetonitrile as aqueous and organic modifiers respectively. Pumps were operated at a flow rate of 0.4 mL/min with gradient elution (time(min)/% B=0.00/40, 1.50/95, 2.50/95, 2.50/40, 3.50/40) spanning over a run time of 3.5 min. Linear regression fit with 1/(nominal concentration) weighting factor was used for plotting the calibration curve.

Underivatized samples were analyzed using Full MS scan (m/z 75-m/z 1125) function to monitor both parent and metabolites. Pumps were operated at a flow rate of 0.3 mL/min with gradient elution [time(min)/% B=0.00/2.50, 0.50/2.5, 6.00/95, 7.00/95, 7.00/2.5, 8.50/2.5] spanning over a run time of 8.5 min. Rest of the analytical conditions were similar to that of derivatized samples.

### Pharmacokinetic (PK) Study

The pharmacokinetic study of selected analogs in C57BL/6 mice was conducted according to protocols reviewed and approved by the Johns Hopkins Institutional Animal Care and Use Committee. Briefly, C57BL/6 mice (weighing between 25-30 g) 6-8 weeks of age, were used for the study. The animals were maintained on a 12 h light-dark cycle with ad libitum access to food (Certified laboratory food: Teklad 18% Protein Extruded Rodent Diet) and water. EL4 mouse lymphoma cells were obtained as a gift from the laboratory of Dr. Jonathan Powell (Johns Hopkins University, Baltimore, MD) and maintained in RPMI 1640 medium 1× (Corning®, Cat. #10-040-CV) with 10% (v/v) Fetal Bovine Serum (Corning®, Cat. #35-011-CV), 1% (v/v), antimycotic/antibiotic (Corning®, Cat. #30-004-CI), 2 mM of L-Glutamine (Corning®, Cat. #25-005-CI) and 10 mM HEPES (Corning®, Cat. #25-060-CI) in a 5% (v/v) CO₂ and 95% (v/v) air incubator prior to subcutaneous (SC) injection (1×10⁶ cells in 0.2 mL of phosphate-buffered saline) on the flank of each mouse. Pharmacokinetic study was performed after tumor grew to a mean volume of around 400 mm³. Prior to dosing, the interscapular region was wiped with alcohol gauze. Analogs were dissolved immediately prior to dosing in ethanol: Tween 80: saline (5:10:85 v/v/v), and was administered to mice as a single SC dose of 2.9 mg/kg (1 mg/kg DON equivalent dose). The mice were euthanized with carbon-dioxide at specified time points post-drug administration, blood samples (approximately 0.8 mL) were collected in heparinized microtubes by cardiac puncture and jejunum as well as tumors were removed and flash frozen on dry ice. Blood samples were centrifuged at a temperature of 4° C. at 3000×g for 10 min. All samples were maintained chilled throughout processing. Plasma samples (approximately 300 μL) were collected in polypropylene tubes and stored at −80° C. until bioanalysis. Flash frozen jejunum and tumor samples were also stored −80° C. until bioanalysis.

TABLE 6

| | Liver stability | Gut Stability | Plasma Stability* | P493B cells | | |
|---|---|---|---|---|---|---|
| | (% remaining | (% remaining | (% remaining | (DON release; | | Solubility |
| Compound | at 60 min) | at 60 min) | at 60 min) | nmol/g) | Efflux | (mg/mL) |
| 13′ | 4 ± 0 | 11 ± 1 | 94 ± 1 (ces1KO) | 10.13 | 23 | 0.24 |
| | | | 0 ± 0 (MP) | | | |
| | | | 96 ± 3 (HP) | | | |
| 5a | 23 ± 0 | 76 ± 0 | 105 ± 1 (PP) | 5.26 | | |
| | | | 89 ± 2 (ces1KO) | | | |
| | | | 0 ± 0 (RP) | | | |
| 5b | 12 ± 1 | | | 10.28 | | ~5 |
| 5c | 0 ± 0 | | | 4.88 | | ~2 |
| 5d | 76 ± 0 | 98 ± 2 | | 0.85 | 2 | |
| 5e | 0 ± 0 | | | | | |
| 5f | 0 ± 0 | | | 0.34 | | ~5 |
| 5g | 2 ± 0 | 93 ± 1 | | 0.31 | 1 | |
| 5h | 93 ± 1 | 100 ± 1 | | Undetectable | 2 | |
| 5i | 97 ± 0 | 110 ± 2 | | 0.36 | 1 | |
| 5j | 78 ± 1 | 104 ± 0 | | 0.0 | | ~4 |
| 5k | 83 ± 2 | | | 6.99 | 1 | |
| 5l | 12 ± 0 | | | | | |
| 5m | 57 ± 0 | 87 ± 2 | | 2.58 | 5 | |
| 8a | 5 ± 1 | 12 ± 1 | | 42 | | |
| 8b | 1 ± 0 | 6 ± 1 | | 32 | | |
| 8c | 13 ± 0 | 13 ± 7 | | 27 | | |
| 8d | 2 ± 0 | 9 ± 1 | | | | |
| 8e | 7 ± 0 | 9 ± 0 | | 14 | | |
| 8f | 2 ± 0 | 2 ± 0 | | 55 | | |
| 8g | 1 ± 0 | 1 ± 0 | | | | |
| 8h | | | | | | |
| 8i | 0 ± 0 | 0 ± 0 | | | | |
| 8j | 2 ± 0 | | | 1.64 | | |
| 8k | 1 ± 0 | 3 ± 0 | | | | |
| 8l | 0 ± 0 | 2 ± 0 | | | | |
| 8m | 0 ± 0 | | | | | |
| 8n | 0 ± 0 | | 69 ± 3 (ces1KO) | 148.4 | 49 | |
| 11a | 95 ± 3 | 88 ± 4 | 62 ± 1 (MP) | 1.23 | 41 | |
| 11b | 80 ± 3 | 79 ± 4 | 68 ± 2 (MP) | 29.84 | 33 | 0.17 |
| | | | 101 ± 0 (HP) | | | |
| | | | 3 ± 0 (RP) | | | |
| 11c | 80 ± 2 | 95 ± 1 | | 4.99 | 106 | |
| 11d | 92 ± 2 | 93 ± 2 | | 0.26 | 2 | 4.3 |
| 12a | 75 ± 1 | 104 ± 2 | | 53.99 | 80 | |
| 12b | 98 ± 2 | 90 ± 2 | 92 ± 1 (MP) | 8.02 | 3.48 | 8.8 |
| 12c | 89 ± 0 | | | 3.13 | 5.37 | |
| 12d | 89 ± 1 | | 88 ± 2 (Ces1KO) | 17.42 | 65.59 | |
| | | | 10 ± 2 (MP) | | | |
| 12g | 85 ± 1 | 93 ± 3 | | 0.01 | 1 | |
| 12h | 50 ± 3 | | | 2.07 | | |
| 12k | 89 ± 1 | | | 0.34 | | |
| 13a | 95 ± 1 | 99 ± 2 | | 29.96 | 32 | |
| 14a | 88 ± 1 | 96 ± 0 | | 0.53 | | |
| 14b | 90 ± 0 | 99 ± 2 | 100 ± 5 (HP) | 10.95 | 33 | ~10 |
| | | | 73 ± 2 (MP) | | | |
| 14c | 100 ± 1 | 96 ± 1 | | 0.66 | 15 | |
| 14d | 94 ± 1 | 96 ± 1 | | 0.0 | 1.48 | |
| 15a | 81 ± 2 | 90 ± 1 | 93 ± 1 (ces1KO MP) | 50.2 | 48 | |
| | | | 67 ± 1 (MP) | | | |
| 15b | 82 ± 2 | | | 1.37 | 7.31 | |
| 15c | 88 ± 2 | | | 5.53 | | |
| 16a | 87 ± 0 | 87 ± 1 | | 51.9 | 45 | |
| 17a | 103 ± 3 | 96 ± 1 | | 0.3 | | |
| 17b | 91 ± 1 | 98 ± 1 | 96 ± 3 (HP) | 10.12 | 57 | ~10 |
| | | | 82 ± 6 (MP) | | | |
| | | | 33 ± 2 (RP) | | | |
| 18a | 105 ± 2 | 93 ± 1 | | 0.0 | | |
| 18b | 100 ± 3 | 98 ± 2 | | 1.96 | | |
| 19a | 82 ± 0 | | | 45.4 | 24.37 | |
| 20a | 69 ± 1 | | 60 ± 7 (MP) | 51.13 | 61.35 | |
| | | | 102 ± 2 (ces1KO) | | | |
| 21a | 75 ± 4 | | | 51.92 | 46.71 | |
| 22a | 62 ± 1 | | | 76.9 | 52.07 | |
| 23a | 72 ± 0 | | 79 ± 2 (MP) | 76.59 | 32.76 | |
| | | | 70 ± 14 (Ces1KO) | | | |
| 24a | 78 ± 1 | | | 48.09 | — | |
| 25a | 99 ± 1 | 92 ± 4 | | 0.03 | 16 | |
| 26a | 71 ± 0 | | | 0 | | |
| 28a | 0 ± 0 | | | 9.2 | 21 | |
| 28b | 2 ± 0 | | | 2.23 | 10 | |
| 28c | 1 ± 0 | | | 2.06 | 11 | |
| 30a | | | | 2.89 | | |
| 30b | | | 42 ± 1 (HP) | 2.05 | | |
| 31a | 58 ± 2 | 79 ± 7 | 100 ± 2 (HP) | 0.86 | 1 | |
| | | | 6 ± 1 (MP | | | |
| 31b | 103 ± 4 | 90 ± 2 | | 0.6 | 1 | |
| 31c | 89 ± 4 | | | 0 | | |
| 31d | 90 ± 1 | | | 0.47 | | |
| 31e | 39 ± 0 | | | 1.14 | | |
| 31f | 49 ± 0 | 89 ± 2 | | 1.65 | | |

*MP = Mouse Plasma; HP = Human Plasma; RP = Rat Plasma; CES1KO = Mouse Plasma from CES1KO mice; PP = Pig Plasma

It is to be understood that the foregoing described embodiments and exemplifications are not intended to be limiting in any respect to the scope of the disclosure, and that the claims presented herein are intended to encompass all embodiments and exemplifications whether or not explicitly presented herein

All patents and publications cited herein are fully incorporated by reference in their entirety.

What is claimed is:

1. A compound of Formula I: 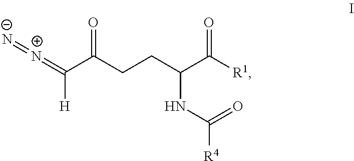
or a pharmaceutically acceptable salt thereof, wherein:
R¹ is selected from the group consisting of —OR² and —NR^{3a}R^{3b};
R² is selected from the group consisting of hydrogen, C₁-C₆ alkyl, C₁-C₆ haloalkyl C₁-C₆ alkenyl, C₃-C₂₀ heteroalkyl, and C₃-C₆ cycloalkyl;
R^{3a} and R^{3b} are independently selected from the group consisting of hydrogen and C₁-C₄ alkyl; or
R^{3a} and R^{3b} taken together with the nitrogen atom to which they are attached from a 4- to 8-membered heterocyclo;
R⁴ is selected from the group consisting of C₁-C₆ alkyl, C₁-C₆ haloalkyl, (amino)C₁-C₆ alkyl, (amino)(aryl)C₁-C₆ alkyl, optionally substituted heteroaryl, —CH(R^{5a})N(R^{6a})C(═O)R^{7a}, —(CH₂)ₘ—N(R^{6c})C(═O)R^{7c}, and —OR⁸;
R^{5a} is selected from the group consisting of hydrogen, optionally substituted C₁-C₆ alkyl, optionally substituted aryl, (heterocyclo)C₁-C₄ alkyl, (aryl)C₁-C₄ alkyl, and (heteroaryl)C₁-C₄ alkyl;
R^{6a} is selected from the group consisting of hydrogen and C₁-C₄ alkyl;
R^{7a} is selected from the group consisting of C₁-C₆ alkyl, C₁-C₆ haloalkyl, optionally substituted C₃-C₈ cycloalkyl, optionally substituted 4- to 10-membered heterocyclo, optionally substituted heteroaryl, (amino)C₁-C₄ alkyl, (heterocyclo)C₁-C₄ alkyl, and —CH(R^{5b})N(R^{6b})C(═O)R^{7b},
R⁸ is optionally substituted 4- to 10-membered heterocyclo;
R^{5b} is selected from the group consisting of hydrogen, optionally substituted C₁-C₆ alkyl, (aryl)C₁-C₄ alkyl, and (heteroaryl)C₁-C₄ alkyl,
R^{6b} is selected from the group consisting of hydrogen and C₁-C₄ alkyl;
R^{7b} is selected from the group consisting of C₁-C₆ alkyl, C₁-C₆ haloalkyl, optionally substituted C₃-C₈ cycloalkyl, optionally substituted 4- to 10-membered heterocyclo, optionally substituted heteroaryl, (amino)C₁-C₄ alkyl, and (heterocyclo)C₁-C₄ alkyl;
R^{6c} is selected from the group consisting of hydrogen and C₁-C₄ alkyl;
R^{7c} is selected from the group consisting of C₁-C₆ alkyl, C₁-C₆ haloalkyl, optionally substituted C₃-C₈ cycloalkyl, optionally substituted 4- to 10-membered heterocyclo, optionally substituted heteroaryl, (amino)C₁-C₄ alkyl, and (heterocyclo)C₁-C₄ alkyl; and
m is 2, 3, 4, or 5,
with the proviso that the compound of Formula I is not a compound of Table 1.

2. The compound of claim 1 of Formula II: 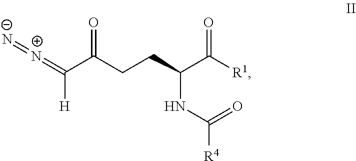
or a pharmaceutically acceptable salt thereof.

3. The compound of claim 1 of Formula III: 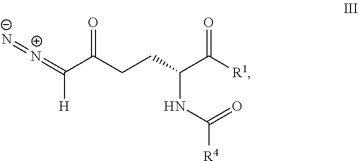
or a pharmaceutically acceptable salt thereof.

4. The compound of any one of claims 1-3, or a pharmaceutically acceptable salt thereof, wherein R¹ is —OR².

5. The compound of claim 4, or a pharmaceutically acceptable salt thereof, wherein R² is selected from the group consisting of hydrogen, —CH₃, —CD₃, —CH₂CH₃, —CH(CH₃)₂, —C(CH₃)₃, —CH(CH₂F)₂, —CH₂CH═CH₂, and —OCH₂CH₂OCH₂CH₂OCH₃.

6. The compound of any one of claims 1-3, or a pharmaceutically acceptable salt thereof, wherein R¹ is —NR^{3a}R^{3b}.

7. The compound of claim 6, or a pharmaceutically acceptable salt thereof, wherein R^{3a} and R^{3b} are independently selected from the group consisting of hydrogen and methyl.

8. The compound of any one of claims 1-7, or a pharmaceutically acceptable salt thereof, wherein R⁴ is C₁-C₆ alkyl.

9. The compound of claim 8, or a pharmaceutically acceptable salt thereof, wherein R⁴ is selected from the group consisting of —CH₃, —CH₂CH₃, and —(CH₂)₄CH₃.

10. The compound of any one of claims 1-7, or a pharmaceutically acceptable salt thereof, wherein R⁴ is C₁-C₆ haloalkyl.

11. The compound of claim 10, or a pharmaceutically acceptable salt thereof, wherein R⁴ is —CHCl₂.

12. The compound of any one of claims 1-7, or a pharmaceutically acceptable salt thereof, wherein R⁴ is (amino)C₁-C₆ alkyl.

13. The compound of claim 12, or a pharmaceutically acceptable salt thereof, wherein R⁴ is —CH₂N(CH₃)₂.

14. The compound of any one of claims 1-7, or a pharmaceutically acceptable salt thereof, wherein R⁴ is optionally substituted heteroaryl.

15. The compound of claim 14, or a pharmaceutically acceptable salt thereof, wherein R⁴ is 2-pyridyl, 3-pyridyl, or 4-pyridyl.

16. The compound of any one of claims 1-7, or a pharmaceutically acceptable salt thereof, wherein R⁴ is —OR⁸.

17. The compound of claim 16, or a pharmaceutically acceptable salt thereof, wherein R⁸ is:

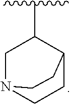 [SMILES: *C1CN2CCC1CC2]

18. The compound of any one of claims 1-7, or a pharmaceutically acceptable salt thereof, wherein R⁴ is —(CH₂)ₘ—N(R^{6c})C(═O)R^{7c}.

19. The compound of claim 18, or a pharmaceutically acceptable salt thereof, wherein m is 3.

20. The compound of claims 18 or 19, or a pharmaceutically acceptable salt thereof, wherein R^{6c} is hydrogen.

21. The compound of any one of claims 18-20, or a pharmaceutically acceptable salt thereof, wherein R^{7c} is 4- to 10-membered heterocyclo.

22. The compound of claim 21, or a pharmaceutically acceptable salt thereof, wherein R^{7c} is:

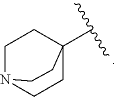 [SMILES: *C12CCN(CC1)CC2]

23. The compound of any one of claims 1-7, or a pharmaceutically acceptable salt thereof, wherein R⁴ is —CH(R^{5a})N(R^{6a})C(═O)R^{7a}.

24. The compound of claim 23, or a pharmaceutically acceptable salt thereof, wherein R⁴ is:

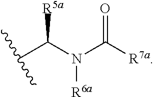

25. The compound of claim 23, or a pharmaceutically acceptable salt thereof, wherein R⁴ is:

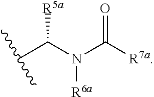

26. The compound of any one of claims 23-25, or a pharmaceutically acceptable salt thereof, wherein R^{6a} is hydrogen.

27. The compound of claims 23 or 26, or a pharmaceutically acceptable salt thereof, wherein R^{5a} is hydrogen.

28. The compound of any one of claims 23-26, or a pharmaceutically acceptable salt thereof, wherein R^{5a} is optionally substituted C₁-C₆ alkyl.

29. The compound of claim 28, or a pharmaceutically acceptable salt thereof, wherein R^{5a} is selected from the group consisting of —CH₃, —CH₂CH(CH₃)₂, and —CH₂C(CH₃)₃.

30. The compound of any one of claims 23-26, or a pharmaceutically acceptable salt thereof, wherein R^{5a} is optionally substituted aryl.

31. The compound of claim 30, or a pharmaceutically acceptable salt thereof, wherein R^{5a} is optionally substituted phenyl.

32. The compound of any one of claims 23-26, or a pharmaceutically acceptable salt thereof, wherein R^{5a} is (heterocyclo)C₁-C₄ alkyl.

33. The compound of any one of claims 23-26, or a pharmaceutically acceptable salt thereof, wherein R^{5a} is (aryl)C₁-C₄ alkyl.

34. The compound of claim 33, wherein R^{5a} is selected from the group consisting of:

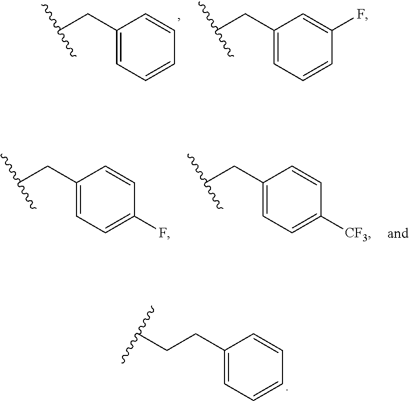

35. The compound of any one of claims 23-26, or a pharmaceutically acceptable salt thereof, wherein R^{5a} is (heteroaryl)C₁-C₄ alkyl.

36. The compound of claim 35, wherein R^{5a} is selected from the group consisting of:

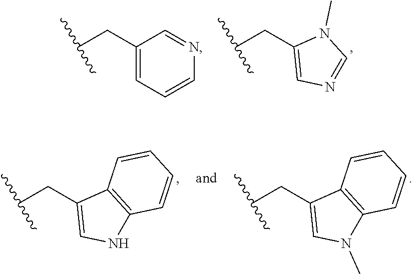

37. The compound of any one of claims 23-36, or a pharmaceutically acceptable salt thereof, wherein R^{7a} is C₁-C₄ alkyl.

38. The compound of claim 37, or a pharmaceutically acceptable salt thereof, wherein R^{7a} is selected from the group consisting of —CH₃, —CH(CH₃)₂, —C(CH₃)₃, —CH₂CH₂CH₃, and —CH₂CH(CH₃)₂.

39. The compound of any one of claims 23-36, or a pharmaceutically acceptable salt thereof, wherein R^{7a} is C₁-C₄ haloalkyl.

40. The compound of claim 39, or a pharmaceutically acceptable salt thereof, wherein R^{7a} is —CHCl₂.

41. The compound of any one of claims 23-36, or a pharmaceutically acceptable salt thereof, wherein R^{7a} is C₃-C₈ cycloalkyl.

42. The compound of claim 41, or a pharmaceutically acceptable salt thereof, wherein R^{7a} is cyclopropyl.

43. The compound of any one of claims 23-36, or a pharmaceutically acceptable salt thereof, wherein R^{7a} is optionally substituted 4- to 10-membered heterocyclo.

44. The compound of claim 43, or a pharmaceutically acceptable salt thereof, wherein R^{7a} is selected from the group consisting of:

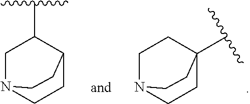 [SMILES: *C1CN2CCC1CC2 ; *C12CCN(CC1)CC2]

45. The compound of any one of claims 23-36, or a pharmaceutically acceptable salt thereof, wherein R^{7a} is (amino)C₁-C₄ alkyl.

46. The compound of claim 45, or a pharmaceutically acceptable salt thereof, wherein R^{7a} is selected from the group consisting of —CH₂N(CH₃)₂ and —CH₂N(CH₂CH₃)₂.

47. The compound of any one of claims 23-36, or a pharmaceutically acceptable salt thereof, wherein R^{7a} is (heterocyclo)C₁-C₄ alkyl.

48. The compound of claim 47, or a pharmaceutically acceptable salt thereof, wherein R^{7a} is selected from the group consisting of

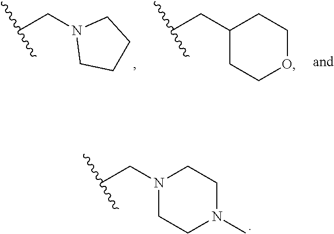

49. The compound of any one of claims 23-36, or a pharmaceutically acceptable salt thereof, wherein R^{7a} is —CH(R^{5b})N(R^{6b})C(═O)R^{7b}.

50. The compound of claim 49, or a pharmaceutically acceptable salt thereof, wherein R^{7a} is —CH₂N(H)C(═O)CH₂N(CH₃)₂.

51. The compound of claim 1, or pharmaceutically acceptable salt thereof, selected from the group consisting of the compounds of Table 2.

52. A pharmaceutical composition comprising the compound of any one of claims 1-51, or a pharmaceutically acceptable salt thereof, and a pharmaceutically acceptable carrier.

53. The pharmaceutical composition of claim 52, wherein the pharmaceutically acceptable carrier comprises water.

54. A method of treating cancer in a subject in need thereof, the method comprising administering a therapeutically effective amount of the compound of any one of claims 1-51, or a pharmaceutically acceptable salt thereof, to the subject.

55. The method of claim 54 further comprising administering an optional therapeutic agent to the subject.

56. The method of claim 55, wherein the optional therapeutic agent is an immune checkpoint inhibitor.

57. The method of claim 56, wherein the immune checkpoint inhibitor is selected from the group consisting of a PD-1 inhibitor, a PD-L1 inhibitor, a CTLA-4 inhibitor, a LAG3 inhibitor, a TIM3 inhibitor, and a cd47 inhibitor.

58. The method of any one of claims 54-57, wherein the cancer is one or more of the cancers of Table 3.

59. The method of any one of claims 54-58, wherein the cancer is a solid tumor.

60. The method of any one of claims 54-58, wherein the cancer is a hematological cancer.

61. The method of claim 60, wherein the hematological cancer is acute lymphocytic leukemia, chronic lymphocytic leukemia (including B-cell chronic lymphocytic leukemia), or acute myeloid leukemia.

62. The method of any one of claims 54-58, wherein the cancer is squamous cell carcinoma of the head and neck, adenocarcinoma squamous cell carcinoma of the esophagus, adenocarcinoma of the stomach, adenocarcinoma of the colon, hepatocellular carcinoma, cholangiocarcinoma of the biliary system, adenocarcinoma of gall bladder, adenocarcinoma of the pancreas, ductal carcinoma in situ of the breast, adenocarcinoma of the breast, adenocarcinoma of the lungs, squamous cell carcinoma of the lungs, transitional cell carcinoma of the bladder, squamous cell carcinoma of the bladder, squamous cell carcinoma of the cervix, adenocarcinoma of the cervix, endometrial carcinoma, penile squamous cell carcinoma, or squamous cell carcinoma of the skin.

63. The method of any one of claims 54-58, wherein the cancer is hepatocellular carcinoma, glioblastoma, lung cancer, breast cancer, head and neck cancer, prostate cancer, melanoma, or colorectal cancer.

64. The method of any one of claims 54-58, wherein the cancer is colorectal cancer, breast cancer, lymphoma, melanoma, kidney cancer, or lung cancer.

65. A kit comprising the compound of any one of claims 1-51 or a pharmaceutically acceptable salt thereof, and instructions for administering the compound or a pharmaceutically acceptable salt thereof, to a subject having a disease, disorder, or condition.
